SEQUENCE LISTING (71 sequences)

SEQ ID NO: 1 - protein, 159 aa, Escherichia coli [sequence omitted]

SEQ ID NO: 2 - protein, 187 aa, Homo sapiens
MVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 3 - protein, 107 aa, Homo sapiens
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 4 - protein, 327 aa, Homo sapiens
MEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQ

SEQ ID NO: 5 - protein, 260 aa, Homo sapiens
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 6 - protein, 595 aa, Homo sapiens [sequence omitted]

SEQ ID NO: 7 - DNA, 1918 bp, Artificial Sequence
  Description of Artificial Sequence Synthetic polynucleotide
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatcactagtgccaccatggcacctaagaaaaagaggaaggttgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagactgggggccttgcttggcaacagcacagacccagctgtgttcacagacctggcatccgtggacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccggatcctga

SEQ ID NO: 8 - protein, 236 aa, Artificial Sequence
  Description of Artificial Sequence Synthetic polypeptide
MAPKKKRKVERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTRLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGS

SEQ ID NO: 9 - DNA, 2404 bp, Artificial Sequence
  Description of Artificial Sequence Synthetic polynucleotide
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatcactagtgccaccatggcacctaagaaaaagaggaaggttgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagactgggggccttgcttggcaacagcacagacccagctgtgttcacagacctggcatccgtggacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccggatccagcggcatctctctgattgcggcgctggcagttgactacgttattggcatggaaaacgcgatgccatggaacctcccggctgacctggcgtggttcaaacgtaacaccctgaacaaacctgtgatcatgggtcgtcacacctgggaatctattggccgtcctctcccgggtcgtaaaaacatcattctgtcttctcagccaggcaccgacgaccgtgttacctgggttaaaagcgttgacgaagcgattgctgcgtgcggtgatgttcctgaaattatggtgatcggcggtggccgtgttatcgaacagttcctgccgaaagcgcagaaactgtacctgacccacatcgacgcggaagttgaaggtgacacccacttcccggactacgaaccggatgattgggagagcgtattctccgaattccatgatgcggatgcgcaaaactctcattcttactgttttgaaatcctggaacgtcgtggatcctga

SEQ ID NO: 10 - protein, 398 aa, Artificial Sequence
  Description of Artificial Sequence Synthetic polypeptide
MAPKKKRKVERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTRLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSSGISLIAALAVDYVIGMENAMPWNLPADLAWFKRNTLNKPVIMGRHTWESIGRPLPGRKNIILSSQPGTDDRVTWVKSVDEAIAACGDVPEIMVIGGGRVIEQFLPKAQKLYLTHIDAEVEGDTHFPDYEPDDWESVFSEFHDADAQNSHSYCFEILERRGS

SEQ ID NO: 11 - DNA, 943 bp, Artificial Sequence
  Description of Artificial Sequence Synthetic polynucleotide
taatgatgggcgcacgagtaatgatgggcggacgactaatgatgggcgcacgagtaatgatgggcgtctagctaatgatgggcgctagagtaatgatgggcggtagactaatgatgggcgctccagtaatgatgggcgttctagctctagagggtatataatgggggccactagtctactaccagatagcttggtaccgagctctgatccagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatcctaa

SEQ ID NO: 12 - protein, 241 aa, Artificial Sequence
  Description of Artificial Sequence Synthetic polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGS

SEQ ID NO: 13 - DNA, 2404 bp, Artificial Sequence
  Description of Artificial Sequence Synthetic polynucleotide
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatcactagtgccaccatggcacctaagaaaaagaggaaggttgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacggatccagcggcatctctctgattgcggcgctggcagttgactacgttattggcatggaaaacgcgatgccatggaacctcccggctgacctggcgtggttcaaacgtaacaccctgaacaaacctgtgatcatgggtcgtcacacctgggaatctattggccgtcctctcccgggtcgtaaaaacatcattctgtcttctcagccaggcaccgacgaccgtgttacctgggttaaaagcgttgacgaagcgattgctgcgtgcggtgatgttcctgaaattatggtgatcggcggtggccgtgttatcgaacagttcctgccgaaagcgcagaaactgtacctgacccacatcgacgcggaagttgaaggtgacacccacttcccggactacgaaccggatgattgggagagcgtattctccgaattccatgatgcggatgcgcaaaactctcattcttactgttttgaaatcctggaacgtcgtggatccctgggggccttgcttggcaacagcacagacccagctgtgttcacagacctggcatccgtggacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccgctagctga

SEQ ID NO: 14 - protein, 398 aa, Artificial Sequence
  Description of Artificial Sequence Synthetic polypeptide
MAPKKKRKVERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINGSSGISLIAALAVDYVIGMENAMPWNLPADLAWFKRNTLNKPVIMGRHTWESIGRPLPGRKNIILSSQPGTDDRVTWVKSVDEAIAACGDVPEIMVIGGGRVIEQFLPKAQKLYLTHIDAEVEGDTHFPDYEPDDWESVFSEFHDADAQNSHSYCFEILERRGSLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSAS

SEQ ID NO: 15 - DNA, 2416 bp, Artificial Sequence
  Description of Artificial Sequence Synthetic polynucleotide
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatcactagtgccaccatggcacctaagaaaaagaggaaggttgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagactgggggccttgcttggcaacagcacagacccagctgtgttcacagacctggcatccgtggacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccggatccggctcaggtagcagcggcatctctctgattgcggcgctggcagttgactacgttattggcatggaaaacgcgatgccatggaacctcccggctgacctggcgtggttcaaacgtaacaccctgaacaaacctgtgatcatgggtcgtcacacctgggaatctattggccgtcctctcccgggtcgtaaaaacatcattctgtcttctcagccaggcaccgacgaccgtgttacctgggttaaaagcgttgacgaagcgattgctgcgtgcggtgatgttcctgaaattatggtgatcggcggtggccgtgttatcgaacagttcctgccgaaagcgcagaaactgtacctgacccacatcgacgcggaagttgaaggtgacacccacttcccggactacgaaccggatgattgggagagcgtattctccgaattccatgatgcggatgcgcaaaactctcattcttactgttttgaaatcctggaacgtcgtggatcctga

SEQ ID NO: 16 - protein, 402 aa, Artificial Sequence
  Description of Artificial Sequence Synthetic polypeptide
MAPKKKRKVERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTRLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSSGISLIAALAVDYVIGMENAMPWNLPADLAWFKRNTLNKPVIMGRHTWESIGRPLPGRKNIILSSQPGTDDRVTWVKSVDEAIAACGDVPEIMVIGGGRVIEQFLPKAQKLYLTHIDAEVEGDTHFPDYEPDDWESVFSEFHDADAQNSHSYCFEILERRGS

SEQ ID NO: 17 - DNA, 3364 bp, Artificial Sequence
  Description of Artificial Sequence Synthetic polynucleotide
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatcactagtgccaccatggcacctaagaaaaagaggaaggttgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagactgggggccttgcttggcaacagcacagacccagctgtgttcacagacctggcatccgtggacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccggatccagcggcatctctctgattgcggcgctggcagttgactacgttattggcatggaaaacgcgatgccatggaacctcccggctgacctggcgtggttcaaacgtaacaccctgaacaaacctgtgatcatgggtcgtcacacctgggaatctattggccgtcctctcccgggtcgtaaaaacatcattctgtcttctcagccaggcaccgacgaccgtgttacctgggttaaaagcgttgacgaagcgattgctgcgtgcggtgatgttcctgaaattatggtgatcggcggtggccgtgttatcgaacagttcctgccgaaagcgcagaaactgtacctgacccacatcgacgcggaagttgaaggtgacacccacttcccggactacgaaccggatgattgggagagcgtattctccgaattccatgatgcggatgcgcaaaactctcattcttactgttttgaaatcctggaacgtcgtggatcctgaatcgggctagcacgcgtttaggatcccttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggctggatcagagctcggtaccaagctatctggtagtagactagtggcccccattatataccctctagagctagaacgcccatcattactggagcgcccatcattagtctaccgcccatcattactctagcgcccatcattagctagacgcccatcattactcgtgcgcccatcattagtcgtccgcccatcattactcgtgcgcccatcatta

SEQ ID NO: 18 - protein, 398 aa, Artificial Sequence; identical to SEQ ID NO: 10
  Description of Artificial Sequence Synthetic polypeptide

SEQ ID NO: 19 - protein, 241 aa, Artificial Sequence; identical to SEQ ID NO: 12
  Description of Artificial Sequence Synthetic polypeptide

SEQ ID NO: 20 - DNA, 1818 bp, Artificial Sequence
  Description of Artificial Sequence Synthetic polynucleotide
taatgatgggcgcacgagtaatgatgggcggacgactaatgatgggcgcacgagtaatgatgggcgtctagctaatgatgggcgctagagtaatgatgggcggtagactaatgatgggcgctccagtaatgatgggcgttctagctctagagggtatataatgggggccactagttctagaggatcaccatgtgccaccaacagctcgtgatcagctggttctccctggtgttcctggctagccccctggtggccatctgggagctcaagaaagacgtgtacgtggtggaactggactggtaccccgacgcccctggcgaaatggtggtgctgacatgcgacacccctgaggaggatggcattacctggaccctcgatcagagctccgaggtgctgggcagcggcaaaaccctgaccatccaggtgaaggagtttggcgatgccggccagtacacatgtcacaagggcggcgaggtgctgagccactccctgctgctgctccacaagaaggaagatggcatctggagcaccgacattctgaaggatcagaaggagcccaagaacaagacattcctgaggtgtgaggccaagaactacagcggcaggtttacctgctggtggctgacaacaatcagcaccgacctcacattctccgtcaagtcctccaggggttcttccgaccctcaaggcgtgacatgcggcgctgccaccctgagcgctgagagagtcaggggcgacaacaaggagtacgagtacagcgtcgaatgtcaggaggacagcgcctgtcccgccgctgaagagagcctgcctatcgaggtgatggtggacgccgtgcacaaactgaagtatgagaattacacctccagcttcttcatcagggacatcatcaaacccgatccccccaagaacctgcagctgaagcctctgaagaacagcagacaggtcgaagtgtcctgggagtaccctgatacctggtccacaccccacagctacttcagcctgaccttttgcgtgcaggtgcagggcaagagcaaaagggagaagaaggacagggtgtttaccgacaagacctccgccacagtgatttgcagaaagaacgcctccatcagcgtgagggcccaggacaggtattacagcagctcctggagcgaatgggctagcgtgccctgtagcggaggaggaggcagcggaggaggaggttctggaggaggcggcagcagaaacctgcctgtcgctacccccgaccccggaatgttcccctgcctgcaccactcccagaacctcctgagggccgtgtccaacatgctgcagaaggctagacagaccctcgaattctacccctgtaccagcgaggagatcgaccatgaggacatcaccaaggataagaccagcaccgtggaggcttgcctgcctctggagctgaccaaaaacgagagctgcctgaacagcagggaaaccagcttcattaccaacggctcctgcctggcctccaggaagacatccttcatgatggccctgtgcctcagcagcatctacgaggacctgaagatgtatcaggtggagtttaagaccatgaatgccaagctgctgatggaccctaagaggcagatcttcctggaccagaatatgctggccgtgattgacgagctgatgcaggccctcaactttaacagcgagaccgtgccccagaaaagcagcctcgaagagcctgacttctacaaaaccaagattaagctgtgtatcctgctgcacgccttcaggatcagggccgtgaccatcgacagggtgatgagctacctgaacgccagcggatcctaa

SEQ ID NO: 21 - protein, 542 aa, Artificial Sequence
  Description of Artificial Sequence Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGS

SEQ ID NO: 22 - DNA, 1107 bp, Artificial Sequence
  Description of Artificial Sequence Synthetic polynucleotide
cagacatgataagatacattgatgagtttggacaaaccacaactagaatgcagtgaaaaaaatgctttatttgtgaaatttgtgatgctattgctttatttgtaaccattataagctgcaataaacaagttcctctcactctctgatattcatttctttgcaagttaggatcccttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggctggatcagagctcggtaccaagctatctggtagtagactagtggcccccattatataccctctagagctagaacgcccatcattactggagcgcccatcattagtctaccgcccatcattactctagcgcccatcattagctagacgcccatcattactcgtgcgcccatcattagtcgtccgcccatcattactcgtgcgcccatcatta

SEQ ID NO: 23 - protein, 241 aa, Artificial Sequence; identical to SEQ ID NO: 12
  Description of Artificial Sequence Synthetic polypeptide

SEQ ID NO: 24 - DNA, 3360 bp, Artificial Sequence
  Description of Artificial Sequence Synthetic polynucleotide
taatgatgggcgcacgagtaatgatgggcggacgactaatgatgggcgcacgagtaatgatgggcgtctagctaatgatgggcgctagagtaatgatgggcggtagactaatgatgggcgctccagtaatgatgggcgttctagctctagagggtatataatgggggccactagtctactaccagatagcttggtaccgagctctgatccagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatcctaaatcgggctagccacgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatcactagtgccaccatggcacctaagaaaaagaggaaggttgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagactgggggccttgcttggcaacagcacagacccagctgtgttcacagacctggcatccgtggacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccggatccagcggcatctctctgattgcggcgctggcagttgactacgttattggcatggaaaacgcgatgccatggaacctcccggctgacctggcgtggttcaaacgtaacaccctgaacaaacctgtgatcatgggtcgtcacacctgggaatctattggccgtcctctcccgggtcgtaaaaacatcattctgtcttctcagccaggcaccgacgaccgtgttacctgggttaaaagcgttgacgaagcgattgctgcgtgcggtgatgttcctgaaattatggtgatcggcggtggccgtgttatcgaacagttcctgccgaaagcgcagaaactgtacctgacccacatcgacgcggaagttgaaggtgacacccacttcccggactacgaaccggatgattgggagagcgtattctccgaattccatgatgcggatgcgcaaaactctcattcttactgttttgaaatcctggaacgtcgtggatcctga

SEQ ID NO: 25 - protein, 241 aa, Artificial Sequence; identical to SEQ ID NO: 12
  Description of Artificial Sequence Synthetic polypeptide

SEQ ID NO: 26 - protein, 398 aa, Artificial Sequence; identical to SEQ ID NO: 10
  Description of Artificial Sequence Synthetic polypeptide

SEQ ID NO: 27 - DNA, 2701 bp, Artificial Sequence
  Description of Artificial Sequence Synthetic polynucleotide
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatcactagtgccaccatggcacctaagaaaaagaggaaggttgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagactgggggccttgcttggcaacagcacagacccagctgtgttcacagacctggcatccgtggacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggatcctga

SEQ ID NO: 28 - protein, 497 aa, Artificial Sequence
  Description of Artificial Sequence Synthetic polypeptide
MAPKKKRKVERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTRLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGS

SEQ ID NO: 29 - DNA, 943 bp, Artificial Sequence; identical to SEQ ID NO: 11
  Description of Artificial Sequence Synthetic polynucleotide

SEQ ID NO: 30 - protein, 241 aa, Artificial Sequence; identical to SEQ ID NO: 12
  Description of Artificial Sequence Synthetic polypeptide

SEQ ID NO: 31 - DNA, 3811 bp, Artificial Sequence
  Description of Artificial Sequence Synthetic polynucleotide
cagacatgataagatacattgatgagtttggacaaaccacaactagaatgcagtgaaaaaaatgctttatttgtgaaatttgtgatgctattgctttatttgtaaccattataagctgcaataaacaagttcctctcactctctgatattcatttctttgcaagttaggatcccttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggctggatcagagctcggtaccaagctatctggtagtagactagtggcccccattatataccctctagagctagaacgcccatcattactggagcgcccatcattagtctaccgcccatcattactctagcgcccatcattagctagacgcccatcattactcgtgcgcccatcattagtcgtccgcccatcattactcgtgcgcccatcattagcacgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatcactagtgccaccatggcacctaagaaaaagaggaaggttgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagactgggggccttgcttggcaacagcacagacccagctgtgttcacagacctggcatccgtggacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggatcctga

SEQ ID NO: 32 - protein, 241 aa, Artificial Sequence; identical to SEQ ID NO: 12
  Description of Artificial Sequence Synthetic polypeptide

SEQ ID NO: 33 - protein, 497 aa, Artificial Sequence; identical to SEQ ID NO: 28
  Description of Artificial Sequence Synthetic polypeptide

SEQ ID NO: 34 - DNA, 2701 bp, Artificial Sequence; identical to SEQ ID NO: 27
  Description of Artificial Sequence Synthetic polynucleotide

SEQ ID NO: 35 - protein, 497 aa, Artificial Sequence; identical to SEQ ID NO: 28
  Description of Artificial Sequence Synthetic polypeptide

SEQ ID NO: 36 - DNA, 943 bp, Artificial Sequence
  Description of Artificial Sequence Synthetic polynucleotide
ttaggatcccttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggctggatcagagctcggtaccaagctatctggtagtagactagtggcccccattatataccctctagagctagaacgcccatcattactggagcgcccatcattagtctaccgcccatcattactctagcgcccatcattagctagacgcccatcattactcgtgcgcccatcattagtcgtccgcccatcattactcgtgcgcccatcatta

SEQ ID NO: 37 - protein, 241 aa, Artificial Sequence; identical to SEQ ID NO: 12
  Description of Artificial Sequence Synthetic polypeptide

SEQ ID NO: 38 - DNA, 720 bp, Artificial Sequence
  Description of Artificial Sequence Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 39 - protein, 239 aa, Artificial Sequence
  Description of Artificial Sequence Synthetic polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 40 - DNA, 2568 bp, Artificial Sequence
  Description of Artificial Sequence Synthetic polynucleotide
atgtctcaccactggggctacggcaagcacaatggacctgagcactggcacaaggacttccctatcgccaagggcgagagacagagccccgtggacatcgatacccacaccgccaaatacgaccccagcctgaagcctctgagcgtgtcctatgatcaggccaccagcctgcgcatcctgaacaatggccacgccttcaacgtggaattcgacgacagccaggacaaggccgtgctgaaaggtggacctctggacggcacctaccggctgatccagttccactttcactggggcagcctggatggccagggatctgaacacaccgtggacaagaagaagtacgccgccgaactgcacctggtgcactggaacaccaaatacggcgacttcggcaaagccgtgcagcagcctgatggactggctgtgctgggcatcttcctgaaagtgggctctgccaagcctggcctgcagaaagtggtggacgtgctggacagcatcaagaccaagggcaagagcgccgacttcaccaacttcgaccctagaggactgctgcccgagagcctggactactggacataccctggcagcctgaccacacctcctctgctggaatgtgtgacctggatcgtcctgaaagagcccatcagcgtcagcagcgaacaggtgctgaagttccggaagctgaacttcaacggcgagggcgagcctgaggaactgatggtggataattggaggcccgctcagcccctgaagaacagacagatcaaggccagcttcaagggcggctctggcggaggatctggaaccgccaagatggaaaccaccttctacgacgacgccctgaacgccagctttctgccttctgagtctggcccctacggctacagcaaccccaagatcctgaagcagagcatgaccctgaacctggccgatcctgtgggcagcctgaaacctcacctgagagccaagaacagcgacctgctgacaagccctgatgtgggcctgctgaaactggctagccccgagctggaacggctgatcatccagtctagcaacggccacatcaccaccacacctacaccaacacagtttctgtgccccaagaacgtgaccgacgagcaagagggattcgccgagggctttgttagagccctggccgaactgcacagccagaataccctgcctagcgtgacatctgccgctcagcctgttaatggcgccggaatggttgctcctgccgtggcttctgttgctggcggatctggatctggcggctttagcgcctctctgcactctgagcctccagtgtacgccaacctgagcaacttcaaccctggcgctcttagctctggtggcggagcaccttcttatggcgctgccggattggcctttcctgctcagcctcagcagcagcaacagcctcctcatcatctgccccagcagatgcctgtgcagcaccctagactgcaggccctgaaagaggaaccccagacagtccctgagatgcccggcgaaacacctcctctgagccccatcgacatggaaagccaagagcggatcaaggccgagcggaagcggatgagaaatagaatcgccgcctccaagtgccggaagaggaagctggaaagaatcgcccggctggaagagaaagtgaaaaccctgaaggcccagaactccgagctggcctctaccgccaacatgctgagagaacaggtggcccagctgaaacagaaagtcatgaaccacgtgaacagcggctgccagctgatgctgacacagcagctgcagaccttcggatccggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaa

SEQ ID NO: 41 - protein, 855 aa, Artificial Sequence
  Description of Artificial Sequence Synthetic polypeptide
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTFGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 42 - DNA, 2568 bp, Artificial Sequence
  Description of Artificial Sequence Synthetic polynucleotide
atgtctcaccactggggctacggcaagcacaatggacctgagcactggcacaaggacttccctatcgccaagggcgagagacagagccccgtggacatcgatacccacaccgccaaatacgaccccagcctgaagcctctgagcgtgtcctatgatcaggccaccagcctgcgcatcctgaacaatggccacgccttcaacgtggaattcgacgacagccaggacaaggccgtgctgaaaggtggacctctggacggcacctaccggctgatccagttccactttcactggggcagcctggatggccagggatctgaacacaccgtggacaagaagaagtacgccgccgaactgcacctggtgcactggaacaccaaatacggcgacttcggcaaagccgtgcagcagcctgatggactggctgtgctgggcatcttcctgaaagtgggctctgccaagcctggccaccagaaagtggtggacgtgctggacagcatcaagaccaagggcaagagcgccgacttcaccaacttcgaccctagaggactgctgcccgagagcctggactactggacataccctggcagcctgaccacacctcctctgctggaatgtgtgacctggatcgtcctgaaagagcccatcagcgtcagcagcgaacaggtgctgaagttccggaagctgaacttcaacggcgagggcgagcctgaggaactgatggtggataattggaggcccgctcagcccctgaagaacagacagatcaaggccagcttcaagggcggctctggcggaggatctggaaccgccaagatggaaaccaccttctacgacgacgccctgaacgccagctttctgccttctgagtctggcccctacggctacagcaaccccaagatcctgaagcagagcatgaccctgaacctggccgatcctgtgggcagcctgaaacctcacctgagagccaagaacagcgacctgctgacaagccctgatgtgggcctgctgaaactggctagccccgagctggaacggctgatcatccagtctagcaacggccacatcaccaccacacctacaccaacacagtttctgtgccccaagaacgtgaccgacgagcaagagggattcgccgagggctttgttagagccctggccgaactgcacagccagaataccctgcctagcgtgacatctgccgctcagcctgttaatggcgccggaatggttgctcctgccgtggcttctgttgctggcggatctggatctggcggctttagcgcctctctgcactctgagcctccagtgtacgccaacctgagcaacttcaaccctggcgctcttagctctggtggcggagcaccttcttatggcgctgccggattggcctttcctgctcagcctcagcagcagcaacagcctcctcatcatctgccccagcagatgcctgtgcagcaccctagactgcaggccctgaaagaggaaccccagacagtccctgagatgcccggcgaaacacctcctctgagccccatcgacatggaaagccaagagcggatcaaggccgagcggaagcggatgagaaatagaatcgccgcctccaagtgccggaagaggaagctggaaagaatcgcccggctggaagagaaagtgaaaaccctgaaggcccagaactccgagctggcctctaccgccaacatgctgagagaacaggtggcccagctgaaacagaaagtcatgaaccacgtgaacagcggctgccagctgatgctgacacagcagctgcagaccttcggatccggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaa

SEQ ID NO: 43 - protein, 855 aa, Artificial Sequence
  Description of Artificial Sequence Synthetic polypeptide
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGGGSGTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTFGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 44 - DNA, 1767 bp, Artificial Sequence
  Description of Artificial Sequence Synthetic polynucleotide
atgaccgccaagatggaaaccaccttctacgacgacgccctgaacgccagctttctgccttctgagtctggcccctacggctacagcaaccccaagatcctgaagcagagcatgaccctgaacctggccgatcctgtgggcagcctgaaacctcacctgagagccaagaacagcgacctgctgacaagccctgatgtgggcctgctgaaactggctagccccgagctggaacggctgatcatccagtctagcaacggccacatcaccaccacacctacaccaacacagtttctgtgccccaagaacgtgaccgacgagcaagagggattcgccgagggctttgttagagccctggccgaactgcacagccagaataccctgcctagcgtgacatctgccgctcagcctgttaatggcgccggaatggttgctcctgccgtggcttctgttgctggcggatctggatctggcggctttagcgcctctctgcactctgagcctccagtgtacgccaacctgagcaacttcaaccctggcgctcttagctctggtggcggagcaccttcttatggcgctgccggattggcctttcctgctcagcctcagcagcagcaacagcctcctcatcatctgccccagcagatgcctgtgcagcaccctagactgcaggccctgaaagaggaaccccagacagtccctgagatgcccggcgaaacacctcctctgagccccatcgacatggaaagccaagagcggatcaaggccgagcggaagcggatgagaaatagaatcgccgcctccaagtgccggaagaggaagctggaaagaatcgcccggctggaagagaaagtgaaaaccctgaaggcccagaactccgagctggcctctaccgccaacatgctgagagaacaggtggcccagctgaaacagaaagtcatgaaccacgtgaacagcggctgccagctgatgctgacacagcagctgcagaccttcggatccggagctactaacttcagcctgctgaagcaggctggagacgtggaggagaaccctggacctttgagcaagggcgaggaggacaacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgccgaggtcaagaccacctacaaggccaagaagcccgtgcagctgcccggcgcctacaacgtcaacatcaagctggacatcacctcccacaacgaggactacaccatcgtggaacagtacgagcgcgccgagggccgccactccaccggcggcatggacgagctgtacaagtaa

SEQ ID NO: 45 - protein, 588 aa, Artificial Sequence
  Description of Artificial Sequence Synthetic polypeptide
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTFGSGATNFSLLKQAGDVEENPGPLSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 46 - DNA, 2634 bp, Artificial Sequence
  Description of Artificial Sequence Synthetic polynucleotide
atgggcacaagcctgctgtgttggatggccctgtgtctgctgggagccgatcatgctgatgcctgtccttacagcaaccccagcctgtgttctggcggcggaggatctgaactgcctacacagggcaccttcagcaacgtgtccaccaatgtgtccccagccaagcctaccaccaccgcttgtccctactccaatcctagcctgtgtagcggaggtggcggaagccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgttctgctgctgagcctggtcatcaccctgtactgcaaccaccggaacaggcggagagtgtgcaagtgccctagacctgtggttggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggaggatccggaatgccgaacccaaggccagggaagccctctgctcctagcctggccctcggccccagccctggcgctagcccctcttggagggcggctccgaaggcttccgacctcctgggtgctaggggccctggtggaaccttccaaggcagggatctgcgaggaggggcgcacgcctctagctcaagcctgaacccgatgcccccctcacagctgcaactgcctaccctgccgctcgtcatggtggcccccagcggcgcaagactgggcccgttgccgcacctgcaagccttgctgcaggaccggccacatttcatgcaccagctcagcaccgtggacgcacatgcaaggacacccgtgctgcaagtccaccccctggagagccctgccatgatcagcctgacgccgcccaccaccgcaaccggcgtgttttcactgaaggcaagacccgggctgccaccgggcatcaacgtggccagcctggaatgggtgagcagggagccagcgctcctgtgtaccttcccaaacccatccgccccaagaaaggacagcaccctgtctgccgtgccccaatcatcttacccgctgctggcgaatggcgtatgcaagtggcccggatgtgaaaaggtgttcgaggagccggaagatttcctgaaacattgccaggccgaccacctgttggacgaaaagggaagggcccaatgcctgcttcagagggagatggtgcagagcttggagcaacaactcgtgctcgagaaggagaagctgagcgccatgcaggcacacctcgccggcaagatggccctgaccaaagccagtagcgtagccagctccgacaagggtagctgttgcatcgtggccgcaggaagtcaaggccccgttgtgcccgcctggagcggtccaagggaggcacccgactcactgttcgccgtgaggaggcatctgtggggcagccacggtaacagcacgttccccgagttcctgcataacatggactacttcaagttccacaacatgcggcctccattcacctacgccacactgataaggtgggctatcctggaggctcccgagaagcaaaggaccctgaacgagatctaccactggttcaccaggatgttcgctttctttaggaaccaccccgcgacctggaaaaacgccataaggcataacttgagccttcacaagtgcttcgtgagggtggagagtgagaaaggtgccgtgtggactgtggatgagttggagttccgcaagaagcgaagccaacgacctagcaggtgtagcaatccaacgcctggaccctaa

SEQ ID NO: 47 - protein, 877 aa, Artificial Sequence
  Description of Artificial Sequence Synthetic polypeptide
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSGATNFSLLKQAGDVEENPGPGGSMSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGMPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 48 - DNA, 2634 bp, Artificial Sequence
  Description of Artificial Sequence Synthetic polynucleotide
atgggcacaagcctgctgtgttggatggccctgtgtctgctgggagccgatcatgctgatgcctgtccttacagcaaccccagcctgtgttctggcggcggaggatctgaactgcctacacagggcaccttcagcaacgtgtccaccaatgtgtccccagccaagcctaccaccaccgcttgtccctactccaatcctagcctgtgtagcggaggtggcggaagccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgttctgctgctgagcctggtcatcaccctgtactgcaaccaccggaacaggcggagagtgtgcaagtgccctagacctgtggttggatctggtgctactaacttcagcctgctgaagcaggctggagacgtggaggagaaccctggacctggaggatccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaaggaggatccggaatgccgaacccaaggccagggaagccctctgctcctagcctggccctcggccccagccctggcgctagcccctcttggagggcggctccgaaggcttccgacctcctgggtgctaggggccctggtggaaccttccaaggcagggatctgcgaggaggggcgcacgcctctagctcaagcctgaacccgatgcccccctcacagctgcaactgcctaccctgccgctcgtcatggtggcccccagcggcgcaagactgggcccgttgccgcacctgcaagccttgctgcaggaccggccacatttcatgcaccagctcagcaccgtggacgcacatgcaaggacacccgtgctgcaagtccaccccctggagagccctgccatgatcagcctgacgccgcccaccaccgcaaccggcgtgttttcactgaaggcaagacccgggctgccaccgggcatcaacgtggccagcctggaatgggtgagcagggagccagcgctcctgtgtaccttcccaaacccatccgccccaagaaaggacagcaccctgtctgccgtgccccaatcatcttacccgctgctggcgaatggcgtatgcaagtggcccggatgtgaaaaggtgttcgaggagccggaagatttcctgaaacattgccaggccgaccacctgttggacgaaaagggaagggcccaatgcctgcttcagagggagatggtgcagagcttggagcaacaactcgtgctcgagaaggagaagctgagcgccatgcaggcacacctcgccggcaagatggccctgaccaaagccagtagcgtagccagctccgacaagggtagctgttgcatcgtggccgcaggaagtcaaggccccgttgtgcccgcctggagcggtccaagggaggcacccgactcactgttcgccgtgaggaggcatctgtggggcagccacggtaacagcacgttccccgagttcctgcataacatggactacttcaagttccacaacatgcggcctccattcacctacgccacactgataaggtgggctatcctggaggctcccgagaagcaaaggaccctgaacgagatctaccactggttcaccaggatgttcgctttctttaggaaccaccccgcgacctggaaaaacgccataaggcataacttgagccttcacaagtgcttcgtgagggtggagagtgagaaaggtgccgtgtggactgtggatgagttggagttccgcaagaagcgaagccaacgacctagcaggtgtagcaatccaacgcctggaccctaa

SEQ ID NO: 49 - protein, 877 aa, Artificial Sequence
  Description of Artificial Sequence Synthetic polypeptide
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSGATNFSLLKQAGDVEENPGPGGSMSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGHQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFKGGSGMPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 50 - DNA, 1845 bp, Artificial Sequence
  Description of Artificial Sequence Synthetic polynucleotide
atgggcacaagcctgctgtgttggatggccctgtgtctgctgggagccgatcatgctgatgcctgtccttacagcaaccccagcctgtgttctggcggcggaggatctgaactgcctacacagggcaccttcagcaacgtgtccaccaatgtgtccccagccaagcctaccaccaccgcttgtccctactccaatcctagcctgtgtagcggaggtggcggaagccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgttctgctgctgagcctggtcatcaccctgtactgcaaccaccggaacaggcggagagtgtgcaagtgccctagacctgtggttggatctggtgctactaacttcagcctgctgaagcaggctggagacgtggaggagaaccctggacctggaggatccggaatgccgaacccaaggccagggaagccctctgctcctagcctggccctcggccccagccctggcgctagcccctcttggagggcggctccgaaggcttccgacctcctgggtgctaggggccctggtggaaccttccaaggcagggatctgcgaggaggggcgcacgcctctagctcaagcctgaacccgatgcccccctcacagctgcaactgcctaccctgccgctcgtcatggtggcccccagcggcgcaagactgggcccgttgccgcacctgcaagccttgctgcaggaccggccacatttcatgcaccagctcagcaccgtggacgcacatgcaaggacacccgtgctgcaagtccaccccctggagagccctgccatgatcagcctgacgccgcccaccaccgcaaccggcgtgttttcactgaaggcaagacccgggctgccaccgggcatcaacgtggccagcctggaatgggtgagcagggagccagcgctcctgtgtaccttcccaaacccatccgccccaagaaaggacagcaccctgtctgccgtgccccaatcatcttacccgctgctggcgaatggcgtatgcaagtggcccggatgtgaaaaggtgttcgaggagccggaagatttcctgaaacattgccaggccgaccacctgttggacgaaaagggaagggcccaatgcctgcttcagagggagatggtgcagagcttggagcaacaactcgtgctcgagaaggagaagctgagcgccatgcaggcacacctcgccggcaagatggccctgaccaaagccagtagcgtagccagctccgacaagggtagctgttgcatcgtggccgcaggaagtcaaggccccgttgtgcccgcctggagcggtccaagggaggcacccgactcactgttcgccgtgaggaggcatctgtggggcagccacggtaacagcacgttccccgagttcctgcataacatggactacttcaagttccacaacatgcggcctccattcacctacgccacactgataaggtgggctatcctggaggctcccgagaagcaaaggaccctgaacgagatctaccactggttcaccaggatgttcgctttctttaggaaccaccccgcgacctggaaaaacgccataaggcataacttgagccttcacaagtgcttcgtgagggtggagagtgagaaaggtgccgtgtggactgtggatgagttggagttccgcaagaagcgaagccaacgacctagcaggtgtagcaatccaacgcctggaccctaa

SEQ ID NO: 51 - protein, 614 aa, Artificial Sequence
  Description of Artificial Sequence Synthetic polypeptide
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSGATNFSLLKQAGDVEENPGPGGSGMPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 52 - DNA, 990 bp, Artificial Sequence
  Description of Artificial Sequence Synthetic polynucleotide
accgccaagatggaaaccaccttctacgacgacgccctgaacgccagctttctgccttctgagtctggcccctacggctacagcaaccccaagatcctgaagcagagcatgaccctgaacctggccgatcctgtgggcagcctgaaacctcacctgagagccaagaacagcgacctgctgacaagccctgatgtgggcctgctgaaactggctagccccgagctggaacggctgatcatccagtctagcaacggccacatcaccaccacacctacaccaacacagtttctgtgccccaagaacgtgaccgacgagcaagagggattcgccgagggctttgttagagccctggccgaactgcacagccagaataccctgcctagcgtgacatctgccgctcagcctgttaatggcgccggaatggttgctcctgccgtggcttctgttgctggcggatctggatctggcggctttagcgcctctctgcactctgagcctccagtgtacgccaacctgagcaacttcaaccctggcgctcttagctctggtggcggagcaccttcttatggcgctgccggattggcctttcctgctcagcctcagcagcagcaacagcctcctcatcatctgccccagcagatgcctgtgcagcaccctagactgcaggccctgaaagaggaaccccagacagtccctgagatgcccggcgaaacacctcctctgagccccatcgacatggaaagccaagagcggatcaaggccgagcggaagcggatgagaaatagaatcgccgcctccaagtgccggaagaggaagctggaaagaatcgcccggctggaagagaaagtgaaaaccctgaaggcccagaactccgagctggcctctaccgccaacatgctgagagaacaggtggcccagctgaaacagaaagtcatgaaccacgtgaacagcggctgccagctgatgctgacacagcagctgcagaccttc

SEQ ID NO: 53 - protein, 330 aa, Homo sapiens
TAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 54 - DNA, 471 bp, Artificial Sequence
  Description of Artificial Sequence Synthetic polynucleotide
atgggcacaagcctgctgtgttggatggccctgtgtctgctgggagccgatcatgctgatgcctgtccttacagcaaccccagcctgtgttctggcggcggaggatctgaactgcctacacagggcaccttcagcaacgtgtccaccaatgtgtccccagccaagcctaccaccaccgcttgtccctactccaatcctagcctgtgtagcggaggtggcggaagccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgttctgctgctgagcctggtcatcaccctgtactgcaaccaccggaacaggcggagagtgtgcaagtgccctagacctgtggtt

SEQ ID NO: 55 - protein, 157 aa, Artificial Sequence
  Description of Artificial Sequence Synthetic polypeptide
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 56 - DNA, 1293 bp, Artificial Sequence
  Description of Artificial Sequence Synthetic polynucleotide
atgccgaacccaaggccagggaagccctctgctcctagcctggccctcggccccagccctggcgctagcccctcttggagggcggctccgaaggcttccgacctcctgggtgctaggggccctggtggaaccttccaaggcagggatctgcgaggaggggcgcacgcctctagctcaagcctgaacccgatgcccccctcacagctgcaactgcctaccctgccgctcgtcatggtggcccccagcggcgcaagactgggcccgttgccgcacctgcaagccttgctgcaggaccggccacatttcatgcaccagctcagcaccgtggacgcacatgcaaggacacccgtgctgcaagtccaccccctggagagccctgccatgatcagcctgacgccgcccaccaccgcaaccggcgtgttttcactgaaggcaagacccgggctgccaccgggcatcaacgtggccagcctggaatgggtgagcagggagccagcgctcctgtgtaccttcccaaacccatccgccccaagaaaggacagcaccctgtctgccgtgccccaatcatcttacccgctgctggcgaatggcgtatgcaagtggcccggatgtgaaaaggtgttcgaggagccggaagatttcctgaaacattgccaggccgaccacctgttggacgaaaagggaagggcccaatgcctgcttcagagggagatggtgcagagcttggagcaacaactcgtgctcgagaaggagaagctgagcgccatgcaggcacacctcgccggcaagatggccctgaccaaagccagtagcgtagccagctccgacaagggtagctgttgcatcgtggccgcaggaagtcaaggccccgttgtgcccgcctggagcggtccaagggaggcacccgactcactgttcgccgtgaggaggcatctgtggggcagccacggtaacagcacgttccccgagttcctgcataacatggactacttcaagttccacaacatgcggcctccattcacctacgccacactgataaggtgggctatcctggaggctcccgagaagcaaaggaccctgaacgagatctaccactggttcaccaggatgttcgctttctttaggaaccaccccgcgacctggaaaaacgccataaggcataacttgagccttcacaagtgcttcgtgagggtggagagtgagaaaggtgccgtgtggactgtggatgagttggagttccgcaagaagcgaagccaacgacctagcaggtgtagcaatccaacgcctggaccc

SEQ ID NO: 57 - protein, 431 aa, Artificial Sequence
  Description of Artificial Sequence Synthetic polypeptide
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 58 - protein, 8 aa, Artificial Sequence
  Description of Artificial Sequence Synthetic peptide
GGSGGGSG

SEQ ID NO: 59 - protein, 9 aa, Artificial Sequence
  Description of Artificial Sequence Synthetic peptide
GGSGGGSGG

SEQ ID NO: 60 - DNA, 2503 bp, Artificial Sequence
  Description of Artificial Sequence Synthetic polynucleotide
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatcactagtgccaccatggcacctaagaaaaagaggaaggttgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagactgggggccttgcttggcaacagcacagacccagctgtgttcacagacctggcatccgtggacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccggaggtagtggtggaggcagtggtggtgttggttcgctaaactgcatcgtcgctgtgtcccagaacatgggcatcggcaagaacggggacctgccctggccaccgctcaggaatgaattcagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgttattaaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgatggatcctga

SEQ ID NO: 61 - protein, 431 aa, Artificial Sequence
  Description of Artificial Sequence Synthetic polypeptide
MAPKKKRKVERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTRLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGGSGGGSGGVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVIKEAMNHPGHLKLFVTRIMQDFESDTFFPEIDLEKYKLLPEYPGVLSDVQEEKGIKYKFEVYEKNDGS

SEQ ID NO: 62 - DNA, 2680 bp, Artificial Sequence
  Description of Artificial Sequence Synthetic polynucleotide
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatcactagtgccaccatggcacctaagaaaaagaggaaggttgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagactgggggccttgcttggcaacagcacagacccagctgtgttcacagacctggcatccgtggacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccggaggtagtggtggaggcagtggtggttcactggcgctcagccttactgccgaccaaatggtatcagctcttctggacgcagaacccccaattctttattccgagtacgaccccacacgcccgttcagtgaagcttccatgatgggcctccttacgaaccttgccgaccgggaactcgtgcacatgatcaattgggcgaagcgggtgccggggttcgtagatttgacacttcacgaccaagttcatctcttggaatgtgcttggatggagatattgatgatcggactcgtgtggaggtcaatggagcatcctggtaaacttcttttcgcacccaatctgctcttggatagaaatcagggtaagtgcgtcgagggtggcgttgaaatcttcgacatgctccttgcgacatccagccgattccgaatgatgaatcttcaaggagaggaatttgtctgtcttaagagcattatactcctcaatagtggagtttacaccttcttgtcctctacactgaaatcacttgaggaaaaagatcacatacatagggtgttggataaaatcacggatacactcatacatctgatggcaaaagcaggattgaccctgcaacagcagcaccgacgactggcccaactgctgttgatccttagccatatcagacacatgtctaacaaaaggatggaacatttgtacagcatgaaatgtaagaacgtagtgccactgtccgatttgttgctggaaatgctggacgctcatcggctcggatcctga

SEQ ID NO: 63 - protein, 490 aa, Artificial Sequence
  Description of Artificial Sequence Synthetic polypeptide
MAPKKKRKVERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTRLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGGSGGGSGGSLALSLTADQMVSALLDAEPPILYSEYDPTRPFSEASMMGLLTNLADRELVHMINWAKRVPGFVDLTLHDQVHLLECAWMEILMIGLVWRSMEHPGKLLFAPNLLLDRNQGKCVEGGVEIFDMLLATSSRFRMMNLQGEEFVCLKSIILLNSGVYTFLSSTLKSLEEKDHIHRVLDKITDTLIHLMAKAGLTLQQQHRRLAQLLLILSHIRHMSNKRMEHLYSMKCKNVVPLSDLLLEMLDAHRLGS

SEQ ID NO: 64 - DNA, 2404 bp, Artificial Sequence; identical to SEQ ID NO: 9
  Description of Artificial Sequence Synthetic polynucleotide

SEQ ID NO: 65 - protein, 398 aa, Artificial Sequence; identical to SEQ ID NO: 10
  Description of Artificial Sequence Synthetic polypeptide

SEQ ID NO: 66 - DNA, 1918 bp, Artificial Sequence; identical to SEQ ID NO: 7
  Description of Artificial Sequence Synthetic polynucleotide

SEQ ID NO: 67 - protein, 236 aa, Artificial Sequence; identical to SEQ ID NO: 8
  Description of Artificial Sequence Synthetic polypeptide

SEQ ID NO: 68 - DNA, 7733 bp, Artificial Sequence
  Description of Artificial Sequence Synthetic polynucleotide
tttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcattgatcacgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatccactagtgatatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 69 - DNA, 7733 bp, Artificial Sequence
  Description of Artificial Sequence Synthetic polynucleotide
tttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagactagtgatatcggatcccgggacgtggtcgacgctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 70 - DNA, 12 bp, Artificial Sequence
  Description of Artificial Sequence Synthetic oligonucleotide
taatgatgggcg

SEQ ID NO: 71 - protein, 875 aa, Homo sapiens
MERAGPSFGQQRQQQQPQQQKQQQRDQDSVEAWLDDHWDFTFSYFVRKATREMVNAWFAERVHTIPVCKEGIRGHTESCSCPLQQSPRADNSAPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

(19) United States
(12) Patent Application Publication
(10) Pub. No.: US 2023/0056856 A1
(43) Pub. Date: Feb. 23, 2023

(54) COMPOSITIONS AND METHODS FOR TUNABLE REGULATION OF TRANSCRIPTION

(71) Applicant: OBSIDIAN THERAPEUTICS, INC., Cambridge, MA (US)

(72) Inventors: Vipin Suri, Belmont, MA (US); Mara Christine Inniss, Beverly, MA (US); Samantha Fleury, Houston, TX (US)

(73) Assignee: OBSIDIAN THERAPEUTICS, INC., Cambridge, MA (US)

(21) Appl. No.: 17/791,091

(22) PCT Filed: Jan. 8, 2021
(86) PCT No.: PCT/US2021/012845

§ 371 Date: Jul. 6, 2022

Related U.S. Application Data
(60) Provisional application No. 62/958,693, filed on Jan. 8, 2020.
(60) Provisional application No. 62/959,859, filed on Jan. 10, 2020.

(51) Int. Cl. C12N 15/63; C12N 9/06; C12N 5/0783; A61K 48/00; A61K 35/17; C07K 14/72

(52) U.S. Cl. CPC C12N 15/635; C12N 9/003; C12N 5/0638; A61K 48/0066; A61K 35/17; C07K 14/721; C12Y 402/01001; C12Y 105/01003; C12Y 502/01008; C12Y 301/04017; C07K 2319/81

(57) ABSTRACT

The present disclosure provides compositions and methods related to transcription factor systems. Such systems provide for modular and tunable protein expression driven by regulated transcriptional activity.

### CROSS REFERENCE TO RELATED APPLICATIONS

This application claims benefit of priority to U.S. Provisional Application No. 62/958,693, filed Jan. 8, 2020 and U.S. Provisional Application No. 62/959,859, filed Jan. 10, 2020. The entire contents of the aforementioned applications are incorporated herein by reference in their entireties.

### REFERENCE TO THE SEQUENCE LISTING

The instant application contains a Sequence Listing which has been submitted electronically in ASCII format and is hereby incorporated by reference in its entirety. Said ASCII copy, created on Jan. 8, 2021, is named 268052_483267_SL.txt and is 241,815 bytes in size.

### FIELD

The present disclosure relates to systems, compositions and methods for tunable protein expression driven by regulated transcriptional activity. Provided in the present disclosure are modular transcription factor systems, polynucleotides of transcription factor systems, polypeptides, vectors, cells, compositions and methods for use in regulation of transcription and regulated protein expression driven by regulated transcriptional activity.

### BACKGROUND

Gene and cell therapies are revolutionizing medicine and offering new promise for the treatment of previously intractable conditions. However, most current technologies do not allow titration of the timing or levels of target protein induction. This has rendered many potential gene and cell therapy applications difficult or impossible to safely and effectively deploy.

Inadequate exogenous and/or endogenous gene control is a critical issue in many gene and cell therapy settings. This lack of tunability also makes it difficult to safely express proteins with narrow or uncertain therapeutic windows or those requiring more titrated or transient expression.

One approach to regulated protein expression or function is the use of drug responsive domains (DRDs). Drug responsive domains are small protein domains that can be appended to a target protein of interest. DRDs render the attached protein of interest unstable in the absence of a DRD-binding ligand and the protein of interest is rapidly degraded by the ubiquitin-proteasome system of the cell. However, when a specific small molecule DRD-binding ligand binds to the DRD, the attached protein of interest is stabilized, and protein function is achieved.

DRD technology forms the basis of a new class of cell and gene therapies that can deliver tunable and temporal control of gene expression and function, expanding the universe of protein therapeutics that can be safely and effectively incorporated into cell and gene therapy modalities. However, current DRD technology produces fusion proteins in which the protein of interest is joined to a DRD, which may be unsuitable for some indications. Thus, there remains a need to develop cell and gene therapies in which native proteins of interest can be expressed in a regulated manner.

### SUMMARY

The present invention provides modified cells, nucleic acid molecules, vectors, and cell and gene therapies in which the timing or levels of a native therapeutic protein can be regulated through administration of an oral small molecule drug.

Additionally, the present disclosure provides compositions, systems and methods for tunable regulation of transcription. The compositions relate to transcription factor systems and agents that induce transcriptional activity of a polynucleotide encoding a protein of interest. Compositions provided by the present disclosure include nucleic acid molecules, polypeptides, and cells related to transcription factor systems. Methods related to transcription factor systems that are provided by the present disclosure include methods of producing modified cells and methods of treating or preventing disease.

Provided herein are transcription factor systems. A transcription factor system of the present disclosure is a combination of one or more polynucleotides that comprise (1) one or more nucleic acid sequences that encode a transcription factor that is able to bind to a specific polynucleotide binding site and activate transcription; (2) a nucleic acid sequence that encodes a drug responsive domain (DRD), wherein the transcription factor, or part thereof, is operably linked to the DRD; and (3) a nucleic acid sequence that encodes a payload and is operably linked to an inducible promoter comprising the specific polynucleotide binding site.

The present disclosure provides modified cells related to transcription factor systems.

In some aspects, the present disclosure provides a modified cell that may regulate expression or transcription of a payload. The modified cell comprises: a first polynucleotide that comprises a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD). At least one of the transcription factor activation domain, the transcription factor DNA binding domain, or the combination of the transcription factor activation domain and the transcription factor DNA binding domain is operably linked to the DRD. The transcription factor activation domain and the transcription factor DNA binding domain interact to form a transcription factor that is able to activate transcription of a fourth nucleic acid sequence upon binding to the specific polynucleotide binding site, the fourth nucleic acid sequence encoding a protein of interest and being operably linked to either the specific polynucleotide binding site, an exogenous inducible promoter comprising the specific polynucleotide binding site, or both. In some embodiments, the protein of interest is a heterologous protein. In some embodiments, the fourth nucleic acid sequence is located on the first polynucleotide. In some embodiments, the modified cell further comprises a second polynucleotide that comprises the fourth nucleic acid sequence.

In some aspects, the present disclosure provides a modified cell comprising a polynucleotide that comprises a first nucleic acid sequence encoding a drug responsive domain (DRD) and a second nucleic acid sequence encoding a transcription factor. The transcription factor is operably linked to the DRD and is able to bind to a specific polynucleotide binding site and activate transcription of a third nucleic acid sequence encoding a protein of interest, the third nucleic acid sequence being operably linked to either the specific polynucleotide binding site, an exogenous inducible promoter comprising the specific polynucleotide binding site, or both. In some embodiments, the protein of interest is a heterologous protein. In some embodiments, the third nucleic acid sequence is located on the polynucleotide that comprises the first nucleic acid sequence and the second nucleic acid sequence. In some embodiments, the modified cell further comprises a second polynucleotide that comprises the third nucleic acid sequence.

In another aspect, the present disclosure provides a modified cell comprising (a) a first polynucleotide comprising a first nucleic acid sequence encoding a transcription factor that is able to bind to a specific polynucleotide binding site and activate transcription, and a second nucleic acid sequence encoding a drug responsive domain (DRD); wherein the transcription factor, or part thereof, is operably linked to the DRD; and (b) a second polynucleotide comprising a third nucleic acid sequence encoding a protein of interest, said third nucleic acid sequence being operably linked to an exogenous inducible promoter comprising the specific polynucleotide binding site.

In another aspect, the present disclosure provides a modified cell comprising (a) a polynucleotide comprising a first nucleic acid sequence encoding a transcription factor able to bind to a specific polynucleotide binding site and activate transcription of a second nucleic acid sequence encoding a protein of interest; wherein the second nucleic acid sequence is operably linked to an exogenous inducible promoter comprising the specific polynucleotide binding site; and (b) a third nucleic acid sequence encoding a drug responsive domain (DRD); wherein the transcription factor is operably linked to the DRD.

In another aspect, the present disclosure provides a modified cell comprising (a) a first polynucleotide comprising a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD; and (b) a second polynucleotide comprising a fourth nucleic acid sequence that encodes a protein of interest, said fourth nucleic acid sequence being operably linked to an exogenous inducible promoter comprising the specific polynucleotide binding site; wherein the transcription factor activation domain and the transcription factor DNA binding domain interact to form a transcription factor that is able to activate transcription upon binding to the specific polynucleotide binding site.

In another aspect, the present disclosure provides a modified cell comprising (a) a first polynucleotide comprising a nucleic acid sequence encoding a transcription factor activation domain; (b) a second polynucleotide comprising a nucleic acid sequence encoding a transcription factor DNA binding domain that binds to a specific polynucleotide binding site located on an exogenous inducible promoter; and (c) a third polynucleotide comprising a nucleic acid sequence encoding a drug responsive domain (DRD); wherein at least one of the transcription factor activation domain, the transcription factor DNA binding domain, or the combination of the transcription factor activation domain and the transcription factor DNA binding domain is operably linked to the DRD. In one aspect, the transcription factor activation domain and the transcription factor DNA binding domain interact to form a transcription factor that is able to bind to the specific polynucleotide binding site and activate transcription of a nucleic acid sequence encoding a protein of interest, said nucleic acid sequence being operably linked to the exogenous inducible promoter.

In various embodiments, one or more of the transcription factor DNA binding domain, the transcription factor activation domain and the DRD is derived from a parent protein. In some embodiments, the transcription factor DNA binding domain is derived from a parent protein selected from the group consisting of: ZFHD1, Cas9, Cas12, and TAL. In some embodiments, the transcription factor activation domain is derived from a parent protein, wherein the parent protein is p65. In some embodiments, the DRD is derived from a parent protein selected from the group comprising: human carbonic anhydrase 2 (CA2), human DHFR, E. coli DHFR (ecDHFR), human estrogen receptor (ER), FKBP, human protein FKBP, and human PDE5.

In some embodiments, the DRD is stabilized in the presence of a ligand selected from the group comprising: Acetazolamide (ACZ), Methotrexate (MTX), and Trimethoprim (TMP). In some embodiments, the DRD is responsive to or interacts with a ligand selected from the group comprising: Acetazolamide (ACZ), Methotrexate (MTX), and Trimethoprim (TMP).

In some embodiments, the protein of interest is a wild-type protein.

In some embodiments, the protein of interest is a therapeutic protein.

In some embodiments, the protein of interest is selected from the group consisting of a cytokine, an antibody, or an antigen binding fragment thereof, a coagulation factor, an enzyme, a gene editing protein, a T cell receptor (TCR) and a chimeric antigen receptor (CAR).

In some embodiments, the protein of interest is selected from the group consisting of IL2, IL12, IL15, Cas9, ZFN, and Cre.

In some embodiments, the protein of interest is a secreted protein.

In some embodiments, the cell is a T cell, a natural killer cell (NK cell), or a tumor infiltrating lymphocyte (TIL).

In some embodiments, the cell is a stem cell, a liver cell, a blood cell, a pancreatic cell, a neuronal cell, an ocular cell, a muscle cell, or a bone cell.

Also provided by the present disclosure are nucleic acid molecules related to transcription factor systems.

In one aspect, the present disclosure provides a nucleic acid molecule comprising (a) a first nucleic acid sequence that encodes a transcription factor DNA binding domain that binds to a specific polynucleotide binding site; and (b) a second nucleic acid sequence that encodes a drug responsive domain (DRD). In some embodiments, the nucleic acid molecule further comprises (c) a third nucleic acid sequence that encodes a transcription factor activation domain; wherein either (i) the transcription factor DNA binding domain is operably linked to the DRD; (ii) the transcription factor activation domain is operably linked to the DRD; or (iii) the combination of the transcription factor DNA binding domain and the transcription factor activation domain is operably linked to the DRD. In some embodiments, the transcription factor DNA binding domain is derived from a parent protein selected from the group consisting of: ZFHD1, Cas9, Cas12, and TAL. In some embodiments, the transcription factor activation domain is derived from a parent protein, wherein said parent protein is p65.

In one aspect, the present disclosure provides a nucleic acid molecule comprising (a) a first nucleic acid sequence encoding a transcription factor able to bind to a specific polynucleotide binding site and activate transcription; and (b) a second nucleic acid sequence encoding a drug responsive domain (DRD); wherein the transcription factor is operably linked to the DRD. In some embodiments, the nucleic acid molecule further comprises (c) a third nucleic acid sequence that encodes a protein of interest, the third nucleic acid sequence being operably linked to either the specific polynucleotide binding site, an exogenous inducible promoter comprising the specific polynucleotide binding site, or both.

In some embodiments, the specific polynucleotide binding site is located on an exogenous inducible promoter.

In some embodiments, the DRD is derived from a parent protein selected from the group comprising: human carbonic anhydrase 2 (CA2), human DHFR, ecDHFR, human estrogen receptor (ER), FKBP, human protein FKBP, and human PDE5.

In some embodiments, the DRD is stabilized in the presence of a ligand selected from the group comprising: Acetazolamide (ACZ), Methotrexate (MTX), and Trimethoprim (TMP). In some embodiments, the DRD is responsive to or interacts with a ligand selected from the group comprising: Acetazolamide (ACZ), Methotrexate (MTX), and Trimethoprim (TMP).

In some embodiments, the protein of interest is a wild-type protein.

In some embodiments, the protein of interest is a therapeutic protein.

In some embodiments, the protein of interest is selected from the group consisting of a cytokine, an antibody, a coagulation factor, an enzyme, a gene editing protein, a T cell receptor (TCR) and a chimeric antigen receptor (CAR).

In some embodiments, the protein of interest is selected from the group consisting of IL2, IL12, IL15, Cas9, ZFN, and Cre.

In some embodiments, the protein of interest is a secreted protein.

Also provided herein are vectors comprising nucleic acid molecules described herein. Vectors provided by the present disclosure include a plasmid or a viral vector. In some aspects, the viral vector is derived from an adenovirus, adeno-associated virus (AAV), alphavirus, flavivirus, herpes virus, measles virus, rhabdovirus, retrovirus, lentivirus, Newcastle disease virus (NDV), poxvirus, and picornavirus. In some aspects, the viral vector is selected from the group consisting of a lentivirus vector, a gamma retrovirus vector, adeno-associated virus (AAV) vector, adenovirus vector, and a herpes virus vector.

Also provided by the present disclosure are a first polynucleotide and second polynucleotide comprising nucleic acid sequences encoding one or more components of transcription factor systems.

In one aspect, the present disclosure provides a first polynucleotide and second polynucleotide, the first polynucleotide comprising: a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD); wherein at least one of the transcription factor activation domain, the transcription factor DNA binding domain, or the combination of the transcription factor activation domain and the transcription factor DNA binding domain is operably linked to the DRD; and a second polynucleotide comprising: a fourth nucleic acid sequence that encodes a protein of interest, the fourth nucleic acid sequence being operably linked to an inducible promoter comprising the specific polynucleotide binding site; wherein the transcription factor activation domain and the transcription factor DNA binding domain interact to form a transcription factor that is able to activate transcription upon binding to the specific polynucleotide binding site, and wherein the first polynucleotide and the second polynucleotide are each carried in a single vector, or the first polynucleotide and the second polynucleotide are carried in separate vectors.

In one aspect, the present disclosure provides a first polynucleotide and second polynucleotide, the first polynucleotide comprising: a first nucleic acid sequence that encodes a transcription factor and a second nucleic acid sequence that encodes a drug responsive domain (DRD), wherein the transcription factor is operably linked to the DRD and wherein the transcription factor is able to activate transcription upon binding to a specific polynucleotide binding site; and a second polynucleotide comprising: a third nucleic acid sequence that encodes a protein of interest, the third nucleic acid sequence being operably linked to an inducible promoter comprising the specific polynucleotide binding site; wherein the first polynucleotide and the second polynucleotide are each carried in a single vector, or the first polynucleotide and the second polynucleotide are carried in separate vectors.

In some embodiments, the DRD is derived from a parent protein selected from the group comprising: human carbonic anhydrase 2 (CA2), human DI-FR, ecDHFR, human estrogen receptor (ER), FKBP, human protein FKBP, and human PDE5. In some embodiments, the DRD is stabilized in the presence of a ligand selected from the group comprising: Acetazolamide (ACZ), Methotrexate (MTX), and Trimethoprim (TMP).

In some embodiments, the protein of interest is a wild-type protein. In some embodiments, the protein of interest is a therapeutic protein. In some embodiments, the protein of interest is selected from the group consisting of a cytokine, an antibody, a coagulation factor, an enzyme, a gene editing protein, a T cell receptor (TCR) and a chimeric antigen receptor (CAR). In some embodiments, the protein of interest is selected from the group consisting of IL2, IL12, IL15, Cas9, ZFN, and Cre. In some embodiments, the protein of interest is a secreted protein.

Also provided by the present disclosure are methods related to transcription factor systems.

In one aspect, the present disclosure provides a method of producing a modified cell, said method comprising introducing into a cell a nucleic acid molecule comprising: (a) a first nucleic acid sequence that encodes a transcription factor DNA binding domain that binds to a specific polynucleotide binding site; and (b) a second nucleic acid sequence that encodes a drug responsive domain (DRD). In one embodiment, the nucleic acid molecule further comprises a third nucleic acid sequence that encodes a transcription factor activation domain. In some embodiments, either (i) the transcription factor DNA binding domain is operably linked to the DRD; (ii) the transcription factor activation domain is operably linked to the DRD; or (iii) the combination of the transcription factor DNA binding domain and the transcription factor activation domain is operably linked to the DRD.

In some embodiments, the method further comprises introducing into the cell: a fourth nucleic acid sequence encoding a protein of interest, said fourth nucleic acid sequence being operably linked to an inducible promoter comprising the specific polynucleotide binding site. In some embodiments, the protein of interest is a heterologous protein. In one embodiment, the fourth nucleic acid sequence is on the same nucleic acid molecule as the first, second and third nucleic acid sequences. In one embodiment, the fourth nucleic acid sequence is on a different nucleic acid molecule than the first, second and third nucleic acid sequences.

In some embodiments, the protein of interest is selected from the group consisting of a cytokine, an antibody, or an antigen binding fragment thereof, a coagulation factor, an enzyme, a gene editing protein, a T cell receptor (TCR) and a chimeric antigen receptor (CAR).

In some embodiments, the protein of interest is selected from the group consisting of IL2, IL12, IL15, Cas9, ZFN, and Cre.

In some embodiments, the protein of interest is a secreted protein.

In some embodiments, the nucleic acid molecule is introduced into the cell by a plasmid or a viral vector. In one embodiment, the viral vector is derived from an adenovirus, adeno-associated virus (AAV), alphavirus, flavivirus, herpes virus, measles virus, rhabdovirus, retrovirus, lentivirus, Newcastle disease virus (NDV), poxvirus, and picornavirus. In one embodiment, the viral vector is selected from the group consisting of a lentivirus vector, a gamma retrovirus vector, adeno-associated virus (AAV) vector, adenovirus vector, and a herpes virus vector.

In some embodiments, the nucleic acid molecule is introduced into the cell by a non-viral delivery method.

In some embodiments, the cell is a T cell, a natural killer cell (NK cell), or a tumor infiltrating lymphocyte (TIL). In some embodiments, the cell is a stem cell, a liver cell, a blood cell, a pancreatic cell, a neuronal cell, an ocular cell, a muscle cell, or a bone cell.

Also provided by the present disclosure are methods related to treating or preventing disease.

In one aspect, the present disclosure provides a method for treating or preventing a disease in a subject in need thereof, the method comprising: (a) providing a population of cells; (b) introducing at least one nucleic acid molecule into at least one cell in the population of cells, wherein the at least one nucleic acid molecule comprises: (i) a first polynucleotide comprising a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD), wherein at least one of the transcription factor activation domain and the transcription factor DNA binding domain is operably linked to the DRD; and (ii) a second polynucleotide that comprises a fourth nucleic acid sequence that encodes a protein of interest that prevents or treats the disease, or a symptom thereof, said fourth nucleic acid sequence being operably linked to an exogenous inducible promoter comprising the specific polynucleotide binding site; (c) delivering the cell into the subject; and (d) administering a ligand to the subject that stabilizes the DRD sufficiently to enable expression of the at least one of the transcription factor activation domain and the transcription factor DNA binding domain in an amount sufficient to form a transcription factor that binds to the specific polynucleotide binding site and enables expression of the protein of interest in the cell; wherein expression of the protein of interest is regulated by the presence of ligand in the subject, and the amount and/or duration of ligand administration is sufficient to produce a therapeutically effective amount of the protein of interest.

In one aspect, the present disclosure provides a method for introducing a modified cell into a subject in need of disease treatment or prevention, the method comprising: (a) providing a population of cells; (b) introducing at least one nucleic acid molecule into at least one cell in the population of cells, wherein the at least one nucleic acid molecule comprises: (i) a first polynucleotide comprising a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD), wherein at least one of the transcription factor activation domain and the transcription factor DNA binding domain is operably linked to the DRD; and (ii) a second polynucleotide that comprises a fourth nucleic acid sequence that encodes a protein of interest that treats the disease, the fourth nucleic acid sequence being operably linked to an exogenous inducible promoter comprising the specific polynucleotide binding site; and (c) delivering the cell into the subject.

In one aspect, the present disclosure provides a method for introducing a modified cell into a subject in need of disease treatment or prevention, the method comprising: (a) providing a population of cells; (b) introducing at least one nucleic acid molecule or first polynucleotide and second polynucleotide of any of the above-listed aspects into at least one cell in the population of cells; and delivering the cell into the subject.

In one embodiment, the present disclosure provides a method for genetically modifying one or more cells in a subject in need of disease treatment or prevention, the method comprising: (a) introducing at least one nucleic acid molecule into at least one cell of the subject, wherein the at least one nucleic acid molecule comprises: (i) a first polynucleotide comprising a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD), wherein at least one of the transcription factor activation domain and the transcription factor DNA binding domain, upon expression in the cell, is operably linked to the DRD; and (ii) a second polynucleotide that comprises a fourth nucleic acid sequence that encodes a protein of interest that treats the disease, the fourth nucleic acid sequence being operably linked to an exogenous inducible promoter comprising the specific polynucleotide binding site.

In one aspect, the present disclosure provides a method for genetically modifying one or more cells in a subject in need of disease treatment or prevention, the method comprising: (a) introducing at least one nucleic acid molecule into at least one cell of the subject, wherein the at least one nucleic acid molecule comprises: (i) a first polynucleotide comprising a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD), wherein at least one of the transcription factor activation domain and the transcription factor DNA binding domain, upon expression in the cell, is operably linked to the DRD; and (ii) a second polynucleotide that comprises a fourth nucleic acid sequence that encodes a protein of interest that treats the disease, the fourth nucleic acid sequence being operably linked to an exogenous inducible promoter comprising the specific polynucleotide binding site; and (b) administering a ligand to the subject that stabilizes the DRD sufficiently to enable expression of at least one of the transcription factor activation domain and the transcription factor DNA binding domain in an amount sufficient to form a transcription factor that binds to the specific polynucleotide binding site and enables expression of the protein of interest in the cell; wherein expression of the protein of interest is regulated by the presence of ligand in the subject, and the amount and/or duration of ligand administration is sufficient to produce a therapeutically effective amount of the protein of interest.

In one aspect, the present disclosure provides a method for treating a disease in a subject in need thereof, the method comprising: (a) providing a population of cells; (b) introducing at least one of a first nucleic acid molecule and at least one of a second nucleic acid molecule into at least one cell in the population of cells, wherein: (i) the first nucleic acid molecule comprises a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD), wherein at least one of the transcription factor activation domain and the transcription factor DNA binding domain, upon expression in the cell, is operably linked to the DRD; and (ii) the second nucleic acid molecule comprises a fourth nucleic acid sequence that encodes a protein of interest that treats the disease, the fourth nucleic acid sequence being operably linked to an exogenous inducible promoter comprising the specific polynucleotide binding site; (c) delivering the cell into the subject; and (d) administering a ligand to the subject that stabilizes the DRD sufficiently to enable expression of the transcription factor activation domain and the transcription factor DNA binding domain in an amount sufficient to form a transcription factor that binds to the specific polynucleotide binding site and enables expression of the protein of interest in the cell; wherein expression of the protein of interest is regulated by the presence of ligand in the subject, and the amount and/or duration of ligand administration is sufficient to produce a therapeutically effective amount of the protein of interest.

In one aspect, the present disclosure provides a method for treating a disease in a subject in need thereof, the method comprising: (a) providing a population of cells; (b) introducing at least one of a first nucleic acid molecule and at least one of a second nucleic acid molecule into at least one cell in the population of cells, wherein: (i) the first nucleic acid molecule comprises a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD), wherein at least one of the transcription factor activation domain and the transcription factor DNA binding domain, upon expression in the cell, is operably linked to the DRD; and (ii) the second nucleic acid molecule comprises a fourth nucleic acid sequence that encodes a protein of interest that prevents and/or treats the disease, the fourth nucleic acid sequence being operably linked to an exogenous inducible promoter comprising the specific polynucleotide binding site; and (c) delivering the cell into the subject.

In a related embodiment, the present disclosure provides a method for preventing and/or treating a disease in a subject in need thereof. The method comprises: (a) providing a population of cells; (b) introducing at least one of a first nucleic acid molecule and at least one of a second nucleic acid molecule into at least one cell in the population of cells. In this method example, the first nucleic acid molecule comprises a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD). At least one of the transcription factor activation domain and the transcription factor DNA binding domain, upon expression in the cell, is operably linked to the DRD; and the second nucleic acid molecule comprises a fourth nucleic acid sequence that encodes a protein of interest that prevents and/or treats the disease in a subject in need thereof. The fourth nucleic acid sequence is operably linked to an exogenous inducible promoter comprising the specific polynucleotide binding site. The method also includes the steps (c) delivering the cell into the subject; and (d) administering a ligand to the subject that stabilizes the DRD sufficiently to enable expression of the transcription factor activation domain and the transcription factor DNA binding domain in an amount sufficient to form a transcription factor that binds to the specific polynucleotide binding site and enables expression of the protein of interest in the cell. In this method example, the expression of the protein of interest is regulated by the presence of ligand in the subject, and the amount and/or duration of ligand administration is sufficient to produce a therapeutically effective amount of the protein of interest.

In related embodiments, methods of treatment and prevention of the present disclosure can be accomplished by introducing a single vector into a cell, wherein the vector carries a first nucleic acid molecule and a second nucleic acid molecule, wherein: (i) the first nucleic acid molecule comprises a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD), wherein the transcription factor activation domain and/or the transcription factor DNA binding domain, upon expression in the cell, is operably linked to the DRD; and the second nucleic acid molecule comprises a fourth nucleic acid sequence that encodes a protein of interest that treats or prevents the disease, the fourth nucleic acid sequence being operably linked to an exogenous inducible promoter comprising the specific polynucleotide binding site.

In some alternate embodiments, methods of treatment and prevention of the present disclosure can be accomplished by introducing a first vector and a second vector into a cell, wherein the first vector comprises a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD), wherein the transcription factor activation domain and/or the transcription factor DNA binding domain, upon expression in the cell, is operably linked to the DRD, and the second vector comprises a fourth nucleic acid sequence that encodes a protein of interest that prevents and/or treats the disease, the fourth nucleic acid sequence being operably linked to an exogenous inducible promoter comprising the specific polynucleotide binding site.

In some embodiments, the nucleic acid molecule is introduced into the cell by a plasmid or a viral vector. In some aspects, the viral vector is derived from an adenovirus, adeno-associated virus (AAV), alphavirus, flavivirus, herpes virus, measles virus, rhabdovirus, retrovirus, lentivirus, Newcastle disease virus (NDV), poxvirus, and picornavirus. In some aspects, the viral vector is selected from the group consisting of a lentivirus vector, a gamma retrovirus vector, adeno-associated virus (AAV) vector, adenovirus vector, and a herpes virus vector.

In some embodiments, the nucleic acid molecule is introduced into the cell by a non-viral delivery method.

Also provided by the present disclosure is a system for the tunable expression of a protein of interest in a cell, the system comprising: (a) a first polynucleotide encoding a transcription factor linked to a drug response domain (DRD), the transcription factor selectively transcribes a polynucleotide sequence encoding the protein of interest; (b) a second polynucleotide comprising an exogenous transcription factor binding site positioned upstream from and adjacent to a nucleic acid sequence encoding the protein of interest; (c) introducing the first polynucleotide and the second polynucleotide to the cell under conditions to stably integrate the first polynucleotide and the second polynucleotide into the genome of the cell; (d) tuning the expression of the transcription factor by adding a ligand which stabilizes the DRD; wherein the transcription factor specifically binds to a transcription factor binding site positioned upstream from and adjacent to the polynucleotide sequence which encodes the protein of interest, and wherein the expression of the protein of interest is regulated by the quantity of transcription factor present in the cell.

The present disclosure also provides pharmaceutical compositions that include the compositions described herein and a pharmaceutically acceptable excipient.

### BRIEF DESCRIPTION OF THE DRAWINGS

FIG. 1A-FIG. 1B depict schematic illustrations of a transcription factor system design scheme. FIG. 1A shows a schematic of a transcription factor construct, referred to as “DRD-TF construct”, comprising a nucleic acid sequence encoding a transcription factor DNA binding domain, a transcription factor activation domain, and a drug responsive domain (DRD). FIG. 1B shows a schematic of a payload construct, comprising an inducible promoter that comprises binding sites for the transcription factor DNA binding domain.

FIG. 2A-FIG. 2B show ligand-dependent activity of transcription factor systems comprising DRD-regulated transcription factors with different DRDs. FIG. 2A shows a western blot of lysates from untransfected (“mock”) HEK293T cells and HEK293T cells transfected with a construct encoding a constitutive transcription factor (construct ZFHD-055; “Cons.”) or a construct encoding a transcription factor operably linked to a DRD derived from CA2, ecDHFR, ER, or hDHFR parent protein. Details on each construct and ligand treatment conditions are provided in Table 4 and Table 6. The top panel of the western blot shows bands for the endogenous p65, the transcription factor and DRD polypeptide encoded by each of the DRD-TF constructs, and the transcription factor polypeptide encoded by the constitutive construct ZFHD-055. FIG. 2B shows quantification of the western blot in FIG. 2A, normalized with the constitutive condition set to 1.0.

FIG. 3A-FIG. 3E shows ligand-dependent activity of a transcription factor system comprising an ecDHFR DRD-regulated transcription factor. FIG. 3A shows a schematic of the transcription factor construct ZFHD-005. FIG. 3B shows a schematic of the payload construct ZFHD-007. FIG. 3C shows a schematic of a constitutive transcription factor construct ZFHD-004. FIG. 3D shows a western blot of lysates from U2OS cells with stable integration of the indicated constructs, treated with 10 μM TMP or 0.1% DMSO. The band appearing at approximately 60 kDa represents endogenous p65. The band appearing at approximately 44.3 kDa represents the transcription factor and DRD polypeptide encoded by the transcription factor construct ZFHD-005. The band appearing at approximately 26.5 kDa represents the transcription factor polypeptide encoded by construct ZFHD-004. FIG. 3E shows GFP median fluorescence intensity (MFI) as assessed by flow cytometry on U2OS cells with stable integration of the indicated constructs, treated with 10 μM TMP or 0.1% DMSO.

FIG. 4A-FIG. 4C shows dose-response to ligand of a transcription factor system comprising an ecDHFR DRD-regulated transcription factor. FIG. 4A shows a western blot of lysates from U2OS cells with stably integrated constructs ZFHD-005 and ZFHD-007, treated with DMSO or the indicated concentrations of TMP. The lane labeled “U2OS” represents untransduced U2OS cells treated with TMP. The band appearing at approximately 60 kDa represents endogenous p65. The band appearing at approximately 44.3 kDa represents the transcription factor and DRD polypeptide encoded by the transcription factor construct ZFHD-005. FIG. 4B shows quantification of the indicated “ZFHD-005 polypeptide” bands in the western blot of FIG. 4A. Fluorescence is normalized to endogenous p65. FIG. 4C shows GFP median fluorescence intensity (MFI) as assessed by flow cytometry on U2OS cells with stably integrated constructs ZFHD-005 and ZFHD-007, treated with the indicated concentrations of TMP. The highest concentration of TMP used in FIG. 4C is 33 μM. Data shown is for 3 replicates. Error bars represent standard deviation.

FIG. 5A-FIG. 5B shows ligand-dependent activity of a transcription factor system comprising an ecDHFR DRD-regulated transcription factor in T cells. FIG. 5A shows a western blot of lysates from untransduced T cells or T cells transduced with virus (OTLV-ZFHD-005 or OTLV-ZFHD-007) and treated with TMP or DMSO. The band appearing at approximately 60 kDa represents endogenous p65. The band appearing at approximately 44.3 kDa (indicated by the arrow) represents the transcription factor and DRD polypeptide encoded by the transcription factor construct ZFHD-005. FIG. 5B shows GFP median fluorescence intensity (MFI) as assessed by flow cytometry of untransduced T cells or T cells transduced with virus made from the indicated constructs and treated with TMP or DMSO. Data shown is for 3 replicates. Error bars represent standard deviation from the mean.

FIG. 6A-FIG. 6D shows ligand-dependent activity of a transcription factor system comprising a CA2 DRD-regulated transcription factor in ARPE-19 cells. FIG. 6A shows a schematic of the transcription factor construct ZFHD-019. FIG. 6B shows a western blot of lysates from untransduced ARPE-19 cells or ARPE-19 cells with stably integrated constructs ZFHD-019 and ZFHD-007, treated with 10 μM ACZ or 1% DMSO. The band appearing at approximately 60 kDa represents endogenous p65. The band appearing at approximately 55.8 kDa represents the transcription factor and DRD polypeptide encoded by the transcription factor construct ZFHD-019.

FIG. 6C shows quantification of the indicated “ZFHD-019 polypeptide” band in the western blot of FIG. 6B. Fluorescence is normalized to endogenous p65. FIG. 6D shows GFP mean fluorescence intensity (MFI) as assessed by flow cytometry of untransduced ARPE-19 cells or ARPE-19 cells with stable integration of the indicated constructs, either untreated or treated with 10 μM ACZ or 1% DMSO. Data shown is for 3 replicates. Error bars represent standard deviation from the mean. The untransduced ARPE-19 cells and ARPE-19 cells with stable integration of construct ZFHD-007 shown on the graph were treated with DMSO.

FIG. 7A-FIG. 7B shows dose-response to ligand of a transcription factor system comprising a CA2 DRD-regulated transcription factor. FIG. 7A shows a western blot of lysates from ARPE-19 cells with stably integrated constructs ZFHD-007 and ZFHD-019, treated with the indicated concentrations of ACZ. The band appearing at approximately 60 kDa represents endogenous p65. The band appearing at approximately 55.8 kDa represents the transcription factor and DRD polypeptide encoded by the transcription factor construct ZFHD-019. FIG. 7B shows quantification of the indicated “ZFHD-019 polypeptide” bands in the western blot of FIG. 7A. Fluorescence is normalized to the endogenous P65 band.

FIG. 8 shows dose-response to ligand of a transcription factor system comprising a CA2 DRD-regulated transcription factor. The graph shows GFP median fluorescence intensity (MFI) as assessed by flow cytometry of U2OS cells with stable integration of constructs ZFHD-007 and ZFHD-019, treated with the indicated concentrations of ACZ. Data shown is for 2 replicates. Error bars represent standard deviation.

FIG. 9A-FIG. 9C shows ligand-dependent activity of a transcription factor system comprising a CA2 DRD-regulated transcription factor in Jurkat cells. FIG. 9A shows a schematic of the transcription factor construct ZFHD-048. FIG. 9B shows a schematic of the payload construct ZFHD-022. FIG. 9C shows GFP median fluorescence intensity (MFI) as assessed by flow cytometry on Jurkat cells with stable integration of constructs ZFHD-048 and ZFHD-022, treated with DMSO (0.1%) or ACZ (10 μM final concentration). The data presented is for cells that were positive for the transduction marker.

FIG. 10A-FIG. 10F shows ligand-dependent activity of single vector transcription factor systems comprising ecDHFR DRD-regulated transcription factors. FIG. 10A shows a schematic of construct ZFHD-012. FIG. 10B shows a schematic of construct ZFHD-018. FIG. 10C and FIG. 10D show western blots of lysates from U2OS cells transduced with lentiviruses made from the indicated constructs and treated with 10 μM TMP or 0.1% DMSO. The band appearing at approximately 44.3 kDa represents the transcription factor and DRD polypeptide encoded by the indicated constructs. In the single vector constructs, there is a stop codon at the end of the EGFP sequence and a stop codon at the end of the transcription factor-DRD sequence, thus resulting in the approximate 44.3 kDa bands representative of the transcription factor and DRD polypeptide. FIG. 10E and FIG. 10F show GFP median fluorescence intensity (MFI) as assessed by flow cytometry for U2OS cells transduced with lentiviruses made from the indicated constructs and treated with 10 μM TMP or 0.1% DMSO.

FIG. 11A-FIG. 11B shows ligand-dependent activity of a single vector transcription factor system comprising a CA2 DRD-regulated transcription factor. FIG. 11A shows a schematic of the single vector system, depicted as construct ZFHD-036. FIG. 11B show GFP median fluorescence intensity (MFI) as assessed by flow cytometry for Jurkat cells transduced with lentiviruses made from the indicated constructs and treated with 10 μM ACZ or 0.1% DMSO. ZFHD-036.1 and ZFHD-036.2 on the graph represent two cell lines, each transduced with lentivirus made from construct ZFHD-036.

FIG. 12A-FIG. 12B shows ligand-dependent activity of transcription factor systems comprising variants of transcription factor constructs. FIG. 12A shows schematic diagrams of transcription factor construct variants. FIG. 12B shows GFP median fluorescence intensity (MFI) as assessed by flow cytometry on U2OS cells with stable integration of the indicated constructs, treated with either 0.1% DMSO or 10 μM TMP.

FIG. 13 shows ligand-response timecourse analyses of transcription factor systems comprising variants of transcription factor constructs. The graph shows GFP median fluorescence intensity (MFI) as assessed by flow cytometry for U2OS cells with stable integration of the indicated constructs, treated for the indicated time periods with either 0.1% DMSO or 10 μM TMP.

FIG. 14A-FIG. 14D shows ligand-dependent activity of transcription factor systems comprising variants of payload constructs. FIG. 14A shows a schematic diagram of payload construct ZFHD-007. FIG. 14B shows a schematic diagram of payload construct ZFHD-017. FIG. 14C-FIG. 14D show GFP median fluorescence intensity (MFI) as assessed by flow cytometry on U2OS cells with stable integration of the indicated constructs, treated with either 0.1% DMSO or 10 μM TMP.

FIG. 15 shows ligand-dependent activity of a transcription factor system comprising a payload construct encoding a secreted IL12 payload. The graph shows concentration of secreted IL12 in supernatants collected from U2OS cells with stable integration of the indicated constructs, treated with either 0.1% DMSO or 10 μM TMP.

FIG. 16A-FIG. 16D show ligand-dependent regulation of different transcription factors operably linked to a DRD derived from a parent CA2 protein. FIG. 16A shows a western blot of lysates from untransfected (“mock”) HEK293T cells and HEK293T cells transfected with the following constructs: (1) cjun-001 (“001”), (2) cjun-002 (“002”), or (3) cjun-003 (“003”). Each construct-transfected cell population is shown after treatment with DMSO or ACZ (indicated by “+” symbol). The labeled bands “c-Jun-001 and -002 polypeptides” identify the CA2-linker-C-jun polypeptides encoded by the cjun-001 and cjun-002 constructs. The labeled bands “c-Jun-003 polypeptide” identify the c-Jun polypeptide encoded by construct cjun-003. FIG. 16B shows quantification of the western blot in FIG. 16A. FIG. 16C shows a western blot of lysates from untransfected (“mock”) HEK293T cells and HEK293T cells transfected with the following constructs: (1) FOXP3-013 (“013”), (2) FOXP3-014 (“014”), or (3) FOXP3-015 (“015”). Each construct-transfected cell population is shown after treatment with DMSO or ACZ (indicated by “+” symbol). The labeled bands “FOXP3-013 and -014 polypeptide” identify the CA2-FOXP3 polypeptides encoded by the FOXP3-013 and FOXP3-014 constructs. The labeled bands “FOXP3-015 polypeptide” identify the FOXP3 polypeptide encoded by construct FOXP3-015. FIG. 16D shows quantification of the western blot in FIG. 16C.

FIG. 17A-FIG. 17B show ligand-dependent regulation of c-Jun transcription factor construct stably integrated in Jurkat cells. FIG. 17A shows a western blot of lysates from untransduced (“mock”) Jurkat cells and Jurkat cells transduced with lentivirus made from constructs cjun-001 (“001”) and cjun-002 (“002”). Each construct-transduced cell line is shown after treatment with DMSO or ACZ (indicated by “+” symbol). Bands for a c-Jun polypeptide and a phosphorylated c-Jun polypeptide are shown. FIG. 17B shows quantification of the western blot in FIG. 17A.

FIG. 18 shows the nucleotide sequence of the pELDS-puro transfer vector (SEQ ID NO: 68).

FIG. 19 shows the nucleotide sequence of the pELNS-puro transfer vector (SEQ ID NO: 69).

### DETAILED DESCRIPTION

### Transcription Factor System

According to the present disclosure, a transcription factor system is a combination of one or more polynucleotides that comprise (1) one or more nucleic acid sequences that encode a transcription factor that is able to bind to a specific polynucleotide binding site and activate transcription; (2) a nucleic acid sequence that encodes a drug responsive domain (DRD), wherein the transcription factor is operably linked to the DRD; and (3) a nucleic acid sequence that encodes a payload and is operably linked to an inducible promoter comprising the specific polynucleotide binding site.

In some embodiments, the combination of one or more polynucleotides of a transcription factor system can be used to modify cells, for example, immune cells useful in the treatment of a disease and create systems for regulatable expression of a protein of interest by regulating the presence of a transcription factor acting on the polynucleotide(s) encoding the payload or protein of interest.

In some embodiments, the combination of one or more polynucleotides of a transcription factor system comprises a polynucleotide that comprises a first nucleic acid sequence encoding the transcription factor and a second nucleic acid sequence encoding the DRD.

The present disclosure also provides a first polynucleotide and second polynucleotide, wherein the first polynucleotide comprises: a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD). In this example, at least one of the transcription factor activation domain, the transcription factor DNA binding domain, or the combination of the transcription factor activation domain and the transcription factor DNA binding domain is operably linked to the DRD exemplified herein. The second polynucleotide comprises: a fourth nucleic acid sequence that encodes a protein of interest, the fourth nucleic acid sequence being operably linked to an inducible promoter comprising the specific polynucleotide binding site. In this example, the transcription factor activation domain and the transcription factor DNA binding domain interact to form a transcription factor that is able to activate transcription upon binding to the specific polynucleotide binding site, and the first polynucleotide and the second polynucleotide are each carried in a single vector, or the first polynucleotide and the second polynucleotide are carried in separate vectors.

In a related example, the present disclosure provides compositions and nucleic acids that are operable to regulate transcription. For example, the present disclosure provides a first polynucleotide and second polynucleotide of the regulatable transcription factor system. The first polynucleotide comprises a first nucleic acid sequence that encodes a transcription factor and a second nucleic acid sequence that encodes a drug responsive domain (DRD), wherein the transcription factor is operably linked to the DRD and wherein the transcription factor is able to activate transcription upon binding to a specific polynucleotide binding site. The second polynucleotide comprises: a third nucleic acid sequence that encodes a protein of interest, the third nucleic acid sequence being operably linked to an inducible promoter comprising the specific polynucleotide binding site; such that the first polynucleotide and the second polynucleotide are each carried in a single vector, or the first polynucleotide and the second polynucleotide are carried in separate vectors.

In some embodiments, the combination of one or more polynucleotides of a transcription factor system comprises a first nucleic acid sequence encoding a transcription factor DNA binding domain that binds to a specific polynucleotide binding site; a second nucleic acid sequence encoding a transcription factor activation domain; and a third nucleic acid sequence encoding the DRD. In some aspects, the combination of one or more polynucleotides of a transcription factor system comprises a polynucleotide that comprises the first, second and third nucleic acid sequences. In some aspects, the combination of one or more polynucleotides of a transcription factor system comprises a polynucleotide that comprises two of the first, second and third nucleic acid sequences. In some aspects, the combination of one or more polynucleotides of a transcription factor system comprises a first polynucleotide comprising the first nucleic acid sequence; a second polynucleotide comprising the second nucleic acid sequence; and a third polynucleotide comprising the third nucleic acid sequence. In one aspect, the transcription factor DNA binding domain is operably linked to the DRD. In another aspect, the transcription factor activation domain is operably linked to the DRD. In another aspect, both the transcription factor DNA binding domain and the transcription factor activation domain are operably linked to the DRD. In some aspects, the transcription factor DNA binding domain and the transcription factor activation domain are expressed as a transcription factor fusion protein.

According to the present disclosure, a transcription factor system encodes a transcription factor that can drive expression of a payload. In some embodiments, the transcription factor is encoded by a first nucleic acid sequence that encodes a transcription factor activation domain and a second nucleic acid sequence that encodes a transcription factor DNA binding domain that binds to a specific polynucleotide binding site. The transcription factor activation domain and the transcription factor DNA binding domain interact to form a transcription factor that activates transcription of the nucleic acid sequence encoding the payload upon binding to the specific polynucleotide binding site.

In some embodiments, the specific polynucleotide binding site comprises at least one nucleic acid site with a specific sequence that is recognized and bound by the transcription factor DNA binding domain. In some embodiments, the specific polynucleotide binding site comprises at least two, at least three, at least four, at least five, at least six, at least seven, at least eight, at least nine or at least ten nucleic acid sites that are recognized by the DNA binding domain of the present disclosure. In some embodiments, the specific polynucleotide binding site comprises eight nucleic acid sites that are recognized by the DNA binding domain. In some embodiments, the specific polynucleotide binding site comprises two or more tandem nucleic acid sites, each with a specific sequence that is recognized and bound by the transcription factor DNA binding domain. In some aspects, the tandem nucleic acid sites comprise identical nucleic acid sequences. In some embodiments, the specific polynucleotide binding site comprises tandem repeat nucleic acid sites that are recognized by the DNA binding domain of the present disclosure.

As described herein, a transcription factor or part thereof, is operably linked to a DRD in a transcription factor system of the present disclosure. The presence, absence or an amount of a ligand that binds to or interacts with the DRD, can, upon such binding or interaction modulate the stability of the transcription factor and consequently the function of the transcription factor. Thus, a transcription factor system can exhibit ligand-dependent activity.

In some embodiments, a transcription factor system is present in a cell or a population of cells. In some embodiments, one or more polynucleotides of a transcription factor system are introduced into a cell or population of cells.

### Transcription Factor System Constructs

The combination of one or more polynucleotides of a transcription factor system may also be referred to herein as a combination of one or more nucleic acid constructs. The polynucleotides or nucleic acid constructs may comprise different arrangements of nucleic acid sequences, and/or may be uniquely combined as part of a transcription factor system, so long as the resulting combination of polynucleotides or nucleic acid constructs comprises (1) one or more nucleic acid sequences that encode a transcription factor that is able to bind to a specific polynucleotide binding site and activate transcription; (2) a nucleic acid sequence that encodes a drug responsive domain (DRD), wherein the transcription factor is operably linked to the DRD; and (3) a nucleic acid sequence that encodes a payload and is operably linked to an inducible promoter comprising the specific polynucleotide binding site.

In some embodiments, a transcription factor system comprises multiple constructs. In some embodiments, a transcription factor system comprises a transcription factor construct and a payload construct. In one aspect, the transcription factor construct comprises a nucleic acid sequence that encodes a transcription factor. In one aspect, the transcription factor construct comprises a nucleic acid sequence that encodes a transcription factor activation domain and a nucleic acid sequence that encodes transcription factor DNA binding domain.

In some embodiments, a transcription factor system comprises a single construct. The single construct comprises nucleic acid sequences encoding the transcription factor, DRD, and payload of the transcription factor system. In some embodiments, such a single construct transcription factor system may be introduced into a cell on a single nucleic acid molecule, such as a plasmid or vector. A transcription factor system comprising a single construct may be referred to herein as a single vector transcription factor system.

In addition to comprising the nucleic acid sequences described herein for a transcription factor system, nucleic acid constructs of the present disclosure may comprise additional nucleic acid sequences. Additional nucleic acid sequences of constructs include, but are not limited to, regulatory elements, polyadenylation sequences, linkers, and cleavage sites.

In some embodiments, a transcription factor construct may comprise nucleic acid sequences encoding: a promoter, a transcription factor DNA binding domain, a transcription factor activation domain, and a DRD. In some embodiments, the nucleic acid sequence encoding the DRD is adjacent to a nucleic acid sequence encoding at least one of the transcription factor domains. In some embodiments, the nucleic acid sequence encoding the DRD is positioned between a nucleic acid sequence encoding the transcription factor DNA binding domain and the transcription factor activation domain.

In some embodiments, a transcription factor construct may comprise nucleic acid sequences encoding: a promoter, a transcription factor DNA binding domain, a transcription factor activation domain, a linker, and a DRD. In some aspects, the linker is positioned between a nucleic acid sequence encoding a transcription factor domain and the nucleic acid sequence encoding the DRD.

In some embodiments, a promoter in a transcription factor construct is EF1a. In some embodiments, the encoded transcription factor DNA binding domain in a transcription factor construct is ZFHD1. In some embodiments, the encoded transcription factor activation domain in a transcription factor construct is p65.

In some embodiments, a payload construct may comprise nucleic acid sequences encoding: a specific polynucleotide binding site comprising at least one nucleic acid site with a specific sequence recognized and bound by the transcription factor DNA binding domain, a promoter, and payload. An exemplary binding site comprises eight (8) nucleic acid sites that are recognized by a ZFHID1 DNA binding domain.

In some embodiments, a construct of the present disclosure, such as a transcription factor construct or a payload construct, is integrated into a plasmid or viral vector. In some embodiments, the plasmid or viral vector comprises one or more regulatory elements that become operably linked to one or more components of the construct that is integrated into the plasmid or viral vector. In some embodiments, the plasmid or viral vector comprises regulatory elements well known in the art, including for example promoters, introns, spacers, stuffer sequences, and the like. In some embodiments, a transcription factor construct is integrated into a plasmid or viral vector such that the components of the transcription factor construct are operably linked to regulatory elements of the plasmid or viral vector. In some embodiments, such a transcription factor construct comprises nucleic acid sequences encoding: a transcription factor DNA binding domain, a transcription factor activation domain, and a DRD, and is integrated into the plasmid or viral vector such that a promoter sequence in the plasmid or viral vector drives expression of the transcription factor DNA binding domain, transcription factor activation domain, and DRD. Such a promoter may be selected from a constitutive promoter, a tissue-specific promoter, a cell-specific promoter, a cell differentiation-specific promoter, and/or a disease-specific promoter. Optionally, the promoter may be selected from EF1a, CMV, EFS, RSV, SFFV, PGK, CAG, and SV40.

### Components of Transcription Factor Systems

As stated above, the polynucleotides or nucleic acid constructs of a transcription factor system may comprise different arrangements of nucleic acid sequences, and/or may be uniquely combined as part of a transcription factor system, so long as the resulting combination of polynucleotides or nucleic acid constructs comprises (1) one or more nucleic acid sequences that encode a transcription factor that is able to bind to a specific polynucleotide binding site and activate transcription; (2) a nucleic acid sequence that encodes a drug responsive domain (DRD), wherein the transcription factor is operably linked to the DRD; and (3) a nucleic acid sequence that encodes a payload and is operably linked to an inducible promoter comprising the specific polynucleotide binding site. In this way, the transcription factor system is a modular system and each component of the transcription factor system can be selected separately.

The nucleic acid sequence that encodes a drug responsive domain (DRD) may be selected from sequences of DRDs described in more detail in the “Drug responsive domains (DRDs)” section below.

The one or more nucleic acid sequences that encode a transcription factor may be selected from one or more sequences that encode an existing transcription factor, an engineered transcription factor that has been derived from an existing transcription factor, or an engineered transcription factor that comprises a DNA binding domain and an activation domain. As used herein, an “engineered transcription factor that has been derived from an existing transcription factor” refers to an engineered transcription factor that originates at least in part from the parent (native) transcription factor molecule or sequence and retains the ability to bind a specific polynucleotide binding site and activate transcription. For example, an engineered transcription factor may be derived from a parent transcription factor comprising one or more zinc finger domains capable of sequence-specific contacts with DNA. An engineered TAL effector transcription factor may be designed to comprise a TAL effector repetitive regions recognizing a specific DNA binding site, a mammalian nuclear localization signal (NLS) and a synthetic transcription activation domain. If the transcription factor is an engineered transcription factor that comprises a DNA binding domain and an activation domain, both the DNA binding domain and the activation domain may be separately selected and combined to form the complete transcription factor.

The transcription factor DNA binding domain may be derived from an existing nucleic acid binding protein. For example, a DNA-binding sequence or domain of an existing DNA binding protein may be used as or further modified to generate the transcription factor DNA binding domain of the present disclosure.

In some aspects, the transcription factor DNA binding domain is derived from a parent protein selected from the group consisting of: ZFHD1, Cas9, Cas12, and TAL.

In some embodiments, the transcription factor DNA binding domain is derived from a ZFHD1 parent protein. ZFHD1 is a zinc finger-homeodomain fusion protein designed by Pomerantz, J. L., et al. (Pomerantz, J. L., et al. “Structure-Based Design of Transcription Factors.” Science, vol. 267, no. 5194, 1995). ZFHD1 comprises fingers 1 and 2 of Zif268, a gly-gly-arg-arg linker, and the OCT-1 homeodomain. ZFHD1 can bind to a nucleic acid sequence comprising the sequence TAATGATGGGCG (SEQ ID NO: 70). In some embodiments, the transcription factor DNA binding domain consists of or comprises the amino acid sequence of ZFHD1.

In some embodiments, the present disclosure provides methods of regulating target genes and their corresponding functional proteins (e.g., payload or protein of interest) using a Cas/guide RNA system. It is to be understood that one of skill will be able to design suitable guide RNA for forming a co-localization complex with a target nucleic acid including a target gene as described herein.

Various Cas proteins are known to those of skill in the art and include CasI (Cas3), Cas IA (Cas8a), CasIB (Cas8b), CasIC (Cas8c), CasID (Cas10d), CasIE (Cse1), CasIF (Csy1), CasIU, CasII (Cas9), CasIIA (Csn2), CasIIB (Cas4), CasIIC, CasIII (Cas10), CasIIIA (Csm2), CasIIIB (Cmr5), CasIIIC, CasIIBD, CasIV (Csf1), CasIVA, CasIVB, CasV (Cpf1), C2c2, and C2c1 and the like.

In some embodiments, the transcription factor DNA binding domain is derived from a Cas protein selected from the group consisting of C2C1, C2C3, Cpf1 (also referred to as Cas12a), Cas12b, Cas12c, Cas12d, Cas12e, Cas13a, Cas13b, Cas13c, Cas13d, Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

According to one aspect, the Cas9 protein includes the sequence as set forth for naturally occurring Cas9 from S. aureus, S. thermophiles, S. pyogenes or Neisseria meningitidis Cas9 and protein sequences having at least 30%, 40%, 50%, 60%, 70%, 80%, 90%, 95%, 98% or 99% homology thereto and being a DNA binding protein, such as an RNA guided DNA binding protein.

According to one aspect, the Cas12 protein includes the sequence as set forth for naturally occurring Cas12 from Francisella novicida, Acidaminococcus sp., Lachnospiraceae sp., or Prevotella sp. and protein sequences having at least 30%, 40%, 50%, 60%, 70%, 80%, 90%, 95%, 98% or 99% homology thereto and being a DNA binding protein, such as an RNA guided DNA binding protein.

In some embodiments, the transcription factor DNA binding domain is derived from a parent Cas protein, such as a parent Cas9 or Cas12 protein. In some embodiments, the transcription factor DNA binding domain is or comprises a Cas9 that has been modified to lack nuclease activity. In some embodiments, the transcription factor DNA binding domain is or comprises a Cas12 that has been modified to lack nuclease activity.

Naturally occurring Cas9 comprises two nuclease domains: an HNH-like nuclease domain that cleaves the DNA strand complementary to the guide RNA sequence (target strand), and a RuvC-like nuclease domain that cleaves the DNA strand opposite the complementary strand (nontarget strand). By mutating both the HNH and RuvC nuclease domains (resulting in the so-called “dead Cas9” or “dCas9”), the resulting dCas9 retains its RNA-guided DNA targeting ability but loses its endonuclease activity. In some embodiments, the transcription factor DNA binding domain is a dCas9 comprising mutated HNH and RuvC nuclease domains. In some embodiments, the transcription factor DNA binding domain is a dCas9 comprising mutated HNH and RuvC nuclease domains and is derived from a parent S. aureus, S. thermophiles, S. pyogenes or Neisseria meningitidis Cas9.

Naturally occurring Cas12 (e.g., Cas12a and Cas12b) comprises a RuvC-like domain that cleaves DNA. By mutating the RuvC nuclease domain, a catalytically dead Cas12 (having deactivation of DNase activity, also referred to herein as “dCas12”) may be derived from a parent Cas12 protein. In some embodiments, the transcription factor DNA binding domain is or comprises a catalytically dead Cas12 (dCas12).

In some embodiments, the transcription factor DNA binding domain is derived from a parent protein that is a Type II Cas homolog. Cas9 is an example of a Type II Cas protein. In some embodiments, the transcription factor DNA binding domain is or comprises a Type II Cas homolog that lacks nuclease activity or has been modified to lack nuclease activity. In some embodiments, the transcription factor DNA binding domain is or comprises a Type II Cas homolog comprising mutated HNH and RuvC nuclease domains.

In accordance with an exemplary embodiment, Cas9 is altered or otherwise modified to inactivate the nuclease activity. Such alteration or modification includes altering one or more amino acids to inactivate the nuclease activity or the nuclease domain. Such modification includes removing the polypeptide sequence or polypeptide sequences exhibiting nuclease activity, i.e., the nuclease domain, such that the polypeptide sequence or polypeptide sequences exhibiting nuclease activity, i.e. nuclease domain, are absent from the Cas9 DNA binding protein. Other modifications to inactivate nuclease activity will be readily apparent to one of skill in the art. Accordingly, a nuclease-null DNA binding protein includes polypeptide sequences modified to inactivate nuclease activity or removal of a polypeptide sequence or sequences to inactivate nuclease activity. The nuclease-null DNA binding protein retains the ability to bind to DNA even though the nuclease activity has been inactivated. Accordingly, the DNA binding protein includes the polypeptide sequence or sequences required for DNA binding but may lack the one or more or all of the nuclease sequences exhibiting nuclease activity. See Jinek et al., (2012) Science 337, 816-821. A Cas9 protein lacking nuclease activity is referred to as a nuclease-null Cas9 (“Cas9Nuc”, “dead Cas9” or “dCas9”) and exhibits reduced or eliminated nuclease activity, or nuclease activity is absent or substantially absent within levels of detection. According to this aspect, nuclease activity for a Cas9Nuc may be undetectable using known assays, i.e. below the level of detection of known assays.

In some embodiments, the transcription factor DNA binding domain is derived from a Cas9 parent protein. In some embodiments, the transcription factor DNA binding domain comprises a Cas9 having mutated nuclease domains (referred to as “dead Cas9” or “dCas9”). The resulting dCas9 retains its RNA-guided DNA targeting ability but loses its endonuclease activity. In some embodiments, the transcription factor DNA binding domain is a dCas9.

The present disclosure provides for the use of guide RNA to target a Cas protein, for example a nuclease-null Cas9 operably linked to a DRD, to a polynucleotide binding sequence as described herein. Such guide RNA can be readily designed by those of skill in the art when knowing the particular polynucleotide binding sequence. A guide RNA may include one or more of a spacer sequence, a tracr mate sequence and a tracr sequence. The term spacer sequence is understood by those of skill in the art and may include any polynucleotide having sufficient complementarity with a polynucleotide binding sequence to hybridize with the polynucleotide binding sequence and direct sequence-specific binding of a CRISPR complex to the polynucleotide binding sequence. The guide RNA may be formed from a spacer sequence covalently connected to a tracr mate sequence (which may be referred to as a crRNA) and a separate tracr sequence, wherein the tracr mate sequence is hybridized to a portion of the tracr sequence. According to certain aspects, the tracr mate sequence and the tracr sequence are connected or linked such as by covalent bonds by a linker sequence, which construct may be referred to as a fusion of the tracr mate sequence and the tracr sequence. The linker sequence referred to herein is a sequence of nucleotides, referred to herein as a nucleic acid sequence, which connect the tracr mate sequence and the tracr sequence. Accordingly, a guide RNA may be a two component species (i.e., separate crRNA and tracr RNA which hybridize together) or a unimolecular species (i.e., a crRNA-tracr RNA fusion, often termed a sgRNA).

In some embodiments, the guide RNA may be delivered directly to a cell as a native species by methods known to those of skill in the art, including injection or lipofection, or as transcribed from its cognate DNA, with the cognate DNA introduced into cells through electroporation, transient and stable transfection (including lipofection) and viral transduction.

In some embodiments, a transcription factor system comprises one or more polynucleotides encoding a DRD-regulated transcription factor, wherein the transcription factor comprises a DNA binding domain that is or comprises a nuclease-null Cas9. When the DRD stabilizing ligand is added, the DRD and transcription factor is stabilized and the nuclease-null Cas9 is expressed and available to bind to the guide RNA. Upon binding with the guide RNA, the Cas9-gRNA system binds to the polynucleotide binding sequence which is operably linked to the protein of interest. When the Cas9-gRNA system is bound to the polynucleotide binding sequence, the protein of interest gene is transcribed due to the presence of the transcription factor activation domain. Thus, when the regulatable transcription factor expression construct comprises the Cas9-gRNA system, RNA-guided DNA regulation is effected in cells such as human cells by tethering or connecting DRDs to either a nuclease-null Cas9 or to transcription factor activation domains. Accordingly, aspects of the present disclosure include methods and materials for localizing transcriptional regulatory domains to targeted loci by fusing, connecting or joining a DRD to either Cas9Nuc or to a transcription factor activation domain, or both.

In some embodiments, the transcription factor DNA binding domain is derived from a TAL parent protein. TAL (transcription activator-like) effectors (also referred to as “TALEs”) are proteins secreted by Xanthomonas bacteria to modulate gene expression in host plants and aid bacterial infection. TAL effectors have a repetitive region consisting of tandem repeats of mostly 33 or 34 amino acid residues. Repeat monomers differ from each other mainly in amino acid positions 12 and 13, and there is a strong correlation between unique pairs of amino acids at positions 12 and 13 and the corresponding nucleotide in the TALE-binding site. The transcription factor DNA binding domain of the present disclosure may comprise all or a portion of the repetitive region of a TAL effector that is capable of binding to a specific DNA binding site. In some embodiments, the DNA binding domain comprises a synthetic TAL effector capable of recognizing a desired nucleic acid sequence. Methods for assembling custom TAL effectors are readily available to one of skill in the art. An “engineered TAL effector” refers herein to a polypeptide derived from a parent TAL effector protein, a polypeptide comprising the repetitive region of a TAL effector and/or a synthetic TAL effector or region thereof. In some embodiments, the transcription factor DNA binding domain is an engineered TAL effector capable of binding to a specific nucleic acid site. In some embodiments, the transcription factor DNA binding domain is derived from a zinc finger protein parent protein. In some embodiments, the parent zinc finger protein may a C2H2 zinc finger protein. In some embodiments, the transcription factor DNA binding domain may comprise one or more zinc finger domains that make sequence-specific contacts with DNA. In some embodiments, the transcription factor DNA binding domain may comprise at least two zinc finger domains, at least three zinc finger domains, at least four zinc finger domains, or at least five zinc finger domains that form a zinc finger array capable of specifically recognizing a DNA site. In some embodiments, the transcription factor DNA binding domain comprises a three-finger array. An engineered DNA binding domain comprising one or more zinc finger domains is referred to herein as an “engineered zinc finger binding protein”.

In some embodiments, the transcription factor DNA binding domain may be selected from an engineered zinc finger binding protein, engineered TAL effector, or other natural or engineered DNA binding domain.

Zinc finger and TALE DNA binding domains can be “engineered” to bind to a predetermined nucleotide sequence, for example via engineering (altering one or more amino acids) of the recognition region of a naturally occurring zinc finger or TALE protein. Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 8,586,526; 6,140,081; 6,453,242; 6,534,261 and 8,586,526; see also WO 98/53058; WO 98/53059; WO 98/53060; WO 02/016536 and WO 03/016496, the disclosures of these references as it pertains to the design and selection of DNA binding proteins derived from existing ZFP and/or TALE proteins and binding data related thereto, is incorporated by reference herein it their entireties.

The activation domain of an engineered transcription factor according to the present disclosure may be derived from a region or domain of an existing transcription factor. In some embodiments the activation domain is a region of an existing transcription factor that is capable of transcription activation. In some embodiments, the transcription factor activation domain may be selected from the activation domains of p65, VP64, p300, SAM, VPR, or other activation domains. In some embodiments, the activation domain is derived from the carboxy terminal region of the human transcription factor NF-κβ p65 protein (referred to herein as “p65”). In some embodiments, the activation domain comprises the carboxy terminal region of the human transcription factor NF-κβ p65 protein.

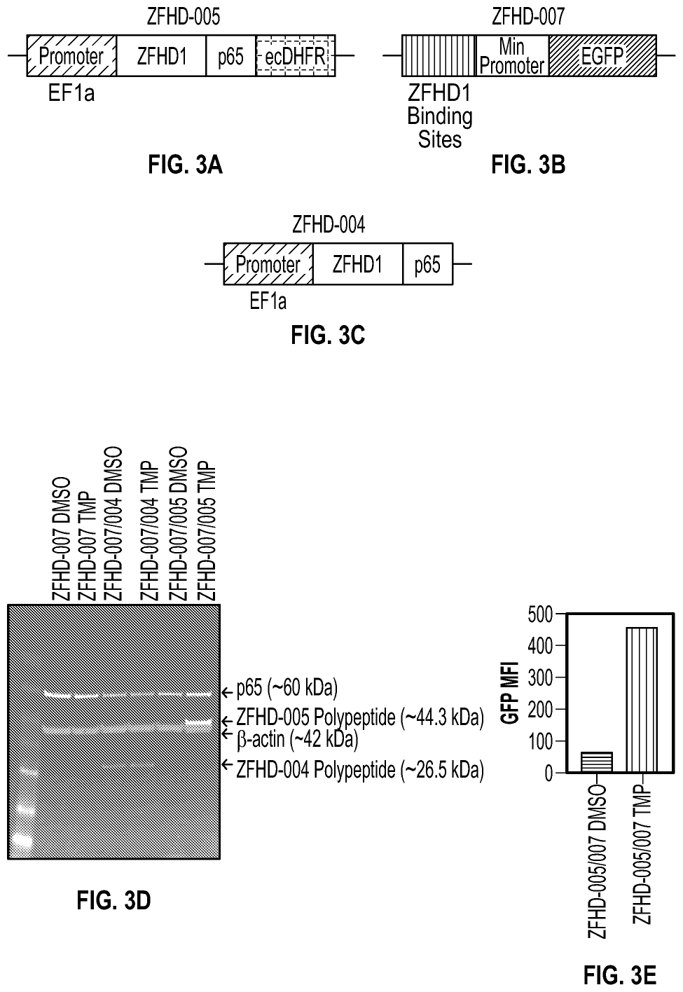

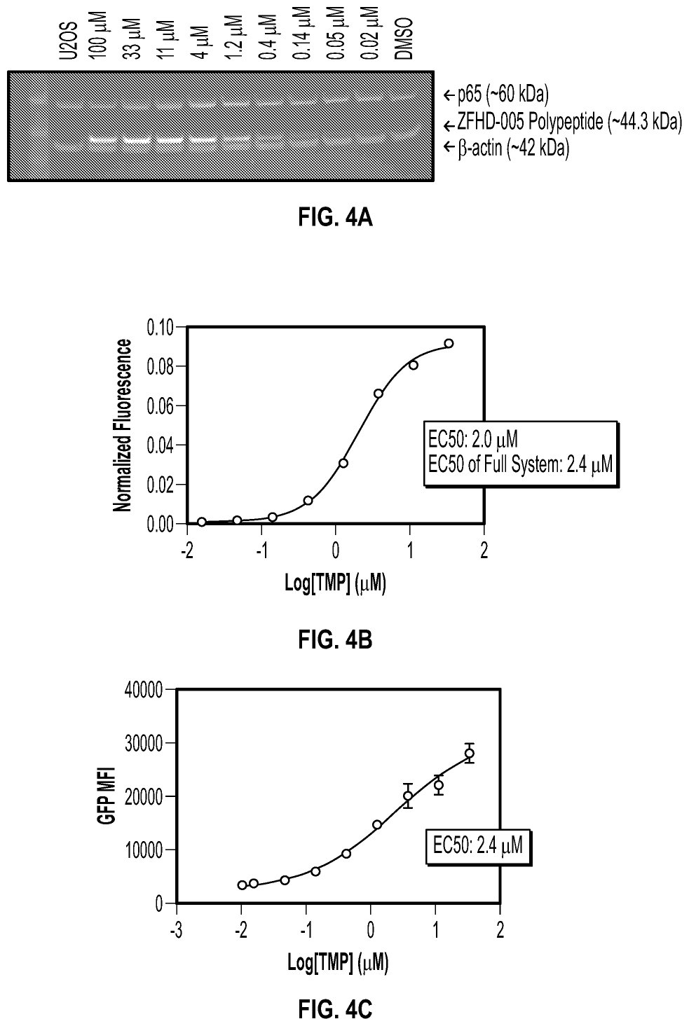

A consideration in the design of the transcription factor system provided herein is that the encoded transcription factor is able to bind to a specific polynucleotide binding site and that the nucleic acid sequence that encodes the payload is operably linked to an inducible promoter comprising the specific polynucleotide binding site. In various embodiments, the inducible promoter is an exogenous inducible promoter. Pairs of transcription factors (including engineered transcription factors) and their corresponding polynucleotide binding sites are known in the art. Also known are DNA binding domains of DNA binding proteins along with their corresponding polynucleotide binding sites and method for identifying new DNA binding domain sequences and corresponding polynucleotide binding sites that can be used for the design of synthetic transcription factors and corresponding synthetic promoters. For example, Khalil A. S., et al. provide zinc finger arrays that can be used as core building blocks for constructing synthetic transcription factors and further provide corresponding nucleic acid binding sequences that can be inserted within synthetic promoters and recognized by the zinc finger arrays (Khalil A. S., et al. Cell 2012, 150, 647-658, incorporated by reference in its entirety). Khalil A. S., et al. also identify synthetic transcription factor-promoter pairs and provide design strategies to modify transcriptional output by changing the promoters (e.g, multimerizing zinc finger binding sequences to create promoters with repeat operators) and changing the synthetic transcription factors (e.g., by creating variants). Any of the transcription factor-promoter pairs or engineered zinc finger arrays and their corresponding nucleic acid binding sites disclosed by Khalil A. S., et al. can be used for the transcription factor systems of the present disclosure. As an example, FIG. 3A of Khalil A. S., et al. provides a library of amino acid residues of the recognitions helices for zinc finger arrays and the corresponding DNA binding sequences which can be used in the design of a transcription factor DNA binding domain and the specific polynucleotide binding site of the present disclosure. One of skill in the art would be able to modify the transcription factors or zinc finger array sequences provided by Khalil, A. S., et al. by cloning the sequences of these transcription factors or arrays into the constructs of a transcription factor system provided herein. As another example, Zhang, F., et al. describe methods for design and production of engineered TAL effectors with corresponding nucleic acid binding sites. These can be used in the preparation of engineered transcription factors and their specific polynucleotide binding site. Any of the TAL effectors provided by Zhang, F., et al. may be used to prepare a transcription factor DNA binding domain in a transcription factor system of the present disclosure. For example, Zhang, F., et al. disclose construction of 17 artificial TAL effectors to target specific DNA binding sites and also provide in FIG. 2a the sequences of TAL effector repeat regions and corresponding nucleic acid binding sequences. The TAL effectors or DNA binding parts thereof disclosed in Zhang, F., et al. can be used to construct the DNA binding domains as well as the corresponding nucleic acid binding sequences for the inducible promoters of the present disclosure. One of skill in the art would recognize that there are several options for selection and design of the DNA binding domains of the present disclosure. In addition to selection of recognized DNA binding proteins and domains known in the art, the DNA binding domain of the present disclosure may be designed based on the frameworks of existing DNA binding proteins. For example, methods for selecting DNA binding domains based on the Cys₂His₂ zinc finger protein framework is available to the skilled artisan (Pabo, C. O., et al. Annu. Rev. Biochem. 2001. 70:313-40).

In some embodiments, the inducible promoter that is operably linked to the nucleic acid sequence that encodes the payload comprises a minimal promoter (also referred to as a “min promoter” or “core promoter”) and the specific polynucleotide binding site. In this scenario, both the minimal promoter and the specific polynucleotide binding site are operably linked to the nucleic acid sequence that encodes the payload. The term “minimal promoter” refers to a minimal structure that enables the formation of the initiation complex. A minimal promoter may comprise an RNA polymerase binding site, TATA box and transcription start site. A minimal promoter may be coupled with one or more response elements (such as enhancers or transcription factor binding sites) to generate an inducible promoter. Additional details regarding minimal promoters and coupling of minimal promoters to response elements is provided by Ede and colleagues (Ede et al., ACS Synth Biol. 2016 May 20; 5(5): 395-404). In some embodiments, the inducible promoter of a transcription factor system or component thereof of the present disclosure comprises a minimal promoter selected from the following minimal promoters: minCMV, CMV53 (minCMV with the addition of an upstream GC box), minSV40 (minimal simian virus 40 promoter), miniTK (the −33 to +32 region of the Herpes simplex thymidine kinase promoter), MLP (the −38 to +6 region of the adenovirus major late promoter), pJB42CAT5 (a minimal promoter derived from the human junB gene), YB_TATA (a synthetic minimal promoter developed by Benenson and colleagues (Hansen, J. et al. Proc Natl Acad Sci USA. 2014; 111:15705-15710)), and the TATA box alone.

As described above, the specific polynucleotide binding site may comprise at least one nucleic acid site with a specific sequence that is recognized and bound by the transcription factor DNA binding domain. In some embodiments, the specific polynucleotide binding site comprises two or more nucleic acid sites, each with a specific sequence that is recognized and bound by the transcription factor DNA binding domain. Pairing of DNA binding domains with their corresponding polynucleotide binding sites is discussed above.

The nucleic acid sequence that encodes a payload may be selected to encode any payload or protein of interest. Additional details regarding payloads are provided in the “Payloads” section below.

Exemplary nucleic acid constructs that may be used individually (as a single construct) or in combination as part of a transcription factor system are described in Table 1. An asterisk (“*”) in Table 1 indicates the translation of a stop codon.

TABLE 1
Exemplary constructs of transcription factor systems

| | | | NA | | AA |
|---|---|---|---|---|---|
| Con- | | | SEQ | | SEQ |
| struct | Construct | | ID | | ID |
| Name | Description | NA Sequence | NO | AA Sequence | NO |
| ZFHD- | EF1a | cgtgaggctccggtgcccgtcagtgggcagagcgcac | 7 | MAPKKKRKVERPYACPVESCDRRFSRSDELTRHIRIH | 8 |
| 004 | promoter; | atcgcccacagtccccgagaagttggggggaggggtc | | TGQKPFQCRICMRNFSRSDHLTTHIRTHTGGGRRRKK | |
| | ZFHD1 DNA | ggcaattgaaccggtgcctagagaaggtggcgcgggg | | RTSIETNIRVALEKSFLENQKPTSEEITMIADQLNME | |
| | binding | taaactgggaaagtgatgtcgtgtactggctccgcct | | KEVIRVWFCNRRQKEKRINTRLGALLGNSTDPAVFTD | |
| | domain; p65 | ttttcccgagggtgggggagaaccgtatataagtgca | | LASVDNSEFQQLLNQGIPVAPHTTEPMLMEYPEAITR | |
| | activation | gtagtcgccgtgaacgttctttttcgcaacgggtttg | | LVTGAQRPPDPAPAPLGAPGLPNGLLSGDEDFSSIAD | |
| | domain | ccgccagaacacaggtaagtgccgtgtgtggttcccg | | MDFSALLSQISSGS | |
| | | cgggcctggcctctttacgggttatggcccttgcgtg | | | |
| | | ccttgaattacttccacctggctgcagtacgtgattc | | | |
| | | ttgatcccgagcttcgggttggaagtgggtgggagag | | | |
| | | ttcgaggccttgcgcttaaggagccccttcgcctcgt | | | |
| | | gcttgagttgaggcctggcctgggcgctggggccgcc | | | |
| | | gcgtgcgaatctggtggcaccttcgcgcctgtctcgc | | | |
| | | tgctttcgataagtctctagccatttaaaatttttga | | | |
| | | tgacctgctgcgacgctttttttctggcaagatagtc | | | |
| | | ttgtaaatgcgggccaagatctgcacactggtatttc | | | |
| | | ggtttttggggccgcgggcggcgacggggcccgtgcg | | | |
| | | tcccagcgcacatgttcggcgaggcggggcctgcgag | | | |
| | | cgcggccaccgagaatcggacgggggtagtctcaagc | | | |
| | | tggccggcctgctctggtgcctggcctcgcgccgccg | | | |
| | | tgtatcgccccgccctgggcggcaaggctggcccggt | | | |
| | | cggcaccagttgcgtgagcggaaagatggccgcttcc | | | |
| | | cggccctgctgcagggagctcaaaatggaggacgcgg | | | |
| | | cgctcgggagagcgggcgggtgagtcacccacacaaa | | | |
| | | ggaaaagggcctttccgtcctcagccgtcgcttcatg | | | |
| | | tgactccactgagtaccgggcgccgtccaggcacctc | | | |
| | | gattagttctcgagcttttggagtacgtcgtctttag | | | |
| | | gttggggggaggggttttatgcgatggagtttcccca | | | |
| | | cactgagtgggtggagactgaagttaggccagcttgg | | | |
| | | cacttgatgtaattctccttggaatttgccctttttg | | | |
| | | agtttggatcttggttcattctcaagcctcagacagt | | | |
| | | ggttcaaagtttttttcttccatttcaggtgtcgtga | | | |
| | | tctagaggatcACTAGTgccaccatgGCACCTAAGaa | | | |
| | | aAAGAGGAAGGTTgaacgcccatatgcttgccctgtc | | | |
| | | gagtcctgcgatcgccgcttttctcgctcggatgagc | | | |
| | | ttacccgccatatccgcatccacacaggccagaagcc | | | |
| | | cttccagtgtcgaatctgcatgcgtaacttcagtcgt | | | |
| | | agtgaccaccttaccacccacatccgcacccacacag | | | |
| | | gcggcggccgcaggaggaagaaacgcaccagcataga | | | |
| | | gaccaacatccgtgtggccttagagaagagtttcttg | | | |
| | | gagaatcaaaagcctacctcggaagagatcactatga | | | |
| | | ttgctgatcagctcaatatggaaaaagaggtgattcg | | | |
| | | tgtttggttctgtaaccgccgccagaaagaaaaaaga | | | |
| | | atcaacactagactgggggccttgcttggcaacagca | | | |
| | | cagacccagctgtgttcacagacctggcatccGTGga | | | |
| | | caactccgagtttcagcagctgctgaaccagggcata | | | |
| | | cctgtggccccccacacaactgagcccatgctgatgg | | | |
| | | agtaccctgaggctataactcgcctagtgacaggggc | | | |
| | | ccagaggccccccgacccagctcctgctccactgggg | | | |
| | | gccccggggctccccaatggcctcctttcaggagatg | | | |
| | | aagacttctcctccattgcggacatggacttctcagc | | | |
| | | cctgctgagtcagatcagctccggatcctga | | | |
| ZFHD- | EF1a | cgtgaggctccggtgcccgtcagtgggcagagcgcac | 9 | MAPKKKRKVERPYACPVESCDRRFSRSDELTRHIRIH | 10 |
| 005 | promoter; | atcgcccacagtccccgagaagttggggggaggggtc | | TGQKPFQCRICMRNFSRSDHLTTHIRTHTGGGRRRKK | |
| | ZFHD1 DNA | ggcaattgaaccggtgcctagagaaggtggcgcgggg | | RTSIETNIRVALEKSFLENQKPTSEEITMIADQLNME | |
| | binding | taaactgggaaagtgatgtcgtgtactggctccgcct | | KEVIRVWFCNRRQKEKRINTRLGALLGNSTDPAVFTD | |
| | domain; p65 | ttttcccgagggtgggggagaaccgtatataagtgca | | LASVDNSEFQQLLNQGIPVAPHTTEPMLMEYPEAITR | |
| | activation | gtagtcgccgtgaacgttctttttcgcaacgggtttg | | LVTGAQRPPDPAPAPLGAPGLPNGLLSGDEDFSSIAD | |
| | domain; | ccgccagaacacaggtaagtgccgtgtgtggttcccg | | MDFSALLSQISSGSSGISLIAALAVDYVIGMENAMPW | |
| | ecDHFR | cgggcctggcctctttacgggttatggcccttgcgtg | | NLPADLAWFKRNTLNKPVIMGRHTWESIGRPLPGRKN | |
| | (R12Y, | ccttgaattacttccacctggctgcagtacgtgattc | | IILSSQPGTDDRVTWVKSVDEAIAACGDVPEIMVIGG | |
| | Y100I) | ttgatcccgagcttcgggttggaagtgggtgggagag | | GRVIEQFLPKAQKLYLTHIDAEVEGDTHFPDYEPDDW | |
| | | ttcgaggccttgcgcttaaggagccccttcgcctcgt | | ESVFSEFHDADAQNSHSYCFEILERRGS | |
| | | gcttgagttgaggcctggcctgggcgctggggccgcc | | | |
| | | gcgtgcgaatctggtggcaccttcgcgcctgtctcgc | | | |
| | | tgctttcgataagtctctagccatttaaaatttttga | | | |
| | | tgacctgctgcgacgctttttttctggcaagatagtc | | | |
| | | ttgtaaatgcgggccaagatctgcacactggtatttc | | | |
| | | ggtttttggggccgcgggcggcgacggggcccgtgcg | | | |
| | | tcccagcgcacatgttcggcgaggcggggcctgcgag | | | |
| | | cgcggccaccgagaatcggacgggggtagtctcaagc | | | |
| | | tggccggcctgctctggtgcctggcctcgcgccgccg | | | |
| | | tgtatcgccccgccctgggcggcaaggctggcccggt | | | |
| | | cggcaccagttgcgtgagcggaaagatggccgcttcc | | | |
| | | cggccctgctgcagggagctcaaaatggaggacgcgg | | | |
| | | cgctcgggagagcgggcgggtgagtcacccacacaaa | | | |
| | | ggaaaagggcctttccgtcctcagccgtcgcttcatg | | | |
| | | tgactccactgagtaccgggcgccgtccaggcacctc | | | |
| | | gattagttctcgagcttttggagtacgtcgtctttag | | | |
| | | gttggggggaggggttttatgcgatggagtttcccca | | | |
| | | cactgagtgggtggagactgaagttaggccagcttgg | | | |
| | | cacttgatgtaattctccttggaatttgccctttttg | | | |
| | | agtttggatcttggttcattctcaagcctcagacagt | | | |
| | | ggttcaaagtttttttcttccatttcaggtgtcgtga | | | |
| | | tctagaggatcACTAGTgccaccatgGCACCTAAGaa | | | |
| | | aAAGAGGAAGGTTgaacgcccatatgcttgccctgtc | | | |
| | | gagtcctgcgatcgccgcttttctcgctcggatgagc | | | |
| | | ttacccgccatatccgcatccacacaggccagaagcc | | | |
| | | cttccagtgtcgaatctgcatgcgtaacttcagtcgt | | | |
| | | agtgaccaccttaccacccacatccgcacccacacag | | | |
| | | gcggcggccgcaggaggaagaaacgcaccagcataga | | | |
| | | gaccaacatccgtgtggccttagagaagagtttcttg | | | |
| | | gagaatcaaaagcctacctcggaagagatcactatga | | | |
| | | ttgctgatcagctcaatatggaaaaagaggtgattcg | | | |
| | | tgtttggttctgtaaccgccgccagaaagaaaaaaga | | | |
| | | atcaacactagactgggggccttgcttggcaacagca | | | |
| | | cagacccagctgtgttcacagacctggcatccGTGga | | | |
| | | caactccgagtttcagcagctgctgaaccagggcata | | | |
| | | cctgtggccccccacacaactgagcccatgctgatgg | | | |
| | | agtaccctgaggctataactcgcctagtgacaggggc | | | |
| | | ccagaggccccccgacccagctcctgctccactgggg | | | |
| | | gccccggggctccccaatggcctcctttcaggagatg | | | |
| | | aagacttctcctccattgcggacatggacttctcagc | | | |
| | | cctgctgagtcagatcagctccggatccagcggcatc | | | |
| | | tctctgattgcggcgctggcagttgactacgttattg | | | |
| | | gcatggaaaacgcgatgccatggaacctcccggctga | | | |
| | | cctggcgtggttcaaacgtaacaccctgaacaaacct | | | |
| | | gtgatcatgggtcgtcacacctgggaatctattggcc | | | |
| | | gtcctctcccgggtcgtaaaaacatcattctgtcttc | | | |
| | | tcagccaggcaccgacgaccgtgttacctgggttaaa | | | |
| | | agcgttgacgaagcgattgctgcgtgcggtgatgttc | | | |
| | | ctgaaattatggtgatcggcggtggccgtgttatcga | | | |
| | | acagttcctgccgaaagcgcagaaactgtacctgacc | | | |
| | | cacatcgacgcggaagttgaaggtgacacccacttcc | | | |
| | | cggactacgaaccggatgattgggagagcgtattctc | | | |
| | | cgaattccatgatgcggatgcgcaaaactctcattct | | | |
| | | tactgttttgaaatcctggaacgtcgtggatcctga | | | |
| ZFHD- | 8xZFHD1 BS- | taatgatgggcgcacgagtaatgatgggcggacgact | 11 | MVSKGEELFTGVVPILVELDGDVNGHKFSVSGEGEGD | 12 |
| 007 | min | aatgatgggcgcacgagtaatgatgggcgtctagcta | | ATYGKLTLKFICTTGKLPVPWPTLVTTLTYGVQCFSR | |
| | promoter; | atgatgggcgctagagtaatgatgggcggtagactaa | | YPDHMKQHDFFKSAMPEGYVQERTIFFKDDGNYKTRA | |
| | EGFP | tgatgggcgctccagtaatgatgggcgttctagcTCT | | EVKFEGDTLVNRIELKGIDFKEDGNILGHKLEYNYNS | |
| | | AGAGGGTATATAATGGGGGCCACTAGTCTACTACCAG | | HNVYIMADKQKNGIKVNFKIRHNIEDGSVQLADHYQQ | |
| | | AtAGCTTGGTACCGAGCTCtGATCCAGCCACCatggt | | NTPIGDGPVLLPDNHYLSTQSALSKDPNEKRDHMVLL | |
| | | gagcaagggcgaggagctgttcaccggggtggtgccc | | EFVTAAGITLGMDELYKGS | |
| | | atcctggtcgagctggacggcgacgtaaacggccaca | | | |
| | | agttcagcgtgtccggcgagggcgagggcgatgccac | | | |
| | | ctacggcaagctgaccctgaagttcatctgcaccacc | | | |
| | | ggcaagctgcccgtgccctggcccaccctcgtgacca | | | |
| | | ccctgacctacggcgtgcagtgcttcagccgctaccc | | | |
| | | cgaccacatgaagcagcacgacttcttcaagtccgcc | | | |
| | | atgcccgaaggctacgtccaggagcgcaccatcttct | | | |
| | | tcaaggacgacggcaactacaagacccgcgccgaggt | | | |
| | | gaagttcgagggcgacaccctggtgaaccgcatcgag | | | |
| | | ctgaagggcatcgacttcaaggaggacggcaacatcc | | | |
| | | tggggcacaagctggagtacaactacaacagccacaa | | | |
| | | cgtctatatcatggccgacaagcagaagaacggcatc | | | |
| | | aaggtgaacttcaagatccgccacaacatcgaggacg | | | |
| | | gcagcgtgcagctcgccgaccactaccagcagaacac | | | |
| | | ccccatcggcgacggccccgtgctgctgcccgacaac | | | |
| | | cactacctgagcacccagtccgccctgagcaaagacc | | | |
| | | ccaacgagaagcgcgatcacatggtcctgctggagtt | | | |
| | | cgtgaccgccgccgggatcactctcggcatggacgag | | | |
| | | ctgtacaagggatcctaa | | | |
| ZFHD- | EF1a | cgtgaggctccggtgcccgtcagtgggcagagcgcac | 13 | MAPKKKRKVERPYACPVESCDRRFSRSDELTRHIRIH | 14 |
| 008 | promoter; | atcgcccacagtccccgagaagttggggggaggggtc | | TGQKPFQCRICMRNFSRSDHLTTHIRTHTGGGRRRKK | |
| | ZFHDIDNA | ggcaattgaaccggtgcctagagaaggtggcgcgggg | | RTSIETNIRVALEKSFLENQKPTSEEITMIADQLNME | |
| | binding | taaactgggaaagtgatgtcgtgtactggctccgcct | | KEVIRVWFCNRRQKEKRINGSSGISLIAALAVDYVIG | |
| | domain; | ttttcccgagggtgggggagaaccgtatataagtgca | | MENAMPWNLPADLAWFKRNTLNKPVIMGRHTWESIGR | |
| | ecDHFR, | gtagtcgccgtgaacgttctttttcgcaacgggtttg | | PLPGRKNIILSSQPGTDDRVTWVKSVDEAIAACGDVP | |
| | (R12Y | ccgccagaacacaggtaagtgccgtgtgtggttcccg | | EIMVIGGGRVIEQFLPKAQKLYLTHIDAEVEGDTHFP | |
| | Y100I); p65 | cgggcctggcctctttacgggttatggcccttgcgtg | | DYEPDDWESVFSEFHDADAQNSHSYCFEILERRGSLG | |
| | activation | ccttgaattacttccacctggctgcagtacgtgattc | | ALLGNSTDPAVFTDLASVDNSEFQQLLNQGIPVAPHT | |
| | domain | ttgatcccgagcttcgggttggaagtgggtgggagag | | TEPMLMEYPEAITRLVTGAQRPPDPAPAPLGAPGLPN | |
| | | ttcgaggccttgcgcttaaggagccccttcgcctcgt | | GLLSGDEDFSSIADMDFSALLSQISSAS | |
| | | gcttgagttgaggcctggcctgggcgctggggccgcc | | | |
| | | gcgtgcgaatctggtggcaccttcgcgcctgtctcgc | | | |
| | | tgctttcgataagtctctagccatttaaaatttttga | | | |
| | | tgacctgctgcgacgctttttttctggcaagatagtc | | | |
| | | ttgtaaatgcgggccaagatctgcacactggtatttc | | | |
| | | ggtttttggggccgcgggcggcgacggggcccgtgcg | | | |
| | | tcccagcgcacatgttcggcgaggcggggcctgcgag | | | |
| | | cgcggccaccgagaatcggacgggggtagtctcaagc | | | |
| | | tggccggcctgctctggtgcctggcctcgcgccgccg | | | |
| | | tgtatcgccccgccctgggcggcaaggctggcccggt | | | |
| | | cggcaccagttgcgtgagcggaaagatggccgcttcc | | | |
| | | cggccctgctgcagggagctcaaaatggaggacgcgg | | | |
| | | cgctcgggagagcgggcgggtgagtcacccacacaaa | | | |
| | | ggaaaagggcctttccgtcctcagccgtcgcttcatg | | | |
| | | tgactccactgagtaccgggcgccgtccaggcacctc | | | |
| | | gattagttctcgagcttttggagtacgtcgtctttag | | | |
| | | gttggggggaggggttttatgcgatggagtttcccca | | | |
| | | cactgagtgggtggagactgaagttaggccagcttgg | | | |
| | | cacttgatgtaattctccttggaatttgccctttttg | | | |
| | | agtttggatcttggttcattctcaagcctcagacagt | | | |
| | | ggttcaaagtttttttcttccatttcaggtgtcgtga | | | |
| | | tctagaggatcACTAGTgccaccatgGCACCTAAGaa | | | |
| | | aAAGAGGAAGGTTgaacgcccatatgcttgccctgtc | | | |
| | | gagtcctgcgatcgccgcttttctcgctcggatgagc | | | |
| | | ttacccgccatatccgcatccacacaggccagaagcc | | | |
| | | cttccagtgtcgaatctgcatgcgtaacttcagtcgt | | | |
| | | agtgaccaccttaccacccacatccgcacccacacag | | | |
| | | gcggcggccgcaggaggaagaaacgcaccagcataga | | | |
| | | gaccaacatccgtgtggccttagagaagagtttcttg | | | |
| | | gagaatcaaaagcctacctcggaagagatcactatga | | | |
| | | ttgctgatcagctcaatatggaaaaagaggtgattcg | | | |
| | | tgtttggttctgtaaccgccgccagaaagaaaaaaga | | | |
| | | atcaacggatccagcggcatctctctgattgcggcgc | | | |
| | | tggcagttgactacgttattggcatggaaaacgcgat | | | |
| | | gccatggaacctcccggctgacctggcgtggttcaaa | | | |
| | | cgtaacaccctgaacaaacctgtgatcatgggtcgtc | | | |
| | | acacctgggaatctattggccgtcctctcccgggtcg | | | |
| | | taaaaacatcattctgtcttctcagccaggcaccgac | | | |
| | | gaccgtgttacctgggttaaaagcgttgacgaagcga | | | |
| | | ttgctgcgtgcggtgatgttcctgaaattatggtgat | | | |
| | | cggcggtggccgtgttatcgaacagttcctgccgaaa | | | |
| | | gcgcagaaactgtacctgacccacatcgacgcggaag | | | |
| | | ttgaaggtgacacccacttcccggactacgaaccgga | | | |
| | | tgattgggagagcgtattctccgaattccatgatgcg | | | |
| | | gatgcgcaaaactctcattcttactgttttgaaatcc | | | |
| | | tggaacgtcgtggatccctgggggccttgcttggcaa | | | |
| | | cagcacagacccagctgtgttcacagacctggcatcc | | | |
| | | GTGgacaactccgagtttcagcagctgctgaaccagg | | | |
| | | gcatacctgtggccccccacacaactgagcccatgct | | | |
| | | gatggagtaccctgaggctataactcgcctagtgaca | | | |
| | | ggggcccagaggccccccgacccagctcctgctccac | | | |
| | | tgggggccccggggctccccaatggcctcctttcagg | | | |
| | | agatgaagacttctcctccattgcggacatggacttc | | | |
| | | tcagccctgctgagtcagatcagctccGCTAGCtga | | | |
| ZFHD- | EF1a | cgtgaggctccggtgcccgtcagtgggcagagcgcac | 15 | MAPKKKRKVERPYACPVESCDRRFSRSDELTRHIRIH | 16 |
| 009 | promoter; | atcgcccacagtccccgagaagttggggggaggggtc | | TGQKPFQCRICMRNFSRSDHLTTHIRTHTGGGRRRKK | |
| | ZFHD1 DNA | ggcaattgaaccggtgcctagagaaggtggcgcgggg | | RTSIETNIRVALEKSFLENQKPTSEEITMIADQLNME | |
| | binding | taaactgggaaagtgatgtcgtgtactggctccgcct | | KEVIRVWFCNRRQKEKRINTRLGALLGNSTDPAVFTD | |
| | domain; p65 | ttttcccgagggtgggggagaaccgtatataagtgca | | LASVDNSEFQQLLNQGIPVAPHTTEPMLMEYPEAITR | |
| | activation | gtagtcgccgtgaacgttctttttcgcaacgggtttg | | LVTGAQRPPDPAPAPLGAPGLPNGLLSGDEDFSSIAD | |
| | domain; | ccgccagaacacaggtaagtgccgtgtgtggttcccg | | MDFSALLSQISSGSGSGSSG1SLIAALAVDYVIGMEN | |
| | linker; | cgggcctggcctctttacgggttatggcccttgcgtg | | AMPWNLPADLAWFKRNTLNKPVIMGRHTWESIGRPLP | |
| | ecDHFR | ccttgaattacttccacctggctgcagtacgtgattc | | GRKNIILSSQPGTDDRVTWVKSVDEAIAACGDVPEIM | |
| | (R12Y, | ttgatcccgagcttcgggttggaagtgggtgggagag | | VIGGGRVIEQFLPKAQKLYLTHIDAEVEGDTHFPDYE | |
| | Y100I) | ttcgaggccttgcgcttaaggagccccttcgcctcgt | | PDDWESVFSEFHDADAQNSHSYCFEILERRGS | |
| | | gcttgagttgaggcctggcctgggcgctggggccgcc | | | |
| | | gcgtgcgaatctggtggcaccttcgcgcctgtctcgc | | | |
| | | tgctttcgataagtctctagccatttaaaatttttga | | | |
| | | tgacctgctgcgacgctttttttctggcaagatagtc | | | |
| | | ttgtaaatgcgggccaagatctgcacactggtatttc | | | |
| | | ggtttttggggccgcgggcggcgacggggcccgtgcg | | | |
| | | tcccagcgcacatgttcggcgaggcggggcctgcgag | | | |
| | | cgcggccaccgagaatcggacgggggtagtctcaagc | | | |
| | | tggccggcctgctctggtgcctggcctcgcgccgccg | | | |
| | | tgtatcgccccgccctgggcggcaaggctggcccggt | | | |
| | | cggcaccagttgcgtgagcggaaagatggccgcttcc | | | |
| | | cggccctgctgcagggagctcaaaatggaggacgcgg | | | |
| | | cgctcgggagagcgggcgggtgagtcacccacacaaa | | | |
| | | ggaaaagggcctttccgtcctcagccgtcgcttcatg | | | |
| | | tgactccactgagtaccgggcgccgtccaggcacctc | | | |
| | | gattagttctcgagcttttggagtacgtcgtctttag | | | |
| | | gttggggggaggggttttatgcgatggagtttcccca | | | |
| | | cactgagtgggtggagactgaagttaggccagcttgg | | | |
| | | cacttgatgtaattctccttggaatttgccctttttg | | | |
| | | agtttggatcttggttcattctcaagcctcagacagt | | | |
| | | ggttcaaagtttttttcttccatttcaggtgtcgtga | | | |
| | | tctagaggatcACTAGTgccaccatgGCACCTAAGaa | | | |
| | | aAAGAGGAAGGTTgaacgcccatatgcttgccctgtc | | | |
| | | gagtcctgcgatcgccgcttttctcgctcggatgagc | | | |
| | | ttacccgccatatccgcatccacacaggccagaagcc | | | |
| | | cttccagtgtcgaatctgcatgcgtaacttcagtcgt | | | |
| | | agtgaccaccttaccacccacatccgcacccacacag | | | |
| | | gcggcggccgcaggaggaagaaacgcaccagcataga | | | |
| | | gaccaacatccgtgtggccttagagaagagtttcttg | | | |
| | | gagaatcaaaagcctacctcggaagagatcactatga | | | |
| | | ttgctgatcagctcaatatggaaaaagaggtgattcg | | | |
| | | tgtttggttctgtaaccgccgccagaaagaaaaaaga | | | |
| | | atcaacactagactgggggccttgcttggcaacagca | | | |
| | | cagacccagctgtgttcacagacctggcatccGTGga | | | |
| | | caactccgagtttcagcagctgctgaaccagggcata | | | |
| | | cctgtggccccccacacaactgagcccatgctgatgg | | | |
| | | agtaccctgaggctataactcgcctagtgacaggggc | | | |
| | | ccagaggccccccgacccagctcctgctccactgggg | | | |
| | | gccccggggctccccaatggcctcctttcaggagatg | | | |
| | | aagacttctcctccattgcggacatggacttctcagc | | | |
| | | cctgctgagtcagatcagctccggatccGGCTCAGGT | | | |
| | | AGCagcggcatctctctgattgcggcgctggcagttg | | | |
| | | actacgttattggcatggaaaacgcgatgccatggaa | | | |
| | | cctcccggctgacctggcgtggttcaaacgtaacacc | | | |
| | | ctgaacaaacctgtgatcatgggtcgtcacacctggg | | | |
| | | aatctattggccgtcctctcccgggtcgtaaaaacat | | | |
| | | cattctgtcttctcagccaggcaccgacgaccgtgtt | | | |
| | | acctgggttaaaagcgttgacgaagcgattgctgcgt | | | |
| | | gcggtgatgttcctgaaattatggtgatcggcggtgg | | | |
| | | ccgtgttatcgaacagttcctgccgaaagcgcagaaa | | | |
| | | ctgtacctgacccacatcgacgcggaagttgaaggtg | | | |
| | | acacccacttcccggactacgaaccggatgattggga | | | |
| | | gagcgtattctccgaattccatgatgcggatgcgcaa | | | |
| | | aactctcattcttactgttttgaaatcctggaacgtc | | | |
| | | gtggatcctga | | | |
| ZFHD- | EF1a | cgtgaggctccggtgcccgtcagtgggcagagcgcac | 17 | Transcription Factor Polypeptide: | 18 |
| 012 | ZFHD1 DNA | atcgcccacagtccccgagaagtgaaagtgatgtcgt | | MAPKKKRKVERPYACPVESCDRRFSRSDELTRHIRIH | |
| | binding | gtactggctccgcctttttcccgagggtgggggagaa | | TGQKPFQCRICMRNFSRSDHLTTHIRTHTGGGRRRKK | |
| | domain; p65 | ccgtatataagtgcagtagtcgccgtgaacgttcttt | | RTSIETNIRVALEKSFLENQKPTSEEITMIADQLNME | |
| | activation | ttcgcaacgggtttgccgccagaacacaggtaagtgc | | KEVIRVWFCNRRQKEKRINTRLGALLGNSTDPAVFTD | |
| | domain; | cgtgtgtggttcccgcgggcctggcctctttacgggt | | LASVDNSEFQQLLNQGIPVAPHTTEPMLMEYPEAITR | |
| | ecDHFR | tatggcccttgcgtgccttgaattacttccacctggc | | LVTGAQRPPDPAPAPLGAPGLPNGLLSGDEDFSSIAD | |
| | (R12Y, | tgcagtacgtgattcttgatcccgagcttcgggttgg | | MDFSALLSQISSGSSGISLIAALAVDYVIGMENAMPW | |
| | Y100I); | aagtgggtgggagagttcgaggccttgcgcttaagga | | NLPADLAWFKRNTLNKPVIMGRHTWESIGRPLPGRKN | |
| | EGFP; Min | gccccttcgcctcgtgcttgagttgaggcctggcctg | | IILSSQPGTDDRVTWVKSVDEAIAACGDVPEIMVIGG | |
| | promoter; | ggcgctggggccgccgcgtgcgaatctggtggcacct | | GRVIEQFLPKAQKLYLTHIDAEVEGDTHFPDYEPDDW | |
| | 8xZFHD1 | tcgcgcctgtctcgctgctttcgataagtctctagcc | | ESVFSEFHDADAQNSHSYCFEILERRGS | |
| | binding | atttaaaatttttgatgacctgctgcgacgctttttt | | EGFP polypeptide: | 19 |
| | sites | tctggcaagatagtcttgtaaatgcgggccaagatct | | MVSKGEELFTGVVPILVELDGDVNGHKFSVSGEGEGD | |
| | | gcacactggtatttcggtttttggggccgcgggcggc | | ATYGKLTLKFICTTGKLPVPWPTLVTTLTYGVQCFSR | |
| | | gacggggcccgtgcgtcccagcgcacatgttcggcga | | YPDHMKQHDFFKSAMPEGYVQERTIFFKDDGNYKTRA | |
| | | ggcggggcctgcgagcgcggccaccgagaatcggacg | | EVKFEGDTLVNRIELKGIDFKEDGNILGHKLEYNYNS | |
| | | ggggtagtctcaagctggccggcctgctctggtgcct | | HNVYIMADKQKNGIKVNFKIRHNIEDGSVQLADHYQQ | |
| | | ggcctcgcgccgccgtgtatcgccccgccctgggcgg | | NTPIGDGPVLLPDNHYLSTQSALSKDPNEKRDHMVLL | |
| | | caaggctggcccggtcggcaccagttgcgtgagcgga | | EFVTAAGITLGMDELYKGS | |
| | | aagatggccgcttcccggccctgctgcagggagctca | | | |
| | | aaatggaggacgcggcgctcgggagagcgggcgggtg | | | |
| | | agtcacccacacaaaggaaaagggcctttccgtcctc | | | |
| | | agccgtcgcttcatgtgactccactgagtaccgggcg | | | |
| | | ccgtccaggcacctcgattagttctcgagcttttgga | | | |
| | | gtacgtcgtctttaggttggggggaggggttttatgc | | | |
| | | gatggagtttccccacactgagtgggtggagactgaa | | | |
| | | gttaggccagcttggcacttgatgtaattctccttgg | | | |
| | | aatttgccctttttgagtttggatcttggttcattct | | | |
| | | caagcctcagacagtggttcaaagtttttttcttcca | | | |
| | | tttcaggtgtcgtgatctagaggatcACTAGTgccac | | | |
| | | catgGCACCTAAGaaaAAGAGGAAGGTTgaacgccca | | | |
| | | tatgcttgccctgtcgagtcctgcgatcgccgctttt | | | |
| | | ctcgctcggatgagcttacccgccatatccgcatcca | | | |
| | | cacaggccagaagcccttccagtgtcgaatctgcatg | | | |
| | | cgtaacttcagtcgtagtgaccaccttaccacccaca | | | |
| | | tccgcacccacacaggcggcggccgcaggaggaagaa | | | |
| | | acgcaccagcatagagaccaacatccgtgtggcctta | | | |
| | | gagaagagtttcttggagaatcaaaagcctacctcgg | | | |
| | | aagagatcactatgattgctgatcagctcaatatgga | | | |
| | | aaaagaggtgattcgtgtttggttctgtaaccgccgc | | | |
| | | cagaaagaaaaaagaatcaacactagactgggggcct | | | |
| | | tgcttggcaacagcacagacccagctgtggggggagg | | | |
| | | ggtcggcaattgaaccggtgcctagagaaggtggcgc | | | |
| | | ggggtaaactggtgttcacagacctggcatccGTGga | | | |
| | | caactccgagtttcagcagctgctgaaccagggcata | | | |
| | | cctgtggccccccacacaactgagcccatgctgatgg | | | |
| | | agtaccctgaggctataactcgcctagtgacaggggc | | | |
| | | ccagaggccccccgacccagctcctgctccactgggg | | | |
| | | gccccggggctccccaatggcctcctttcaggagatg | | | |
| | | aagacttctcctccattgcggacatggacttctcagc | | | |
| | | cctgctgagtcagatcagctccggatccagcggcatc | | | |
| | | tctctgattgcggcgctggcagttgactacgttattg | | | |
| | | gcatggaaaacgcgatgccatggaacctcccggctga | | | |
| | | cctggcgtggttcaaacgtaacaccctgaacaaacct | | | |
| | | gtgatcatgggtcgtcacacctgggaatctattggcc | | | |
| | | gtcctctcccgggtcgtaaaaacatcattctgtcttc | | | |
| | | tcagccaggcaccgacgaccgtgttacctgggttaaa | | | |
| | | agcgttgacgaagcgattgctgcgtgcggtgatgttc | | | |
| | | ctgaaattatggtgatcggcggtggccgtgttatcga | | | |
| | | acagttcctgccgaaagcgcagaaactgtacctgacc | | | |
| | | cacatcgacgcggaagttgaaggtgacacccacttcc | | | |
| | | cggactacgaaccggatgattgggagagcgtattctc | | | |
| | | cgaattccatgatgcggatgcgcaaaactctcattct | | | |
| | | tactgttttgaaatcctggaacgtcgtggatcctgaA | | | |
| | | TCGGGCTAGCacgcgtttaggatcccttgtacagctc | | | |
| | | gtccatgccgagagtgatcccggcggcggtcacgaac | | | |
| | | tccagcaggaccatgtgatcgcgcttctcgttggggt | | | |
| | | ctttgctcagggcggactgggtgctcaggtagtggtt | | | |
| | | gtcgggcagcagcacggggccgtcgccgatgggggtg | | | |
| | | ttctgctggtagtggtcggcgagctgcacgctgccgt | | | |
| | | cctcgatgttgtggcggatcttgaagttcaccttgat | | | |
| | | gccgttcttctgcttgtcggccatgatatagacgttg | | | |
| | | tggctgttgtagttgtactccagcttgtgccccagga | | | |
| | | tgttgccgtcctccttgaagtcgatgcccttcagctc | | | |
| | | gatgcggttcaccagggtgtcgccctcgaacttcacc | | | |
| | | tcggcgcgggtcttgtagttgccgtcgtccttgaaga | | | |
| | | agatggtgcgctcctggacgtagccttcgggcatggc | | | |
| | | ggacttgaagaagtcgtgctgcttcatgtggtcgggg | | | |
| | | tagcggctgaagcactgcacgccgtaggtcagggtgg | | | |
| | | tcacgagggtgggccagggcacgggcagcttgccggt | | | |
| | | ggtgcagatgaacttcagggtcagcttgccgtaggtg | | | |
| | | gcatcgccctcgccctcgccggacacgctgaacttgt | | | |
| | | ggccgtttacgtcgccgtccagctcgaccaggatggg | | | |
| | | caccaccccggtgaacagctcctcgcccttgctcacc | | | |
| | | atGGTGGCTGGATCaGAGCTCGGTACCAAGCTaTCTG | | | |
| | | GTAGTAGACTAGTGGCCCCCATTATATACCCTCTAGA | | | |
| | | gctagaacgcccatcattactggagcgcccatcatta | | | |
| | | gtctaccgcccatcattactctagcgcccatcattag | | | |
| | | ctagacgcccatcattactcgtgcgcccatcattagt | | | |
| | | cgtccgcccatcattactcgtgcgcccatcatta | | | |
| ZFHD- | pELDS- | taatgatgggcgcacgagtaatgatgggcggacgact | 20 | MCHQQLVISWFSLVFLASPLVAIWELKKDVYVVELDW | 21 |
| 013 | 8xZFHD1 BS- | aatgatgggcgcacgagtaatgatgggcgtctagcta | | YPDAPGEMVVLTCDTPEEDGITWTLDQSSEVLGSGKT | |
| | min | atgatgggcgctagagtaatgatgggcggtagactaa | | LTIQVKEFGDAGQYTCHKGGEVLSHSLLLLHKKEDGI | |
| | promoter- | tgatgggcgctccagtaatgatgggcgttctagcTCT | | WSTDILKDQKEPKNKTFLRCEAKNYSGRFTCWWLTTI | |
| | IL12 | AGAGGGTATATAATGGGGGCCACTAGTtctagaggat | | STDLTFSVKSSRGSSDPQGVTCGAATLSAERVRGDNK | |
| | | caccatgtgccaccaacagctcgtgatcagctggttc | | EYEYSVECQEDSACPAAEESLPIEVMVDAVHKLKYEN | |
| | | tccctggtgttcctggctagccccctggtggccatct | | YTSSFFIRDIIKPDPPKNLQLKPLKNSRQVEVSWEYP | |
| | | gggagctcaagaaagacgtgtacgtggtggaactgga | | DTWSTPHSYFSLTFCVQVQGKSKREKKDRVFTDKTSA | |
| | | ctggtaccccgacgcccctggcgaaatggtggtgctg | | TVICRKNASISVRAQDRYYSSSWSEWASVPCSGGGGS | |
| | | acatgcgacacccctgaggaggatggcattacctgga | | GGGGSGGGGSRNLPVATPDPGMFPCLHHSQNLLRAVS | |
| | | ccctcgatcagagctccgaggtgctgggcagcggcaa | | NMLQKARQTLEFYPCTSEEIDHEDITKDKTSTVEACL | |
| | | aaccctgaccatccaggtgaaggagtttggcgatgcc | | PLELTKNESCLNSRETSFITNGSCLASRKTSFMMALC | |
| | | ggccagtacacatgtcacaagggcggcgaggtgctga | | LSSIYEDLKMYQVEFKTMNAKLLMDPKRQIFLDQNML | |
| | | gccactccctgctgctgctccacaagaaggaagatgg | | AVIDELMQALNFNSETVPQKSSLEEPDFYKTKIKLCI | |
| | | catctggagcaccgacattctgaaggatcagaaggag | | LLHAFRIRAVTIDRVMSYLNASGS | |
| | | cccaagaacaagacattcctgaggtgtgaggccaaga | | | |
| | | actacagcggcaggtttacctgctggtggctgacaac | | | |
| | | aatcagcaccgacctcacattctccgtcaagtcctcc | | | |
| | | aggggttcttccgaccctcaaggcgtgacatgcggcg | | | |
| | | ctgccaccctgagcgctgagagagtcaggggcgacaa | | | |
| | | caaggagtacgagtacagcgtcgaatgtcaggaggac | | | |
| | | agcgcctgtcccgccgctgaagagagcctgcctatcg | | | |
| | | aggtgatggtggacgccgtgcacaaactgaagtatga | | | |
| | | gaattacacctccagcttcttcatcagggacatcatc | | | |
| | | aaacccgatccccccaagaacctgcagctgaagcctc | | | |
| | | tgaagaacagcagacaggtcgaagtgtcctgggagta | | | |
| | | ccctgatacctggtccacaccccacagctacttcagc | | | |
| | | ctgaccttttgcgtgcaggtgcagggcaagagcaaaa | | | |
| | | gggagaagaaggacagggtgtttaccgacaagacctc | | | |
| | | cgccacagtgatttgcagaaagaacgcctccatcagc | | | |
| | | gtgagggcccaggacaggtattacagcagctcctgga | | | |
| | | gcgaatgggctagcgtgccctgtagcggaggaggagg | | | |
| | | cagcggaggaggaggttctggaggaggcggcagcaga | | | |
| | | aacctgcctgtcgctacccccgaccccggaatgttcc | | | |
| | | cctgcctgcaccactcccagaacctcctgagggccgt | | | |
| | | gtccaacatgctgcagaaggctagacagaccctcgaa | | | |
| | | ttctacccctgtaccagcgaggagatcgaccatgagg | | | |
| | | acatcaccaaggataagaccagcaccgtggaggcttg | | | |
| | | cctgcctctggagctgaccaaaaacgagagctgcctg | | | |
| | | aacagcagggaaaccagcttcattaccaacggctcct | | | |
| | | gcctggcctccaggaagacatccttcatgatggccct | | | |
| | | gtgcctcagcagcatctacgaggacctgaagatgtat | | | |
| | | caggtggagtttaagaccatgaatgccaagctgctga | | | |
| | | tggaccctaagaggcagatcttcctggaccagaatat | | | |
| | | gctggccgtgattgacgagctgatgcaggccctcaac | | | |
| | | tttaacagcgagaccgtgccccagaaaagcagcctcg | | | |
| | | aagagcctgacttctacaaaaccaagattaagctgtg | | | |
| | | tatcctgctgcacgccttcaggatcagggccgtgacc | | | |
| | | atcgacagggtgatgagctacctgaacgccagcGGAT | | | |
| | | CCtaa | | | |
| ZFHD- | SV40 polyA; | cagacatgataagatacattgatgagtttggacaaac | 22 | MVSKGEELFTGVVPILVELDGDVNGHKFSVSGEGEGD | 23 |
| 017 | EGFP; Min | cacaactagaatgcagtgaaaaaaatgctttatttgt | | ATYGKLTLKFICTTGKLPVPWPTLVTTLTYGVQCFSR | |
| | promoter; | gaaatttgtgatgctattgctttatttgtaaccatta | | YPDHMKQHDFFKSAMPEGYVQERTIFFKDDGNYKTRA | |
| | 8xZFHD1 BS | taagctgcaataaacaagttcctctcactctctgata | | EVKFEGDTLVNRIELKGIDFKEDGNILGHKLEYNYNS | |
| | | ttcatttctttgcaagttaggatcccttgtacagctc | | HNVYIMADKQKNGIKVNFKIRHNIEDGSVQLADHYQQ | |
| | | gtccatgccgagagtgatcccggcggcggtcacgaac | | NTPIGDGPVLLPDNHYLSTQSALSKDPNEKRDHMVLL | |
| | | tccagcaggaccatgtgatcgcgcttctcgttggggt | | EFVTAAGITLGMDELYKGS | |
| | | ctttgctcagggcggactgggtgctcaggtagtggtt | | | |
| | | gtcgggcagcagcacggggccgtcgccgatgggggtg | | | |
| | | ttctgctggtagtggtcggcgagctgcacgctgccgt | | | |
| | | cctcgatgttgtggcggatcttgaagttcaccttgat | | | |
| | | gccgttcttctgcttgtcggccatgatatagacgttg | | | |
| | | tggctgttgtagttgtactccagcttgtgccccagga | | | |
| | | tgttgccgtcctccttgaagtcgatgcccttcagctc | | | |
| | | gatgcggttcaccagggtgtcgccctcgaacttcacc | | | |
| | | tcggcgcgggtcttgtagttgccgtcgtccttgaaga | | | |
| | | agatggtgcgctcctggacgtagccttcgggcatggc | | | |
| | | ggacttgaagaagtcgtgctgcttcatgtggtcgggg | | | |
| | | tagcggctgaagcactgcacgccgtaggtcagggtgg | | | |
| | | tcacgagggtgggccagggcacgggcagcttgccggt | | | |
| | | ggtgcagatgaacttcagggtcagcttgccgtaggtg | | | |
| | | gcatcgccctcgccctcgccggacacgctgaacttgt | | | |
| | | ggccgtttacgtcgccgtccagctcgaccaggatggg | | | |
| | | caccaccccggtgaacagctcctcgcccttgctcacc | | | |
| | | atGGTGGCTGGATCaGAGCTCGGTACCAAGCTaTCTG | | | |
| | | GTAGTAGACTAGTGGCCCCCATTATATACCCTCTAGA | | | |
| | | gctagaacgcccatcattactggagcgcccatcatta | | | |
| | | gtctaccgcccatcattactctagcgcccatcattag | | | |
| | | ctagacgcccatcattactcgtgcgcccatcattagt | | | |
| | | cgtccgcccatcattactcgtgcgcccatcatta | | | |
| ZFHD- | 8xZFHD1 | taatgatgggcgcacgagtaatgatgggcggacgact | 24 | EGFP Polypeptide: | 25 |
| 018 | Min | aatgatgggcgcacgagtaatggctccagtaatgatg | | MVSKGEELFTGVVPILVELDGDVNGHKFSVSGEGEGD | |
| | promoter; | ggcgttctagcTCTAGAGGGTATATAATGGGGGCCAC | | ATYGKLTLKFICTTGKLPVPWPTLVTTLTYGVQCFSR | |
| | EGFP; | TAGTCTACTACCAGAtAGCTTGGTACCGAGCTCtGAT | | YPDHMKQHDFFKSAMPEGYVQERTIFFKDDGNYKTRA | |
| | linker; | CCAGCCACCatggtgagcaagggcgaggagctgttca | | EVKFEGDTLVNRIELKGIDFKEDGNILGHKLEYNYNS | |
| | EF1a | ccggggtggtgcccatcctggtcgagctggacggcga | | HNVYIMADKQKNGIKVNFKIRHNIEDGSVQLADHYQQ | |
| | promoter; | cgtaaacggccacaagttcagcgtgtccggcgagggc | | NTPIGDGPVLLPDNHYLSTQSALSKDPNEKRDHMVLL | |
| | ZFHD1 DNA | gagggcgatgccacctacggcaagctgaccctgaagt | | EFVTAAGITLGMDELYKGS | |
| | binding | tcatctgcaccaccggcaagctgcccgtgccctggcc | | Transcription Factor Polypeptide: | 26 |
| | domain; p65 | caccctcgtgaccaccctgacctacggcgtgcagtgc | | MAPKKKRKVERPYACPVESCDRRFSRSDELTRHIRIH | |
| | activation | ttcagccgctaccccgaccacatgaagcagcacgact | | TGQKPFQCRICMRNFSRSDHLTTHIRTHTGGGRRRKK | |
| | domain; | tcttcaagtccgccatgcccgaaggctacgtccagga | | RTSIETNIRVALEKSFLENQKPTSEEITMIADQLNME | |
| | ecDHFR | gcgcaccatcttcttcaaggacgacggcaactacaag | | KEVIRVWFCNRRQKEKRINTRLGALLGNSTDPAVFTD | |
| | (R12Y, | acccgcgccgaggtgaagttcgagggcgacaccctgg | | LASVDNSEFQQLLNQGIPVAPHTTEPMLMEYPEAITR | |
| | Y100I) | tgaaccgcatcgagctgaagggcatcgacttcaagga | | LVTGAQRPPDPAPAPLGAPGLPNGLLSGDEDFSSIAD | |
| | | ggacggcaacatcctggggcacaagctggagtacaac | | MDFSALLSQISSGSSGISLIAALAVDYVIGMENAMPW | |
| | | tacaacagccacaacgtctatatcatggccgacaagc | | NLPADLAWFKRNTLNKPVIMGRHTWESIGRPLPGRKN | |
| | | agaagaacggcatcaaggtgaacttcaagatccgcca | | IILSSQPGTDDRVTWVKSVDEAIAACGDVPEIMVIGG | |
| | | caacatcgaggacggcagcgtgcagctcgccgaccac | | GRVIEQFLPKAQKLYLTHIDAEVEGDTHFPDYEPDDW | |
| | | taccagcagaacacccccatcggcgacggccccgtgc | | ESVFSEFHDADAQNSHSYCFEILERRGS | |
| | | tgctgcccgacaaccactacctgagcacccagtccgc | | | |
| | | cctgagcaaagaccccaacgagaagcgcgatcacatg | | | |
| | | gtcctgctggagttcgtgaccgccgccgggatcactc | | | |
| | | tcggcatggacgagctgtacaagggatcctaaATCGG | | | |
| | | GCTAGCcacgtgaggctccggtgcccgtcagtgggca | | | |
| | | gagcgcacatcgcccacagtccccgagaagttggggg | | | |
| | | gaggggtcggcaattgaaccggtgcctagagaaggtg | | | |
| | | gcgcggggtaaactgggaaagtgatgtcgtgtactgg | | | |
| | | ctccgcctttttcccgagggtgggggagaaccgtata | | | |
| | | taagtgcagtagtcgccgtgaacgttctttttcgcaa | | | |
| | | cgggtttgccgccagaacacaggtaagtgccgtgtgt | | | |
| | | ggttcccgcgggcctggcctctttacgggttatggcc | | | |
| | | cttgcgtgccttgaattacttccacctggctgcagta | | | |
| | | cgtgattcttgatcccgagcatgggcgtctagctaat | | | |
| | | gatgggcgctagagtaatgatgggcggtagactaatg | | | |
| | | atgggcttcgggttggaagtgggtgggagagttcgag | | | |
| | | gccttgcgcttaaggagccccttcgcctcgtgcttga | | | |
| | | gttgaggcctggcctgggcgctggggccgccgcgtgc | | | |
| | | gaatctggtggcaccttcgcgcctgtctcgctgcttt | | | |
| | | cgataagtctctagccatttaaaatttttgatgacct | | | |
| | | gctgcgacgctttttttctggcaagatagtcttgtaa | | | |
| | | atgcgggccaagatctgcacactggtatttcggtttt | | | |
| | | tggggccgcgggcggcgacggggcccgtgcgtcccag | | | |
| | | cgcacatgttcggcgaggcggggcctgcgagcgcggc | | | |
| | | caccgagaatcggacgggggtagtctcaagctggccg | | | |
| | | gcctgctctggtgcctggcctcgcgccgccgtgtatc | | | |
| | | gccccgccctgggcggcaaggctggcccggtcggcac | | | |
| | | cagttgcgtgagcggaaagatggccgcttcccggccc | | | |
| | | tgctgcagggagctcaaaatggaggacgcggcgctcg | | | |
| | | ggagagcgggcgggtgagtcacccacacaaaggaaaa | | | |
| | | gggcctttccgtcctcagccgtcgcttcatgtgactc | | | |
| | | cactgagtaccgggcgccgtccaggcacctcgattag | | | |
| | | ttctcgagcttttggagtacgtcgtctttaggttggg | | | |
| | | gggaggggttttatgcgatggagtttccccacactga | | | |
| | | gtgggtggagactgaagttaggccagcttggcacttg | | | |
| | | atgtaattctccttggaatttgccctttttgagtttg | | | |
| | | gatcttggttcattctcaagcctcagacagtggttca | | | |
| | | aagtttttttcttccatttcaggtgtcgtgatctaga | | | |
| | | ggatcACTAGTgccaccatgGCACCTAAGaaaAAGAG | | | |
| | | GAAGGTTgaacgcccatatgcttgccctgtcgagtcc | | | |
| | | tgcgatcgccgcttttctcgctcggatgagcttaccc | | | |
| | | gccatatccgcatccacacaggccagaagcccttcca | | | |
| | | gtgtcgaatctgcatgcgtaacttcagtcgtagtgac | | | |
| | | caccttaccacccacatccgcacccacacaggcggcg | | | |
| | | gccgcaggaggaagaaacgcaccagcatagagaccaa | | | |
| | | catccgtgtggccttagagaagagtttcttggagaat | | | |
| | | caaaagcctacctcggaagagatcactatgattgctg | | | |
| | | atcagctcaatatggaaaaagaggtgattcgtgtttg | | | |
| | | gttctgtaaccgccgccagaaagaaaaaagaatcaac | | | |
| | | actagactgggggccttgcttggcaacagcacagacc | | | |
| | | cagctgtgttcacagacctggcatccGTGgacaactc | | | |
| | | cgagtttcagcagctgctgaaccagggcatacctgtg | | | |
| | | gccccccacacaactgagcccatgctgatggagtacc | | | |
| | | ctgaggctataactcgcctagtgacaggggcccagag | | | |
| | | gccccccgacccagctcctgctccactgggggccccg | | | |
| | | gggctccccaatggcctcctttcaggagatgaagact | | | |
| | | tctcctccattgcggacatggacttctcagccctgct | | | |
| | | gagtcagatcagctccggatccagcggcatctctctg | | | |
| | | attgcggcgctggcagttgactacgttattggcatgg | | | |
| | | aaaacgcgatgccatggaacctcccggctgacctggc | | | |
| | | gtggttcaaacgtaacaccctgaacaaacctgtgatc | | | |
| | | atgggtcgtcacacctgggaatctattggccgtcctc | | | |
| | | tcccgggtcgtaaaaacatcattctgtcttctcagcc | | | |
| | | aggcaccgacgaccgtgttacctgggttaaaagcgtt | | | |
| | | gacgaagcgattgctgcgtgcggtgatgttcctgaaa | | | |
| | | ttatggtgatcggcggtggccgtgttatcgaacagtt | | | |
| | | cctgccgaaagcgcagaaactgtacctgacccacatc | | | |
| | | gacgcggaagttgaaggtgacacccacttcccggact | | | |
| | | acgaaccggatgattgggagagcgtattctccgaatt | | | |
| | | ccatgatgcggatgcgcaaaactctcattcttactgt | | | |
| | | tttgaaatcctggaacgtcgtggatcctga | | | |
| ZFHD- | EF1a | cgtgaggctccggtgcccgtcagtgggcagagcgcac | 27 | MAPKKKRKVERPYACPVESCDRRFSRSDELTRHIRIH | 28 |
| 019 | promoter; | atcgcccacagtccccgagaagttggggggaggggtc | | TGQKPFQCRICMRNFSRSDHLTTHIRTHTGGGRRRKK | |
| | ZFHD1 DNA | ggcaattgaaccggtgcctagagaaggtggcgcgggg | | RTSIETNIRVALEKSFLENQKPTSEEITMIADQLNME | |
| | binding | taaactgggaaagtgatgtcgtgtactggctccgcct | | KEVIRVWFCNRRQKEKRINTRLGALLGNSTDPAVFTD | |
| | domain; p65 | ttttcccgagggtgggggagaaccgtatataagtgca | | LASVDNSEFQQLLNQGIPVAPHTTEPMLMEYPEAITR | |
| | activation | gtagtcgccgtgaacgttctttttcgcaacgggtttg | | LVTGAQRPPDPAPAPLGAPGLPNGLLSGDEDFSSIAD | |
| | domain; | ccgccagaacacaggtaagtgccgtgtgtggttcccg | | MDFSALLSQISSGSSHHWGYGKHNGPEHWHKDFPIAK | |
| | CA2(L156H) | cgggcctggcctctttacgggttatggcccttgcgtg | | GERQSPVDIDTHTAKYDPSLKPLSVSYDQATSLRILN | |
| | DRD | ccttgaattacttccacctggctgcagtacgtgattc | | NGHAFNVEFDDSQDKAVLKGGPLDGTYRLIQFHFHWG | |
| | | ttgatcccgagcttcgggttggaagtgggtgggagag | | SLDGQGSEHTVDKKKYAAELHLVHWNTKYGDFGKAVQ | |
| | | ttcgaggccttgcgcttaaggagccccttcgcctcgt | | QPDGLAVLGIFLKVGSAKPGHQKVVDVLDSIKTKGKS | |
| | | gcttgagttgaggcctggcctgggcgctggggccgcc | | ADFTNFDPRGLLPESLDYWTYPGSLTTPPLLECVTWI | |
| | | gcgtgcgaatctggtggcaccttcgcgcctgtctcgc | | VLKEPISVSSEQVLKFRKLNFNGEGEPEELMVDNWRP | |
| | | tgctttcgataagtctctagccatttaaaatttttga | | AQPLKNRQIKASFKGS | |
| | | tgacctgctgcgacgctttttttctggcaagatagtc | | | |
| | | ttgtaaatgcgggccaagatctgcacactggtatttc | | | |
| | | ggtttttggggccgcgggcggcgacggggcccgtgcg | | | |
| | | tcccagcgcacatgttcggcgaggcggggcctgcgag | | | |
| | | cgcggccaccgagaatcggacgggggtagtctcaagc | | | |
| | | tggccggcctgctctggtgcctggcctcgcgccgccg | | | |
| | | tgtatcgccccgccctgggcggcaaggctggcccggt | | | |
| | | cggcaccagttgcgtgagcggaaagatggccgcttcc | | | |
| | | cggccctgctgcagggagctcaaaatggaggacgcgg | | | |
| | | cgctcgggagagcgggcgggtgagtcacccacacaaa | | | |
| | | ggaaaagggcctttccgtcctcagccgtcgcttcatg | | | |
| | | tgactccactgagtaccgggcgccgtccaggcacctc | | | |
| | | gattagttctcgagcttttggagtacgtcgtctttag | | | |
| | | gttggggggaggggttttatgcgatggagtttcccca | | | |
| | | cactgagtgggtggagactgaagttaggccagcttgg | | | |
| | | cacttgatgtaattctccttggaatttgccctttttg | | | |
| | | agtttggatcttggttcattctcaagcctcagacagt | | | |
| | | ggttcaaagtttttttcttccatttcaggtgtcgtga | | | |
| | | tctagaggatcACTAGTgccaccatgGCACCTAAGaa | | | |
| | | aAAGAGGAAGGTTgaacgcccatatgcttgccctgtc | | | |
| | | gagtcctgcgatcgccgcttttctcgctcggatgagc | | | |
| | | ttacccgccatatccgcatccacacaggccagaagcc | | | |
| | | cttccagtgtcgaatctgcatgcgtaacttcagtcgt | | | |
| | | agtgaccaccttaccacccacatccgcacccacacag | | | |
| | | gcggcggccgcaggaggaagaaacgcaccagcataga | | | |
| | | gaccaacatccgtgtggccttagagaagagtttcttg | | | |
| | | gagaatcaaaagcctacctcggaagagatcactatga | | | |
| | | ttgctgatcagctcaatatggaaaaagaggtgattcg | | | |
| | | tgtttggttctgtaaccgccgccagaaagaaaaaaga | | | |
| | | atcaacactagactgggggccttgcttggcaacagca | | | |
| | | cagacccagctgtgttcacagacctggcatccGTGga | | | |
| | | caactccgagtttcagcagctgctgaaccagggcata | | | |
| | | cctgtggccccccacacaactgagcccatgctgatgg | | | |
| | | agtaccctgaggctataactcgcctagtgacaggggc | | | |
| | | ccagaggccccccgacccagctcctgctccactgggg | | | |
| | | gccccggggctccccaatggcctcctttcaggagatg | | | |
| | | aagacttctcctccattgcggacatggacttctcagc | | | |
| | | cctgctgagtcagatcagctccggatccTCCCATCAC | | | |
| | | TGGGGGTACGGCAAACACAACGGACCTGAGCACTGGC | | | |
| | | ATAAGGACTTCCCCATTGCCAAGGGAGAGCGCCAGTC | | | |
| | | CCCTGTTGACATCGACACTCATACAGCCAAGTATGAC | | | |
| | | CCTTCCCTGAAGCCCCTGTCTGTTTCCTATGATCAAG | | | |
| | | CAACTTCCCTGAGGATCCTCAACAATGGTCATGCTTT | | | |
| | | CAACGTGGAGTTTGATGACTCTCAGGACAAAGCAGTG | | | |
| | | CTCAAGGGAGGACCCCTGGATGGCACTTACAGATTGA | | | |
| | | TTCAGTTTCACTTTCACTGGGGTTCACTTGATGGACA | | | |
| | | AGGTTCAGAGCATACTGTGGATAAAAAGAAATATGCT | | | |
| | | GCAGAACTTCACTTGGTTCACTGGAACACCAAATATG | | | |
| | | GGGATTTTGGGAAAGCTGTGCAGCAACCTGATGGACT | | | |
| | | GGCCGTTCTAGGTATTTTTTTGAAGGTTGGCAGCGCT | | | |
| | | AAACCGGGCCATCAGAAAGTTGTTGATGTGCTGGATT | | | |
| | | CCATTAAAACAAAGGGCAAGAGTGCTGACTTCACTAA | | | |
| | | CTTCGATCCTCGTGGCCTCCTTCCTGAATCCCTGGAT | | | |
| | | TACTGGACCTACCCAGGCTCACTGACCACCCCTCCTC | | | |
| | | TTCTGGAATGTGTGACCTGGATTGTGCTCAAGGAACC | | | |
| | | CATCAGCGTCAGCAGCGAGCAGGTGTTGAAATTCCGT | | | |
| | | AAACTTAACTTCAATGGGGAGGGTGAACCCGAAGAAC | | | |
| | | TGATGGTGGACAACTGGCGCCCAGCTCAGCCACTGAA | | | |
| | | GAACAGGCAAATCAAAGCTTCCTTCAAAggatcctga | | | |
| ZFHD- | 8xZFHD1 BS- | taatgatgggcgcacgagtaatgatgggcggacgact | 29 | MVSKGEELFTGVVPILVELDGDVNGHKFSVSGEGEGD | 30 |
| 022 | min | aatgatgggcgcacgagtaatgatgggcgtctagcta | | ATYGKLTLKFICTTGKLPVPWPTLVTTLTYGVQCFSR | |
| | promoter; | atgatgggcgctagagtaatgatgggcggtagactaa | | YPDHMKQHDFFKSAMPEGYVQERTIFFKDDGNYKTRA | |
| | EGFP | tgatgggcgctccagtaatgatgggcgttctagcTCT | | EVKFEGDTLVNRIELKGIDFKEDGNILGHKLEYNYNS | |
| | | AGAGGGTATATAATGGGGGCCACTAGTCTACTACCAG | | HNVYIMADKQKNGIKVNFKIRHNIEDGSVQLADHYQQ | |
| | | AtAGCTTGGTACCGAGCTCtGATCCAGCCACCatggt | | NTPIGDGPVLLPDNHYLSTQSALSKDPNEKRDHMVLL | |
| | | gagcaagggcgaggagctgttcaccggggtggtgccc | | EFVTAAGITLGMDELYKGS | |
| | | atcctggtcgagctggacggcgacgtaaacggccaca | | | |
| | | agttcagcgtgtccggcgagggcgagggcgatgccac | | | |
| | | ctacggcaagctgaccctgaagttcatctgcaccacc | | | |
| | | ggcaagctgcccgtgccctggcccaccctcgtgacca | | | |
| | | ccctgacctacggcgtgcagtgcttcagccgctaccc | | | |
| | | cgaccacatgaagcagcacgacttcttcaagtccgcc | | | |
| | | atgcccgaaggctacgtccaggagcgcaccatcttct | | | |
| | | tcaaggacgacggcaactacaagacccgcgccgaggt | | | |
| | | gaagttcgagggcgacaccctggtgaaccgcatcgag | | | |
| | | ctgaagggcatcgacttcaaggaggacggcaacatcc | | | |
| | | tggggcacaagctggagtacaactacaacagccacaa | | | |
| | | cgtctatatcatggccgacaagcagaagaacggcatc | | | |
| | | aaggtgaacttcaagatccgccacaacatcgaggacg | | | |
| | | gcagcgtgcagctcgccgaccactaccagcagaacac | | | |
| | | ccccatcggcgacggccccgtgctgctgcccgacaac | | | |
| | | cactacctgagcacccagtccgccctgagcaaagacc | | | |
| | | ccaacgagaagcgcgatcacatggtcctgctggagtt | | | |
| | | cgtgaccgccgccgggatcactctcggcatggacgag | | | |
| | | ctgtacaagggatcctaa | | | |
| ZFHD- | SV40 polyA; | cagacatgataagatacattgatgagtttggacaaac | 31 | EGFP Polypeptide: | 32 |
| 036 | promoter; | cacaactagaatgcagtgaaaaaacaagttcctctca | | MVSKGEELFTGVVPILVELDGDVNGHKFSVSGEGEGD | |
| | 8xZFHD1 BS; | ctctctgatattcatttctttgcaagttaggatccct | | ATYGKLTLKFICTTGKLPVPWPTLVTTLTYGVQCFSR | |
| | EF1a | tgtacagctcgtccatgccgagagtgatcccggcggc | | YPDHMKQHDFFKSAMPEGYVQERTIFFKDDGNYKTRA | |
| | promoter; | ggtcacgaactccagcaggaccatgtgatcgcgcttc | | EVKFEGDTLVNRIELKGIDFKEDGNILGHKLEYNYNS | |
| | ZFHD1 DNA | tcgttggggtctttgctcagggcggactgggtgctca | | HNVYIMADKQKNGIKVNFKIRHNIEDGSVQLADHYQQ | |
| | binding | ggtagtggttgtcgggcagcagcacggggccgtcgcc | | NTPIGDGPVLLPDNHYLSTQSALSKDPNEKRDHMVLL | |
| | domain; p65 | gatgggggtgttctgctggtagtggtcggcgagctgc | | EFVTAAGITLGMDELYKGS | |
| | activation | acgctgccgtcctcgatgttgtggcggatcttgaagt | | Transcription Factor Polypeptide: | 33 |
| | domain; | tcaccttgatgccgttcttctgcttgtcggccatgat | | MAPKKKRKVERPYACPVESCDRRFSRSDELTRHIRIH | |
| | CA2(L156H) | atagacgttgtggctgttgtagttgtactccagcttg | | TGQKPFQCRICMRNFSRSDHLTTHIRTHTGGGRRRKK | |
| | | tgccccaggatgttgccgtcctccttgaagtcgatgc | | RTSIETNIRVALEKSFLENQKPTSEEITMIADQLNME | |
| | | ccttcagctcgatgcggttcaccagggtgtcgccctc | | KEVIRVWFCNRRQKEKRINTRLGALLGNSTDPAVFTD | |
| | | gaacttcacctcggcgcgggtcttgtagttgccgtcg | | LASVDNSEFQQLLNQGIPVAPHTTEPMLMEYPEAITR | |
| | | tccttgaagaagatggtgcgctcctggacgtagcctt | | LVTGAQRPPDPAPAPLGAPGLPNGLLSGDEDFSSIAD | |
| | | cgggcatggcggacttgaagaagtcgtgctgcttcat | | MDFSALLSQISSGSSHHWGYGKHNGPEHWHKDFPIAK | |
| | | gtggtcggggtagcggctgaagcactgcacgccgtag | | GERQSPVDIDTHTAKYDPSLKPLSVSYDQATSLRILN | |
| | | gtcagggtggtcacgagggtgggccagggcacgggca | | NGHAFNVEFDDSQDKAVLKGGPLDGTYRLIQFHFHWG | |
| | | gcttgccggtggtgcagatgaacttcagggtcagctt | | SLDGQGSEHTVDKKKYAAELHLVHWNTKYGDFGKAVQ | |
| | | gccgtaggtggcatcgccctcgccctcgccggacacg | | QPDGLAVLGIFLKVGSAKPGHQKVVDVLDSIKTKGKS | |
| | | ctgaacttgtggccgtttacgtcgccgtccagctcga | | ADFTNFDPRGLLPESLDYWTYPGSLTTPPLLECVTWI | |
| | | ccaggatgggcaccaccccggtgaacagctcctcgcc | | VLKEPISVSSEQVLKFRKLNFNGEGEPEELMVDNWRP | |
| | | cttgctcaccatGGTGGCTGGATCaGAGCTCGGTACC | | AQPLKNRQIKASFKGS | |
| | | AAGCTaTCTGGTAGTAGACTAGTGGCCCCCATTATAT | | | |
| | | ACCCTCTAGAgctagaacgcccatcattactggagcg | | | |
| | | cccatcattagtctaccgcccatcattactctagcgc | | | |
| | | ccatcattagctagacgcccatcattactcgtgcgcc | | | |
| | | catcattagtcgtccgcccatcattactcgtgcgccc | | | |
| | | atcattaGcacgtgaggctccggtgcccgtcagtggg | | | |
| | | cagagcgcacatcgcccacagtccccgagaagttggg | | | |
| | | gggaggggtcggcaattgaaccggtgcctagagaagg | | | |
| | | tggcgcggggtaaactgggaaagtgatgtcgtgtact | | | |
| | | ggctccgcctttttcccgagggtgggggagaaccgta | | | |
| | | tataagtgcagtagtcgccgtgaacgttctttttcgc | | | |
| | | aacgggtttgccgccagaacacaggtaagtgccgtgt | | | |
| | | gtggttcccgcgggcctggcctctttacgggttatgg | | | |
| | | cccttgcgtgccttgaattacttccacctggctgcag | | | |
| | | tacgtgattcttgatcccgagcttcgggttggaagtg | | | |
| | | ggtgggagagttcgaggccttgcgcttaaggagcccc | | | |
| | | ttcgcctcgtgcttgagttgaggcctggcctgggcgc | | | |
| | | tggggccgccgcgtgcgaatctggtggcaccttcgcg | | | |
| | | cctgtctcgctgctttcgataagtctctagccattta | | | |
| | | aaatttttgatgacctgctgcgacgctttttttctgg | | | |
| | | caagatagtcttgtaaatgcgggccaagatctgcaca | | | |
| | | ctggtatttcggtttttggggccgcgggcggcgacgg | | | |
| | | ggcccgtgcgtcccagcgcacatgttcggcgaggcgg | | | |
| | | ggcctgcgagcgcggccaccgagaatcggacgggggt | | | |
| | | agtctcaagctggccggcctgctctggtgcctggcct | | | |
| | | cgcgccgccgtgtatcgccccgccctgggcggcaagg | | | |
| | | ctggcccggtcggcaccagttgcgtgagcggaaagat | | | |
| | | ggccgcttcccggccctgctgcagggagctcaaaatg | | | |
| | | gaggacgcggcgctcgggagagcgggcgggtgagtca | | | |
| | | cccacacaaaggaaaagggcctttccgtcctcagccg | | | |
| | | tcgcttcatgtgactccactgagtaccgggcgccgtc | | | |
| | | caggcacctcgattagttctcgagcttttggagtacg | | | |
| | | tcgtctttaggttggggggaggggttttatgcgatgg | | | |
| | | agtttccccacactgagtgggtggagactgaagttag | | | |
| | | gccagcttggcacaaatgctttatttgtgaaatttgt | | | |
| | | gatgctattgctttatttgtaaccattataagctgca | | | |
| | | atattgatgtaattctccttggaatttgccctttttg | | | |
| | | agtttggatcttggttcattctcaagcctcagacagt | | | |
| | | ggttcaaagtttttttcttccatttcaggtgtcgtga | | | |
| | | tctagaggatcACTAGTgccaccatgGCACCTAAGaa | | | |
| | | aAAGAGGAAGGTTgaacgcccatatgcttgccctgtc | | | |
| | | gagtcctgcgatcgccgcttttctcgctcggatgagc | | | |
| | | ttacccgccatatccgcatccacacaggccagaagcc | | | |
| | | cttccagtgtcgaatctgcatgcgtaacttcagtcgt | | | |
| | | agtgaccaccttaccacccacatccgcacccacacag | | | |
| | | gcggcggccgcaggaggaagaaacgcaccagcataga | | | |
| | | gaccaacatccgtgtggccttagagaagagtttcttg | | | |
| | | gagaatcaaaagcctacctcggaagagatcactatga | | | |
| | | ttgctgatcagctcaatatggaaaaagaggtgattcg | | | |
| | | tgtttggttctgtaaccgccgccagaaagaaaaaaga | | | |
| | | atcaacactagactgggggccttgcttggcaacagca | | | |
| | | cagacccagctgtgttcacagacctggcatccGTGga | | | |
| | | caactccgagtttcagcagctgctgaaccagggcata | | | |
| | | cctgtggccccccacacaactgagcccatgctgatgg | | | |
| | | agtaccctgaggctataactcgcctagtgacaggggc | | | |
| | | ccagaggccccccgacccagctcctgctccactgggg | | | |
| | | gccccggggctccccaatggcctcctttcaggagatg | | | |
| | | aagacttctcctccattgcggacatggacttctcagc | | | |
| | | cctgctgagtcagatcagctccggatccTCCCATCAC | | | |
| | | TGGGGGTACGGCAAACACAACGGACCTGAGCACTGGC | | | |
| | | ATAAGGACTTCCCCATTGCCAAGGGAGAGCGCCAGTC | | | |
| | | CCCTGTTGACATCGACACTCATACAGCCAAGTATGAC | | | |
| | | CCTTCCCTGAAGCCCCTGTCTGTTTCCTATGATCAAG | | | |
| | | CAACTTCCCTGAGGATCCTCAACAATGGTCATGCTTT | | | |
| | | CAACGTGGAGTTTGATGACTCTCAGGACAAAGCAGTG | | | |
| | | CTCAAGGGAGGACCCCTGGATGGCACTTACAGATTGA | | | |
| | | TTCAGTTTCACTTTCACTGGGGTTCACTTGATGGACA | | | |
| | | AGGTTCAGAGCATACTGTGGATAAAAAGAAATATGCT | | | |
| | | GCAGAACTTCACTTGGTTCACTGGAACACCAAATATG | | | |
| | | GGGATTTTGGGAAAGCTGTGCAGCAACCTGATGGACT | | | |
| | | GGCCGTTCTAGGTATTTTTTTGAAGGTTGGCAGCGCT | | | |
| | | AAACCGGGCCATCAGAAAGTTGTTGATGTGCTGGATT | | | |
| | | CCATTAAAACAAAGGGCAAGAGTGCTGACTTCACTAA | | | |
| | | CTTCGATCCTCGTGGCCTCCTTCCTGAATCCCTGGAT | | | |
| | | TACTGGACCTACCCAGGCTCACTGACCACCCCTCCTC | | | |
| | | TTCTGGAATGTGTGACCTGGATTGTGCTCAAGGAACC | | | |
| | | CATCAGCGTCAGCAGCGAGCAGGTGTTGAAATTCCGT | | | |
| | | AAACTTAACTTCAATGGGGAGGGTGAACCCGAAGAAC | | | |
| | | TGATGGTGGACAACTGGCGCCCAGCTCAGCCACTGAA | | | |
| | | GAACAGGCAAATCAAAGCTTCCTTCAAAggatcctga | | | |
| ZFHD- | EF1a | cgtgaggctccggtgcccgtcagtgggcagagcgcac | 34 | MAPKKKRKVERPYACPVESCDRRFSRSDELTRHIRIH | 35 |
| 048 | promoter; | atcgcccacagtccccgagaagttggggggaggggtc | | TGQKPFQCRICMRNFSRSDHLTTHIRTHTGGGRRRKK | |
| | ZFHD1 DNA | ggcaattgaaccggtgcctagagaaggtggcgcgggg | | RTSIETNIRVALEKSFLENQKPTSEEITMIADQLNME | |
| | binding | taaactgggaaagtgatgtcgtgtactggctccgcct | | KEVIRVWFCNRRQKEKRINTRLGALLGNSTDPAVFTD | |
| | domain; p65 | ttttcccgagggtgggggagaaccgtatataagtgca | | LASVDNSEFQQLLNQGIPVAPHTTEPMLMEYPEAITR | |
| | activation | gtagtcgccgtgaacgttctttttcgcaacgggtttg | | LVTGAQRPPDPAPAPLGAPGLPNGLLSGDEDFSSIAD | |
| | domain; | ccgccagaacacaggtaagtgccgtgtgtggttcccg | | MDFSALLSQISSGSSHHWGYGKHNGPEHWHKDFPIAK | |
| | CA2(L156H) | cgggcctggcctctttacgggttatggcccttgcgtg | | GERQSPVDIDTHTAKYDPSLKPLSVSYDQATSLRILN | |
| | | ccttgaattacttccacctggctgcagtacgtgattc | | NGHAFNVEFDDSQDKAVLKGGPLDGTYRLIQFHFHWG | |
| | | ttgatcccgagcttcgggttggaagtgggtgggagag | | SLDGQGSEHTVDKKKYAAELHLVHWNTKYGDFGKAVQ | |
| | | ttcgaggccttgcgcttaaggagccccttcgcctcgt | | QPDGLAVLGIFLKVGSAKPGHQKVVDVLDSIKTKGKS | |
| | | gcttgagttgaggcctggcctgggcgctggggccgcc | | ADFTNFDPRGLLPESLDYWTYPGSLTTPPLLECVTWI | |
| | | gcgtgcgaatctggtggcaccttcgcgcctgtctcgc | | VLKEPISVSSEQVLKFRKLNFNGEGEPEELMVDNWRP | |
| | | tgctttcgataagtctctagccatttaaaatttttga | | AQPLKNRQIKASFKGS* | |
| | | tgacctgctgcgacgctttttttctggcaagatagtc | | | |
| | | ttgtaaatgcgggccaagatctgcacactggtatttc | | | |
| | | ggtttttggggccgcgggcggcgacggggcccgtgcg | | | |
| | | tcccagcgcacatgttcggcgaggcggggcctgcgag | | | |
| | | cgcggccaccgagaatcggacgggggtagtctcaagc | | | |
| | | tggccggcctgctctggtgcctggcctcgcgccgccg | | | |
| | | tgtatcgccccgccctgggcggcaaggctggcccggt | | | |
| | | cggcaccagttgcgtgagcggaaagatggccgcttcc | | | |
| | | cggccctgctgcagggagctcaaaatggaggacgcgg | | | |
| | | cgctcgggagagcgggcgggtgagtcacccacacaaa | | | |
| | | ggaaaagggcctttccgtcctcagccgtcgcttcatg | | | |
| | | tgactccactgagtaccgggcgccgtccaggcacctc | | | |
| | | gattagttctcgagcttttggagtacgtcgtctttag | | | |
| | | gttggggggaggggttttatgcgatggagtttcccca | | | |
| | | cactgagtgggtggagactgaagttaggccagcttgg | | | |
| | | cacttgatgtaattctccttggaatttgccctttttg | | | |
| | | agtttggatcttggttcattctcaagcctcagacagt | | | |
| | | ggttcaaagtttttttcttccatttcaggtgtcgtga | | | |
| | | tctagaggatcACTAGTgccaccatgGCACCTAAGaa | | | |
| | | aAAGAGGAAGGTTgaacgcccatatgcttgccctgtc | | | |
| | | gagtcctgcgatcgccgcttttctcgctcggatgagc | | | |
| | | ttacccgccatatccgcatccacacaggccagaagcc | | | |
| | | cttccagtgtcgaatctgcatgcgtaacttcagtcgt | | | |
| | | agtgaccaccttaccacccacatccgcacccacacag | | | |
| | | gcggcggccgcaggaggaagaaacgcaccagcataga | | | |
| | | gaccaacatccgtgtggccttagagaagagtttcttg | | | |
| | | gagaatcaaaagcctacctcggaagagatcactatga | | | |
| | | ttgctgatcagctcaatatggaaaaagaggtgattcg | | | |
| | | tgtttggttctgtaaccgccgccagaaagaaaaaaga | | | |
| | | atcaacactagactgggggccttgcttggcaacagca | | | |
| | | cagacccagctgtgttcacagacctggcatccGTGga | | | |
| | | caactccgagtttcagcagctgctgaaccagggcata | | | |
| | | cctgtggccccccacacaactgagcccatgctgatgg | | | |
| | | agtaccctgaggctataactcgcctagtgacaggggc | | | |
| | | ccagaggccccccgacccagctcctgctccactgggg | | | |
| | | gccccggggctccccaatggcctcctttcaggagatg | | | |
| | | aagacttctcctccattgcggacatggacttctcagc | | | |
| | | cctgctgagtcagatcagctccggatccTCCCATCAC | | | |
| | | TGGGGGTACGGCAAACACAACGGACCTGAGCACTGGC | | | |
| | | ATAAGGACTTCCCCATTGCCAAGGGAGAGCGCCAGTC | | | |
| | | CCCTGTTGACATCGACACTCATACAGCCAAGTATGAC | | | |
| | | CCTTCCCTGAAGCCCCTGTCTGTTTCCTATGATCAAG | | | |
| | | CAACTTCCCTGAGGATCCTCAACAATGGTCATGCTTT | | | |
| | | CAACGTGGAGTTTGATGACTCTCAGGACAAAGCAGTG | | | |
| | | CTCAAGGGAGGACCCCTGGATGGCACTTACAGATTGA | | | |
| | | TTCAGTTTCACTTTCACTGGGGTTCACTTGATGGACA | | | |
| | | AGGTTCAGAGCATACTGTGGATAAAAAGAAATATGCT | | | |
| | | GCAGAACTTCACTTGGTTCACTGGAACACCAAATATG | | | |
| | | GGGATTTTGGGAAAGCTGTGCAGCAACCTGATGGACT | | | |
| | | GGCCGTTCTAGGTATTTTTTTGAAGGTTGGCAGCGCT | | | |
| | | AAACCGGGCCATCAGAAAGTTGTTGATGTGCTGGATT | | | |
| | | CCATTAAAACAAAGGGCAAGAGTGCTGACTTCACTAA | | | |
| | | CTTCGATCCTCGTGGCCTCCTTCCTGAATCCCTGGAT | | | |
| | | TACTGGACCTACCCAGGCTCACTGACCACCCCTCCTC | | | |
| | | TTCTGGAATGTGTGACCTGGATTGTGCTCAAGGAACC | | | |
| | | CATCAGCGTCAGCAGCGAGCAGGTGTTGAAATTCCGT | | | |
| | | AAACTTAACTTCAATGGGGAGGGTGAACCCGAAGAAC | | | |
| | | TGATGGTGGACAACTGGCGCCCAGCTCAGCCACTGAA | | | |
| | | GAACAGGCAAATCAAAGCTTCCTTCAAAggatcctga | | | |
| ZFHD- | EGFP; Min | ttaggatcccttgtacagctcgtccatgccgagagtg | 36 | MVSKGEELFTGVVPILVELDGDVNGHKFSVSGEGEGD | 37 |
| 010 | promoter; | atcccggcggcggtcacgaactccagcaggaccatgt | | ATYGKLTLKFICTTGKLPVPWPTLVTTLTYGVQCFSR | |
| | 8xZFHD1 BS | gatcgcgcttctcgttggggtctttgctcagggcgga | | YPDHMKQHDFFKSAMPEGYVQERTIFFKDDGNYKTRA | |
| | | ctgggtgctcaggtagtggttgtcgggcagcagcacg | | EVKFEGDTLVNRIELKGIDFKEDGNILGHKLEYNYNS | |
| | | gggccgtcgccgatgggggtgttctgctggtagtggt | | HNVYIMADKQKNGIKVNFKIRHNIEDGSVQLADHYQQ | |
| | | cggcgagctgcacgctgccgtcctcgatgttgtggcg | | NTPIGDGPVLLPDNHYLSTQSALSKDPNEKRDHMVLL | |
| | | gatcttgaagttcaccttgatgccgttcttctgcttg | | EFVTAAGITLGMDELYKGS | |
| | | tcggccatgatatagacgttgtggctgttgtagttgt | | | |
| | | actccagcttgtgccccaggatgttgccgtcctcctt | | | |
| | | gaagtcgatgcccttcagctcgatgcggttcaccagg | | | |
| | | gtgtcgccctcgaacttcacctcggcgcgggtcttgt | | | |
| | | agttgccgtcgtccttgaagaagatggtgcgctcctg | | | |
| | | gacgtagccttcgggcatggcggacttgaagaagtcg | | | |
| | | tgctgcttcatgtggtcggggtagcggctgaagcact | | | |
| | | gcacgccgtaggtcagggtggtcacgagggtgggcca | | | |
| | | gggcacgggcagcttgccggtggtgcagatgaacttc | | | |
| | | agggtcagcttgccgtaggtggcatcgccctcgccct | | | |
| | | cgccggacacgctgaacttgtggccgtttacgtcgcc | | | |
| | | gtccagctcgaccaggatgggcaccaccccggtgaac | | | |
| | | agctcctcgcccttgctcaccatGGTGGCTGGATCaG | | | |
| | | AGCTCGGTACCAAGCTaTCTGGTAGTAGACTAGTGGC | | | |
| | | CCCCATTATATACCCTCTAGAgctagaacgcccatca | | | |
| | | ttactggagcgcccatcattagtctaccgcccatcat | | | |
| | | tactctagcgcccatcattagctagacgcccatcatt | | | |
| | | actcgtgcgcccatcattagtcgtccgcccatcatta | | | |
| | | ctcgtgcgcccatcatta | | | |
| EGFP- | EGFP | atggtgagcaagggcgaggagctgttcaccggggtgg | 38 | MVSKGEELFTGVVPILVELDGDVNGHKFSVSGEGEGD | 39 |
| 001 | | tgcccatcctggtcgagctggacggcgacgtaaacgg | | ATYGKLTLKFICTTGKLPVPWPTLVTTLTYGVQCFSR | |
| (con- | | ccacaagttcagcgtgtccggcgagggcgagggcgat | | YPDHMKQHDFFKSAMPEGYVQERTIFFKDDGNYKTRA | |
| trol | | gccacctacggcaagctgaccctgaagttcatctgca | | EVKFEGDTLVNRIELKGIDFKEDGNILGHKLEYNYNS | |
| con- | | ccaccggcaagctgcccgtgccctggcccaccctcgt | | HNVYIMADKQKNGIKVNFKIRHNIEDGSVQLADHYQQ | |
| struct) | | gaccaccctgacctacggcgtgcagtgcttcagccgc | | NTPIGDGPVLLPDNHYLSTQSALSKDPNEKRDHMVLL | |
| | | taccccgaccacatgaagcagcacgacttcttcaagt | | EFVTAAGITLGMDELYK* | |
| | | ccgccatgcccgaaggctacgtccaggagcgcaccat | | | |
| | | cttcttcaaggacgacggcaactacaagacccgcgcc | | | |
| | | gaggtgaagttcgagggcgacaccctggtgaaccgca | | | |
| | | tcgagctgaagggcatcgacttcaaggaggacggcaa | | | |
| | | catcctggggcacaagctggagtacaactacaacagc | | | |
| | | cacaacgtctatatcatggccgacaagcagaagaacg | | | |
| | | gcatcaaggtgaacttcaagatccgccacaacatcga | | | |
| | | ggacggcagcgtgcagctcgccgaccactaccagcag | | | |
| | | aacacccccatcggcgacggccccgtgctgctgcccg | | | |
| | | acaaccactacctgagcacccagtccgccctgagcaa | | | |
| | | agaccccaacgagaagcgcgatcacatggtcctgctg | | | |
| | | gagttcgtgaccgccgccgggatcactctcggcatgg | | | |
| | | acgagctgtacaagtaa | | | |

Additional illustrative constructs comprising structurally different transcription factor components are provided in Table 2. An asterisk (“*”) in Table 2 indicates the translation of a stop codon. Corresponding control constructs not comprising a regulated transcription factor as well as separate construct components are also provided. As indicated in the description for constructs cjun-001 and cjun-002, a peptide linker is positioned between the CA2 component and the c-Jun component of each construct. Additionally, all constructs comprise a P2A peptide.

TABLE 2
Constructs and construct components comprising different transcription factors

| | | | NA | | AA |
|---|---|---|---|---|---|
| | | | SEQ | | SEQ |
| | | | ID | | ID |
| Construct | Description | NA Sequence | NO. | AA Sequence | NO. |
| cjun-001 | CA2(WT)-GGSGGGSG | ATGTCTCACCACTGGGGCTACGGCAAGCACAATGGACC | 40 | MSHHWGYGKHNGPEHWHKDFPIAKG | 41 |
| | (SEQ ID NO: 58)- | TGAGCACTGGCACAAGGACTTCCCTATCGCCAAGGGCG | | ERQSPVDIDTHTAKYDPSLKPLSVS | |
| | cjun-p2A-mCherry | AGAGACAGAGCCCCGTGGACATCGATACCCACACCGCC | | YDQATSLRILNNGHAFNVEFDDSQD | |
| | | AAATACGACCCCAGCCTGAAGCCTCTGAGCGTGTCCTA | | KAVLKGGPLDGTYRLIQFHFHWGSL | |
| | | TGATCAGGCCACCAGCCTGCGCATCCTGAACAATGGCC | | DGQGSEHTVDKKKYAAELHLVHWNT | |
| | | ACGCCTTCAACGTGGAATTCGACGACAGCCAGGACAAG | | KYGDFGKAVQQPDGLAVLGIFLKVG | |
| | | GCCGTGCTGAAAGGTGGACCTCTGGACGGCACCTACCG | | SAKPGLQKVVDVLDSIKTKGKSADF | |
| | | GCTGATCCAGTTCCACTTTCACTGGGGCAGCCTGGATG | | TNFDPRGLLPESLDYWTYPGSLTTP | |
| | | GCCAGGGATCTGAACACACCGTGGACAAGAAGAAGTAC | | PLLECVTWIVLKEPISVSSEQVLKF | |
| | | GCCGCCGAACTGCACCTGGTGCACTGGAACACCAAATA | | RKLNFNGEGEPEELMVDNWRPAQPL | |
| | | CGGCGACTTCGGCAAAGCCGTGCAGCAGCCTGATGGAC | | KNRQIKASFKGGSGGGSGTAKMETT | |
| | | TGGCTGTGCTGGGCATCTTCCTGAAAGTGGGCTCTGCC | | FYDDALNASFLPSESGPYGYSNPKI | |
| | | AAGCCTGGCCTGCAGAAAGTGGTGGACGTGCTGGACAG | | LKQSMTLNLADPVGSLKPHLRAKNS | |
| | | CATCAAGACCAAGGGCAAGAGCGCCGACTTCACCAACT | | DLLTSPDVGLLKLASPELERLIIQS | |
| | | TCGACCCTAGAGGACTGCTGCCCGAGAGCCTGGACTAC | | SNGHITTTPTPTQFLCPKNVTDEQE | |
| | | TGGACATACCCTGGCAGCCTGACCACACCTCCTCTGCT | | GFAEGFVRALAELHSQNTLPSVTSA | |
| | | GGAATGTGTGACCTGGATCGTCCTGAAAGAGCCCATCA | | AQPVNGAGMVAPAVASVAGGSGSGG | |
| | | GCGTCAGCAGCGAACAGGTGCTGAAGTTCCGGAAGCTG | | FSASLHSEPPVYANLSNFNPGALSS | |
| | | AACTTCAACGGCGAGGGCGAGCCTGAGGAACTGATGGT | | GGGAPSYGAAGLAFPAQPQQQQQPP | |
| | | GGATAATTGGAGGCCCGCTCAGCCCCTGAAGAACAGAC | | HHLPQQMPVQHPRLQALKEEPQTVP | |
| | | AGATCAAGGCCAGCTTCAAGggcggctctggcggagga | | EMPGETPPLSPIDMESQERIKAERK | |
| | | tctggaACCGCCAAGATGGAAACCACCTTCTACGACGA | | RMRNRIAASKCRKRKLERIARLEEK | |
| | | CGCCCTGAACGCCAGCTTTCTGCCTTCTGAGTCTGGCC | | VKTLKAQNSELASTANMLREQVAQL | |
| | | CCTACGGCTACAGCAACCCCAAGATCCTGAAGCAGAGC | | KQKVMNHVNSGCQLMLTQQLQTFGS | |
| | | ATGACCCTGAACCTGGCCGATCCTGTGGGCAGCCTGAA | | GATNFSLLKQAGDVEENPGPLSKGE | |
| | | ACCTCACCTGAGAGCCAAGAACAGCGACCTGCTGACAA | | EDNMAIIKEFMRFKVHMEGSVNGHE | |
| | | GCCCTGATGTGGGCCTGCTGAAACTGGCTAGCCCCGAG | | FEIEGEGEGRPYEGTQTAKLKVTKG | |
| | | CTGGAACGGCTGATCATCCAGTCTAGCAACGGCCACAT | | GPLPFAWDILSPQFMYGSKAYVKHP | |
| | | CACCACCACACCTACACCAACACAGTTTCTGTGCCCCA | | ADIPDYLKLSFPEGFKWERVMNFED | |
| | | AGAACGTGACCGACGAGCAAGAGGGATTCGCCGAGGGC | | GGVVTVTQDSSLQDGEFIYKVKLRG | |
| | | TTTGTTAGAGCCCTGGCCGAACTGCACAGCCAGAATAC | | TNFPSDGPVMQKKTMGWEASSERMY | |
| | | CCTGCCTAGCGTGACATCTGCCGCTCAGCCTGTTAATG | | PEDGALKGEIKQRLKLKDGGHYDAE | |
| | | GCGCCGGAATGGTTGCTCCTGCCGTGGCTTCTGTTGCT | | VKTTYKAKKPVQLPGAYNVNIKLDI | |
| | | GGCGGATCTGGATCTGGCGGCTTTAGCGCCTCTCTGCA | | TSHNEDYTIVEQYERAEGRHSTGGM | |
| | | CTCTGAGCCTCCAGTGTACGCCAACCTGAGCAACTTCA | | DELYK* | |
| | | ACCCTGGCGCTCTTAGCTCTGGTGGCGGAGCACCTTCT | | | |
| | | TATGGCGCTGCCGGATTGGCCTTTCCTGCTCAGCCTCA | | | |
| | | GCAGCAGCAACAGCCTCCTCATCATCTGCCCCAGCAGA | | | |
| | | TGCCTGTGCAGCACCCTAGACTGCAGGCCCTGAAAGAG | | | |
| | | GAACCCCAGACAGTCCCTGAGATGCCCGGCGAAACACC | | | |
| | | TCCTCTGAGCCCCATCGACATGGAAAGCCAAGAGCGGA | | | |
| | | TCAAGGCCGAGCGGAAGCGGATGAGAAATAGAATCGCC | | | |
| | | GCCTCCAAGTGCCGGAAGAGGAAGCTGGAAAGAATCGC | | | |
| | | CCGGCTGGAAGAGAAAGTGAAAACCCTGAAGGCCCAGA | | | |
| | | ACTCCGAGCTGGCCTCTACCGCCAACATGCTGAGAGAA | | | |
| | | CAGGTGGCCCAGCTGAAACAGAAAGTCATGAACCACGT | | | |
| | | GAACAGCGGCTGCCAGCTGATGCTGACACAGCAGCTGC | | | |
| | | AGACCTTCggatccggagctactaacttcagcctgctg | | | |
| | | aagcaggctggagacgtggaggagaaccctggaccttt | | | |
| | | gagcaagggcgaggaggacaacatggccatcatcaagg | | | |
| | | agttcatgcgcttcaaggtgcacatggagggctccgtg | | | |
| | | aacggccacgagttcgagatcgagggcgagggcgaggg | | | |
| | | ccgcccctacgagggcacccagaccgccaagctgaagg | | | |
| | | tgaccaagggcggccccctgcccttcgcctgggacatc | | | |
| | | ctgtcccctcagttcatgtacggctccaaggcctacgt | | | |
| | | gaagcaccccgccgacatccccgactacttgaagctgt | | | |
| | | ccttccccgagggcttcaagtgggagcgcgtgatgaac | | | |
| | | ttcgaggacggcggcgtggtgaccgtgacccaggactc | | | |
| | | ctccctgcaggacggcgagttcatctacaaggtgaagc | | | |
| | | tgcgcggcaccaacttcccctccgacggccccgtaatg | | | |
| | | cagaagaagaccatgggctgggaggcctcctccgagcg | | | |
| | | gatgtaccccgaggacggcgccctgaagggcgagatca | | | |
| | | agcagaggctgaagctgaaggacggcggccactacgac | | | |
| | | gccgaggtcaagaccacctacaaggccaagaagcccgt | | | |
| | | gcagctgcccggcgcctacaacgtcaacatcaagctgg | | | |
| | | acatcacctcccacaacgaggactacaccatcgtggaa | | | |
| | | cagtacgagcgcgccgagggccgccactccaccggcgg | | | |
| | | catggacgagctgtacaagtaa | | | |
| cjun-002 | CA2(L156H)- | ATGTCTCACCACTGGGGCTACGGCAAGCACAATGGACC | 42 | MSHHWGYGKHNGPEHWHKDFPIAKG | 43 |
| | GGSGGGSG | TGAGCACTGGCACAAGGACTTCCCTATCGCCAAGGGCG | | ERQSPVDIDTHTAKYDPSLKPLSVS | |
| | (SEQ ID NO: 58)- | AGAGACAGAGCCCCGTGGACATCGATACCCACACCGCC | | YDQATSLRILNNGHAFNVEFDDSQD | |
| | cjun-p2A-mCherry | AAATACGACCCCAGCCTGAAGCCTCTGAGCGTGTCCTA | | KAVLKGGPLDGTYRLIQFHFHWGSL | |
| | | TGATCAGGCCACCAGCCTGCGCATCCTGAACAATGGCC | | DGQGSEHTVDKKKYAAELHLVHWNT | |
| | | ACGCCTTCAACGTGGAATTCGACGACAGCCAGGACAAG | | KYGDFGKAVQQPDGLAVLG1FLKVG | |
| | | GCCGTGCTGAAAGGTGGACCTCTGGACGGCACCTACCG | | SAKPGHQKVVDVLDSIKTKGKSADF | |
| | | GCTGATCCAGTTCCACTTTCACTGGGGCAGCCTGGATG | | TNFDPRGLLPESLDYWTYPGSLTTP | |
| | | GCCAGGGATCTGAACACACCGTGGACAAGAAGAAGTAC | | PLLECVTWIVLKEPISVSSEQVLKF | |
| | | GCCGCCGAACTGCACCTGGTGCACTGGAACACCAAATA | | RKLNFNGEGEPEELMVDNWRPAQPL | |
| | | CGGCGACTTCGGCAAAGCCGTGCAGCAGCCTGATGGAC | | KNRQIKASFKGGSGGGSGTAKMETT | |
| | | TGGCTGTGCTGGGCATCTTCCTGAAAGTGGGCTCTGCC | | FYDDALNASFLPSESGPYGYSNPKI | |
| | | AAGCCTGGCCacCAGAAAGTGGTGGACGTGCTGGACAG | | LKQSMTLNLADPVGSLKPHLRAKNS | |
| | | CATCAAGACCAAGGGCAAGAGCGCCGACTTCACCAACT | | DLLTSPDVGLLKLASPELERLIIQS | |
| | | TCGACCCTAGAGGACTGCTGCCCGAGAGCCTGGACTAC | | SNGHITTTPTPTQFLCPKNVTDEQE | |
| | | TGGACATACCCTGGCAGCCTGACCACACCTCCTCTGCT | | GFAEGFVRALAELHSQNTLPSVTSA | |
| | | GGAATGTGTGACCTGGATCGTCCTGAAAGAGCCCATCA | | AQPVNGAGMVAPAVASVAGGSGSGG | |
| | | GCGTCAGCAGCGAACAGGTGCTGAAGTTCCGGAAGCTG | | FSASLHSEPPVYANLSNFNPGALSS | |
| | | AACTTCAACGGCGAGGGCGAGCCTGAGGAACTGATGGT | | GGGAPSYGAAGLAFPAQPQQQQQPP | |
| | | GGATAATTGGAGGCCCGCTCAGCCCCTGAAGAACAGAC | | HHLPQQMPVQHPRLQALKEEPQTVP | |
| | | AGATCAAGGCCAGCTTCAAGggcggctctggcggagga | | EMPGETPPLSPIDMESQERIKAERK | |
| | | tctggaACCGCCAAGATGGAAACCACCTTCTACGACGA | | RMRNRIAASKCRKRKLERIARLEEK | |
| | | CGCCCTGAACGCCAGCTTTCTGCCTTCTGAGTCTGGCC | | VKTLKAQNSELASTANMLREQVAQL | |
| | | CCTACGGCTACAGCAACCCCAAGATCCTGAAGCAGAGC | | KQKVMNHVNSGCQLMLTQQLQTFGS | |
| | | ATGACCCTGAACCTGGCCGATCCTGTGGGCAGCCTGAA | | GATNFSLLKQAGDVEENPGPLSKGE | |
| | | ACCTCACCTGAGAGCCAAGAACAGCGACCTGCTGACAA | | EDNMAIIKEFMRFKVHMEGSVNGHE | |
| | | GCCCTGATGTGGGCCTGCTGAAACTGGCTAGCCCCGAG | | FEIEGEGEGRPYEGTQTAKLKVTKG | |
| | | CTGGAACGGCTGATCATCCAGTCTAGCAACGGCCACAT | | GPLPFAWDILSPQFMYGSKAYVKHP | |
| | | CACCACCACACCTACACCAACACAGTTTCTGTGCCCCA | | ADIPDYLKLSFPEGFKWERVMNFED | |
| | | AGAACGTGACCGACGAGCAAGAGGGATTCGCCGAGGGC | | GGVVTVTQDSSLQDGEFIYKVKLRG | |
| | | TTTGTTAGAGCCCTGGCCGAACTGCACAGCCAGAATAC | | TNFPSDGPVMQKKTMGWEASSERMY | |
| | | CCTGCCTAGCGTGACATCTGCCGCTCAGCCTGTTAATG | | PEDGALKGEIKQRLKLKDGGHYDAE | |
| | | GCGCCGGAATGGTTGCTCCTGCCGTGGCTTCTGTTGCT | | VKTTYKAKKPVQLPGAYNVNIKLDI | |
| | | GGCGGATCTGGATCTGGCGGCTTTAGCGCCTCTCTGCA | | TSHNEDYTIVEQYERAEGRHSTGGM | |
| | | CTCTGAGCCTCCAGTGTACGCCAACCTGAGCAACTTCA | | DELYK* | |
| | | ACCCTGGCGCTCTTAGCTCTGGTGGCGGAGCACCTTCT | | | |
| | | TATGGCGCTGCCGGATTGGCCTTTCCTGCTCAGCCTCA | | | |
| | | GCAGCAGCAACAGCCTCCTCATCATCTGCCCCAGCAGA | | | |
| | | TGCCTGTGCAGCACCCTAGACTGCAGGCCCTGAAAGAG | | | |
| | | GAACCCCAGACAGTCCCTGAGATGCCCGGCGAAACACC | | | |
| | | TCCTCTGAGCCCCATCGACATGGAAAGCCAAGAGCGGA | | | |
| | | TCAAGGCCGAGCGGAAGCGGATGAGAAATAGAATCGCC | | | |
| | | GCCTCCAAGTGCCGGAAGAGGAAGCTGGAAAGAATCGC | | | |
| | | CCGGCTGGAAGAGAAAGTGAAAACCCTGAAGGCCCAGA | | | |
| | | ACTCCGAGCTGGCCTCTACCGCCAACATGCTGAGAGAA | | | |
| | | CAGGTGGCCCAGCTGAAACAGAAAGTCATGAACCACGT | | | |
| | | GAACAGCGGCTGCCAGCTGATGCTGACACAGCAGCTGC | | | |
| | | AGACCTTCggatccggagctactaacttcagcctgctg | | | |
| | | aagcaggctggagacgtggaggagaaccctggaccttt | | | |
| | | gagcaagggcgaggaggacaacatggccatcatcaagg | | | |
| | | agttcatgcgcttcaaggtgcacatggagggctccgtg | | | |
| | | aacggccacgagttcgagatcgagggcgagggcgaggg | | | |
| | | ccgcccctacgagggcacccagaccgccaagctgaagg | | | |
| | | tgaccaagggcggccccctgcccttcgcctgggacatc | | | |
| | | ctgtcccctcagttcatgtacggctccaaggcctacgt | | | |
| | | gaagcaccccgccgacatccccgactacttgaagctgt | | | |
| | | ccttccccgagggcttcaagtgggagcgcgtgatgaac | | | |
| | | ttcgaggacggcggcgtggtgaccgtgacccaggactc | | | |
| | | ctccctgcaggacggcgagttcatctacaaggtgaagc | | | |
| | | tgcgcggcaccaacttcccctccgacggccccgtaatg | | | |
| | | cagaagaagaccatgggctgggaggcctcctccgagcg | | | |
| | | gatgtaccccgaggacggcgccctgaagggcgagatca | | | |
| | | agcagaggctgaagctgaaggacggcggccactacgac | | | |
| | | gccgaggtcaagaccacctacaaggccaagaagcccgt | | | |
| | | gcagctgcccggcgcctacaacgtcaacatcaagctgg | | | |
| | | acatcacctcccacaacgaggactacaccatcgtggaa | | | |
| | | cagtacgagcgcgccgagggccgccactccaccggcgg | | | |
| | | catggacgagctgtacaagtaa | | | |
| cjun-003 | cjun-p2A-mCherry | atgACCGCCAAGATGGAAACCACCTTCTACGACGACGC | 44 | MTAKMETTFYDDALNASFLPSESGP | 45 |
| | | CCTGAACGCCAGCTTTCTGCCTTCTGAGTCTGGCCCCT | | YGYSNPKILKQSMTLNLADPVGSLK | |
| | | ACGGCTACAGCAACCCCAAGATCCTGAAGCAGAGCATG | | PHLRAKNSDLLTSPDVGLLKLASPE | |
| | | ACCCTGAACCTGGCCGATCCTGTGGGCAGCCTGAAACC | | LERLHQSSNGHITTTPTPTQFLCPK | |
| | | TCACCTGAGAGCCAAGAACAGCGACCTGCTGACAAGCC | | NVTDEQEGFAEGFVRALAELHSQNT | |
| | | CTGATGTGGGCCTGCTGAAACTGGCTAGCCCCGAGCTG | | LPSVTSAAQPVNGAGMVAPAVASVA | |
| | | GAACGGCTGATCATCCAGTCTAGCAACGGCCACATCAC | | GGSGSGGFSASLHSEPPVYANLSNF | |
| | | CACCACACCTACACCAACACAGTTTCTGTGCCCCAAGA | | NPGALSSGGGAPSYGAAGLAFPAQP | |
| | | ACGTGACCGACGAGCAAGAGGGATTCGCCGAGGGCTTT | | QQQQQPPHHLPQQMPVQHPRLQALK | |
| | | GTTAGAGCCCTGGCCGAACTGCACAGCCAGAATACCCT | | EEPQTVPEMPGETPPLSPIDMESQE | |
| | | GCCTAGCGTGACATCTGCCGCTCAGCCTGTTAATGGCG | | RIKAERKRMRNRIAASKCRKRKLER | |
| | | CCGGAATGGTTGCTCCTGCCGTGGCTTCTGTTGCTGGC | | IARLEEKVKTLKAQNSELASTANML | |
| | | GGATCTGGATCTGGCGGCTTTAGCGCCTCTCTGCACTC | | REQVAQLKQKVMNHVNSGCQLMLTQ | |
| | | TGAGCCTCCAGTGTACGCCAACCTGAGCAACTTCAACC | | QLQTFGSGATNFSLLKQAGDVEENP | |
| | | CTGGCGCTCTTAGCTCTGGTGGCGGAGCACCTTCTTAT | | GPLSKGEEDNMAIIKEFMRFKVHME | |
| | | GGCGCTGCCGGATTGGCCTTTCCTGCTCAGCCTCAGCA | | GSVNGHEFEIEGEGEGRPYEGTQTA | |
| | | GCAGCAACAGCCTCCTCATCATCTGCCCCAGCAGATGC | | KLKVTKGGPLPFAWDILSPQFMYGS | |
| | | CTGTGCAGCACCCTAGACTGCAGGCCCTGAAAGAGGAA | | KAYVKHPADIPDYLKLSFPEGFKWE | |
| | | CCCCAGACAGTCCCTGAGATGCCCGGCGAAACACCTCC | | RVMNFEDGGVVTVTQDSSLQDGEFI | |
| | | TCTGAGCCCCATCGACATGGAAAGCCAAGAGCGGATCA | | YKVKLRGTNFPSDGPVMQKKTMGWE | |
| | | AGGCCGAGCGGAAGCGGATGAGAAATAGAATCGCCGCC | | ASSERMYPEDGALKGEIKQRLKLKD | |
| | | TCCAAGTGCCGGAAGAGGAAGCTGGAAAGAATCGCCCG | | GGHYDAEVKTTYKAKKPVQLPGAYN | |
| | | GCTGGAAGAGAAAGTGAAAACCCTGAAGGCCCAGAACT | | VNIKLDITSHNEDYTIVEQYERAEG | |
| | | CCGAGCTGGCCTCTACCGCCAACATGCTGAGAGAACAG | | RHSTGGMDELYK* | |
| | | GTGGCCCAGCTGAAACAGAAAGTCATGAACCACGTGAA | | | |
| | | CAGCGGCTGCCAGCTGATGCTGACACAGCAGCTGCAGA | | | |
| | | CCTTCggatccggagctactaacttcagcctgctgaag | | | |
| | | caggctggagacgtggaggagaaccctggacctttgag | | | |
| | | caagggcgaggaggacaacatggccatcatcaaggagt | | | |
| | | tcatgcgcttcaaggtgcacatggagggctccgtgaac | | | |
| | | ggccacgagttcgagatcgagggcgagggcgagggccg | | | |
| | | cccctacgagggcacccagaccgccaagctgaaggtga | | | |
| | | ccaagggcggccccctgcccttcgcctgggacatcctg | | | |
| | | tcccctcagttcatgtacggctccaaggcctacgtgaa | | | |
| | | gcaccccgccgacatccccgactacttgaagctgtcct | | | |
| | | tccccgagggcttcaagtgggagcgcgtgatgaacttc | | | |
| | | gaggacggcggcgtggtgaccgtgacccaggactcctc | | | |
| | | cctgcaggacggcgagttcatctacaaggtgaagctgc | | | |
| | | gcggcaccaacttcccctccgacggccccgtaatgcag | | | |
| | | aagaagaccatgggctgggaggcctcctccgagcggat | | | |
| | | gtaccccgaggacggcgccctgaagggcgagatcaagc | | | |
| | | agaggctgaagctgaaggacggcggccactacgacgcc | | | |
| | | gaggtcaagaccacctacaaggccaagaagcccgtgca | | | |
| | | gctgcccggcgcctacaacgtcaacatcaagctggaca | | | |
| | | tcacctcccacaacgaggactacaccatcgtggaacag | | | |
| | | tacgagcgcgccgagggccgccactccaccggcggcat | | | |
| | | ggacgagctgtacaagtaa | | | |
| FOXP3- | RQR8-P2A-CA2wt- | ATGGGCACAAGCCTGCTGTGTTGGATGGCCCTGTGTCT | 46 | MGTSLLCWMALCLLGADHADACPYS | 47 |
| 013 | FOXP3FL | GCTGGGAGCCGATCATGCTGATGCCTGTCCTTACAGCA | | NPSLCSGGGGSELPTQGTFSNVSTN | |
| | | ACCCCAGCCTGTGTTCTGGCGGCGGAGGATCTGAACTG | | VSPAKPTTTACPYSNPSLCSGGGGS | |
| | | CCTACACAGGGCACCTTCAGCAACGTGTCCACCAATGT | | PAPRPPTPAPTIASQPLSLRPEACR | |
| | | GTCCCCAGCCAAGCCTACCACCACCGCTTGTCCCTACT | | PAAGGAVHTRGLDFACDIYIWAPLA | |
| | | CCAATCCTAGCCTGTGTAGCGGAGGTGGCGGAAGCCCT | | GTCGVLLLSLVITLYCNHRNRRRVC | |
| | | GCTCCTAGACCTCCTACACCAGCTCCTACAATCGCCAG | | KCPRPVVGSGATNFSLLKQAGDVEE | |
| | | CCAGCCTCTGTCTCTGAGGCCAGAAGCTTGTAGACCTG | | NPGPGGSMSHHWGYGKHNGPEHWHK | |
| | | CTGCTGGCGGAGCCGTGCATACAAGAGGACTGGATTTC | | DFPIAKGERQSPVDIDTHTAKYDPS | |
| | | GCCTGCGACATCTACATCTGGGCCCCTCTGGCTGGAAC | | LKPLSVSYDQATSLRILNNGHAFNV | |
| | | ATGTGGCGTTCTGCTGCTGAGCCTGGTCATCACCCTGT | | EFDDSQDKAVLKGGPLDGTYRLIQF | |
| | | ACTGCAACCACCGGAACAGGCGGAGAGTGTGCAAGTGC | | HFHWGSLDGQGSEHTVDKKKYAAEL | |
| | | CCTAGACCTGTGGTTGGATCTGGTgctactaacttcag | | HLVHWNTKYGDFGKAVQQPDGLAVL | |
| | | cctgctgaagcaggctggagacgtggaggagaaccctg | | GIFLKVGSAKPGLQKVVDVLDSIKT | |
| | | gacctGGAGGATCCatgTCCCATCACTGGGGGTACGGC | | KGKSADFTNFDPRGLLPESLDYWTY | |
| | | AAACACAACGGACCTGAGCACTGGCATAAGGACTTCCC | | PGSLTTPPLLECVTWIVLKEPISVS | |
| | | CATTGCCAAGGGAGAGCGCCAGTCCCCTGTTGACATCG | | SEQVLKFRKLNFNGEGEPEELMVDN | |
| | | ACACTCATACAGCCAAGTATGACCCTTCCCTGAAGCCC | | WRPAQPLKNRQIKASFKGGSGMPNP | |
| | | CTGTCTGTTTCCTATGATCAAGCAACTTCCCTGAGGAT | | RPGKPSAPSLALGPSPGASPSWRAA | |
| | | CCTCAACAATGGTCATGCTTTCAACGTGGAGTTTGATG | | PKASDLLGARGPGGTFQGRDLRGGA | |
| | | ACTCTCAGGACAAAGCAGTGCTCAAGGGAGGACCCCTG | | HASSSSLNPMPPSQLQLPTLPLVMV | |
| | | GATGGCACTTACAGATTGATTCAGTTTCACTTTCACTG | | APSGARLGPLPHLQALLQDRPHFMH | |
| | | GGGTTCACTTGATGGACAAGGTTCAGAGCATACTGTGG | | QLSTVDAHARTPVLQVHPLESPAMI | |
| | | ATAAAAAGAAATATGCTGCAGAACTTCACTTGGTTCAC | | SLTPPTTATGVFSLKARPGLPPGIN | |
| | | TGGAACACCAAATATGGGGATTTTGGGAAAGCTGTGCA | | VASLEWVSREPALLCTFPNPSAPRK | |
| | | GCAACCTGATGGACTGGCCGTTCTAGGTATTTTTTTGA | | DSTLSAVPQSSYPLLANGVCKWPGC | |
| | | AGGTTGGCAGCGCTAAACCGGGCCTTCAGAAAGTTGTT | | EKVFEEPEDFLKHCQADHLLDEKGR | |
| | | GATGTGCTGGATTCCATTAAAACAAAGGGCAAGAGTGC | | AQCLLQREMVQSLEQQLVLEKEKLS | |
| | | TGACTTCACTAACTTCGATCCTCGTGGCCTCCTTCCTG | | AMQAHLAGKMALTKASSVASSDKGS | |
| | | AATCCCTGGATTACTGGACCTACCCAGGCTCACTGACC | | CCIVAAGSQGPVVPAWSGPREAPDS | |
| | | ACCCCTCCTCTTCTGGAATGTGTGACCTGGATTGTGCT | | LFAVRRHLWGSHGNSTFPEFLHNMD | |
| | | CAAGGAACCCATCAGCGTCAGCAGCGAGCAGGTGTTGA | | YFKFHNMRPPFTYATLIRWAILEAP | |
| | | AATTCCGTAAACTTAACTTCAATGGGGAGGGTGAACCC | | EKQRTLNEIYHWFTRMFAFFRNHPA | |
| | | GAAGAACTGATGGTGGACAACTGGCGCCCAGCTCAGCC | | TWKNAIRHNLSLHKCFVRVESEKGA | |
| | | ACTGAAGAACAGGCAAATCAAAGCTTCCTTCAAAGGAG | | VWTVDELEFRKKRSQRPSRCSNPTP | |
| | | GATCCGGAatgccgaacccaaggccagggaagccctct | | GP* | |
| | | gctcctagcctggccctcggccccagccctggcgctag | | | |
| | | cccctcttggagggcggctccgaaggcttccgacctcc | | | |
| | | tgggtgctaggggccctggtggaaccttccaaggcagg | | | |
| | | gatctgcgaggaggggcgcacgcctctagctcaagcct | | | |
| | | gaacccgatgcccccctcacagctgcaactgcctaccc | | | |
| | | tgccgctcgtcatggtggcccccagcggcgcaagactg | | | |
| | | ggcccgttgccgcacctgcaagccttgctgcaggaccg | | | |
| | | gccacatttcatgcaccagctcagcaccgtggacgcac | | | |
| | | atgcaaggacacccgtgctgcaagtccaccccctggag | | | |
| | | agccctgccatgatcagcctgacgccgcccaccaccgc | | | |
| | | aaccggcgtgttttcactgaaggcaagacccgggctgc | | | |
| | | caccgggcatcaacgtggccagcctggaatgggtgagc | | | |
| | | agggagccagcgctcctgtgtaccttcccaaacccatc | | | |
| | | cgccccaagaaaggacagcaccctgtctgccgtgcccc | | | |
| | | aatcatcttacccgctgctggcgaatggcgtatgcaag | | | |
| | | tggcccggatgtgaaaaggtgttcgaggagccggaaga | | | |
| | | tttcctgaaacattgccaggccgaccacctgttggacg | | | |
| | | aaaagggaagggcccaatgcctgcttcagagggagatg | | | |
| | | gtgcagagcttggagcaacaactcgtgctcgagaagga | | | |
| | | gaagctgagcgccatgcaggcacacctcgccggcaaga | | | |
| | | tggccctgaccaaagccagtagcgtagccagctccgac | | | |
| | | aagggtagctgttgcatcgtggccgcaggaagtcaagg | | | |
| | | ccccgttgtgcccgcctggagcggtccaagggaggcac | | | |
| | | ccgactcactgttcgccgtgaggaggcatctgtggggc | | | |
| | | agccacggtaacagcacgttccccgagttcctgcataa | | | |
| | | catggactacttcaagttccacaacatgcggcctccat | | | |
| | | tcacctacgccacactgataaggtgggctatcctggag | | | |
| | | gctcccgagaagcaaaggaccctgaacgagatctacca | | | |
| | | ctggttcaccaggatgttcgctttctttaggaaccacc | | | |
| | | ccgcgacctggaaaaacgccataaggcataacttgagc | | | |
| | | cttcacaagtgcttcgtgagggtggagagtgagaaagg | | | |
| | | tgccgtgtggactgtggatgagttggagttccgcaaga | | | |
| | | agcgaagccaacgacctagcaggtgtagcaatccaACG | | | |
| | | CCTggacccTAA | | | |
| FOXP3- | RQR8-P2A- | ATGGGCACAAGCCTGCTGTGTTGGATGGCCCTGTGTCT | 48 | MGTSLLCWMALCLLGADHADACPYS | 49 |
| 014 | CA2[L156H]- | GCTGGGAGCCGATCATGCTGATGCCTGTCCTTACAGCA | | NPSLCSGGGGSELPTQGTFSNVSTN | |
| | FOXP3FL | ACCCCAGCCTGTGTTCTGGCGGCGGAGGATCTGAACTG | | VSPAKPTTTACPYSNPSLCSGGGGS | |
| | | CCTACACAGGGCACCTTCAGCAACGTGTCCACCAATGT | | PAPRPPTPAPTIASQPLSLRPEACR | |
| | | GTCCCCAGCCAAGCCTACCACCACCGCTTGTCCCTACT | | PAAGGAVHTRGLDFACDIYIWAPLA | |
| | | CCAATCCTAGCCTGTGTAGCGGAGGTGGCGGAAGCCCT | | GTCGVLLLSLVITLYCNHRNRRRVC | |
| | | GCTCCTAGACCTCCTACACCAGCTCCTACAATCGCCAG | | KCPRPVVGSGATNFSLLKQAGDVEE | |
| | | CCAGCCTCTGTCTCTGAGGCCAGAAGCTTGTAGACCTG | | NPGPGGSMSHHWGYGKHNGPEHWHK | |
| | | CTGCTGGCGGAGCCGTGCATACAAGAGGACTGGATTTC | | DFPIAKGERQSPVDIDTHTAKYDPS | |
| | | GCCTGCGACATCTACATCTGGGCCCCTCTGGCTGGAAC | | LKPLSVSYDQATSLRILNNGHAFNV | |
| | | ATGTGGCGTTCTGCTGCTGAGCCTGGTCATCACCCTGT | | EFDDSQDKAVLKGGPLDGTYRLIQF | |
| | | ACTGCAACCACCGGAACAGGCGGAGAGTGTGCAAGTGC | | HFHWGSLDGQGSEHTVDKKKYAAEL | |
| | | CCTAGACCTGTGGTTGGATCTGGTgctactaacttcag | | HLVHWNTKYGDFGKAVQQPDGLAVL | |
| | | cctgctgaagcaggctggagacgtggaggagaaccctg | | GIFLKVGSAKPGHQKVVDVLDSIKT | |
| | | gacctGGAGGATCCatgTCCCATCACTGGGGGTACGGC | | KGKSADFTNFDPRGLLPESLDYWTY | |
| | | AAACACAACGGACCTGAGCACTGGCATAAGGACTTCCC | | PGSLTTPPLLECVTWIVLKEPISVS | |
| | | CATTGCCAAGGGAGAGCGCCAGTCCCCTGTTGACATCG | | SEQVLKFRKLNFNGEGEPEELMVDN | |
| | | ACACTCATACAGCCAAGTATGACCCTTCCCTGAAGCCC | | WRPAQPLKNRQIKASFKGGSGMPNP | |
| | | CTGTCTGTTTCCTATGATCAAGCAACTTCCCTGAGGAT | | RPGKPSAPSLALGPSPGASPSWRAA | |
| | | CCTCAACAATGGTCATGCTTTCAACGTGGAGTTTGATG | | PKASDLLGARGPGGTFQGRDLRGGA | |
| | | ACTCTCAGGACAAAGCAGTGCTCAAGGGAGGACCCCTG | | HASSSSLNPMPPSQLQLPTLPLVMV | |
| | | GATGGCACTTACAGATTGATTCAGTTTCACTTTCACTG | | APSGARLGPLPHLQALLQDRPHFMH | |
| | | GGGTTCACTTGATGGACAAGGTTCAGAGCATACTGTGG | | QLSTVDAHARTPVLQVHPLESPAMI | |
| | | ATAAAAAGAAATATGCTGCAGAACTTCACTTGGTTCAC | | SLTPPTTATGVFSLKARPGLPPGIN | |
| | | TGGAACACCAAATATGGGGATTTTGGGAAAGCTGTGCA | | VASLEWVSREPALLCTFPNPSAPRK | |
| | | GCAACCTGATGGACTGGCCGTTCTAGGTATTTTTTTGA | | DSTLSAVPQSSYPLLANGVCKWPGC | |
| | | AGGTTGGCAGCGCTAAACCGGGCCATCAGAAAGTTGTT | | EKVFEEPEDFLKHCQADHLLDEKGR | |
| | | GATGTGCTGGATTCCATTAAAACAAAGGGCAAGAGTGC | | AQCLLQREMVQSLEQQLVLEKEKLS | |
| | | TGACTTCACTAACTTCGATCCTCGTGGCCTCCTTCCTG | | AMQAHLAGKMALTKASSVASSDKGS | |
| | | AATCCCTGGATTACTGGACCTACCCAGGCTCACTGACC | | CCIVAAGSQGPVVPAWSGPREAPDS | |
| | | ACCCCTCCTCTTCTGGAATGTGTGACCTGGATTGTGCT | | LFAVRRHLWGSHGNSTFPEFLHNMD | |
| | | CAAGGAACCCATCAGCGTCAGCAGCGAGCAGGTGTTGA | | YFKFHNMRPPFTYATLIRWAILEAP | |
| | | AATTCCGTAAACTTAACTTCAATGGGGAGGGTGAACCC | | EKQRTLNEIYHWFTRMFAFFRNHPA | |
| | | GAAGAACTGATGGTGGACAACTGGCGCCCAGCTCAGCC | | TWKNAIRHNLSLHKCFVRVESEKGA | |
| | | ACTGAAGAACAGGCAAATCAAAGCTTCCTTCAAAGGAG | | VWTVDELEFRKKRSQRPSRCSNPTP | |
| | | GATCCGGAatgccgaacccaaggccagggaagccctct | | GP* | |
| | | gctcctagcctggccctcggccccagccctggcgctag | | | |
| | | cccctcttggagggcggctccgaaggcttccgacctcc | | | |
| | | tgggtgctaggggccctggtggaaccttccaaggcagg | | | |
| | | gatctgcgaggaggggcgcacgcctctagctcaagcct | | | |
| | | gaacccgatgcccccctcacagctgcaactgcctaccc | | | |
| | | tgccgctcgtcatggtggcccccagcggcgcaagactg | | | |
| | | ggcccgttgccgcacctgcaagccttgctgcaggaccg | | | |
| | | gccacatttcatgcaccagctcagcaccgtggacgcac | | | |
| | | atgcaaggacacccgtgctgcaagtccaccccctggag | | | |
| | | agccctgccatgatcagcctgacgccgcccaccaccgc | | | |
| | | aaccggcgtgttttcactgaaggcaagacccgggctgc | | | |
| | | caccgggcatcaacgtggccagcctggaatgggtgagc | | | |
| | | agggagccagcgctcctgtgtaccttcccaaacccatc | | | |
| | | cgccccaagaaaggacagcaccctgtctgccgtgcccc | | | |
| | | aatcatcttacccgctgctggcgaatggcgtatgcaag | | | |
| | | tggcccggatgtgaaaaggtgttcgaggagccggaaga | | | |
| | | tttcctgaaacattgccaggccgaccacctgttggacg | | | |
| | | aaaagggaagggcccaatgcctgcttcagagggagatg | | | |
| | | gtgcagagcttggagcaacaactcgtgctcgagaagga | | | |
| | | gaagctgagcgccatgcaggcacacctcgccggcaaga | | | |
| | | tggccctgaccaaagccagtagcgtagccagctccgac | | | |
| | | aagggtagctgttgcatcgtggccgcaggaagtcaagg | | | |
| | | ccccgttgtgcccgcctggagcggtccaagggaggcac | | | |
| | | ccgactcactgttcgccgtgaggaggcatctgtggggc | | | |
| | | agccacggtaacagcacgttccccgagttcctgcataa | | | |
| | | catggactacttcaagttccacaacatgcggcctccat | | | |
| | | tcacctacgccacactgataaggtgggctatcctggag | | | |
| | | gctcccgagaagcaaaggaccctgaacgagatctacca | | | |
| | | ctggttcaccaggatgttcgctttctttaggaaccacc | | | |
| | | ccgcgacctggaaaaacgccataaggcataacttgagc | | | |
| | | cttcacaagtgcttcgtgagggtggagagtgagaaagg | | | |
| | | tgccgtgtggactgtggatgagttggagttccgcaaga | | | |
| | | agcgaagccaacgacctagcaggtgtagcaatccaACG | | | |
| | | CCTggacccTAA | | | |
| FOXP3- | RQR8-P2A- | ATGGGCACAAGCCTGCTGTGTTGGATGGCCCTGTGTCT | 50 | MGTSLLCWMALCLLGADHADACPYS | 51 |
| 015 | FOXP3FL | GCTGGGAGCCGATCATGCTGATGCCTGTCCTTACAGCA | | NPSLCSGGGGSELPTQGTFSNVSTN | |
| | | ACCCCAGCCTGTGTTCTGGCGGCGGAGGATCTGAACTG | | VSPAKPTTTACPYSNPSLCSGGGGS | |
| | | CCTACACAGGGCACCTTCAGCAACGTGTCCACCAATGT | | PAPRPPTPAPTIASQPLSLRPEACR | |
| | | GTCCCCAGCCAAGCCTACCACCACCGCTTGTCCCTACT | | PAAGGAVHTRGLDFACDIYIWAPLA | |
| | | CCAATCCTAGCCTGTGTAGCGGAGGTGGCGGAAGCCCT | | GTCGVLLLSLVITLYCNHRNRRRVC | |
| | | GCTCCTAGACCTCCTACACCAGCTCCTACAATCGCCAG | | KCPRPVVGSGATNFSLLKQAGDVEE | |
| | | CCAGCCTCTGTCTCTGAGGCCAGAAGCTTGTAGACCTG | | NPGPGGSGMPNPRPGKPSAPSLALG | |
| | | CTGCTGGCGGAGCCGTGCATACAAGAGGACTGGATTTC | | PSPGASPSWRAAPKASDLLGARGPG | |
| | | GCCTGCGACATCTACATCTGGGCCCCTCTGGCTGGAAC | | GTFQGRDLRGGAHASSSSLNPMPPS | |
| | | ATGTGGCGTTCTGCTGCTGAGCCTGGTCATCACCCTGT | | QLQLPTLPLVMVAPSGARLGPLPHL | |
| | | ACTGCAACCACCGGAACAGGCGGAGAGTGTGCAAGTGC | | QALLQDRPHFMHQLSTVDAHARTPV | |
| | | CCTAGACCTGTGGTTGGATCTGGTgctactaacttcag | | LQVHPLESPAMISLTPPTTATGVFS | |
| | | cctgctgaagcaggctggagacgtggaggagaaccctg | | LKARPGLPPGINVASLEWVSREPAL | |
| | | gacctGGAGGATCCGGAatgccgaacccaaggccaggg | | LCTFPNPSAPRKDSTLSAVPQSSYP | |
| | | aagccctctgctcctagcctggccctcggccccagccc | | LLANGVCKWPGCEKVFEEPEDFLKH | |
| | | tggcgctagcccctcttggagggcggctccgaaggctt | | CQADHLLDEKGRAQCLLQREMVQSL | |
| | | ccgacctcctgggtgctaggggccctggtggaaccttc | | EQQLVLEKEKLSAMQAHLAGKMALT | |
| | | caaggcagggatctgcgaggaggggcgcacgcctctag | | KASSVASSDKGSCCIVAAGSQGPVV | |
| | | ctcaagcctgaacccgatgcccccctcacagctgcaac | | PAWSGPREAPDSLFAVRRHLWGSHG | |
| | | tgcctaccctgccgctcgtcatggtggcccccagcggc | | NSTFPEFLHNMDYFKFHNMRPPFTY | |
| | | gcaagactgggcccgttgccgcacctgcaagccttgct | | ATLIRWAILEAPEKQRTLNEIYHWF | |
| | | gcaggaccggccacatttcatgcaccagctcagcaccg | | TRMFAFFRNHPATWKNAIRHNLSLH | |
| | | tggacgcacatgcaaggacacccgtgctgcaagtccac | | KCFVRVESEKGAVWTVDELEFRKKR | |
| | | cccctggagagccctgccatgatcagcctgacgccgcc | | SQRPSRCSNPTPGP* | |
| | | caccaccgcaaccggcgtgttttcactgaaggcaagac | | | |
| | | ccgggctgccaccgggcatcaacgtggccagcctggaa | | | |
| | | tgggtgagcagggagccagcgctcctgtgtaccttccc | | | |
| | | aaacccatccgccccaagaaaggacagcaccctgtctg | | | |
| | | ccgtgccccaatcatcttacccgctgctggcgaatggc | | | |
| | | gtatgcaagtggcccggatgtgaaaaggtgttcgagga | | | |
| | | gccggaagatttcctgaaacattgccaggccgaccacc | | | |
| | | tgttggacgaaaagggaagggcccaatgcctgcttcag | | | |
| | | agggagatggtgcagagcttggagcaacaactcgtgct | | | |
| | | cgagaaggagaagctgagcgccatgcaggcacacctcg | | | |
| | | ccggcaagatggccctgaccaaagccagtagcgtagcc | | | |
| | | agctccgacaagggtagctgttgcatcgtggccgcagg | | | |
| | | aagtcaaggccccgttgtgcccgcctggagcggtccaa | | | |
| | | gggaggcacccgactcactgttcgccgtgaggaggcat | | | |
| | | ctgtggggcagccacggtaacagcacgttccccgagtt | | | |
| | | cctgcataacatggactacttcaagttccacaacatgc | | | |
| | | ggcctccattcacctacgccacactgataaggtgggct | | | |
| | | atcctggaggctcccgagaagcaaaggaccctgaacga | | | |
| | | gatctaccactggttcaccaggatgttcgctttcttta | | | |
| | | ggaaccaccccgcgacctggaaaaacgccataaggcat | | | |
| | | aacttgagccttcacaagtgcttcgtgagggtggagag | | | |
| | | tgagaaaggtgccgtgtggactgtggatgagttggagt | | | |
| | | tccgcaagaagcgaagccaacgacctagcaggtgtagc | | | |
| | | aatccaACGCCTggacccTAA | | | |
| cJun | cJun construct | ACCGCCAAGATGGAAACCACCTTCTACGACGACGCCCT | 52 | TAKMETTFYDDALNASFLPSESGPY | 53 |
| | component | GAACGCCAGCTTTCTGCCTTCTGAGTCTGGCCCCTACG | | GYSNPKILKQSMTLNLADPVGSLKP | |
| | | GCTACAGCAACCCCAAGATCCTGAAGCAGAGCATGACC | | HLRAKNSDLLTSPDVGLLKLASPEL | |
| | | CTGAACCTGGCCGATCCTGTGGGCAGCCTGAAACCTCA | | ERLIIQSSNGHITTTPTPTQFLCPK | |
| | | CCTGAGAGCCAAGAACAGCGACCTGCTGACAAGCCCTG | | NVTDEQEGFAEGFVRALAELHSQNT | |
| | | ATGTGGGCCTGCTGAAACTGGCTAGCCCCGAGCTGGAA | | LPSVTSAAQPVNGAGMVAPAVASVA | |
| | | CGGCTGATCATCCAGTCTAGCAACGGCCACATCACCAC | | GGSGSGGFSASLHSEPPVYANLSNF | |
| | | CACACCTACACCAACACAGTTTCTGTGCCCCAAGAACG | | NPGALSSGGGAPSYGAAGLAFPAQP | |
| | | TGACCGACGAGCAAGAGGGATTCGCCGAGGGCTTTGTT | | QQQQQPPHHLPQQMPVOHPRLQALK | |
| | | AGAGCCCTGGCCGAACTGCACAGCCAGAATACCCTGCC | | EEPQTVPEMPGETPPLSPIDMESQE | |
| | | TAGCGTGACATCTGCCGCTCAGCCTGTTAATGGCGCCG | | RIKAERKRMRNRIAASKCRKRKLER | |
| | | GAATGGTTGCTCCTGCCGTGGCTTCTGTTGCTGGCGGA | | IARLEEKVKTLKAQNSELASTANML | |
| | | TCTGGATCTGGCGGCTTTAGCGCCTCTCTGCACTCTGA | | REQVAQLKQKVMNHVNSGCQLMLTQ | |
| | | GCCTCCAGTGTACGCCAACCTGAGCAACTTCAACCCTG | | QLQTF | |
| | | GCGCTCTTAGCTCTGGTGGCGGAGCACCTTCTTATGGC | | | |
| | | GCTGCCGGATTGGCCTTTCCTGCTCAGCCTCAGCAGCA | | | |
| | | GCAACAGCCTCCTCATCATCTGCCCCAGCAGATGCCTG | | | |
| | | TGCAGCACCCTAGACTGCAGGCCCTGAAAGAGGAACCC | | | |
| | | CAGACAGTCCCTGAGATGCCCGGCGAAACACCTCCTCT | | | |
| | | GAGCCCCATCGACATGGAAAGCCAAGAGCGGATCAAGG | | | |
| | | CCGAGCGGAAGCGGATGAGAAATAGAATCGCCGCCTCC | | | |
| | | AAGTGCCGGAAGAGGAAGCTGGAAAGAATCGCCCGGCT | | | |
| | | GGAAGAGAAAGTGAAAACCCTGAAGGCCCAGAACTCCG | | | |
| | | AGCTGGCCTCTACCGCCAACATGCTGAGAGAACAGGTG | | | |
| | | GCCCAGCTGAAACAGAAAGTCATGAACCACGTGAACAG | | | |
| | | CGGCTGCCAGCTGATGCTGACACAGCAGCTGCAGACCT | | | |
| | | TC | | | |
| RQR8 | RQR8 construct | ATGGGCACAAGCCTGCTGTGTTGGATGGCCCTGTGTCT | 54 | MGTSLLCWMALCLLGADHADACPYS | 55 |
| | component | GCTGGGAGCCGATCATGCTGATGCCTGTCCTTACAGCA | | NPSLCSGGGGSELPTQGTFSNVSTN | |
| | | ACCCCAGCCTGTGTTCTGGCGGCGGAGGATCTGAACTG | | VSPAKPTTTACPYSNPSLCSGGGGS | |
| | | CCTACACAGGGCACCTTCAGCAACGTGTCCACCAATGT | | PAPRPPTPAPTIASQPLSLRPEACR | |
| | | GTCCCCAGCCAAGCCTACCACCACCGCTTGTCCCTACT | | PAAGGAVHTRGLDFACDIYIWAPLA | |
| | | CCAATCCTAGCCTGTGTAGCGGAGGTGGCGGAAGCCCT | | GTCGVLLLSLVITLYCNHRNRRRVC | |
| | | GCTCCTAGACCTCCTACACCAGCTCCTACAATCGCCAG | | KCPRPVV | |
| | | CCAGCCTCTGTCTCTGAGGCCAGAAGCTTGTAGACCTG | | | |
| | | CTGCTGGCGGAGCCGTGCATACAAGAGGACTGGATTTC | | | |
| | | GCCTGCGACATCTACATCTGGGCCCCTCTGGCTGGAAC | | | |
| | | ATGTGGCGTTCTGCTGCTGAGCCTGGTCATCACCCTGT | | | |
| | | ACTGCAACCACCGGAACAGGCGGAGAGTGTGCAAGTGC | | | |
| | | CCTAGACCTGTGGTT | | | |
| FOXP3 | FOXP3 (FOXP3FL) | atgccgaacccaaggccagggaagccctctgctcctag | 56 | MPNPRPGKPSAPSLALGPSPGASPS | 57 |
| (FOXP3FL) | construct | cctggccctcggccccagccctggcgctagcccctctt | | WRAAPKASDLLGARGPGGTFQGRDL | |
| | component | ggagggcggctccgaaggcttccgacctcctgggtgct | | RGGAHASSSSLNPMPPSQLQLPTLP | |
| | | aggggccctggtggaaccttccaaggcagggatctgcg | | LVMVAPSGARLGPLPHLQALLQDRP | |
| | | aggaggggcgcacgcctctagctcaagcctgaacccga | | HFMHQLSTVDAHARTPVLQVHPLES | |
| | | tgcccccctcacagctgcaactgcctaccctgccgctc | | PAMISLTPPTTATGVFSLKARPGLP | |
| | | gtcatggtggcccccagcggcgcaagactgggcccgtt | | PGINVASLEWVSREPALLCTFPNPS | |
| | | gccgcacctgcaagccttgctgcaggaccggccacatt | | APRKDSTLSAVPQSSYPLLANGVCK | |
| | | tcatgcaccagctcagcaccgtggacgcacatgcaagg | | WPGCEKVFEEPEDFLKHCQADHLLD | |
| | | acacccgtgctgcaagtccaccccctggagagccctgc | | EKGRAQCLLQREMVQSLEQQLVLEK | |
| | | catgatcagcctgacgccgcccaccaccgcaaccggcg | | EKLSAMQAHLAGKMALTKASSVASS | |
| | | tgttttcactgaaggcaagacccgggctgccaccgggc | | DKGSCCIVAAGSQGPVVPAWSGPRE | |
| | | atcaacgtggccagcctggaatgggtgagcagggagcc | | APDSLFAVRRHLWGSHGNSTFPEFL | |
| | | agcgctcctgtgtaccttcccaaacccatccgccccaa | | HNMDYFKFHNMRPPFTYATLIRWAI | |
| | | gaaaggacagcaccctgtctgccgtgccccaatcatct | | LEAPEKQRTLNEIYHWFTRMFAFFR | |
| | | tacccgctgctggcgaatggcgtatgcaagtggcccgg | | NHPATWKNAIRHNLSLHKCFVRVES | |
| | | atgtgaaaaggtgttcgaggagccggaagatttcctga | | EKGAVWTVDELEFRKKRSQRPSRCS | |
| | | aacattgccaggccgaccacctgttggacgaaaaggga | | NPTPGP | |
| | | agggcccaatgcctgcttcagagggagatggtgcagag | | | |
| | | cttggagcaacaactcgtgctcgagaaggagaagctga | | | |
| | | gcgccatgcaggcacacctcgccggcaagatggccctg | | | |
| | | accaaagccagtagcgtagccagctccgacaagggtag | | | |
| | | ctgttgcatcgtggccgcaggaagtcaaggccccgttg | | | |
| | | tgcccgcctggagcggtccaagggaggcacccgactca | | | |
| | | ctgttcgccgtgaggaggcatctgtggggcagccacgg | | | |
| | | taacagcacgttccccgagttcctgcataacatggact | | | |
| | | acttcaagttccacaacatgcggcctccattcacctac | | | |
| | | gccacactgataaggtgggctatcctggaggctcccga | | | |
| | | gaagcaaaggaccctgaacgagatctaccactggttca | | | |
| | | ccaggatgttcgctttctttaggaaccaccccgcgacc | | | |
| | | tggaaaaacgccataaggcataacttgagccttcacaa | | | |
| | | gtgcttcgtgagggtggagagtgagaaaggtgccgtgt | | | |
| | | ggactgtggatgagttggagttccgcaagaagcgaagc | | | |
| | | caacgacctagcaggtgtagcaatccaACGCCTggacc | | | |
| | | c | | | |

### Characterization of Ligand-Dependent Activity of a Transcription Factor System

Ligand-dependent activity of a transcription factor system may be characterized by various methods.

In some embodiments, ligand-dependent activity of a transcription factor system is characterized by ligand-dependent regulation of a transcription factor polypeptide (for example, a transcription factor DNA binding domain, a transcription factor activation domain or both the transcription factor DNA binding domain and transcription factor activation domain), encoded by the transcription factor system. In some embodiments, ligand-dependent activity of a transcription factor system is characterized by ligand dose-dependent regulation of a transcription factor polypeptide encoded by the transcription factor system. In one aspect, a transcription factor polypeptide is a polypeptide comprising a transcription factor activation domain. In another aspect, a transcription factor polypeptide is a polypeptide comprising a transcription factor DNA binding domain. In another aspect, a transcription factor polypeptide is a polypeptide comprising both a transcription factor activation domain and a transcription factor DNA binding domain. Ligand-dependent regulation of a transcription factor polypeptide may be characterized by various methods. In some aspects, ligand-dependent regulation of a transcription factor polypeptide may be assessed by measuring the levels of the transcription factor polypeptide or domain thereof, such as by an immunoassay.

In some embodiments, ligand-dependent activity of a transcription factor system is characterized by ligand-dependent expression of the payload encoded by the transcription factor system. Expression of the payload may be assessed by various methods. In some aspects, expression of the payload is assessed by measuring payload mRNA levels. In some aspects, expression of the payload is assessed by measuring payload polypeptide levels.

In some embodiments, a transcription factor system may be compared to a control transcription factor system that lacks a DRD. In some embodiments, ligand-dependent activity of a transcription factor system may be analyzed or characterized relative to the activity of a transcription factor system comprising a control transcription factor construct that lacks a DRD. An example of a control transcription factor construct is construct ZFHD-004, which is described by the present disclosure (as shown in Table 1).

### Transcription Factors

A transcription factor is a protein that binds to DNA, preferably to a sequence-specific site on the DNA (a transcription factor polynucleotide binding site) located in or near a promoter, which facilitates the binding of the transcription machinery to the promoter, thus activating transcription of the DNA sequence. Such entities are also known as transcription regulator proteins.

In various embodiments, a transcription factor for use in the transcription factor system, compositions and methods described herein includes a transcription factor DNA binding domain and a transcription factor activation domain. In some embodiments, the combination of the transcription factor DNA binding domain and a transcription factor activation domain results in a functional transcription factor. In various embodiments, the transcription factor DNA binding domain and/or the transcription factor activation domain may interact with other transcription regulatory elements.

In some embodiments, transcription factors are exemplified as proteins that recognize and bind to specific short DNA sequences and thereby causally affect gene expression. The recognition of DNA sequences by transcription factors occurs by chemical interactions of the amino acid side chains of a transcription factor protein with base pair residues of the DNA that functions as regulatory sequence. The transcription factors thus “read” the genomic sequence, which mechanism provides the sequence recognition function on which informational aspects of regulatory transactions controlling gene expression depend.

Transcription factors typically consist of DNA-binding domains and effector or activation domains that mediate interactions with other proteins necessary for transcription, including with other transcription factors. Transcription factors execute many functions, including gene activation. They are transcribed in the nucleus, translated in the cytoplasm, and find their target sites in the genomic DNA on reentry into the nucleus, mediated by nuclear localization sites included in all transcription factor protein sequences. Transcription factors include basic domains which cause them to be concentrated nonspecifically in the vicinity of the DNA, facilitating the diffusion-limited discovery of their target sites.

In various embodiments of the present disclosure, a transcription factor system utilizes a transcription factor made up of and/or comprising a transcription factor DNA binding domain and a transcription factor effector or activation domain or protein (used interchangeably herein). The transcription factor activation domain, the transcription factor DNA binding domain, and/or the combination of the transcription factor activation domain and the transcription factor DNA binding domain may be operably linked to the DRD (any of which is a DRD-TF). Upon stabilization of the linked DRD through binding of an exogenous stabilizing ligand, the stabilized DRD-TF is able to transcribe a protein of interest.

The DNA sequence that a transcription factor DNA binding domain binds to is called a transcription factor-binding site or response element, or as used herein interchangeably, a specific polynucleotide binding site; these binding sites are found in or near the promoter of the regulated DNA sequence. A promoter comprising a specific polynucleotide binding site may be an exogenous promoter. In some embodiments, a promoter may be an exogenous inducible promoter. The transcription factor-binding site or specific polynucleotide binding site when incorporated into a transcription factor system containing a protein of interest or payload, is an exogenous nucleic acid sequence.

In various embodiments of the present disclosure, suitable transcription factors useful in the synthesis of transcription factor system can include any known transcription factor for which the transcription factor-binding site is known. Some examples of such transcription factors include (but are not limited to) the STAT family (STATs 1, 2, 3, 4, 5a, 5b, and 6), c-Fos, FosB, Fra-1, Fra-2, c-Jun, JunB and JunD, fos/jun, NF kappa B, HIV-TAT, E2F family, T-Box Gene Family, Helix-Loop-Helix Transcription Factors, Zinc Finger Transcription Factors, e.g. ZFHD1, Oct4, and Zif268, engineered Zinc Finger Transcription Factors, and transcription factors from the following families: bHLH, bZIP, Forkhead, Nuclear receptor, HMG/Sox, Ets, T-box, AT hook, Homeodomain+POU, Myb/SANT, THAP finger, CENPB, E2F, BED ZF, GATA, Rel, CxxC, IRF, SAND, SMAD, HSF, MBD, RFX, CUT+Homeodomain, DM, STAT, ARID/BRIGHT, Grainyhead, MADS box, AP-2, CSD, and Homeodomain+PAX. Exemplary transcription factor DNA binding domains may include one or more DNA binding domains derived from a parent protein selected from the group consisting of: ZFHD1, Cas9, Cas12, and TAL.

In various embodiments, the transcription factor system provides for the tunable transcription of a protein of interest or payload (used interchangeably herein). In various embodiments, the nucleic acid sequence encoding the protein of interest is operably linked to an exogenous inducible promoter comprising a specific polynucleotide binding site, that is, a defined DNA polynucleotide sequence, that specifically binds to the transcription factor DNA binding domain. The transcription factor binding domain, in combination with the transcription factor DNA activation domain, is then able to regulate transcription of the protein of interest.

When a cell or organism comprising a DRD-TF is exposed to an exogenous stabilizing ligand, the DRD-TF is stabilized. The stabilized DRD-TF is then able to bind to the specific polynucleotide binding site to which the DRD-TF binds, and thus regulate transcription of the polynucleotide encoding the protein of interest. In some embodiments, the binding of the stabilized DRD-TF activates transcription of the polynucleotide encoding the protein of interest, which results in protein expression in the cell or organism. In the absence of the exogenous stabilizing ligand, the DRD-TF is degraded and unable to activate transcription. Thus, both the amount and the timing of protein expression can be controlled by administering the exogenous stabilizing ligand to the cell or organism.

In various embodiments, the transcription factor DNA binding domain, the transcription factor activation domain, are typically operably linked or may be separated by one or more intervening sequences, for example, a linker or a cleavage site. In various embodiments, a first polynucleotide may include a first nucleic acid sequence that encodes a transcription factor DNA binding domain; a second nucleic acid sequence that encodes a transcription factor activation domain; and a third nucleic acid sequence that encodes a drug responsive domain (DRD). In such embodiments, the transcription factor activation domain and/or the transcription factor DNA binding domain, upon expression in the cell, is operably linked to the DRD. In addition, the cell will also include a second polynucleotide that comprises a fourth nucleic acid sequence that can be specifically bound by the transcription factor DNA binding domain and a fifth nucleic acid sequence that encodes a protein of interest or payload as described herein.

The transcription factor DNA binding domain, the transcription factor activation domain and the protein of interest or payload may be supplied for the methods of the present disclosure on the same vectors or in separate vectors.

In some embodiments, a vector comprises the polynucleotides described herein. In some embodiments, the vector comprises at least a first nucleic acid sequence that encodes at least one of a transcription factor DNA binding domain and a transcription factor activation domain; and a second nucleic acid sequence that encodes a drug responsive domain (DRD); wherein the transcription factor DNA binding domain and/or the transcription factor activation domain is operably linked to the DRD. Optionally, in some embodiments, a first vector comprises the transcription factor linked to the DRD, and a second vector comprises a protein of interest or payload operably linked to a transcription factor polynucleotide binding site. In a further embodiment, a single vector comprises a first nucleic acid sequence encoding a transcription factor able to bind to a specific polynucleotide binding site and activate transcription; a second nucleic acid sequence encoding a drug responsive domain (DRD); wherein the transcription factor is operably linked to the DRD and optionally a third nucleic acid sequence encoding a protein of interest operably linked to an inducible promoter comprising the transcription factor polynucleotide binding site. In some embodiments, a first vector comprises at least a first nucleic acid sequence that encodes at least one of a transcription factor DNA binding domain and a transcription factor activation domain; and a second nucleic acid sequence that encodes a drug responsive domain (DRD); wherein the transcription factor DNA binding domain and/or the transcription factor activation domain is operably linked to the DRD, and a second vector comprises a third nucleic acid sequence that can be specifically bound by the transcription factor DNA binding domain and a fourth nucleic acid sequence that encodes a protein of interest or payload as described herein.

In some embodiments, the vectors also possess an origin of replication (ori) which permits amplification of the vector, for example in bacteria. Additionally, or alternatively, the vector includes selectable markers such as antibiotic resistance genes, genes for colored markers and suicide genes.

### Drug Responsive Domains (DRDs)

Drug responsive domains (DRDs) are protein domains that are unstable and degraded in the absence of ligand, but whose stability is rescued by binding to a corresponding DRD-binding ligand. The term drug responsive domain (DRD) is interchangeable with the term destabilizing domain (DD). Drug responsive domains (DRDs) can be appended to a polypeptide or protein and can render the attached polypeptide or protein unstable in the absence of a DRD-binding ligand. DRDs convey their destabilizing property to the attached polypeptide or protein via protein degradation. Without wishing to be bound by any theory, in the absence of a DRD-binding ligand, the appended polypeptide or protein is rapidly degraded by the ubiquitin-proteasome system of a cell. A ligand that binds to or interacts with a DRD can, upon such binding or interaction, modulate the stability of the appended polypeptide or protein. When a ligand binds its intended DRD, the instability is reversed and function of the appended polypeptide or protein can be restored. The conditional nature of DRD stability allows a rapid and non-perturbing switch from stable protein to unstable substrate for degradation. Moreover, its dependency on the concentration of its ligand further provides tunable control of degradation rates.

In some embodiments, DRDs of the present disclosure may be derived from known polypeptides that are capable of post-translational regulation of proteins. In some embodiments, DRDs of the present disclosure may be developed or derived from known proteins. Regions or portions or domains of wild type proteins may be utilized as DRDs in whole or in part. They may be combined or rearranged to create new peptides, proteins, regions or domains of which any may be used as DRDs or the starting point for the design of further DRDs.

In some embodiments, a DRD may be derived from a parent protein or from a mutant protein having one, two, three, or more amino acid mutations compared to the parent protein. In some embodiments, the parent protein may be selected from, but is not limited to, FKBP; human protein FKBP; human DHFR (hDHFR); E. coli DHFR (ecDHFR); PDE5 (phosphodiesterase 5); CA2 (Carbonic anhydrase II); and ER (estrogen receptor). Examples of proteins that may be used to develop DRDs and their ligands are listed in Table 3.

TABLE 3
Proteins and their binding ligands

| | | Protein | |
|---|---|---|---|
| | | SEQ ID | |
| Protein | Protein Sequence | NO.: | Ligands |
| E. coli | MISLIAALAVDRVIGMENAMPWNLPADL | 1 | Methotrexate |
| Dihydrofolate | AWFKRNTLNKPVIMGRHTWESIGRPLPG | | (MTX) |
| reductase | RKNIILSSQPGTDDRVTWVKSVDEAIAA | | Trimethoprim |
| (ecDHFR) | CGDVPEIMVIGGGRVYEQFLPKAQKLYL | | (TMP) |
| (Uniprot ID: | THIDAEVEGDTHFPDYEPDDWESVFSEF | | |
| P0ABQ4) | HDADAQNSHSYCFEILERR | | |
| Human | MVGSLNCIVAVSQNMGIGKNGDLPWPPL | 2 | Methotrexate |
| Dihydrofolate | RNEFRYFQRMTTTSSVEGKQNLVIMGKK | | (MTX) |
| reductase | TWFSIPEKNRPLKGRINLVLSRELKEPP | | Trimethoprim |
| (hDHFR) | QGAHFLSRSLDDALKLTEQPELANKVDM | | (TMP) |
| (Uniprot ID: | VWIVGGSSVYKEAMNHPGHLKLFVTRIM | | |
| P00374) | EQDFSDTFFPEIDLEKYKLLPEYPGVLS | | |
| | DVQEEKGIKYKFEVYEKND | | |
| Human FKBP | GVQVETISPGDGRTFPKRGQTCVVHYTG | 3 | Shield-1 |
| (FK506 binding | MLEDGKKFDSSRDRNKPFKFMLGKQEVI | | |
| protein) (Uniprot | RGWEEGVAQMSVGQRAKLTISPDYAYGA | | |
| ID: P62942) | TGHPGIIPPHATLVFDVELLKLE | | |
| Phosphodiesterase | MEETRELQSLAAAVVPSAQTLKITDFSF | 4 | Sildenafil; |
| 5 (PDE5), | SDFELSDLETALCTIRMFTDLNLVQNFQ | | Vardenafil; |
| ligand binding | MKHEVLCRWILSVKKNYRKNVAYHNWRH | | Tadalafil |
| domain (Uniprot | AFNTAQCMFAALKAGKIQNKLTDLEILA | | |
| ID: Uniprot ID | LLIAALSHDLDHRGVNNSYIQRSEHPLA | | |
| O76074) | QLYCHSIMEHHHFDQCLMILNSPGNQIL | | |
| | SGLSIEEYKTTLKIIKQAILATDLALYI | | |
| | KRRGEFFELIRKNQFNLEDPHQKELFLA | | |
| | MLMTACDLSAITKPWPIQQRIAELVATE | | |
| | FFDQGDRERKELNIEPTDLMNREKKNKI | | |
| | PSMQVGFIDAICLQLYEALTHVSEDCFP | | |
| | LLDGCRKNRQKWQALAEQQ | | |
| Phosphodiesterase | MERAGPSFGQQRQQQQPQQQKQQQRDQD | 71 | Sildenafil; |
| 5 (PDE5), foil- | SVEAWLDDHWDFTFSYFVRKATREMVNA | | Vardenafil; |
| length (Uniprot | WFAERVHTIPVCKEGIRGHTESCSCPLQ | | Tadalafil |
| ID: Uniprot ID | QSPRADNSAPGTPTRKISASEFDRPLRP | | |
| O76074) | IVVKDSEGTVSFLSDSEKKEQMPLTPPR | | |
| | FDHDEGDQCSRLLELVKDISSHLDVTAL | | |
| | CHKIFLHIHGLISADRYSLFLVCEDSSN | | |
| | DKFLISRLFDVAEGSTLEEVSNNCIRLE | | |
| | WNKGIVGHVAALGEPLNIKDAYEDPRFN | | |
| | AEVDQITGYKTQSILCMPIKNHREEVVG | | |
| | VAQAINKKSGNGGTFTEKDEKDFAAYLA | | |
| | FCGIVLHNAQLYETSLLENKRNQVLLDL | | |
| | ASLIFEEQQSLEVILKKIAATIISFMQV | | |
| | QKCTIFIVDEDCSDSFSSVFHMECEELE | | |
| | KSSDTLTREHDANKINYMYAQYVKNTME | | |
| | PLNIPDVSKDKRFPWTTENTGNVNQQCI | | |
| | RSLLCTPIKNGKKNKVIGVCQLVNKMEE | | |
| | NTGKVKPFNRNDEQFLEAFVIFCGLGIQ | | |
| | NTQMYEAVERAMAKQMVTLEVLSYHASA | | |
| | AEEETRELQSLAAAVVPSAQTLKITDFS | | |
| | FSDFELSDLETALCTIRMFTDLNLVQNF | | |
| | QMKHEVLCRWILSVKKNYRKNVAYHNWR | | |
| | HAFNTAQCMFAALKAGKIQNKLTDLEIL | | |
| | ALLIAALSHDLDHRGVNNSYIQRSEHPL | | |
| | AQLYCHSIMEHHHFDQCLMILNSPGNQI | | |
| | LSGLSIEEYKTTLKIIKQAILATDLALY | | |
| | IKRRGEFFELIRKNQFNLEDPHQKELFL | | |
| | AMLMTACDLSAITKPWPIQQRIAELVAT | | |
| | EFFDQGDRERKELNIEPTDLMNREKKNK | | |
| | IPSMQVGFIDAICLQLYEALTHVSEDCF | | |
| | PLLDGCRKNRQKWQALAEQQEKMLINGE | | |
| | SGQAKRN | | |
| Carbonic | MSHHWGYGKHNGPEHWHKDFPIAKGERQ | 5 | Celecoxib |
| anhydrase 11 | SPVDIDTHTAKYDPSLKPLSVSYDQATS | | Acetazolamide |
| (CA2) (Uniprot | LRILNNGHAFNVEFDDSQDKAVLKGGPL | | |
| ID: P00918) | DGTYRLIQFHFHWGSLDGQGSEHTVDKK | | |
| | KYAAELHLVHWNTKYGDFGKAVQQPDGL | | |
| | AVLGIFLKVGSAKPGLQKVVDVLDSIKT | | |
| | KGKSADFTNFDPRGLLPESLDYWTYPGS | | |
| | LTTPPLLECVTWIVLKEPISVSSEQVLK | | |
| | FRKLNFNGEGEPEELMVDNWRPAQPLKN | | |
| | RQIKASFK | | |
| (Human estrogen | MTMTLHTKASGMALLHQIQGNELEPLNR | 6 | Bazedoxifene |
| receptor (ER) | PQLKIPLERPLGEVYLDSSKPAVYNYPE | | Raloxifene |
| Uniprot ID: | GAAYEFNAAAAANAQVYGQTGLPYGPGS | | |
| P03372.2) | EAAAFGSNGLGGFPPLNSVSPSPLMLLH | | |
| | PPPQLSPFLQPHGQQVPYYLENEPSGYT | | |
| | VREAGPPAFYRPNSDNRRQGGRERLAST | | |
| | NDKGSMAMESAKETRYCAVCNDYASGYH | | |
| | YGVWSCEGCKAFFKRSIQGHNDYMCPAT | | |
| | NQCTIDKNRRKSCQACRLRKCYEVGMMK | | |
| | GGIRKDRRGGRMLKHKRQRDDGEGRGEV | | |
| | GSAGDMRAANLWPSPLMIKRSKKNSLAL | | |
| | SLTADQMVSALLDAEPPILYSEYDPTRP | | |
| | FSEASMMGLLTNLADRELVHMINWAKRV | | |
| | PGFVDLTLHDQVHLLECAWLEILMIGLV | | |
| | WRSMEHPGKLLFAPNLLLDRNQGKCVEG | | |
| | MVEIFDMLLATSSRFRMMNLQGEEFVCL | | |
| | KSIILLNSGVYTFLSSTLKSLEEKDHIH | | |
| | RVLDKITDTLIHLMAKAGLTLQQQHQRL | | |
| | AQLLLILSHIRHMSNKGMEHLYSMKCKN | | |
| | VVPLYDLLLEMLDAHRLHAPTSRGGASV | | |
| | EETDQSHLATAGSTSSHSLQKYYITGEA | | |
| | EGFPATV | | |

In some embodiments, the sequence of a protein used to develop DRDs may comprise all, part of, or a region thereof of a protein sequence in Table 3. In some embodiments, proteins that may be used to develop DRDs include isoforms of proteins listed in Table 3.

hPDE5 DRDs

In some embodiments, a DRD of the present disclosure is derived from hPDE5. In some embodiments, a DRD of the present disclosure is derived from hPDE5 isoform 2. In some embodiments, a DRD of the present disclosure is derived from hPDE5 isoform 3. In some embodiments, a DRD of the present disclosure is derived from hPDE5 isoform X1.

In some embodiments, a DRD of the present disclosure is derived from a cGMP-specific 3′,5′-cyclic phosphodiesterase (hPDE5) comprising the amino acid sequence of SEQ ID NO. 71.

In some embodiments, a DRD of the present disclosure may include the whole hPDE5 (SEQ ID NO. 71). In some embodiments, DRDs derived from hPDE5 may comprise the catalytic domain of hPDE5 (e.g., 535-860 of SEQ ID NO. 71). In some embodiments, hPDE5 DRDs of the present disclosure may include a methionine at the N terminal of the catalytic domain of hPDE5, i.e. amino acids 535-860 of hPDE5 wild-type (WT).

In some embodiments, a DRD of the present disclosure comprises, in whole or in part, a cGMP-specific 3′,5′-cyclic phosphodiesterase (hPDE5; SEQ ID NO. 71), and further comprises a mutation in the amino acid at position 732 (R732) of SEQ ID NO. 71. In some embodiments, the mutation in the amino acid at position 732 (R732) is selected from the group consisting of R732L, R732A, R732G, R732V, R732I, R732P, R732F, R732W, R732Y, R732H, R732S, R732T, R732D, R732E, R732Q, R732N, R732M, R732C, and R732K.

In some embodiments, a hPDE5 DRD of the present disclosure may further comprise one or more mutations independently selected from the group consisting of H653A, F736A, D764A, D764N, Y612F, Y612W, Y612A, W853F, I821A, Y829A, F787A, D656L, Y728L, M625I, E535D, E536G, Q541R, K555R, F559L, F561L, F564L, F564S, K591E, N587S, K604E, K608E, N609H, K630R, K633E, N636S, N661S, Y676D, Y676N, C677R, H678R, D687A, T712S, D724N, D724G, L738H, N742S, A762S, D764G, D764V, S766F, K795E, L797F, I799T, T802P, S815C, M816A, I824T, C839S, K852E, S560G, V585A, I599V, I648V, S663P, L675P, T711A, F744L, L746S, F755L, L804P, M816T, and F840S.

In some embodiments, a DRD of the present disclosure comprises, in whole or in part, a cGMP-specific 3′,5′-cyclic phosphodiesterase (hPDE5; SEQ ID NO. 71), and further comprises a mutation in the amino acid at position 732 (R732) of SEQ ID NO. 71. In some such embodiments, the DRD further comprises (i) a mutation in the amino acid at position 764 (D764) of SEQ ID NO. 71, wherein the mutation at D764 is selected from D764N and D764A; (ii) a mutation in the amino acid at position 612 (Y612) of SEQ ID NO. 71, wherein the mutation at Y612 is selected from the group consisting of Y612A, Y612F, and Y612W; (iii) an F736A mutation in the amino acid at position 736 (F736) of SEQ ID NO. 71; or (iv) an H653A mutation in the amino acid at position 653 (H653) of SEQ ID NO. 71.

In some embodiments, a DRD of the present disclosure comprises, in whole or in part, a cGMP-specific 3′,5′-cyclic phosphodiesterase (hPDE5; SEQ ID NO. 71), and further comprises a mutation in the amino acid at a position relative to SEQ ID NO. 71, the mutation selected from the group consisting of: W853F, I821A, Y829A, F787A, F736A, D656L, Y728L, M625I, and H653A.

In some embodiments, a hPDE5 DRD of the present disclosure may comprise one or more mutations independently selected from the group consisting of T537A, E539G, V548E, D558G, F559S, E565G, C574N, R577Q, R577W, N583S, Q586R, Q589L, K591R, K591R, L595P, C596R, W615R, F619S, Q623R, K633I, Q635R, N636S, T639S, D640N, E642G, I643T, L646S, A649V, A650T, S652G, H653A, D654G, V660A, V660A, L672P, A673T, C677Y, M681T, E682G, H685R, F686S, Q688R, M691T, S695G, G697D, S702I, I706T, E707K, Y709H, Y709C, I715V, I720V, A722V, D724G, Y728C, K730E, R732L, L738I, I739M, K741N, K741R, F744L, D748N, K752E, K752E, K752E, E753K, L756V, M758T, M760T, A762V, C763R, D764N, D764N, I774V, L781F, L781P, E785K, R794G, M805T, R807G, K812R, I813T, I813T, M816R, Q817R, V818A, F820S, I821V, C825R, Y829C, E830K, L832P, S836L, C846Y, C846S, L856P, L856P, A857T, or E858G.

In some embodiments, a hPDE5 DRD of the present disclosure may comprise two mutations independently selected from E536K, I739W; H678F, S702F; E669G, I700T; G632S, I648T; T639S, M816R; Q586R, D724G; E539G, L738I; L672P, S836L; M691T, D764N; I720V, F820S; E682G, D748N; S652G, Q688R; Y728C, Q817R; H653, R732L; L595P, K741R; R732D, F736S; R732E, F736D; R732V, F736G; R732W, F736G; R732W, F736V; R732L, F736W; R732P, F736Q; R732A, F736A; R732S, F736G; R732T, F736P; R732M, F736H; R732Y, F736M; R732P, F736D; R732P, F736G; R732W, F736L; R732L, F736S; R732D, F736T; R732L, F736V; R732G, F736V; and R732W, F736A.

In some embodiments, a hPDE5 DRD of the present disclosure may comprise two mutations independently selected from Q623R, D654G, K741N; A673T, L756V, C846Y; E642G, G697D, I813T; C677Y, H685R, A722V; Q635R, E753K, I813T; Y709H, K812R, L832P; N583S, K752E, C846S; K591R, I643T, L856P; F619S, V818A, Y829C; and F559S, Y709C, M760T. In some embodiments, a hPDE5 DRD of the present disclosure may comprise two mutations independently selected from S695G, E707K, I739M, C763R; A649V, A650T, K730E, E830K; and R577W, W615R, M805T, I821V.

In some embodiments, a hPDE5 DRD of the present disclosure may comprise multiple mutations independently selected from V660A, L781F, R794G, C825R, E858G; T537A, D558G, I706T, F744L, D764N; R577Q, C596R, V660A, I715V, E785K, L856P; and V548E, Q589L, K633I, M681T, S702I, K752E, L781P, A857T.

hDHFR DRDs

In some embodiments, a DRD of the present disclosure is derived from a human dihydrofolate reductase (hDHFR) protein such as, but not limited to, human dihydrofolate reductase 1 (hDHFR1), human dihydrofolate reductase 2 (hDHFR2), or a fragment or variant thereof.

In some embodiments, the DRD may be derived from a hDHFR protein and include at least one mutation. In some embodiments, the DRD may be derived from a hDHFR protein and include more than one mutation. In some embodiments, the DRD may be derived from a hDHFR protein and include two, three, four or five mutations.

In some embodiments, a DRD of the present disclosure may include the whole hDHFR (SEQ ID NO. 2). In some embodiments, DRDs derived from hDHFR may comprise amino acids 2-187 of the parent hDHFR sequence (e.g., amino acids 2-187 of SEQ ID NO. 2). This is referred to herein as an hDHFR M1del mutation.

In some embodiments, a DRD of the present disclosure comprises a region of or the whole hDHFR (SEQ ID NO. 2), and further comprises a mutation relative to SEQ ID NO. 2 selected from I17V, F59S, N65D, K81R, Y122I, N127Y, M140I, K185E, N186D, and M140I.

In some embodiments, a DRD of the present disclosure comprises a region of or the whole hDHFR (SEQ ID NO. 2), and further comprises two or more mutations relative to SEQ ID NO. 2.

In some embodiments, a hDHFR DRD of the present disclosure comprises two or more mutations selected from (A10V, H88Y); (C7R/Y163C); (117V, Y122I); (Q36H, Y122I); (Q36K, Y122I); (Q36R, Y122I); (Q36S, Y122I); (Q36T, Y122I); (N65H, Y122I); (N65L, Y122I); (N65R, Y122I); (N65W, Y122I); (Q103E, Y122I); (Q103S, Y122I); (N108D; Y122I); (V121A, Y122I); (Y122I, K174N); (Y122I, E162G); (A125F, Y122I); (N127Y, Y122I); (H131R/E144G); (E162G/I176F); (K55R, N65K, Y122I); (Q36E, Q103H, Y122I); (Q36F, N65F, Y122I); and (V 110A/V136M/K177R).

In some embodiments, a hDHFR DRD of the present disclosure comprises two or more mutations selected from (117V, Y122I); (G21T, Y122N); (Q36H, Y122I); (Q36K, Y122I); (Q36R, Y122I); (Q36S, Y122I); (Q36T, Y122I); (N65H, Y122I); (N65L, Y122I); (N65R, Y122I); (N65W, Y122I); (L74N, Y122I); (Q103E, Y122I); (Q103 S, Y122I); (N108D; Y122I); (V121A, Y122I); (Y122I, K174N); (Y122I, E162G); (A125F, Y122I); (N127Y, Y122I); (K55R, N65K, Y122I); (Q36E, Q103H, Y122I); and (Q36F, N65F, Y122I).

In some embodiments, a DRD of the present disclosure comprises, in whole or in part, a human dihydrofolate reductase (hDHFR; SEQ ID NO. 2), and further comprises a Y122I mutation in the amino acid at position 122 (Y122) of SEQ ID NO. 2. In some such embodiments, the DRD further comprises: (i) a Q36K mutation in the amino acid at position 36 (Q36) of SEQ ID NO. 2; (ii) an A125F mutation in the amino acid at position 125 (A125) of SEQ ID NO. 2; or (iii) a N65F mutation in the amino acid at position 65 (N65) of SEQ ID NO. 2 and a substitution of F or K at the amino acid position 36 (Q36) of SEQ ID NO. 2.

In some embodiments, a hDHFR DRD of the present disclosure may comprise one or more mutations independently selected from the group consisting of M1del, V2A, C7R, I8V, V9A, A10T, A10V, Q13R, N14S, G16S, I17N, I17V, K19E, N20D, G21T, G21E, D22S, L23S, P24S, L28P, N30D, N30H, N30S, E31G, E31D, F32M, R33G, R33S, F35L, Q36R, Q36S, Q36K, Q36F, R37G, M38V, M38T, T40A, V44A, K47R, N49S, N49D, M53T, G54R, K56E, K56R, T57A, F59S, I61T, K64R, N65A, N65S, N65D, N65F, L68S, K69E, K69R, R71G, I72T, I72A, I72V, N73G, L74N, V75F, R78G, L80P, K81R, E82G, H88Y, F89L, R92G, S93G, S93R, L94A, D96G, A97T, L98S, K99G, K99R, L100P, E102G, Q103R, P104S, E105G, A107T, A107V, N108D, K109E, K109R, V110A, D111N, M112T, M112V, V113A, W114R, I115V, I115L, V116I, G117D, V121A, Y122C, Y122D, Y122I, K123R, K123E, A125F, M126I, N127R, N127S, N127Y, H128R, H128Y, H131R, L132P, K133E, L134P, F135P, F135L, F135S, F135V, V136M, T137R, R138G, R138I, I139T, I139V, M140I, M140V, Q141R, D142G, F143S, F143L, E144G, D146G, T147A, F148S, F148L, F149L, P150L, E151G, I152V, D153A, D153G, E155G, K156R, Y157R, Y157C, K158E, K158R, L159P, L160P, E162G, Y163C, V166A, S168C, D169G, V170A, Q171R, E172G, E173G, E173A, K174R, I176A, I176F, I176T, K177E, K177R, Y178C, Y178H, F180L, E181G, V182A, Y183C, Y183H, E184R, E184G, K185R, K185del, K185E, N186S, N186D, D187G, and D187N.

In some embodiments, a DRD of the present disclosure comprises hDHFR (C7R, Y163C); hDHFR (E162G, I176F); hDHFR (G21T, Y122I); hDHFR (H131R, E144G); hDHFR (I17V, Y122I; hDHFR (L74N, Y122I; hDHFR (L94A, T147A); hDHFR (M53T, R138I); hDHFR (N127Y, Y122I); hDHFR (Q36K, Y122I); hDHFR (T137R, F143L); hDHFR (T57A, I72A); hDHFR (V121A, Y122I); hDHFR (V75F, Y122I); hDHFR (Y122I, A125F); hDHFR (Y122I, M140I); hDHFR (Y178H, E181G); hDHFR (Y183H, K185E); hDHFR (Amino acid 2-187 of WT) (G21T, Y122I); hDHFR (Amino acid 2-187 of WT) (I17V, Y122I); hDHFR (Amino acid 2-187 of WT) (L74N, Y122I); hDHFR (Amino acid 2-187 of WT) (L94A, T147A); hDHFR (Amino acid 2-187 of WT) (M53T, R138I); hDHFR (Amino acid 2-187 of WT) (N127Y, Y122I); hDHFR (Amino acid 2-187 of WT) (Q36K, Y122I); hDHFR (Amino acid 2-187 of WT) (V121A, Y122I); hDHFR (Amino acid 2-187 of WT) (V75F, Y122I); hDHFR (Amino acid 2-187 of WT) (Y122I, A125F); hDHFR (Amino acid 2-187 of WT) (Y122I, M140I); hDHFR (E31D, F32M, V116I); hDHFR (G21E, I72V, I176T); hDHFR (I8V, K133E, Y163C); hDHFR (K19E, F89L, E181G); hDHFR (L23S, V121A, Y157C); hDHFR (N49D, F59S, D153G); hDHFR (Q36F, N65F, Y122I); hDHFR (Q36F, Y122I, A125F); hDHFR (V110A, V136M, K177R); hDHFR (V9A, S93R, P150L); hDHFR (Y122I, H131R, E144G); hDHFR (G54R, I115L, M140V, S168C); hDHFR (Amino acid 2-187 of WT) (E31D, F32M, V116I); hDHFR (Amino acid 2-187 of WT) (Q36F, N65F, Y122I); hDHFR (Amino acid 2-187 of WT) (Q36F, Y122I, A125F); hDHFR (Amino acid 2-187 of WT) (Y122I, H131R, E144G); hDHFR (V2A, R33G, Q36R, L100P, K185R); hDHFR (D22S, F32M, R33S, Q36S, N65S); hDHFR (Amino acid 2-187 of WT) (D22S, F32M, R33S, Q36S, N65S); hDHFR (I17N, L98S, K99R, M112T, E151G, E162G, E172G); hDHFR (G16S, I17V, F89L, D96G, K123E, M140V, D146G, K156R); hDHFR (K81R, K99R, L100P, E102G, N108D, K123R, H128R, D142G, F180L, K185E); hDHFR (R138G, D142G, F143S, K156R, K158E, E162G, V166A, K177E, Y178C, K185E, N186S); hDHFR (N14S, P24S, F35L, M53T, K56E, R92G, S93G, N127S, H128Y, F135L, F143S, L159P, L160P, E173A, F180L); hDHFR (F35L, R37G, N65A, L68S, K69E, R71G, L80P, K99G, Gi 17D, L132P, I139V, M140I, D142G, D146G, E173G, D187G); hDHFR (L28P, N30H, M38V, V44A, L68S, N73G, R78G, A97T, K99R, A107T, K109R, D111N, L134P, F135V, T147A, I152V, K158R, E172G, V182A, E184R); hDHFR (V2A, I17V, N30D, E31G, Q36R, F59S, K69E, I72T, H88Y, F89L, N108D, K109E, V110A, I115V, Y122D, L132P, F135S, M140V, E144G, T147A, Y157C, V170A, K174R, N186S); hDHFR (L100P, E102G, Q103R, P104S, E105G, N108D, V113A, W114R, Y122C, M126I, N127R, H128Y, L132P, F135P, I139T, F148S, F149L, I152V, D153A, D169G, V170A, I176A, K177R, V182A, K185R, N186S); and hDHFR (A10T, Q13R, N14S, N20D, P24S, N30S, M38T, T40A, K47R, N49S, K56R, I61T, K64R, K69R, I72A, R78G, E82G, F89L, D96G, N108D, M112V, W114R, Y122D, K123E, I139V, Q141R, D142G, F148L, E151G, E155G, Y157R, Q171R, Y183C, E184G, K185del, D187N).

ecDHFR DRDs

In some embodiments, a DRD of the present disclosure is derived from E. coli dihydrofolate reductase (ecDHFR). In some embodiments, the DRD may be derived from an ecDHFR protein and include at least one mutation. In some embodiments, the DRD may be derived from an ecDHFR protein and include more than one mutation. In some embodiments, the DRD may be derived from an ecDHFR protein and include two, three, four or five mutations. In some embodiments, the DRD may be derived from an ecDHFR protein and comprise at least one mutation selected from Y100I, F103L, and G121V. In some embodiments, the DRD may be derived from an ecDHFR protein and comprise at least two mutations selected from R12Y,Y100I; R12H,E129K; H12Y,Y100I; H12L,Y100I; R98H,F103S; M42T,H114R; N18T,A19V; and I61F,T68S.

### FKBP DRDs

In some embodiments, a DRD of the present disclosure is derived from a FK506 binding protein (FKBP) protein or a fragment or variant thereof. In some embodiments, the DRD may be derived from a FKBP protein and include at least one mutation. In some embodiments, the DRD may be derived from a FKBP protein and include more than one mutation. In some embodiments, the DRD may be derived from an FKBP protein and include two, three, four or five mutations.

In some embodiments, a DRD of the present disclosure is derived from, in whole or in part, a human FKBP protein (SEQ ID NO. 3) and comprises at least one mutation selected from F36V, F15S, V24A, H25R, E60G, L106P, D100G, M66T, R71G, D100N, E102G, and K105I. In some embodiments, a FKBP DRD of the present disclosure comprises more than one mutation selected from F36P, L106P; and E31G, F36V, R71G, K105E.

### ER DRDs

In some embodiments, a DRD of the present disclosure is derived from an Estrogen Receptor (ER) protein or a fragment or variant thereof. In some embodiments, the DRD may be derived from an ER protein and include at least one mutation. In some embodiments, the DRD may be derived from an ER protein and include more than one mutation. In some embodiments, the DRD may be derived from an ER protein and include two, three, four or five mutations.

In some embodiments, a DRD of the present disclosure comprises the ligand binding domain of ER (amino acids 305 to 509 of SEQ ID NO: 6). In some embodiments, a DRD may include at least one mutation relative to the ligand binding domain of ER, wherein the mutation occurs at position 413 (N413) and/or at position 502 (Q502). In some embodiments, the mutation is at position N413 and is N413D, N413T, N413H, N413A, N413Q, N413V, N413C, N413K, N413M, N413R, N413S, N413W, N413I, N413E, N413L, N413P, N413F, N413Y or N413G. In some embodiments, the mutation is at position Q502 and is Q502H, Q502D, Q502E, Q502V, Q502A, Q502T, Q502N, Q502K, Q502S, Q502L, Q502Y, Q502W, Q502F, Q502I, Q502G, Q502P, Q502M, or Q502C. In some embodiments, the DRD comprises mutations at position N413 and at position Q502, wherein the mutation at position N413 is selected from N413D, N413T, N413H, N413A, N413Q, N413V, N413C, N413K, N413M, N413R, N413S, N413W, N413I, N413E, N413L, N413P, N413F, N413Y or N413G and the mutation at position Q502 is selected from Q502H, Q502D, Q502E, Q502V, Q502A, Q502T, Q502N, Q502K, Q502S, Q502L, Q502Y, Q502W, Q502F, Q502I, Q502G, Q502P, Q502M, or Q502C.

In some embodiments, the at least one mutation is N413D. In some embodiments, the at least one mutation is N413T. In some embodiments, the at least one mutation is Q502H. In some embodiments, the ER DRD comprises at least two mutations and is N413T, Q502H or N413D, Q502H.

In some embodiments, an ER DRD may further comprise one or more mutations independently selected from L384M, M421G, G521R or Y537S.

In some embodiments, a DRD of the present disclosure comprises the following: ER (aa 305-549 of WT, L384M, N413F, M421G, G521R, Y537S), ER (aa 305-549 of WT, L384M, N413L, M421G, G521R, Y537S), ER (aa 305-549 of WT, L384M, N413Y, M421G, G521R, Y537S), ER (aa 305-549 of WT, L384M, N413H, M421G, G521R, Y537S), ER (aa 305-549 of WT, L384M, N413Q, M421G, G521R, Y537S), ER (aa 305-549 of WT, L384M, N413I, M421G, G521R, Y537S), ER (aa 305-549 of WT, L384M, N413M, M421G, G521R, Y537S), ER (aa 305-549 of WT, L384M, N413K, M421G, G521R, Y537S), ER (aa 305-549 of WT, L384M, N413V, M421G, G521R, Y537S), ER (aa 305-549 of WT, L384M, N413S, M421G, G521R, Y537S), ER (aa 305-549 of WT, L384M, N413C, M421G, G521R, Y537S), ER (aa 305-549 of WT, L384M, N413W, M421G, G521R, Y537S), ER (aa 305-549 of WT, L384M, N413P, M421G, G521R, Y537S), ER (aa 305-549 of WT, L384M, N413R, M421G, G521R, Y537S), ER (aa 305-549 of WT, L384M, N413T, M421G, G521R, Y537S), ER (aa 305-549 of WT, L384M, N413A, M421G, G521R, Y537S), ER (aa 305-549 of WT, L384M, N413E, M421G, G521R, Y537S), ER (aa 305-549 of WT, L384M, N413G, M421G, G521R, Y537S), ER (aa 305-549 of WT, L384M, M421G, Q502F, G521R, Y537S), ER (aa 305-549 of WT, L384M, M421G, Q502L, G521R, Y537S), ER (aa 305-549 of WT, L384M, M421G, Q502Y, G521R, Y537S), ER (aa 305-549 of WT, L384M, M421G, Q502H, G521R, Y537S), ER (aa 305-549 of WT, L384M, M421G, Q502I, G521R, Y537S), ER (aa 305-549 of WT, L384M, M421G, Q502M, G521R, Y537S), ER (aa 305-549 of WT, L384M, M421G, Q502N, G521R, Y537S), ER (aa 305-549 of WT, L384M, M421G, Q502K, G521R, Y537S), ER (aa 305-549 of WT, L384M, M421G, Q502V, G521R, Y537S), ER (aa 305-549 of WT, L384M, M421G, Q502S, G521R, Y537S), ER (aa 305-549 of WT, L384M, M421G, Q502C, G521R, Y537S), ER (aa 305-549 of WT, L384M, M421G, Q502W, G521R, Y537S), ER (aa 305-549 of WT, L384M, M421G, Q502P, G521R, Y537S), ER (aa 305-549 of WT, L384M, M421G, Q502T, G521R, Y537S), ER (aa 305-549 of WT, L384M, M421G, Q502A, G521R, Y537S), ER (aa 305-549 of WT, L384M, M421G, Q502D, G521R, Y537S), ER (aa 305-549 of WT, L384M, M421G, Q502E, G521R, Y537S), and ER (aa 305-549 of WT, L384M, M421G, Q502G, G521R, Y537S).

### CA2 DRDs

In some embodiments, a DRD of the present disclosure may be derived from human carbonic anhydrase 2 (hCA2), which is a member of the carbonic anhydrases, a superfamily of metalloenzymes. In some embodiments, the DRD may be derived from a hCA2 protein and include at least one mutation. In some embodiments, the DRD may be derived from a hCA2 protein and include more than one mutation. In some embodiments, the DRD may be derived from an hCA2 protein and include two, three, four or five mutations.

In some embodiments, a DRD of the present disclosure may be derived from amino acids 1-260 of CA2 (SEQ ID NO. 5). In some embodiments, DRDs are derived from CA2 comprising amino acids 2-260 of the parent CA2 sequence (e.g., amino acids 2-260 of SEQ ID NO. 5). This is referred to herein as a CA2 Ml del mutation. In one embodiment, DRDs derived from CA2 may comprise amino acids 2-237 of the parent CA2 sequence (e.g., amino acids 2-237 of SEQ ID NO. 5).

In some embodiments, a DRD of the present disclosure comprises a region of or the whole human carbonic anhydrase 2 (CA2; SEQ ID NO. 5), and further comprises a mutation relative to SEQ ID NO. 5 selected from E106D, G63D, H122Y, I59N, L156H, L183S, L197P, S56F, S56N, W208S, Y193I, and Y51T.

In some embodiments, a DRD of the present disclosure comprises a region of or the whole human carbonic anhydrase 2 (CA2; SEQ ID NO. 5), and further comprises a mutation relative to SEQ ID NO. 5 selected from A115L, A116Q, A116V, A133L, A133T, A141P, A152D, A152L, A152R, A173C, A173G, A173L, A173T, A23P, A247L, A247S, A257L, A257S, A38P, A38V, A54Q, A54V, A54X, A65L, A65N, A65V, A77I, A77P, A77Q, C205M, C205R, C205V, C205W, C205Y, D101G, D101M, D110I, D129I, D138G, D138M, D138N, D161*, D161M, D161V, D164G, D164I, D174*, D174T, D179E, D179I, D179R, D189G, D189I, D19T, D19V, D242G, D242T, D32T, D34T, D41T, D52I, D52L, D71F, D71G, D71K, D71M, D71S, D71Y, D72I, D72S, D72T, D72X, D75T, D75V, D85M, E106D, E106G, E106S, E117*, E117N, E14N, E186*, E186N, E204A, E204D, E204G, E204N, E213*, E213G, E213N, E220K, E220R, E220S, E233D, E233G, E233R, E235*, E235G, E235N, E237K, E237R, E238*, E238N, E238R, E26S, E69D, E69K, E69S, F130L, F146V, F175I, F175L, F175S, F178L, F178S, F20L, F20S, F225I, F225L, F225S, F225Y, F230I, F230L, F230S, F259L, F259S, F66S, F70I, F70L, F95Y, G102D, G104R, G104V, G128R, G12D, G12E, G131E, G131R, G131W, G139D, G144D, G144V, G150A, G150S, G150W, G155A, G155C, G155D, G155S, G170A, G170D, G182A, G182W, G195A, G195R, G232R, G232W, G234L, G234V, G25E, G63D, G63V, G81E, G81V, G82D, G86A, G86D, G98V, H107I, H107Q, H119T, H119Y, H122T, H122Y, H15L, H15T, H15Y, H17D, H17I, H36I, H36Q, H64M, H94T, H96T, I145F, I145M, I166H, I166L, I209D, I209L, I215H, I215S, I22L, I255N, I255S, I33S, I59F, I59N, I59S, I91F, K111E, K111N, K112R, K113I, K113N, K126N, K132E, K132R, K148E, K148R, K153*, K153N, K158E, K158N, K167*, K169N, K169R, K171Q, K171R, K18R, K212N, K212Q, K212R, K212W, K224E, K224N, K227*, K227N, K24R, K251E, K251R, K256Q, K260F, K260L, K260Q, K39S, K45N, K45S, K80M, K80R, L118F, L120W, L140V, L140W, L143*, L147*, L147F, L156F, L156H, L156P, L156Q, L163A, L163W, L183P, L183S, L184F, L184P, L188P, L188W, L197*, L197M, L197P, L197R, L197T, L202F, L202H, L202I, L202P, L202R, L202S, L203P, L203S, L203W, L211*, L211A, L211S, L223*, L223I, L223V, L228F, L228H, L228T, L239*, L239F, L239T, L250*, L250P, L250T, L44*, L44M, L47C, L47V, L57*, L57X, L60S, L79F, L79S, L84W, L90*, L90V, M240D, M240L, M240R, M240W, N11D, N11K, N124T, N177*, N177T, N229*, N229T, N231D, N231F, N231K, N231L, N231M, N231Q, N231T, N243Q, N243T, N252E, N252T, N61R, N61T, N61Y, N62K, N62M, N67D, N67T, P137L, P13A, P13H, P13L, P13S, P154L, P154R, P154T, P180L, P180S, P185L, P185S, P185V, P194Q, P200A, P200L, P200S, P200T, P201A, P201L, P201R, P201S, P214T, P236L, P236T, P246L, P246Q, P249A, P249F, P249H, P249I, P249X, P30L, P30S, P42L, P83A, Q103K, Q135S, Q136N, Q157R, Q157S, Q221A, Q221R, Q248F, Q248L, Q248S, Q254A, Q254K, Q28S, Q53H, Q53K, Q53N, Q74R, Q92H, Q92S, R181H, R181S, R181V, R226H, R226P, R226V, R245A, R253G, R253Q, R27A, R58G, R89D, R89F, R89I, R89X, R89Y, S105L, S105Q, S151A, S1511, S151Q, S165F, S165P, S172E, S172V, S1871, S187P, S196H, S196L, S216A, S216Q, S218A, S218Q, S219A, S219Q, S258F, S258P, S29C, S29P, S43P, S43T, S48L, S50P, S56F, S56N, S56P, S56X, S73L, S73N, S73X, S99H, T108L, T125I, T125P, T168K, T168N, T168Q, T176H, T176L, T192D, T192F, T192I, T192N, T192P, T192X, T198D, T1981, T198P, T199A, T199H, T199P, T207D, T2071, T207P, T207S, T35I, T35L, T37Q, T55L, T87L, V109M, V109W, V121F, V134C, V134F, V142F, V149G, V149L, V159L, V159S, V160C, V160L, V162A, V162C, V206*, V206C, V206M, V210C, V217L, V217R, V217S, V222A, V222C, V222G, V241G, V241W, V241X, V31L, V49F, V68L, V68W, V78C, W123G, W123R, W16G, W191*, W191G, W191L, W208G, W208L, W208S, W244*, W244G, W244L, W97C, W97G, Y114H, Y114M, Y127M, Y190*, Yl90L, Yl90T, Y193C, Y193F, Y193I, Y193L, Y193T, Y193V, Y193X, Y40M, Y51F, Y51M, Y51T, Y51X, Y88T, K9N, and S29A. As used herein “*” indicates the translation of the stop codon and X indicates any amino acid.

In some embodiments, a DRD of the present disclosure comprises a region of or the whole human carbonic anhydrase 2 (CA2; SEQ ID NO. 5), and further comprises two or more mutations relative to SEQ ID NO. 5.

In some embodiments, a DRD of the present disclosure comprises CA2 (aa 2-260 of WT, R27L, H122Y), CA2 (aa 2-260 of WT, T87I, H122Y), CA2 (aa 2-260 of WT, H122Y, N252D), CA2 (aa 2-260 of WT, D72F, V241F), CA2 (aa 2-260 of WT, V241F, P249L), CA2 (aa 2-260 of WT, D72F, P249L), CA2 (aa 2-260 of WT, D71L, L250R), CA2 (aa 2-260 of WT, D72F, P249F), CA2 (aa 2-260 of WT, T55K, G63N, Q248N), CA2 (aa 2-260 of WT, L156H, A257del, S258del, F259del, K260del), CA2 (aa 2-260 of WT, L156H, S2del, H3del, H4del, W5del), CA2 (aa 2-260 of WT, W4Y, L156H), CA2 (aa 2-260 of WT, L156H, G234del, E235del, P236del), CA2 (aa 2-260 of WT, L156H, F225L), CA2 (aa 2-260 of WT, D70N, D74N, D100N, L156H), (CA2 (aa 2-260 of WT, I59N, G102R), CA2 (aa 2-260 of WT, G63D, E69V, N231I), CA2 (aa 2-260 of WT, R27L, T871, H122Y, N252D), CA2 (aa 2-260 of WT, D72F, V241F, P249L), CA2 (aa 2-260 of WT, D71L, T87N, L250R), CA2 (aa 2-260 of WT, L156H, S172C, F178Y, E186D), CA2 (aa 2-260 of WT, A77I, P249F), CA2 (aa 2-260 of WT, E106D, C205S), CA2 (aa 2-260 of WT, C205S, W208S), CA2 (aa 2-260 of WT, S73N, R89Y), CA2 (aa 2-260 of WT, D71K, T192F), CA2 (aa 2-260 of WT, S73N, R89F), CA2 (aa 2-260 of WT, G63D, M240L), CA2 (aa 2-260 of WT, V134F, L228F), or CA2 (aa 2-260 of WT, S56F, D71S).

In some embodiments, a DRD of the present disclosure comprises CA2 (aa 2-260 of WT, R27L, H122Y), CA2 (aa 2-260 of WT, T87I, H122Y), CA2 (aa 2-260 of WT, H122Y, N252D), CA2 (aa 2-260 of WT, D72F, V241F), CA2 (aa 2-260 of WT, V241F, P249L), CA2 (aa 2-260 of WT, D72F, P249L), CA2 (aa 2-260 of WT, D71L, L250R), CA2 (aa 2-260 of WT, D72F, P249F), CA2 (aa 2-260 of WT, T55K, G63N, Q248N), CA2 (aa 2-260 of WT, L156H, A257del, S258del, F259del, K260del), CA2 (aa 2-260 of WT, L156H, S2del, H3del, H4del, W5del), CA2 (aa 2-260 of WT, W4Y, L156H), CA2 (aa 2-260 of WT, L156H, G234del, E235del, P236del), CA2 (aa 2-260 of WT, L156H, F225L), CA2 (aa 2-260 of WT, D70N, D74N, D100N, L156H), (CA2 (aa 2-260 of WT, I59N, G102R), CA2 (aa 2-260 of WT, G63D, E69V, N231I), CA2 (aa 2-260 of WT, R27L, T87I, H122Y, N252D), CA2 (aa 2-260 of WT, D72F, V241F, P249L), CA2 (aa 2-260 of WT, D71L, T87N, L250R), CA2 (aa 2-260 of WT, L156H, S172C, F178Y, E186D), CA2 (aa 2-260 of WT, D71F, N231F), CA2 (aa 2-260 of WT, A77I, P249F), CA2 (aa 2-260 of WT, D71K, P249H), CA2 (aa 2-260 of WT, D72F, P249H), CA2 (aa 2-260 of WT, Q53N, N61Y), CA2 (aa 2-260 of WT, E106D, C205S), CA2 (aa 2-260 of WT, C205S, W208S), CA2 (aa 2-260 of WT, S73N, R89Y), CA2 (aa 2-260 of WT, D71K, T192F), CA2 (aa 2-260 of WT, Y193L, K260L), CA2 (aa 2-260 of WT, D71F, V241F, P249L), CA2 (aa 2-260 of WT, L147F, Q248F), CA2 (aa 2-260 of WT, D52I, S258P), CA2 (aa 2-260 of WT, D72S, T192N), CA2 (aa 2-260 of WT, D179E, T192I), CA2 (aa 2-260 of WT, S56N, Q103K), CA2 (aa 2-260 of WT, D71Y, Q248L), CA2 (aa 2-260 of WT, S73N, R89F), CA2 (aa 2-260 of WT, D71K, N231L, E235G, L239F), CA2 (aa 2-260 of WT, D72F, P249I), CA2 (aa 2-260 of WT, D72X, V241X, P249X), CA2 (aa 2-260 of WT, A54X, S56X, L57X, T192X), CA2 (aa 2-260 of WT, Y193V, K260F), CA2 (aa 2-260 of WT, G63D, M240L), CA2 (aa 2-260 of WT, V134F, L228F), CA2 (aa 2-260 of WT, D71G, N231K), CA2 (aa 2-260 of WT, S56F, D71S), CA2 (aa 2-260 of WT, D52L, G128R, Q248F), CA2 (aa 2-260 of WT, S73X, R89X), CA2 (aa 2-260 of WT, Y51X, D72X, V241X, P249X), CA2 (aa 2-260 of WT, D72I, W97C), CA2 (aa 2-260 of WT, D71K, T192F, N231F), CA2 (aa 2-260 of WT, H36Q, S43T, Y51F, N67D, G131W, R226H), CA2 (aa 2-260 of WT, F70I, F146V), CA2 (aa 2-260 of WT, K45N, V68L, Hi 19Y, K169R, D179E), CA2 (aa 2-260 of WT, H15L, A54V, K111E, E220K, F225I), CA2 (aa 2-260 of WT, P13S, P83A, D101G, K111N, F230I), CA2 (aa 2-260 of WT, G63D, W123R, E220K), CA2 (aa 2-260 of WT, N11D, E69K, G86D, V109M, K113I, T125I, D138G, G155S), CA2 (aa 2-260 of WT, I59N, G102R, A173T), CA2 (aa 2-260 of WT, L79F, P180S), CA2 (aa 2-260 of WT, A77P, G102R, D138N), CA2 (aa 2-260 of WT, F20L, K45N, G63D, E69V, N231I), CA2 (aa 2-260 of WT, T199N, L202P, L228F), CA2 (aa 2-260 of WT, K9N, H122Y, T168K), CA2 (aa 2-260 of WT, Q53H, L90V, Q92H, G131E), CA2 (aa 2-260 of WT, L44M, L47V, N62K, E69D), CA2 (aa 2-260 of WT, D75V, K169N, F259L), CA2 (aa 2-260 of WT, T207S, V222A, N231D), CA2 (aa 2-260 of WT, I59F, V206M, G232R), CA2 (aa 2-260 of WT, P13A, A133T), CA2 (aa 2-260 of WT, I59N, R89I), CA2 (aa 2-260 of WT, A65N, G86D, G131R, G155D, K158N, V162A, G170D, P236L), CA2 (aa 2-260 of WT, G12R, H15Y, D19V), CA2 (aa 2-260 of WT, A65V, F95Y, E106G, H107Q, I145M, F175I), CA2 (aa 2-260 of WT, G63D, E69V, N231I), CA2 (aa 2-260 of WT, S29A, C205S) and/or CA2 (aa 2-260 of WT, S29C, C205S).

In some embodiments, a DRD of the present disclosure comprises, in whole or in part, a human carbonic anhydrase 2 (CA2; SEQ ID NO. 5), and further comprises a H122Y mutation in the amino acid at position 122 (H122) of SEQ ID NO. 5. In some such embodiments, the DRD further comprises: (i) a R27L mutation in the amino acid at position 27 (R27) of SEQ ID NO. 5; (ii) a T87I mutation in the amino acid at position 87 (T87) of SEQ ID NO. 5; (iii) a N252D mutation in the amino acid at position 252 (N252) of SEQ ID NO. 5; or a combination of (i), (ii) and/or (iii).

In some embodiments, a DRD of the present disclosure comprises, in whole or in part, a human carbonic anhydrase 2 (CA2; SEQ ID NO. 5), and further comprises an E106D mutation in the amino acid at position 106 (E106) of SEQ ID NO. 5. In some such embodiments, the DRD further comprises a C205S mutation in the amino acid at position 205 (C205) of SEQ ID NO. 5.

In some embodiments, a DRD of the present disclosure comprises, in whole or in part, a human carbonic anhydrase 2 (CA2; SEQ ID NO. 5), and further comprises a W208S mutation in the amino acid at position 208 (W208) of SEQ ID NO. 5. In some such embodiments, the DRD further comprises a C205S mutation in the amino acid at position 205 (C205) of SEQ ID NO. 5.

In some embodiments, a DRD of the present disclosure comprises, in whole or in part, a human carbonic anhydrase 2 (CA2; SEQ ID NO. 5), and further comprises a I59N mutation in the amino acid at position 59 (I59) of SEQ ID NO. 5. In some such embodiments, the DRD further comprises a G102R mutation in the amino acid at position 102 (G102) of SEQ ID NO. 5.

In some embodiments, a DRD of the present disclosure comprises, in whole or in part, a human carbonic anhydrase 2 (CA2; SEQ ID NO. 5), and further comprises a L156H mutation in the amino acid at position 156 (L156) of SEQ ID NO. 5. In some such embodiments, the DRD further comprises (i) a W4Y mutation in the amino acid at position 4 (W4) of SEQ ID NO. 5; (ii) a F225L mutation in the amino acid at position 225 (F225) of SEQ ID NO. 5; (iii) a deletion of amino acids at positions 257-260 of SEQ ID NO. 5; (iv) a deletion of amino acids at positions 1-5 of SEQ ID NO. 5; or (v) a deletion of amino acids G234, E235 and P236 of SEQ ID NO. 5.

In some embodiments, a DRD of the present disclosure comprises, in whole or in part, a human carbonic anhydrase 2 (CA2; SEQ ID NO. 5), and further comprises four mutations relative to SEQ ID NO. 5, the mutations corresponding to: (i) L156H, S172C, F178Y, and E186D; or (ii) D70N, D74N, D100N, and L156H.

In some embodiments, a DRD of the present disclosure comprises, in whole or in part, a human carbonic anhydrase 2 (CA2; SEQ ID NO. 5), and further comprises a first mutation and a second mutation relative to SEQ ID NO. 5, wherein: (i) the first mutation is a S73N mutation in the amino acid at position 73 (S73) of SEQ ID NO. 5; and (ii) the second mutation is a substitution of F or Y at the amino acid position 89 (R89) of SEQ ID NO. 5.

In some embodiments, a DRD of the present disclosure comprises, in whole or in part, a human carbonic anhydrase 2 (CA2; SEQ ID NO. 5), and further comprises a substitution of N or F at the amino acid position 56 (S56) of SEQ ID NO. 5. In some such embodiments, the DRD comprises two substitutions relative to SEQ ID NO. 5 that correspond to S56F and D71S.

In some embodiments, a DRD of the present disclosure comprises, in whole or in part, a human carbonic anhydrase 2 (CA2; SEQ ID NO. 5), and further comprises one or more substitutions relative to SEQ ID NO. 5, wherein at least one substitution is a substitution of D or N at the amino acid position 63 (G63) of SEQ ID NO. 5, and wherein the one or more substitutions correspond to: (i) G63D; (ii) G63D and M240L; (iii) G63D, E69V and N231I; or (iv) T55K, G63N and Q248N.

In some embodiments, a DRD of the present disclosure comprises, in whole or in part, a human carbonic anhydrase 2 (CA2; SEQ ID NO. 5), and further comprises two or more substitutions relative to SEQ ID NO. 5, wherein one of the two or more substitutions is a substitution of L or K at the amino acid position 71 (D71) of SEQ ID NO. 5, and wherein the two or more substitutions correspond to: (i) D71L and T87N; (ii) D71L and L250R; (iii) D71L, T87N and L250R; or (iv) D71K and T192F.

In some embodiments, a DRD of the present disclosure comprises, in whole or in part, a human carbonic anhydrase 2 (CA2; SEQ ID NO. 5), and further comprises two or more substitutions relative to SEQ ID NO. 5, wherein at least one of the two or more substitutions is: (i) a substitution of F at the amino acid position 241 (V241) of SEQ ID NO. 5; or (ii) a substitution of F or L at the amino acid position 249 (P249) of SEQ ID NO. 5; and wherein the two or more substitutions correspond to: (i) D72F and V241F; (ii) D72F and P249L; (iii) D72F and P249F; (iv) D72F, V241F and P249L; (v) A77I and P249F; or (vi) V241F and P249L.

In some embodiments, a DRD of the present disclosure comprises, in whole or in part, a human carbonic anhydrase 2 (CA2; SEQ ID NO. 5), and further comprises one or more substitutions relative to SEQ ID NO. 5, selected from Y51T, L183S, Y193I, L197P and the combination of V134F and L228F.

The amino acid sequences of the DRDs encompassed in the present disclosure have at least about 70% identity, preferably at least about 75% or 80% identity, more preferably at least about 85%, 86%, 87%, 88%, 89% or 90% identity, and further preferably at least about 91%, 92%, 93%, 94%, 95%, 96%, 97%, 98% or 99% identity to the amino acid sequence of a parent protein from which it is derived. In some embodiments, the amino acid sequence of the DRDs encompassed in the present disclosure have at least about 70% identity, preferably at least about 75% or 80% identity, more preferably at least about 85%, 86%, 87%, 88%, 89% or 90% identity, and further preferably at least about 91%, 92%, 93%, 94%, 95%, 96%, 97%, 98% or 99% identity to the amino acid sequence of a parent protein (for example, a parent protein having an amino acid sequence of any one of SEQ ID Nos: 1, 2, 3, 4, 5, 6, and 71) from which it is derived.

Examples of DRDs of the present disclosure include those derived from: human carbonic anhydrase 2 (CA2), human DHFR, ecDHFR, human estrogen receptor (ER), FKBP, human protein FKBP, and human PDE5. Suitable DRDs, which may be referred to as destabilizing domains or ligand binding domains, are also known in the art. See, e.g., WO2018/161000; WO2018/231759; WO2019/241315; U.S. Pat. Nos. 8,173,792; 8,530,636; WO2018/237323; WO2017/181119; US2017/0114346; US2019/0300864; WO2017/156238; Miyazaki et al., J Am Chem Soc, I34:3942 (2012); Banaszynski et al. (2006) Cell 126:995-1004; Stankunas, K. et al. (2003) Mol. Cell 12:1615-1624; Banaszynski et al. (2008) Nat. Med. 14:1123-1127; Iwamoto et al. (2010) Chem. Biol. 17:981-988; Armstrong et al. (2007) Nat. Methods 4:1007-1009; Madeira da Silva et al. (2009) Proc. Natl. Acad. Sci. USA 106:7583-7588; Pruett-Miller et al. (2009) PLoS Genet. 5:e1000376; and Feng et al. (2015) Elife 4:e10606.

As provided above in the “Transcription Factor System” section, the combination of one or more polynucleotides of a transcription factor system comprises a nucleic acid sequence that encodes a drug responsive domain (DRD), wherein the transcription factor (for example, a transcription factor DNA binding domain, a transcription factor activation domain, or both) is operably linked to the DRD. The nucleic acid sequence that encodes a DRD may be selected from sequences of DRDs described herein. Constructs comprising DRD sequences are provided in Table 1 above. Additional constructs comprising different DRDs are provided in Table 4. An asterisk (“*”) in Table 4 indicates the translation of a stop codon.

TABLE 4
Transcription factor constructs with different DRDs

| | | | NA | | AA |
|---|---|---|---|---|---|
| Con- | | | SEQ | | SEQ |
| struct | Construct | | ID | | ID |
| Name | Description | NA Sequence | NO | AA Sequence | NO |
| ZFHD- | ZFHD1-p65- | cgtgaggctccggtgcccgtcagtgggcagagcgcacatcgcccaca | 60 | MAPKKKRKVERPYACPVESCDRRFSRSD | 61 |
| 059 | GGSGGGSGG | gtccccgagaagttggggggaggggtcggcaattgaaccggtgccta | | ELTRHIRIHTGQKPFQCRICMRNFSRSD | |
| | (SEQ ID NO: | gagaaggtggcgcggggtaaactgggaaagtgatgtcgtgtactggc | | HLTTHIRTHTGGGRRRKKRTSIETNIRV | |
| | 59)-hDHFR | tccgcctttttcccgagggtgggggagaaccgtatataagtgcagta | | ALEKSFLENQKPTSEEITMIADQLNMEK | |
| | (Y122I) | gtcgccgtgaacgttctttttcgcaacgggtttgccgccagaacaca | | EVIRVWFCNRRQKEKRINTRLGALLGNS | |
| | | ggtaagtgccgtgtgtggttcccgcgggcctggcctctttacgggtt | | TDPAVFTDLASVDNSEFQQLLNQGIPVA | |
| | | atggcccttgcgtgccttgaattacttccacctggctgcagtacgtg | | PHTTEPMLMEYPEAITRLVTGAQRPPDP | |
| | | attcttgatcccgagcttcgggttggaagtgggtgggagagttcgag | | APAPLGAPGLPNGLLSGDEDFSSIADMD | |
| | | gccttgcgcttaaggagccccttcgcctcgtgcttgagttgaggcct | | FSALLSQISSGGSGGGSGGVGSLNCIVA | |
| | | ggcctgggcgctggggccgccgcgtgcgaatctggtggcaccttcgc | | VSQNMGIGKNGDLPWPPLRNEFRYFQRM | |
| | | gcctgtctcgctgctttcgataagtctctagccatttaaaatttttg | | TTTSSVEGKQNLVIMGKKTWFSIPEKNR | |
| | | atgacctgctgcgacgctttttttctggcaagatagtcttgtaaatg | | PLKGRINLVLSRELKEPPQGAHFLSRSL | |
| | | cgggccaagatctgcacactggtatttcggtttttggggccgcgggc | | DDALKLTEQPELANKVDMVWIVGGSSVI | |
| | | ggcgacggggcccgtgcgtcccagcgcacatgttcggcgaggcgggg | | KEAMNHPGHLKLFVTRIMQDFESDTFFP | |
| | | cctgcgagcgcggccaccgagaatcggacgggggtagtctcaagctg | | EIDLEKYKLLPEYPGVLSDVQEEKGIKY | |
| | | gccggcctgctctggtgcctggcctcgcgccgccgtgtatcgccccg | | KFEVYEKNDGS* | |
| | | ccctgggcggcaaggctggcccggtcggcaccagttgcgtgagcgga | | | |
| | | aagatggccgcttcccggccctgctgcagggagctcaaaatggagga | | | |
| | | cgcggcgctcgggagagcgggcgggtgagtcacccacacaaaggaaa | | | |
| | | agggcctttccgtcctcagccgtcgcttcatgtgactccactgagta | | | |
| | | ccgggcgccgtccaggcacctcgattagttctcgagcttttggagta | | | |
| | | cgtcgtctttaggttggggggaggggttttatgcgatggagtttccc | | | |
| | | cacactgagtgggtggagactgaagttaggccagcttggcacttgat | | | |
| | | gtaattctccttggaatttgccctttttgagtttggatcttggttca | | | |
| | | ttctcaagcctcagacagtggttcaaagtttttttcttccatttcag | | | |
| | | gtgtcgtgatctagaggatcACTAGTgccaccatgGCACCTAAGaaa | | | |
| | | AAGAGGAAGGTTgaacgcccatatgcttgccctgtcgagtcctgcga | | | |
| | | tcgccgcttttctcgctcggatgagcttacccgccatatccgcatcc | | | |
| | | acacaggccagaagcccttccagtgtcgaatctgcatgcgtaacttc | | | |
| | | agtcgtagtgaccaccttaccacccacatccgcacccacacaggcgg | | | |
| | | cggccgcaggaggaagaaacgcaccagcatagagaccaacatccgtg | | | |
| | | tggccttagagaagagtttcttggagaatcaaaagcctacctcggaa | | | |
| | | gagatcactatgattgctgatcagctcaatatggaaaaagaggtgat | | | |
| | | tcgtgtttggttctgtaaccgccgccagaaagaaaaaagaatcaaca | | | |
| | | ctagactgggggccttgcttggcaacagcacagacccagctgtgttc | | | |
| | | acagacctggcatccGTGgacaactccgagtttcagcagctgctgaa | | | |
| | | ccagggcatacctgtggccccccacacaactgagcccatgctgatgg | | | |
| | | agtaccctgaggctataactcgcctagtgacaggggcccagaggccc | | | |
| | | cccgacccagctcctgctccactgggggccccggggctccccaatgg | | | |
| | | cctcctttcaggagatgaagacttctcctccattgcggacatggact | | | |
| | | tctcagccctgctgagtcagatcagctccggaggtagtggtggaggc | | | |
| | | agtggtGGTgttggttcgctaaactgcatcgtcgctgtgtcccagaa | | | |
| | | catgggcatcggcaagaacggggacctgccctggccaccgctcagga | | | |
| | | atgaattcagatatttccagagaatgaccacaacctcttcagtagaa | | | |
| | | ggtaaacagaatctggtgattatgggtaagaagacctggttctccat | | | |
| | | tcctgagaagaatcgacctttaaagggtagaattaatttagttctca | | | |
| | | gcagagaactcaaggaacctccacaaggagctcattttctttccaga | | | |
| | | agtctagatgatgccttaaaacttactgaacaaccagaattagcaaa | | | |
| | | taaagtagacatggtctggatagttggtggcagttctgttattaagg | | | |
| | | aagccatgaatcacccaggccatcttaaactatttgtgacaaggatc | | | |
| | | atgcaagactttgaaagtgacacgttttttccagaaattgatttgga | | | |
| | | gaaatataaacttctgccagaatacccaggtgttctctctgatgtcc | | | |
| | | aggaggagaaaggcattaagtacaaatttgaagtatatgagaagaat | | | |
| | | gatggatcctga | | | |
| ZFHD- | ZFHDl-p65- | cgtgaggctccggtgcccgtcagtgggcagagcgcacatcgcccaca | 62 | MAPKKKRKVERPYACPVESCDRRFSRSD | 63 |
| 060 | GGSGGGSGG | gtccccgagaagttggggggaggggtcggcaattgaaccggtgccta | | ELTRHIRIHTGQKPFQCRICMRNFSRSD | |
| | (SEQ ID NO: | gagaaggtggcgcggggtaaactgggaaagtgatgtcgtgtactggc | | HLTTHIRTHTGGGRRRKKRTSIETNIRV | |
| | 59)-ER | tccgcctttttcccgagggtgggggagaaccgtatataagtgcagta | | ALEKSFLENQKPTSEEITMIADQLNMEK | |
| | (Q502R) | gtcgccgtgaacgttctttttcgcaacgggtttgccgccagaacaca | | EVIRVWFCNRRQKEKRINTRLGALLGNS | |
| | | ggtaagtgccgtgtgtggttcccgcgggcctggcctctttacgggtt | | TDPAVFTDLASVDNSEFQQLLNQGIPVA | |
| | | atggcccttgcgtgccttgaattacttccacctggctgcagtacgtg | | PHTTEPMLMEYPEAITRLVTGAQRPPDP | |
| | | attcttgatcccgagcttcgggttggaagtgggtgggagagttcgag | | APAPLGAPGLPNGLLSGDEDFSSIADMD | |
| | | gccttgcgcttaaggagccccttcgcctcgtgcttgagttgaggcct | | FSALLSQISSGGSGGGSGGSLALSLTAD | |
| | | ggcctgggcgctggggccgccgcgtgcgaatctggtggcaccttcgc | | QMVSALLDAEPPILYSEYDPTRPFSEAS | |
| | | gcctgtctcgctgctttcgataagtctctagccatttaaaatttttg | | MMGLLTNLADRELVHMINWAKRVPGFVD | |
| | | atgacctgctgcgacgctttttttctggcaagatagtcttgtaaatg | | LTLHDQVHLLECAWMEILMIGLVWRSME | |
| | | cgggccaagatctgcacactggtatttcggtttttggggccgcgggc | | HPGKLLFAPNLLLDRNQGKCVEGGVEIF | |
| | | ggcgacggggcccgtgcgtcccagcgcacatgttcggcgaggcgggg | | DMLLATSSRFRMMNLQGEEFVCLKSIIL | |
| | | cctgcgagcgcggccaccgagaatcggacgggggtagtctcaagctg | | LNSGVYTFLSSTLKSLEEKDHIHRVLDK | |
| | | gccggcctgctctggtgcctggcctcgcgccgccgtgtatcgccccg | | ITDTLIHLMAKAGLTLQQQHRRLAQLLL | |
| | | ccctgggcggcaaggctggcccggtcggcaccagttgcgtgagcgga | | ILSHIRHMSNKRMEHLYSMKCKNVVPLS | |
| | | aagatggccgcttcccggccctgctgcagggagctcaaaatggagga | | DLLLEMLDAHRLGS* | |
| | | cgcggcgctcgggagagcgggcgggtgagtcacccacacaaaggaaa | | | |
| | | agggcctttccgtcctcagccgtcgcttcatgtgactccactgagta | | | |
| | | ccgggcgccgtccaggcacctcgattagttctcgagcttttggagta | | | |
| | | cgtcgtctttaggttggggggaggggttttatgcgatggagtttccc | | | |
| | | cacactgagtgggtggagactgaagttaggccagcttggcacttgat | | | |
| | | gtaattctccttggaatttgccctttttgagtttggatcttggttca | | | |
| | | ttctcaagcctcagacagtggttcaaagtttttttcttccatttcag | | | |
| | | gtgtcgtgatctagaggatcACTAGTgccaccatgGCACCTAAGaaa | | | |
| | | AAGAGGAAGGTTgaacgcccatatgcttgccctgtcgagtcctgcga | | | |
| | | tcgccgcttttctcgctcggatgagcttacccgccatatccgcatcc | | | |
| | | acacaggccagaagcccttccagtgtcgaatctgcatgcgtaacttc | | | |
| | | agtcgtagtgaccaccttaccacccacatccgcacccacacaggcgg | | | |
| | | cggccgcaggaggaagaaacgcaccagcatagagaccaacatccgtg | | | |
| | | tggccttagagaagagtttcttggagaatcaaaagcctacctcggaa | | | |
| | | gagatcactatgattgctgatcagctcaatatggaaaaagaggtgat | | | |
| | | tcgtgtttggttctgtaaccgccgccagaaagaaaaaagaatcaaca | | | |
| | | ctagactgggggccttgcttggcaacagcacagacccagctgtgttc | | | |
| | | acagacctggcatccGTGgacaactccgagtttcagcagctgctgaa | | | |
| | | ccagggcatacctgtggccccccacacaactgagcccatgctgatgg | | | |
| | | agtaccctgaggctataactcgcctagtgacaggggcccagaggccc | | | |
| | | cccgacccagctcctgctccactgggggccccggggctccccaatgg | | | |
| | | cctcctttcaggagatgaagacttctcctccattgcggacatggact | | | |
| | | tctcagccctgctgagtcagatcagctccggaggtagtggtggaggc | | | |
| | | agtggtGGTTCACTGGCGCTCAGCCTTACTGCCGACCAAATGGTATC | | | |
| | | AGCTCTTCTGGACGCAGAACCCCCAATTCTTTATTCCGAGTACGACC | | | |
| | | CCACACGCCCGTTCAGTGAAGCTTCCATGATGGGCCTCCTTACGAAC | | | |
| | | CTTGCCGACCGGGAACTCGTGCACATGATCAATTGGGCGAAGCGGGT | | | |
| | | GCCGGGGTTCGTAGATTTGACACTTCACGACCAAGTTCATCTCTTGG | | | |
| | | AATGTGCTTGGATGGAGATATTGATGATCGGACTCGTGTGGAGGTCA | | | |
| | | ATGGAGCATCCTGGTAAACTTCTTTTCGCACCCAATCTGCTCTTGGA | | | |
| | | TAGAAATCAGGGTAAGTGCGTCGAGGGTGGCGTTGAAATCTTCGACA | | | |
| | | TGCTCCTTGCGACATCCAGCCGATTCCGAATGATGAATCTTCAAGGA | | | |
| | | GAGGAATTTGTCTGTCTTAAGAGCATTATACTCCTCAATAGTGGAGT | | | |
| | | TTACACCTTCTTGTCCTCTACACTGAAATCACTTGAGGAAAAAGATC | | | |
| | | ACATACATAGGGTGTTGGATAAAATCACGGATACACTCATACATCTG | | | |
| | | ATGGCAAAAGCAGGATTGACCCTGCAACAGCAGCACCgACGACTGGC | | | |
| | | CCAACTGCTGTTGATCCTTAGCCATATCAGACACATGTCTAACAAAA | | | |
| | | GGATGGAACATTTGTACAGCATGAAATGTAAGAACGTAGTGCCACTG | | | |
| | | TCCGATTTGTTGCTGGAAATGCTGGACGCTCATCGGCTCggatcctg | | | |
| | | a | | | |
| ZFHD- | ZFHD1-p65- | cgtgaggctccggtgcccgtcagtgggcagagcgcacatcgcccaca | 64 | MAPKKKRKVERPYACPVESCDRRFSRSD | 65 |
| 054 | ecDHFR | gtccccgagaagttggggggaggggtcggcaattgaaccggtgccta | | ELTRHIRIHTGQKPFQCRICMRNFSRSD | |
| | (R12Y, | gagaaggtggcgcggggtaaactgggaaagtgatgtcgtgtactggc | | HLTTHIRTHTGGGRRRKKRTSIETNIRV | |
| | Y100I) | tccgcctttttcccgagggtgggggagaaccgtatataagtgcagta | | ALEKSFLENQKPTSEEITMIADQLNMEK | |
| | | gtcgccgtgaacgttctttttcgcaacgggtttgccgccagaacaca | | EVIRVWFCNRRQKEKRINTRLGALLGNS | |
| | | ggtaagtgccgtgtgtggttcccgcgggcctggcctctttacgggtt | | TDPAVFTDLASVDNSEFQQLLNQGIPVA | |
| | | atggcccttgcgtgccttgaattacttccacctggctgcagtacgtg | | PHTTEPMLMEYPEAITRLVTGAQRPPDP | |
| | | attcttgatcccgagcttcgggttggaagtgggtgggagagttcgag | | APAPLGAPGLPNGLLSGDEDFSSIADMD | |
| | | gccttgcgcttaaggagccccttcgcctcgtgcttgagttgaggcct | | FSALLSQISSGSSGISLIAALAVDYVIG | |
| | | ggcctgggcgctggggccgccgcgtgcgaatctggtggcaccttcgc | | MENAMPWNLPADLAWFKRNTLNKPVIMG | |
| | | gcctgtctcgctgctttcgataagtctctagccatttaaaatttttg | | RHTWESIGRPLPGRKNIILSSQPGTDDR | |
| | | atgacctgctgcgacgctttttttctggcaagatagtcttgtaaatg | | VTWVKSVDEAIAACGDVPEIMVIGGGRV | |
| | | cgggccaagatctgcacactggtatttcggtttttggggccgcgggc | | IEQFLPKAQKLYLTHIDAEVEGDTHFPD | |
| | | ggcgacggggcccgtgcgtcccagcgcacatgttcggcgaggcgggg | | YEPDDWESVFSEFHDADAQNSHSYCFEI | |
| | | cctgcgagcgcggccaccgagaatcggacgggggtagtctcaagctg | | LERRGS* | |
| | | gccggcctgctctggtgcctggcctcgcgccgccgtgtatcgccccg | | | |
| | | ccctgggcggcaaggctggcccggtcggcaccagttgcgtgagcgga | | | |
| | | aagatggccgcttcccggccctgctgcagggagctcaaaatggagga | | | |
| | | cgcggcgctcgggagagcgggcgggtgagtcacccacacaaaggaaa | | | |
| | | agggcctttccgtcctcagccgtcgcttcatgtgactccactgagta | | | |
| | | ccgggcgccgtccaggcacctcgattagttctcgagcttttggagta | | | |
| | | cgtcgtctttaggttggggggaggggttttatgcgatggagtttccc | | | |
| | | cacactgagtgggtggagactgaagttaggccagcttggcacttgat | | | |
| | | gtaattctccttggaatttgccctttttgagtttggatcttggttca | | | |
| | | ttctcaagcctcagacagtggttcaaagtttttttcttccatttcag | | | |
| | | gtgtcgtgatctagaggatcACTAGTgccaccatgGCACCTAAGaaa | | | |
| | | AAGAGGAAGGTTgaacgcccatatgcttgccctgtcgagtcctgcga | | | |
| | | tcgccgcttttctcgctcggatgagcttacccgccatatccgcatcc | | | |
| | | acacaggccagaagcccttccagtgtcgaatctgcatgcgtaacttc | | | |
| | | agtcgtagtgaccaccttaccacccacatccgcacccacacaggcgg | | | |
| | | cggccgcaggaggaagaaacgcaccagcatagagaccaacatccgtg | | | |
| | | tggccttagagaagagtttcttggagaatcaaaagcctacctcggaa | | | |
| | | gagatcactatgattgctgatcagctcaatatggaaaaagaggtgat | | | |
| | | tcgtgtttggttctgtaaccgccgccagaaagaaaaaagaatcaaca | | | |
| | | ctagactgggggccttgcttggcaacagcacagacccagctgtgttc | | | |
| | | acagacctggcatccGTGgacaactccgagtttcagcagctgctgaa | | | |
| | | ccagggcatacctgtggccccccacacaactgagcccatgctgatgg | | | |
| | | agtaccctgaggctataactcgcctagtgacaggggcccagaggccc | | | |
| | | cccgacccagctcctgctccactgggggccccggggctccccaatgg | | | |
| | | cctcctttcaggagatgaagacttctcctccattgcggacatggact | | | |
| | | tctcagccctgctgagtcagatcagctccggatccagcggcatctct | | | |
| | | ctgattgcggcgctggcagttgactacgttattggcatggaaaacgc | | | |
| | | gatgccatggaacctcccggctgacctggcgtggttcaaacgtaaca | | | |
| | | ccctgaacaaacctgtgatcatgggtcgtcacacctgggaatctatt | | | |
| | | ggccgtcctctcccgggtcgtaaaaacatcattctgtcttctcagcc | | | |
| | | aggcaccgacgaccgtgttacctgggttaaaagcgttgacgaagcga | | | |
| | | ttgctgcgtgcggtgatgttcctgaaattatggtgatcggcggtggc | | | |
| | | cgtgttatcgaacagttcctgccgaaagcgcagaaactgtacctgac | | | |
| | | ccacatcgacgcggaagttgaaggtgacacccacttcccggactacg | | | |
| | | aaccggatgattgggagagcgtattctccgaattccatgatgcggat | | | |
| | | gcgcaaaactctcattcttactgttttgaaatcctggaacgtcgtgg | | | |
| | | atcctga | | | |
| ZFHD- | ZFHD1-p65 | cgtgaggctccggtgcccgtcagtgggcagagcgcacatcgcccaca | 66 | MAPKKKRKVERPYACPVESCDRRFSRSD | 67 |
| 055 | | gtccccgagaagttggggggaggggtcggcaattgaaccggtgccta | | ELTRHIRIHTGQKPFQCRICMRNFSRSD | |
| | | gagaaggtggcgcggggtaaactgggaaagtgatgtcgtgtactggc | | HLTTHIRTHTGGGRRRKKRTSIETNIRV | |
| | | tccgcctttttcccgagggtgggggagaaccgtatataagtgcagta | | ALEKSFLENQKPTSEEITMIADQLNMEK | |
| | | gtcgccgtgaacgttctttttcgcaacgggtttgccgccagaacaca | | EVIRVWFCNRRQKEKRINTRLGALLGNS | |
| | | ggtaagtgccgtgtgtggttcccgcgggcctggcctctttacgggtt | | TDPAVFTDLASVDNSEFQQLLNQGIPVA | |
| | | atggcccttgcgtgccttgaattacttccacctggctgcagtacgtg | | PHTTEPMLMEYPEAITRLVTGAQRPPDP | |
| | | attcttgatcccgagcttcgggttggaagtgggtgggagagttcgag | | APAPLGAPGLPNGLLSGDEDFSSIADMD | |
| | | gccttgcgcttaaggagccccttcgcctcgtgcttgagttgaggcct | | FSALLSQISSGS* | |
| | | ggcctgggcgctggggccgccgcgtgcgaatctggtggcaccttcgc | | | |
| | | gcctgtctcgctgctttcgataagtctctagccatttaaaatttttg | | | |
| | | atgacctgctgcgacgctttttttctggcaagatagtcttgtaaatg | | | |
| | | cgggccaagatctgcacactggtatttcggtttttggggccgcgggc | | | |
| | | ggcgacggggcccgtgcgtcccagcgcacatgttcggcgaggcgggg | | | |
| | | cctgcgagcgcggccaccgagaatcggacgggggtagtctcaagctg | | | |
| | | gccggcctgctctggtgcctggcctcgcgccgccgtgtatcgccccg | | | |
| | | ccctgggcggcaaggctggcccggtcggcaccagttgcgtgagcgga | | | |
| | | aagatggccgcttcccggccctgctgcagggagctcaaaatggagga | | | |
| | | cgcggcgctcgggagagcgggcgggtgagtcacccacacaaaggaaa | | | |
| | | agggcctttccgtcctcagccgtcgcttcatgtgactccactgagta | | | |
| | | ccgggcgccgtccaggcacctcgattagttctcgagcttttggagta | | | |
| | | cgtcgtctttaggttggggggaggggttttatgcgatggagtttccc | | | |
| | | cacactgagtgggtggagactgaagttaggccagcttggcacttgat | | | |
| | | gtaattctccttggaatttgccctttttgagtttggatcttggttca | | | |
| | | ttctcaagcctcagacagtggttcaaagtttttttcttccatttcag | | | |
| | | gtgtcgtgatctagaggatcACTAGTgccaccatgGCACCTAAGaaa | | | |
| | | AAGAGGAAGGTTgaacgcccatatgcttgccctgtcgagtcctgcga | | | |
| | | tcgccgcttttctcgctcggatgagcttacccgccatatccgcatcc | | | |
| | | acacaggccagaagcccttccagtgtcgaatctgcatgcgtaacttc | | | |
| | | agtcgtagtgaccaccttaccacccacatccgcacccacacaggcgg | | | |
| | | cggccgcaggaggaagaaacgcaccagcatagagaccaacatccgtg | | | |
| | | tggccttagagaagagtttcttggagaatcaaaagcctacctcggaa | | | |
| | | gagatcactatgattgctgatcagctcaatatggaaaaagaggtgat | | | |
| | | tcgtgtttggttctgtaaccgccgccagaaagaaaaaagaatcaaca | | | |
| | | ctagactgggggccttgcttggcaacagcacagacccagctgtgttc | | | |
| | | acagacctggcatccGTGgacaactccgagtttcagcagctgctgaa | | | |
| | | ccagggcatacctgtggccccccacacaactgagcccatgctgatgg | | | |
| | | agtaccctgaggctataactcgcctagtgacaggggcccagaggccc | | | |
| | | cccgacccagctcctgctccactgggggccccggggctccccaatgg | | | |
| | | cctcctttcaggagatgaagacttctcctccattgeggacatggact | | | |
| | | tctcagccctgctgagtcagatcagctccggatcctga | | | |

### Stimuli of Transcription Factor Systems

A transcription factor system of the present disclosure can be responsive to a stimulus.

In some embodiments, a stimulus is a ligand. Ligands may be nucleic acid-based, protein-based, lipid based, organic, inorganic or any combination of the foregoing. In some embodiments, ligands may be synthetic molecules. In some embodiments, ligands may be small molecule therapeutic compounds. In some embodiments, ligands may be small molecule drugs previously approved by a regulatory agency, such as the US Food and Drug Administration (FDA).

As described in the present disclosure, a transcription factor system can exhibit ligand-dependent activity. A ligand can bind to a DRD and stabilize a transcription factor or a domain of a transcription factor encoded by the transcription factor system. Ligands that are known to bind candidate DRDs can be tested for their effect on the activity of a transcription factor system.

In some embodiments, a ligand is cell permeable. In some embodiments, a ligand may be designed to be lipophilic to improve cell permeability.

In some embodiments, a ligand is a small molecule. A small molecule ligand may be clinically approved to be safe and have appropriate pharmaceutical kinetics and distribution.

In some embodiments, the ligand may be complexed or bound to one or more other molecules such as, but not limited to, another ligand, a protein, peptide, nucleic acid, lipid, lipid derivative, sterol, steroid, metabolite, metabolite derivative or small molecule. In some embodiments, the ligand stimulus is complexed or bound to one or more different kinds and/or numbers of other molecules. In some embodiments, the ligand stimulus is a multimer of the same kind of ligand. In some embodiments, the ligand stimulus multimer comprises 2, 3, 4, 5, 6, or more monomers.

### CA2 Ligands

In some embodiments, a ligand of the present disclosure binds to carbonic anhydrases. In some embodiments, the ligand binds to and inhibits carbonic anhydrase function and is herein referred to as carbonic anhydrase inhibitor.

In some embodiments, the ligand is a small molecule that binds to carbonic anhydrase 2. In one embodiment, the small molecule is a CA2 inhibitor. Examples of CA2 inhibitors include but are not limited to Celecoxib (also referred to as Celebrex), Valdecoxib, Rofecoxib, Acetazolamide, Methazolamide, Dorzolamide, Brinzolamide, Diclofenamide, Ethoxzolamide, Zonisamide, Dansylamide, and Dichlorphenamide.

In some embodiments, the ligands may comprise portions of small molecules know to mediate binding to CA2. Ligands may also be modified to reduce off-target binding to carbonic anhydrases other than CA2 and increase specific binding to CA2.

In some embodiments, the stimulus may be a ligand that binds to more than one carbonic anhydrase. In one embodiment, the stimulus is a pan carbonic anhydrase inhibitor that may bind to two or more carbonic anhydrases.

### DHFR Ligands

In some embodiments, a ligand of the present disclosure binds to dihydrofolate reductase. In some embodiments, the ligand binds to and inhibits dihydrofolate reductase function and is herein referred to as a dihydrofolate inhibitor.

In some embodiments, the ligand may be a selective inhibitor of human DHFR. Ligands of the disclosure may also be selective inhibitors of dihydrofolate reductases of bacteria and parasitic organisms such as Pneumocystis spp., Toxoplasma spp., Trypanosoma spp., Mycobacterium spp., and Streptococcus spp. Ligands specific to other DHFR may be modified to improve binding to human dihydrofolate reductase.

Examples of dihydrofolate inhibitors include, but are not limited to, Trimethoprim (TMP), Methotrexate (MTX), Pralatrexate, Piritrexim, Pyrimethamine, Talotrexin, Chloroguanide, Pentamidine, Trimetrexate, aminopterin, C1 898 trihydrochloride, Pemetrexed Disodium, Raltitrexed, Sulfaguanidine, Folotyn, Iclaprim and Diaveridine.

In some embodiments, ligands of the present disclosure may include dihydrofolic acid or any of its derivatives that may bind to human DHFR. In some embodiments, the ligands of the present disclosure may be 2,4, diaminohetrocyclic compounds. In some embodiments, the 4-oxo group in dihydrofolate may be modified to generate DHFR inhibitors. In one example, the 4-oxo group may be replaced by 4-amino group. Various diamino heterocycles, including pteridines, quinazolines, pyridopyrimidines, pyrimidines, and triazines, may also be used as scaffolds to develop DHFR inhibitors and may be used according to the present disclosure.

In some embodiments, ligands include TMP-derived ligands containing portions of the ligand known to mediate binding to DHFR. Ligands may also be modified to reduce off-target binding to other folate metabolism enzymes and increase specific binding to DHFR.

### ER Ligands

In some embodiments, a ligand of the present disclosure binds to ER. Ligands may be agonists or antagonists. In some embodiments, the ligand binds to and inhibits ER function and is herein referred to as an ER inhibitor. In some embodiments, the ligand may be a selective inhibitor of human ER. Ligands of the disclosure may also be selective inhibitors of ER of other species. Ligands specific to other ER may be modified to improve binding to human ER.

Ligands may be ER agonists such as but not limited to endogenous estrogen 17b-estradiol (E2) and the synthetic nonsteroidal estrogen diethylstilbestrol (DES). In some embodiments. The ligands may be ER antagonists, such as ICI-164,384, RU486, tamoxifen, 4-hydroxytamoxifen (4-OHT), fulvestrant, oremifene, lasofoxifene, clomifene, femarelle and ormeloxifene and raloxifene (RAL).

In some embodiments, the stimulus of the current disclosure may be ER antagonists such as, but not limited to, Bazedoxifene and/or Raloxifene.

In some embodiments, ligands include Bazedoxifene-derived ligands containing portions of the ligand known to mediate binding to ER. Ligands may also be modified to reduce off-target binding to other folate metabolism enzymes and increase specific binding to ER derived DRDs.

### Phosphodiesterase Ligands

In some embodiments, ligands of the present disclosure bind to phosphodiesterases. In some embodiments, the ligands bind to and inhibit phosphodiesterase function and are herein referred to as phosphodiesterase inhibitors.

In some embodiments, the ligand is a small molecule that binds to phosphodiesterase 5. In one embodiment, the small molecule is a hPDE5 inhibitor. Examples of hPDE5 inhibitors include, but are not limited to, Sildenafil, Vardenafil, Tadalafil, Avanafil, Lodenafil, Mirodenafil, Udenafil, Benzamidenafil, Dasantafil, Beminafil, SLx-2101, LAS 34179, UK-343,664, UK-357903, UK-371800, and BMS-341400.

In some embodiments, ligands include sildenafil-derived ligands containing portions of the ligand known to mediate binding to hPDE5. Ligands may also be modified to reduce off-target binding to phosphodiesterases and increase specific binding to hPDE5.

In some embodiments, the stimulus may be a ligand that binds to more than one phosphodiesterase. In one embodiment, the stimulus is a pan-phosphodiesterase inhibitor that may bind to two or more hPDEs such as Aminophyline, Paraxanthine, Pentoxifylline, Theobromine, Dipyridamole, Theophyline, Zaprinast, Icariin, CDP-840, Etazolate and Glaucine.

In some embodiments, the ligand is a hPDE1 inhibitor. In some embodiments, the ligand is a hPDE2 inhibitor. In some embodiments, the ligand is a hPDE3 inhibitor. In some embodiments, the ligand is a hPDE4 inhibitor. In some embodiments, the ligand is a hPDE6 inhibitor. In some embodiments, the ligand is a hPDE7 inhibitor. In some embodiments, the ligand is a hPDE8 inhibitor. In some embodiments, the ligand is a hPDE9 inhibitor. In some embodiments, the ligand is a hPDE10 inhibitor.

### FKBP Ligands

In some embodiments, ligands of the present disclosure bind to FKBP, including human FKBP. In some embodiments, the ligand is SLF or Shield-1.

### Payloads

Payloads may include any polypeptide or any protein or fragment thereof. A payload may be a wild-type sequence, a fragment of a wild-type sequence and/or comprise one or more mutations. A payload may be a natural protein from an organism genome, or variants, mutants, and derivatives thereof. The natural protein may be from, for example, a mammalian organism, a bacterium, and a virus. A payload may be a protein or polypeptide encoded by a recombinant nucleic acid molecule, a fusion or chimeric polypeptide, or a polypeptide that functions as part of a protein complex.

In one example, a payload may be a polypeptide encoded by a nucleic acid sequence from a human genome.

In some embodiments, a payload may be a variant sequence of a parent polypeptide. In some aspects, the variant sequence may have the same or a similar activity as the reference sequence. Alternatively, the variant may have an altered activity (e.g., increased or decreased) relative to a reference sequence. Generally, variants of a particular polypeptide of the disclosure will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polypeptide as determined by sequence alignment programs known to those skilled in the art.

### Therapeutic Agents as Payloads

In some embodiments, payloads of the present disclosure may be therapeutic agents. For example, a payload may be a cancer therapeutic agent, a therapeutic agent for an autoimmune disease, an immunotherapeutic agent, an anti-inflammatory agent, an anti-pathogen agent or a gene therapy agent. In some aspects, the immunotherapeutic agent may be an antibody and fragments and variants thereof, a T-cell receptor (TCR), a chimeric antigen receptor (CAR), a chimeric switch receptor, an antagonist of a co-inhibitory molecule, an agonist of a co-stimulatory molecule, a cytokine, a cytokine receptor, a chemokine, a chemokine receptor, a metabolic factor, a coagulation factor, an enzyme, a homing receptor and a safety switch.

In some embodiments, payloads of the present disclosure may be immunotherapeutic agents that induce immune responses in an organism. The immunotherapeutic agent may be, but is not limited to, an antibody and fragments and variants thereof, a TCR, a chimeric antigen receptor (CAR), a chimeric switch receptor, a cytokine, chemokine, a cytokine receptor, a chemokine receptor, a cytokine-cytokine receptor fusion polypeptide, or any agent that induces an immune response. In one embodiment, the immunotherapeutic agent induces an anti-cancer immune response in a cell, or in a subject.

### Cytokines, Chemokines and Other Soluble Factors as Payloads

In some embodiments, payloads of the present disclosure may be cytokines, chemokines, growth factors, and soluble proteins produced by immune cells, cancer cells and other cell types, which act as chemical communicators between cells and tissues within the body. These proteins mediate a wide range of physiological functions, from effects on cell growth, differentiation, migration and survival, to a number of effector activities. For example, activated T cells produce a variety of cytokines for cytotoxic function to eliminate tumor cells.

In some embodiments, payloads of the present disclosure may be cytokines, and fragments, variants, analogs and derivatives thereof, including but not limited to interleukins, tumor necrosis factors (TNFs), interferons (IFNs), TGF beta and chemokines. In some embodiments, payloads of the present invention may be cytokines that stimulate immune responses. In other embodiments, payloads of the invention may be antagonists of cytokines that negatively impact anti-cancer immune responses.

In some embodiments, payloads of the present disclosure may be cytokine receptors, recombinant receptors, variants, analogs and derivatives thereof; or signal components of cytokines. In various embodiments, payloads of the present disclosure may include secreted cytokines or membrane bound form of cytokines. An illustrative example of a membrane cytokine, may include a cytokine (for example, an immune stimulatory cytokine, for example, IL12, IL2, IL15 and IL18) that is operably fused, linked or connected to a transmembrane domain, for example, a CD8α transmembrane domain, a B7-1 transmembrane domain, a CD4 transmembrane domain, a CD 28 transmembrane domain, a CTLA-4 transmembrane domain, a PD-1 transmembrane domain, or a human IgG4 Fc region. In various embodiments, the cytokine may be fused or connected to a transmembrane domain via an intervening peptide or protein sequence, such as a linker, a hinge, a transmembrane tail etc.

In one embodiment, payloads of the present disclosure may be cytokines fused to TNF alpha ectodomain. Such payloads are produced as membrane associated cytokines fused to the TNF ectodomain. In one embodiment, the cytokine may be shed from the cell surface by the action of membrane associated proteases, and/or proteases in the extracellular space e.g. MMP9.

In some embodiments, payloads of the present disclosure may be an interleukin (IL) cytokine. Interleukins (ILs) are a class of glycoproteins produced by leukocytes for regulating immune responses. As used herein, the term “interleukin (IL)” refers to an interleukin polypeptide from any species or source and includes the full-length protein as well as fragments or portions of the protein.

In some embodiments, a payload of the disclosure may comprise IL12. IL12 is a heterodimeric protein of two subunits (p35, p40) that is secreted by antigen presenting cells, such as macrophages and dendritic cells. Expression of IL12 requires the simultaneous expression of the two subunits to produce a biologically active heterodimer. In some embodiments, payloads of the disclosure may be the p35 subunit or the p40 subunit.

In some embodiments, a payload of the disclosure may comprise whole or a portion of IL12.

In some embodiments, the IL12 may be a Flexi IL12, wherein both p35 and p40 subunits are encoded by a single cDNA that produces a single chain polypeptide. The single chain polypeptide may be generated by placing the p35 subunit at the N terminus or the C terminus of the single chain polypeptide. Similarly, the p40 subunit may be at the N terminus or C terminus of the single chain polypeptide.

The format of an IL12 payload of the present disclosure may be optimized. In one embodiment, the payload may be a bicistronic IL12 containing p40 and p35 subunits separated by an internal ribosome entry site or a cleavage site such as P2A or Furin to allow independent expression of both subunits from a single vector. In another embodiment, the payload may be the p40 subunit of IL12 or the p35 subunit of IL12.

In some embodiments, the payload may be IL12 that is membrane bound. IL12 may be bound to the membrane by a transmembrane domain. The transmembrane domain may also include an optional hinge domain. In some aspects, the IL12 molecule is extracellular and tethered to the cell by the transmembrane domain. In some aspects, the membrane bound IL12 may be shed or cleaved from the cell surface by the action of proteases. In some embodiments, the transmembrane domain of the present disclosure may be derived either from a natural or from a synthetic source. The transmembrane domain may be derived from any naturally membrane-bound or transmembrane protein. Alternatively, the transmembrane domain of the present disclosure may be synthetic. In some aspects, the synthetic sequence may comprise predominantly hydrophobic residues such as leucine and valine. In some aspects, transmembrane and/or hinge domains that are resistant to the activity of proteases may be selected.

In some embodiments, a payload of the disclosure may comprise IL15. Interleukin 15 is a potent immune stimulatory cytokine and an essential survival factor for T cells and Natural Killer cells.

In some embodiments, a payload of the disclosure may comprise whole or a portion of IL15. Any portion of IL15 that retains one or more functions of full-length or mature I15 may be useful in the present disclosure. Such functions include the promotion of NK cell survival, regulation of NK cell and T cell activation and proliferation as well as the support of NK cell development from hematopoietic stem cells.

In some instances, whole or a portion of the IL15 is linked to the whole or a portion of one or more transmembrane proteins.

An IL15 payload may be designed to be secreted (using e.g. IL2 signal sequence) or membrane bound (using e.g. IgE or CD8a signal sequence).

A unique feature of IL15 mediated activation is the mechanism of trans-presentation in which IL15 is presented as a complex with the alpha subunit of IL15 receptor (IL15Ra) that binds to and activates membrane bound IL15 beta/gamma receptor, either on the same cell or a different cell. In some embodiments, a payload of the present disclosure is a membrane bound IL15. In some embodiments, a payload of the present disclosure may include an IL15/IL15Ra fusion polypeptide. In some embodiments, the payload may be a whole or a portion of IL15 fused to the whole or a portion of IL15Ra. Any portion of IL15 and IL15Ra that retains one or more functions of full-length or mature IL15 or IL15Ra respectively may be used.

In some aspects, the IL15 molecule is extracellular and tethered to the cell by the transmembrane domain. In some aspects, the membrane bound IL15 may be shed or cleaved from the cell surface by the action of proteases.

The whole or a portion of the membrane bound IL15 or IL15/IL15Ra fusion polypeptides of the disclosure may be shed into the extracellular space. Shedding as used herein refers to the release of membrane associated biomolecules from the membrane to which they are tethered. In some instances, shedding may be induced by proteolytic cleavage.

Payloads of the present disclosure may comprise amino acid sequences similar to the amino acid sequence of human IL15, for example, UniProtKB-P40933 (IL15_HUMAN).

In some embodiments, payloads of the present disclosure may be utilized to improve expansion, survival, persistence, and potency of immune cells such as CD8+TEM, natural killer cells and tumor infiltrating lymphocytes (TIL) cells, and CAR T cells used for immunotherapy. In one aspect, the present disclosure provides payloads to minimize toxicity related to cytokine therapy. In some embodiments, a payload of the disclosure may comprise whole or a portion of IL2. Any portion of IL2 that retains one or more functions of full-length or mature IL2 may be useful in the present disclosure.

It is understood in the art that certain gene and/or protein nomenclature for the same gene or protein may be inclusive or exclusive of punctuation such as a dash “−” or symbolic such as Greek letters. Whether these are included or excluded herein, the meaning is not meant to be changed as would be understood by one of skill in the art. For example, IL2, IL-2 and IL 2 refer to the same interleukin. Likewise, IL15, IL 15 and IL-15 refer to the same interleukin. Likewise, TNFalpha, TNFα, TNF-alpha, TNF-α, TNF alpha and TNF α all refer to the same protein.

### Antibodies and Antibody Fragments as Payloads

In some embodiments, payloads of the present disclosure may be antibodies, antibody fragments and variants thereof.

The antibody may be an intact antibody, an antibody light chain, antibody heavy chain, an antibody fragment, an antibody variant, or an antibody derivative.

For the purposes herein, an “antibody” may comprise a heavy and light variable domain as well as an Fc region.

In some embodiments, the payload maybe a monoclonal antibody. As used herein, the term “monoclonal antibody” refers to an antibody obtained from a population of substantially homogeneous cells (or clones), i.e., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen.

In one embodiment, the payload of the present disclosure may be a humanized antibody. As used herein, the term “humanized antibody” refers to a chimeric antibody comprising a minimal portion from one or more non-human (e.g., murine) antibody source(s) with the remainder derived from one or more human immunoglobulin sources. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and/or capacity. In one embodiment, the antibody may be a humanized full-length antibody.

As used herein, the term “antibody variant” refers to a modified antibody (in relation to a native or starting antibody) or a biomolecule resembling a native or starting antibody in structure and/or function (e.g., an antibody mimetic). Antibody variants may be altered in their amino acid sequence, composition or structure as compared to a native antibody. Antibody variants may include, but are not limited to, antibodies with altered isotypes (e.g., IgA, IgD, IgE, IgG1, IgG2, IgG3, IgG4, or IgM), humanized variants, optimized variants, multispecific antibody variants (e.g., bispecific variants), and antibody fragments.

In some embodiments, antibody fragments and variants may comprise antigen binding regions from intact antibodies. Examples of antibody fragments and variants may include, but are not limited to Fab, Fab′, F(ab′)2, and Fv fragments; diabodies; linear antibodies; single-chain antibody molecules such as single chain variable fragment (scFv); and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)2 fragment that has two antigen-binding sites and is still capable of cross-linking with the antigen. Payloads of the present disclosure may comprise one or more of these fragments.

In some embodiments, antibody payloads of the present disclosure may be therapeutic antibodies.

### Chimeric Antigen Receptor Payloads

In some embodiments, payloads of the present disclosure may be chimeric antigen receptors (CARs). As used herein, the term “chimeric antigen receptor (CAR)” refers to a synthetic receptor that mimics a T-cell receptor (TCR) on the surface of T cells. In general, a CAR is composed of an extracellular targeting domain, a transmembrane domain/region and an intracellular signaling/activation domain. Cells such as T cells engineered to express a CAR can be redirected to attack target cells that express a molecule which can be recognized by the targeting moiety of the CAR. In a standard CAR receptor, the components: the extracellular targeting domain, transmembrane domain and intracellular signaling/activation domain, are linearly constructed as a single fusion protein. The extracellular region comprises a targeting domain/moiety (e.g., a scFv) that recognizes a specific tumor antigen or other tumor cell-surface molecules. The intracellular region may contain a signaling domain of a TCR complex (e.g., the signal region of CD3ζ), and/or one or more costimulatory signaling domains, such as those from CD28, 4-1BB (CD137) and OX-40 (CD134). For example, a “first-generation CAR” only has the CD3ζ signaling domain, whereas in an effort to augment T-cell persistence and proliferation, costimulatory intracellular domains are added, giving rise to second generation CARs having a CD3ζ signal domain plus one costimulatory signaling domain, and third generation CARs having CD3ζ signal domain plus two or more costimulatory signaling domains. A CAR, when expressed by a T cell, endows the T cell with antigen specificity determined by the extracellular targeting moiety of the CAR. A fourth-generation CAR includes addition of one or more elements such as homing and suicide genes to develop a more competent and safer architecture of CAR.

In some embodiments, a CAR payload, when transduced into immune cells (e.g., T cells and NK cells), can re-direct the immune cells against the target (e.g., a tumor cell) which expresses a molecule recognized by the extracellular target moiety of the CAR.

### Nucleic Acid Modifying Agents as Payloads

In some embodiments, payloads of the present disclosure may be nucleic acid modifying agents.

In some embodiments, payloads of the present disclosure may be components of gene editing systems. In some embodiments, payloads of the present disclosure may be a Cas protein (CRISPR-associated protein), including Cas9 and Cas12. The Cas protein may be altered or otherwise modified. For example, the Cas protein may be a deadCas9. In some embodiments, the Cas9 protein is an enzymatically active Cas9 protein, a Cas9 protein wild-type protein, a Cas9 protein nickase or a nuclease null or nuclease deficient Cas9 protein. In some embodiments, payloads of the present disclosure may be Zinc finger nucleases, TALEN (Transcription activator-like effector-based nucleases) and meganucleases.

In some embodiments, payloads of the present disclosure may be a recombinase, such as Cre recombinase.

### Agents for Treating Autoimmune Disorders as Payloads

In some embodiments, payloads of the present disclosure may be agents for treating, ameliorating or preventing autoimmune disorders.

In some embodiments, payloads of the present disclosure include anti-cytokines, such as neutralizing antibodies to tumor necrosis factor (TNF)-α, IL-1 and IL-6. In some embodiments, payloads of the present disclosure target B-cell depletion, such as neutralizing antibodies to CD20, CD22, CD28, CTLA-4, and B-lymphocyte stimulator (BLyS).

### Pharmaceutical Compositions and Formulations

The present teachings further comprise pharmaceutical compositions comprising one or more of the transcription factor systems, nucleic acids, polynucleotides, modified cells or payloads of the present disclosure, and optionally at least one pharmaceutically acceptable excipient or inert ingredient.

As used herein the term “pharmaceutical composition” refers to a preparation of one or more of the transcription factor systems, nucleic acids, polynucleotides, modified cells, payloads or transcription factor system components described herein, or pharmaceutically acceptable salts thereof, optionally with other chemical components such as physiologically suitable carriers and excipients.

The term “excipient” or “inactive ingredient” refers to an inert or inactive substance added to a pharmaceutical composition to further facilitate administration of a compound.

In some embodiments, compositions are administered to humans, human patients or subjects. For the purposes of the present disclosure, the phrase “active ingredient” generally refers to any one or more transcription factor system components to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Subjects to which administration of the pharmaceutical compositions is contemplated include, but are not limited to, non-human mammals, including agricultural animals such as cattle, horses, chickens and pigs, domestic animals such as cats, dogs, or research animals such as mice, rats, rabbits, dogs and non-human primates.

A pharmaceutical composition in accordance with the disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient or inert ingredient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

Efficacy of treatment or amelioration of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. A healthcare practitioner skilled in the art may monitor efficacy of treatment or prevention by measuring any one of such parameters, or any combination of parameters. In connection with the administration of compositions of the present disclosure, “effective against” for example a cancer, indicates that administration in a clinically appropriate manner results in a beneficial effect for at least a significant fraction of patients, such as an improvement of symptoms, a cure, a reduction in disease load, reduction in tumor mass or cell numbers, extension of life, improvement in quality of life, or other effect generally recognized as positive by medical doctors familiar with treating the particular type of cancer.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given composition or formulation of the present disclosure can also be judged using an experimental animal model for the given disease as known in the art. When using an experimental animal model, efficacy of treatment is evidenced when a statistically significant change is observed.

### Formulations

The polynucleotide and vector compositions of the present disclosure may be formulated in any manner suitable for delivery. The formulation may be, but is not limited to, nanoparticles, poly (lactic-co-glycolic acid) (PLGA) microspheres, lipidoids, lipoplex, liposome, polymers, carbohydrates (including simple sugars), cationic lipids and combinations thereof.

In one embodiment, the polynucleotide and vector formulation is a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG and PEGylated lipids. In another aspect, the lipid may be a cationic lipid such as, but not limited to, DLin-DMA, DLin-D-DMA, DLin-MC3-DMA, DLin-KC2-DMA and DODMA.

For polynucleotides of the disclosure, the formulation may be selected from any of those taught, for example, in International Application PCT/US2012/069610.

### Inactive Ingredients

In some embodiments, pharmaceutical or other formulations may comprise at least one excipient which is an inactive ingredient. As used herein, the term “inactive ingredient” refers to one or more inactive agents included in formulations. In some embodiments, all, none or some of the inactive ingredients which may be used in the formulations of the present disclosure may be approved by the US Food and Drug Administration (FDA).

### Dosing, Delivery and Administration

The compositions of the disclosure may be delivered to a cell or a subject through one or more routes and modalities. The viral vectors containing one or more transcription factor systems, nucleic acids, polynucleotides, payloads, and other components described herein may be used to deliver them to a cell and/or a subject. Other modalities may also be used such as mRNAs, plasmids, and as recombinant proteins.

### Delivery

### Naked Delivery

Pharmaceutical compositions, transcription factor systems, nucleic acids, polynucleotides, or payloads of the present disclosure may be delivered to cells, tissues, organs and/or organisms in naked form. As used herein in, the term “naked” refers to pharmaceutical compositions, transcription factor systems, nucleic acids, polynucleotides, or payloads delivered free from agents or modifications which promote transfection or permeability. The naked pharmaceutical compositions, transcription factor systems, nucleic acids, polynucleotides, or payloads may be delivered to the cells, tissues, organs and/or organisms using routes of administration known in the art and described herein. In some embodiments, naked delivery may include formulation in a simple buffer such as saline or PBS.

### Formulated Delivery

In some embodiments, pharmaceutical compositions, transcription factor systems, nucleic acids, polynucleotides, or payloads of the present disclosure may be formulated, using methods described herein. Formulations may comprise pharmaceutical compositions, transcription factor systems, nucleic acids, polynucleotides, or payloads which may be modified and/or unmodified. Formulations may further include, but are not limited to, cell penetration agents, pharmaceutically acceptable carriers, delivery agents, bioerodible or biocompatible polymers, solvents, and/or sustained-release delivery depots. Formulations of the present disclosure may be delivered to cells using routes of administration known in the art and described herein.

Pharmaceutical compositions, transcription factor systems, nucleic acids, polynucleotides, or payloads may also be formulated for direct delivery to organs or tissues in any of several ways in the art including, but not limited to, direct soaking or bathing, via a catheter, by gels, powder, ointments, creams, gels, lotions, and/or drops, by using substrates such as fabric or biodegradable materials coated or impregnated with compositions, and the like.

### Delivery to Cells

In another aspect of the disclosure, polynucleotides of a transcription factor system or components thereof and compositions of the disclosure and vectors comprising the polynucleotides may be introduced into cells such as immune effector cells.

In one aspect of the disclosure, polynucleotides of a transcription factor system or components thereof and compositions of the disclosure, may be packaged into plasmids, viral vectors or integrated into viral genomes allowing transient or stable expression of the polynucleotides. Preferable viral vectors are retroviral vectors including lentiviral vectors and gamma retroviral vectors. In order to construct a retroviral vector, a polynucleotide molecule of a transcription factor system is inserted into the viral genome in the place of certain viral sequences to produce a virus that is replication-defective. The recombinant viral vector is then introduced into a packaging cell line containing the gag, pol, and env genes, but without the LTR and packaging components. The recombinant retroviral particles are secreted into the culture media, then collected, optionally concentrated, and used for gene transfer. Lentiviral vectors are especially preferred as they are capable of infecting both dividing and non-dividing cells.

Vectors may also be transferred to cells by non-viral methods by physical methods such as needles, electroporation, sonoporation, hydroporation; chemical carriers such as inorganic particles (e.g. calcium phosphate, silica, gold) and/or chemical methods. In some embodiments, synthetic or natural biodegradable agents may be used for delivery such as cationic lipids, lipid nano emulsions, nanoparticles, peptide based vectors, or polymer based vectors. In some embodiments, vectors may be transferred to cells by temporary membrane disruption, for example, by high speed cell deformation.

In some embodiments, the polypeptides of the disclosure may be delivered to the cell directly. In one embodiment, the polypeptides of the disclosure may be delivered using synthetic peptides comprising an endosomal leakage domain (ELD) fused to a cell penetration domain (CLD). The polypeptides of the disclosure are co introduced into the cell with the ELD-CLD-synthetic peptide. ELDs facilitate the escape of proteins that are trapped in the endosome, into the cytosol. Such domains are derived proteins of microbial and viral origin and have been described in the art. CPDs allow the transport of proteins across the plasma membrane and have also been described in the art. The ELD-CLD fusion proteins synergistically increase the transduction efficiency when compared to the co-transduction with either domain alone. In some embodiments, a histidine rich domain may optionally be added to the shuttle construct as an additional method of allowing the escape of the cargo from the endosome into the cytosol. The shuttle may also include a cysteine residue at the N or C terminus to generate multimers of the fusion peptide. Multimers of the ELD-CLD fusion peptides generated by the addition of cysteine residue to the terminus of the peptide show even greater transduction efficiency when compared to the single fusion peptide constructs. The polypeptides of the disclosure may also be appended to appropriate localization signals to direct the cargo to the appropriate sub-cellular location e.g. nucleus. In some embodiments any of the ELDs, CLDs or the fusion ELD-CLD synthetic peptides taught in the International Patent Publication, WO2016161516 and WO2017175072 may be useful in the present disclosure (the contents of each of which are herein incorporated by reference in their entirety).

Delivery Modalities and/or Vectors

The transcription factor systems or components thereof of the present disclosure may be delivered using one or more modalities. The present disclosure also provides vectors that package polynucleotides of the disclosure encoding transcription factors and parts thereof, DRDs, or payload constructs, and combinations thereof. Vectors of the present disclosure may also be used to deliver the packaged polynucleotides to a cell, a local tissue site or a subject. These vectors may be of any kind, including DNA vectors, RNA vectors, plasmids, viral vectors and particles. Viral vector technology is well known and described in Sambrook et al. (2001, Molecular Cloning: A Laboratory Manual, Cold Spring Harbor Laboratory, New York). Viruses, which are useful as vectors include, but are not limited to an adenovirus, adeno-associated virus (AAV), alphavirus, flavivirus, herpes virus, measles virus, rhabdovirus, retrovirus, lentivirus, Newcastle disease virus (NDV), poxvirus, and picornavirus. In some embodiments, the virus is selected from a lentivirus vector, a gamma retrovirus vector, adeno-associated virus (AAV) vector, adenovirus vector, and a herpes virus vector.

In general, vectors contain an origin of replication functional in at least one organism, a promoter sequence and convenient restriction endonuclease site, and one or more selectable markers e.g. a drug resistance gene.

In some embodiments, the recombinant expression vector may comprise regulatory sequences, such as transcription and translation initiation and termination codons, which are specific to the type of host cell into which the vector is to be introduced.

In some embodiments, the vector of the disclosure may comprise one or more payloads taught herein, wherein the two or more payloads may be included in one ligand response. In this case, the two or more payloads are tuned by the same ligand or responsive agent simultaneously.

### Lentiviral Vehicles/Particles

In some embodiments, lentiviral vehicles/particles may be used as delivery modalities. Lentiviruses are subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia virus, visna-maedi and caprine arthritis encephalitis virus (CAEV).

Typically, lentiviral particles making up the gene delivery vehicle are replication defective on their own (also referred to as “self-inactivating”). Lentiviruses are able to infect both dividing and non-dividing cells by virtue of the entry mechanism through the intact host nuclear envelope. Recombinant lentiviral vehicles/particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non-dividing cells.

Lentiviral particles may be generated by co-expressing the virus packaging elements and the vector genome itself in a producer cell such as human HEK293T cells. These elements are usually provided in three or four separate plasmids. The producer cells are co-transfected with plasmids that encode lentiviral components including the core (i.e. structural proteins) and enzymatic components of the virus, and the envelope protein(s) (referred to as the packaging systems), and a plasmid comprising a foreign transgene to be transferred to the target cell, the vehicle itself (also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neo, DHFR, Gln synthetase or ADA, followed by selection in the presence of the appropriate drug and isolation of clones.

The producer cell produces recombinant viral particles that contain the foreign gene, for example, the transcription factor system components or polynucleotides thereof of the present disclosure. The recombinant viral particles are recovered from the culture media and titrated by standard methods used by those of skill in the art. The recombinant lentiviral vehicles can be used to infect target cells.

Cells that can be used to produce high-titer lentiviral particles may include, but are not limited to, HEK293T cells, 293G cells, STAR cells (Relander et al., Mol. Ther., 2005, 11: 452-459), FreeStyle™ 293 Expression System (ThermoFisher, Waltham, Mass.), and other HEK293T-based producer cell lines (e.g., Stewart et al., Hum Gene Ther. 2011, 22(3):357-369; Lee et al., Biotechnol Bioeng, 2012, 10996): 1551-1560; Throm et al., Blood. 2009, 113(21): 5104-5110; the contents of each of which are incorporated herein by reference in their entirety).

In some aspects, the envelope proteins may be heterologous envelope proteins from other viruses, such as the G protein of vesicular stomatitis virus (VSV G) or baculoviral gp64 envelope proteins. The VSV-G glycoprotein may especially be chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Alagoas virus (VSAV), Vesicular stomatitis Indiana virus (VSIV) and Vesicular stomatitis New Jersey virus (VSNJV) and/or stains provisionally classified in the vesiculovirus genus as Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURY), Klamath virus (KLAV), Kwatta virus (KWAV), La Joya virus (LJV), Malpais Spring virus (MSPV), Mount Elgon bat virus (MEBV), Perinet virus (PERV), Pike fry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV). The gp64 or other baculoviral env protein can be derived from Autographa calfornica nucleopolyhedrovirus (AcMNPV), Anagrapha falcifera nuclear polyhedrosis virus, Bombyx mori nuclear polyhedrosis virus, Choristoneura fumiferana nucleopolyhedrovirus, Orgyia pseudotsugata single capsid nuclear polyhedrosis virus, Epiphyas postvittana nucleopolyhedrovirus, Hyphantria cunea nucleopolyhedrovirus, Galleria mellonella nuclear polyhedrosis virus, Dhori virus, Thogoto virus, Antheraea pemyi nucleopolyhedrovirus or Batken virus. In some aspects, the envelope proteins may be RD114, RD115 or derived from gibbon ape leukemia virus (GaLV) or a baboon retroviral envelope glycoprotein (BaEV).

Other elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof.

Methods for generating recombinant lentiviral particles are discussed in the art, for example, U.S. Pat. Nos. 8,846,385; 7,745,179; 7,629,153; 7,575,924; 7,179,903; and 6,808,905.

Lentivirus vectors used may be selected from, but are not limited to pLVX, pLenti, pLenti6, pLJM1, FUGW, pWPXL, pWPI, pLenti CMV puro DEST, pLJM1-EGFP, pULTRA, pInducer20, pHIV-EGFP, pCW57.1, pTRPE, pELPS, pRRL, and pLionII.

### Adeno-Associated Viral Particles

Delivery of polynucleotides of any of the transcription factor systems, transcription factor constructs, or payload constructs of the present disclosure may be achieved using recombinant adeno-associated viral (rAAV) vectors. Such vectors or viral particles may be designed to utilize any of the known serotype capsids or combinations of serotype capsids.

AAV vectors include not only single stranded vectors but self-complementary AAV vectors (scAAVs). scAAV vectors contain DNA which anneals together to form double stranded vector genome. By skipping second strand synthesis, scAAVs allow for rapid expression in the cell.

The rAAV vectors may be manufactured by standard methods in the art such as by triple transfection, in sf9 insect cells or in suspension cell cultures of human cells such as HEK293 cells.

The transcription factor constructs and payload constructs may be encoded in one or more viral genomes to be packaged in the AAV capsids taught herein.

Such vector or viral genomes may also include, in addition to at least one or two ITRs (inverted terminal repeats), certain regulatory elements necessary for expression from the vector or viral genome. Such regulatory elements are well known in the art and include for example promoters, introns, spacers, stuffer sequences, and the like.

The transcription factor constructs or payload constructs of the disclosure may be administered in one or more or separate AAV particles.

In some embodiments, the transcription factor system constructs may be administered in one or more AAV particles. In some embodiments, more than one transcription factor system construct may be encoded in a viral genome.

### Retroviral Vehicles/Particles (γ-Retroviral Vectors)

In some embodiments, retroviral vehicles/particles may be used to deliver the transcription factor systems, transcription factor constructs or payload constructs of the present disclosure. Retroviral vectors (RVs) allow the permanent integration of a transgene in target cells. In addition to lentiviral vectors based on complex HIV-1/2, retroviral vectors based on simple gamma-retroviruses have been widely used to deliver therapeutic genes and demonstrated clinically as one of the most efficient and powerful gene delivery systems capable of transducing a broad range of cell types. Example species of Gamma retroviruses include the murine leukemia viruses (MLVs) and the feline leukemia viruses (FeLV).

In some embodiments, gamma-retroviral vectors derived from a mammalian gamma-retrovirus such as murine leukemia viruses (MLVs), are recombinant. The MLV families of gamma retroviruses include the ecotropic, amphotropic, xenotropic and polytropic subfamilies. Ecotropic viruses are able to infect only murine cells using mCAT-1 receptor. Examples of ecotropic viruses are Moloney MLV and AKV. Amphotropic viruses infect murine, human and other species through the Pit-2 receptor. One example of an amphotropic virus is the 4070A virus. Xenotropic and polytropic viruses utilize the same (Xpr1) receptor, but differ in their species tropism. Xenotropic viruses such as NZB-9-1 infect human and other species but not murine species, whereas polytropic viruses such as focus-forming viruses (MCF) infect murine, human and other species.

Gamma-retroviral vectors may be produced in packaging cells by co-transfecting the cells with several plasmids including one encoding the retroviral structural and enzymatic (gag-pol) polyprotein, one encoding the envelope (env) protein, and one encoding the vector mRNA comprising polynucleotide encoding the compositions of the present disclosure that is to be packaged in newly formed viral particles.

In some aspects, the recombinant gamma-retroviral vectors are pseudotyped with envelope proteins from other viruses. Envelope glycoproteins are incorporated in the outer lipid layer of the viral particles which can increase/alter the cell tropism. In some aspects, the envelope proteins may be RD 114, RD 115 or derived from gibbon ape leukemia virus (GaLV) or a baboon retroviral envelope glycoprotein (BaEV).

In some embodiments, the recombinant gamma-retroviral vectors are self-inactivating (SIN) gammaretroviral vectors. The vectors are replication incompetent. SIN vectors may harbor a deletion within the 3′ U3 region initially comprising enhancer/promoter activity. Furthermore, the 5′ U3 region may be replaced with strong promoters (needed in the packaging cell line) derived from Cytomegalovirus or RSV, or an internal promotor of choice, and/or an enhancer element. The choice of the internal promotors may be made according to specific requirements of gene expression needed for a particular purpose of the disclosure.

In some embodiments, polynucleotides of the transcription factor systems, transcription factor constructs, or payload constructs are inserted within the recombinant viral genome. The other components of the viral mRNA of a recombinant gamma-retroviral vector may be modified by insertion or removal of naturally occurring sequences (e.g., insertion of an IRES, insertion of a heterologous polynucleotide encoding a polypeptide or inhibitory nucleic acid of interest, shuffling of a more effective promoter from a different retrovirus or virus in place of the wild-type promoter and the like). In some examples, the recombinant gamma-retroviral vectors may comprise modified packaging signal, and/or primer binding site (PBS), and/or 5′-enhancer/promoter elements in the U3-region of the 5′-long terminal repeat (LTR), and/or 3′-SIN elements modified in the U3-region of the 3′-LTR. These modifications may increase the titers and the ability of infection.

Oncolytic Viral vector

In some embodiments, polynucleotides of present disclosure may be packaged into oncolytic viruses. As used herein, the term “oncolytic virus” refers to a virus that preferentially infects and kills cancer cells such as vaccine viruses. An oncolytic virus can occur naturally or can be a genetically modified virus such as oncolytic adenovirus, and oncolytic herpes virus.

In some embodiments, oncolytic vaccine viruses may include viral particles of a thymidine kinase (TK)-deficient, granulocyte macrophage (GM)-colony stimulating factor (CSF)-expressing, replication-competent vaccinia virus vector sufficient to induce oncolysis of cells in the tumor; See e.g., U.S. Pat. No. 9,226,977.

Messenger RNA (mRNA)

In some embodiments, the transcription factor systems, transcription factor constructs, or payload constructs of the disclosure may be designed as messenger RNAs (mRNAs). As used herein, the term “messenger RNA” (mRNA) refers to any polynucleotide which encodes a polypeptide of interest and which is capable of being translated to produce the encoded polypeptide of interest in vitro, in vivo, in situ or ex vivo. Such mRNA molecules may have the structural components or features of any of those taught in International Application number PCT/US2013/030062.

In some embodiments, the transcription factor systems or components thereof may be designed as self-amplifying RNA. “Self-amplifying RNA” as used herein refers to RNA molecules that can replicate in the host resulting in the increase in the amount of the RNA and the protein encoded by the RNA. Such self-amplifying RNA may have structural features or components of any of those taught in International Patent Application Publication No. WO2011005799.

### Dosing

The present disclosure provides methods comprising administering any one or more or component or composition of a transcription factor system to a subject in need thereof. These may be administered to a subject using any amount and any route of administration effective for preventing or treating or imaging a disease, disorder, and/or condition (e.g., a disease, disorder, and/or condition relating to cancer or an autoimmune disease). The exact amount required will vary from subject to subject, depending on the species, age, and general condition of the subject, the severity of the disease, the particular composition, its mode of administration, its mode of activity, and the like.

Compositions in accordance with the disclosure are typically formulated in dosage unit form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, compositions of the disclosure may be used for cancer immunotherapy in varying doses to avoid T cell exhaustion, prevent cytokine release syndrome and minimize toxicity associated with immunotherapy. For example, low doses of the compositions of the present disclosure may be used to initially treat patients with high tumor burden, while patients with low tumor burden may be treated with high and repeated doses of the compositions of the disclosure to ensure recognition of a minimal tumor antigen load. In another instance, the compositions of the present disclosure may be delivered in a pulsatile fashion to reduce tonic T cell signaling and enhance persistence in vivo. In some aspects, toxicity may be minimized by initially using low doses of the compositions of the disclosure, prior to administering high doses. Dosing may be modified if serum markers such as ferritin, serum C-reactive protein, IL6, IFN-7, and TNF-α are elevated.

In some embodiments, the neurotoxicity may be associated with CAR or TIL therapy. Such neurotoxicity may be associated CD19-CARs. Toxicity may be due to excessive T cell infiltration into the brain. In some embodiments, neurotoxicity may be alleviated by preventing the passage of T cells through the blood brain barrier. This can be achieved by the targeted gene deletion of the endogenous alpha-4 integrin inhibitors such as tysabri/natalizumab may also be useful in the present disclosure.

Also provided herein are methods of administering ligands or DRD ligands in accordance with the disclosure to a subject in need thereof. In some embodiments, the ligand is selected from Acetazolamide (ACZ), Methotrexate (MTX), and Trimethoprim (TMP). The ligand may be administered to a subject or to cells, using any amount and any route of administration effective for tuning the transcription factor system, DRD, or payloads of the disclosure. In some embodiments, ACZ may be used with a hCA2 DRD, methotrexate may be used with an hDHFR DRD, and trimethoprim may be used with an ecDHFR DRD. The exact amount required will vary from subject to subject, depending on the species, age, and general condition of the subject, the severity of the disease, the particular composition, its mode of administration, its mode of activity, and the like. The subject may be a human, a mammal, or an animal. Compositions in accordance with the disclosure are typically formulated in unit dosage form for ease of administration and uniformity of dosage. It will be understood, however, that the total daily usage of the compositions of the present disclosure may be decided by the attending physician within the scope of sound medical judgment. In certain embodiments, the ligands in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, from about 10 mg/kg to about 100 mg/kg, from about 50 mg/kg to about 500 mg/kg, from about 100 mg/kg to about 1000 mg/kg, of subject body weight per day, one or more times a day, to obtain the desired effect. In some embodiments, the dosage levels may be 1 mg/kg, 5 mg/kg, I0 mg/kg, 20 mg/kg, 30 mg/kg, 40 mg/kg, 50 mg/kg, 60 mg/kg, 70 mg/kg, 80 mg/kg, 90 mg/kg, I00 mg/kg, I00 mg/kg, I10 mg/kg, I20 mg/kg, I30 mg/kg, 140 mg/kg, I50 mg/kg, I60 mg/kg, I70 mg/kg, I80 mg/kg, I90 mg/kg or mg/kg of subject body weight per day, or one or more times a day, to obtain the desired effect.

The present disclosure provides methods for delivering to a cell or tissue any of the ligands described herein, comprising contacting the cell or tissue with the ligand and can be accomplished in vitro, ex vivo, or in vivo. In certain embodiments, the ligands in accordance with the present disclosure may be administered to cells at dosage levels sufficient to deliver from about 1 nM to about 10 nM, from about 5 nM to about 50 nM, from about 10 nM to about 100 nM, from about 50 nM to about 500 nM, from about 100 nM to about 1000 nM, from about 1 μM to about 10 μM from about 5 μM to about 50 μM from about 10 μM to about 100 μM from about 25 μM to about 250 μM from about 50 μM to about 500 μM. In some embodiments, the ligand may be administered to cells at doses selected from but not limited to 0.00064 μM, 0.0032 μM, 0.016 μM, 0.08 μM, 0.4 μM, 1 μM 2 μM, 10 μM, 50 μM, 75 μM, 100 μM, 150 μM, 175 μM, 200 μM, 250 μM.

The desired dosage of the ligands of the present disclosure may be delivered only once, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact, i.e., single administration event. The desired dosage of the ligand of the present disclosure may be administered as a “pulse dose” or as a “continuous flow”. As used herein, a “pulse dose” is a series of single unit doses of any therapeutic administered with a set frequency over a period of time. As used herein, a “continuous flow” is a dose of therapeutic administered continuously for a period of time in a single route/single point of contact, i.e., continuous administration event. A total daily dose, an amount given or prescribed in 24-hour period, may be administered by any of these methods, or as a combination of these methods, or by any other methods suitable for a pharmaceutical administration.

### Administration

In some embodiments, the compositions for cancer immunotherapy or treatment of autoimmune disease may be administered to cells ex vivo and subsequently administered to the subject. In further embodiments, the cell is selected from a B cell, a T cell, a natural killer cell (NK cell), or a tumor infiltrating lymphocyte (TIL). Immune cells can be isolated and expanded ex vivo using a variety of methods known in the art. For example, methods of isolating cytotoxic T cells are described in U.S. Pat. Nos. 6,805,861 and 6,531,451. Isolation of NK cells is described in U.S. Pat. No. 7,435,596.

In some embodiments, depending upon the nature of the cells, the cells may be introduced into a host organism e.g. a mammal, in a wide variety of ways including by injection, transfusion, infusion, local instillation or implantation. In some aspects, the cells of the disclosure may be introduced at the site of the tumor. The number of cells that are employed will depend upon a number of circumstances, the purpose for the introduction, the lifetime of the cells, the protocol to be used, for example, the number of administrations, the ability of the cells to multiply, or the like. The cells may be in a physiologically-acceptable medium.

In some embodiments, the cells of the disclosure may be administrated in multiple doses to subjects having a disease or condition. The administrations generally effect an improvement in one or more symptoms of cancer or a clinical condition and/or treat or prevent cancer or clinical condition or symptom thereof.

In some embodiments, the compositions for immunotherapy or treatment of autoimmune disease may be administered in vivo. In some embodiments, polynucleotides of the present disclosure comprising transcription factor systems, payloads and compositions of the disclosure may be delivered in vivo to the subject via gene therapy.

### Routes of Delivery

The pharmaceutical compositions, transcription factor systems, nucleic acids, polynucleotides, payloads, vectors and cells of the present disclosure may be administered by any route to achieve a therapeutically effective outcome. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

### Parenteral and Injectable Administration

In some embodiments, pharmaceutical compositions, transcription factor systems, nucleic acids, polynucleotides, payloads, vectors and cells of the present disclosure may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

### Detectable Agents and Labels

The transcription factor systems, nucleic acids, polynucleotides, payloads, vectors and cells of the present disclosure may be associated with or bound to one or more radioactive agents or detectable agents.

These agents include various organic small molecules, inorganic compounds, nanoparticles, enzymes or enzyme substrates, fluorescent materials, luminescent materials (e.g., luminol), bioluminescent materials (e.g., luciferase, luciferin, and aequorin), chemiluminescent materials, radioactive materials (e.g., ¹⁸F, ⁶⁷Ga, ^{81m}Kr, ⁸²Rb, ¹¹¹In, ¹²³I, ¹³³Xe, ²⁰¹Tl, ¹²⁵, ³⁵S, ¹⁴C, ³H, or ^{99m}Tc (e.g., as pertechnetate (technetate (VII), TcO₄⁻)), and contrast agents (e.g., gold (e.g., gold nanoparticles), gadolinium (e.g., chelated Gd), iron oxides (e.g., superparamagnetic iron oxide (SPIO), monocrystalline iron oxide nanoparticles (MIONs), and ultrasmall superparamagnetic iron oxide (USPIO)), manganese chelates (e.g., Mn-DPDP), barium sulfate, iodinated contrast media (iohexol), microbubbles, or perfluorocarbons).

In some embodiments, the detectable agent may be a non-detectable precursor that becomes detectable upon activation (e.g., fluorogenic tetrazine-fluorophore constructs (e.g., tetrazine-BODIPY FL, tetrazine-Oregon Green 488, or tetrazine-BODIPY TMR-X) or enzyme activatable fluorogenic agents (e.g., PROSENSE® (VisEn Medical))). In vitro assays in which the enzyme labeled compositions can be used include, but are not limited to, enzyme linked immunosorbent assays (ELISAs), immunoprecipitation assays, immunofluorescence, enzyme immunoassays (EIA), radioimmunoassays (RIA), and Western blot analysis.

### Applications and Uses

The transcription factor systems, constructs, ligands, or compositions of the present disclosure may be utilized in a large variety of applications including, but not limited to, therapeutics, diagnosis and prognosis, bioengineering, bioprocessing, biomanufacturing, research agents, metabolomics, gene expression, enzyme replacement, etc.

The present disclosure provides methods comprising administering a composition, for example, a pharmaceutical composition comprising one or more components of a transcription factor system to a subject in need thereof.

While there may be several uses that do not involve a medical treatment, for example, to generate cell lines and reagents for scientific research, one use involves the administration of the compositions of the present disclosure to generate in vivo gene therapy or modified cells for adoptive cell therapy, for example, the treatment of cancer, autoimmune diseases and other diseases. In an illustrative method of medical treatment or prevention of a disease, condition or disorder in a subject in need thereof, can include the following steps: (a) providing a population of cells (either human, animal, primary or cell culture, including autologous, allogenic or syngeneic); (b) introducing at least one nucleic acid molecule into at least one cell in the population of cells, wherein the at least one nucleic acid molecule comprises: (i) a first polynucleotide comprising a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD), wherein the transcription factor activation domain and/or the transcription factor DNA binding domain is operably linked to the DRD; and (ii) a second polynucleotide that comprises a fourth nucleic acid sequence that encodes a protein of interest that treats the disease, the fourth nucleic acid sequence being operably linked to an exogenous inducible promoter comprising the specific polynucleotide binding site; (c) delivering the cell into the subject; and (d) administering a ligand to the subject that stabilizes the DRD sufficiently to enable expression of the transcription factor activation domain and the transcription factor DNA binding domain in an amount sufficient to form a transcription factor that binds to the specific polynucleotide binding site and enables expression of the protein of interest in the cell; wherein expression of the protein of interest is regulated by the presence of ligand in the subject, and the amount and/or duration of ligand administration is sufficient to produce a therapeutically effective amount of the protein of interest.

In the above method, the protein of interest can be used to ameliorate, cure, prevent or reduce one or more symptoms of the disease, condition or disorder.

The compositions of the present disclosure may be administered to a subject using any amount and any route of administration effective for preventing or treating or imaging a disease, disorder, and/or condition (e.g., a disease, disorder, and/or condition relating to cancer, autoimmune diseases and other diseases). The exact amount required will vary from subject to subject, depending on the species, age, and general condition of the subject, the severity of the disease, the particular composition, its mode of administration, its mode of activity, and the like.

Compositions in accordance with the disclosure are typically formulated in dosage unit form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Also provided herein, are methods of administering one or more stabilizing ligands (as used herein, the ligand that stabilizes the DRD, may be called a stabilizing ligand or simply a ligand, with the understanding that the ligand is effective in stabilizing the DRD used in the transcription factor systems in accordance with the disclosure) to a subject in need thereof. The ligand may be administered to a subject or to cells, using any amount and any route of administration effective for tuning the amount of transcription factor expression of the present disclosure in a cell comprising the transcription factor system. The exact amount of stabilizing ligand required will vary from subject to subject, depending on the species, age, and general condition of the subject, the severity of the disease, the particular composition, its mode of administration, its mode of activity, and the like. The subject may be a human, a mammal, or an animal.

### Therapeutic Uses

### Cancer Immunotherapy

Cancer immunotherapy aims at the induction or restoration of the reactivity of the immune system towards cancer. Significant advances in immunotherapy research have led to the development of various strategies which may broadly be classified into active immunotherapy and passive immunotherapy. In general, these strategies may be utilized to directly kill cancer cells or to counter the immunosuppressive tumor microenvironment. Active immunotherapy aims at induction of an endogenous, long-lasting tumor-antigen specific immune response. The response can further be enhanced by non-specific stimulation of immune response modifiers such as cytokines. In contrast, passive immunotherapy includes approaches where effector immune molecules such as tumor-antigen specific cytotoxic T cells or antibodies are administered to the host. This approach is short lived and requires multiple applications.

Despite significant advances, the efficacy of current immunotherapy strategies is limited by associated toxicities. These are often related to the narrow therapeutic window associated with immunotherapy, which in part, emerges from the need to push therapy dose to the edge of potentially fatal toxicity to get a clinically meaningful treatment effect. Further, dose expands in vivo since adoptively transferred immune cells continue to proliferate within the patient, often unpredictably.

A major risk involved in immunotherapy is the on-target but off-tumor side effects resulting from T-cell activation in response to normal tissue expression of the tumor associated antigen (TAA). Clinical trials utilizing T cells expressing T-cell receptor against specific TAA reported skin rash, colitis and hearing loss in response to immunotherapy.

Immunotherapy may also produce on target, on-tumor toxicities that emerge when tumor cells are killed in response to the immunotherapy. The adverse effects include tumor lysis syndrome, cytokine release syndrome and the related macrophage activation syndrome. Importantly, these adverse effects may occur during the destruction of tumors, and thus even a successful on-tumor immunotherapy might result in toxicity. Approaches to control immunotherapy via immunotherapeutic agent regulation are thus highly desirable since they have the potential to reduce toxicity and maximize efficacy.

The present disclosure provides systems, compositions, immunotherapeutic agents and methods for immunotherapy. These compositions provide tunable regulation of gene expression and function in immunotherapy, for example for the prevention and treatment of cancer.

In one aspect, the systems, compositions, immunotherapeutic agents and other components of the disclosure can be controlled by a separately added stabilizing ligand, which provides a significant flexibility to regulate cancer immunotherapy. Further, the systems, compositions and the methods of the present disclosure may also be combined with therapeutic agents such as chemotherapeutic agents, small molecules, gene therapy, and antibodies to prevent and/or treat a disease, for example, cancer.

The tunable nature of the systems and compositions of the disclosure has the potential to improve the potency and duration of the efficacy of immunotherapies. Reversibly silencing the biological activity of adoptively transferred cells using compositions of the present disclosure allows maximizing the potential of cell therapy without irretrievably killing and terminating the therapy.

The present disclosure provides methods for fine tuning of immunotherapy after administration to patients. This in turn improves the safety and efficacy of immunotherapy and increases the subject population that may benefit from immunotherapy.

In some embodiments, immune cells of the disclosure may be T cells modified to express a payload or protein of interest, for example, an antigen-specific T cell receptor (TCR), or an antigen specific chimeric antigen receptor (CAR) taught herein (known as CAR T cells). Accordingly, at least one polynucleotide encoding a protein of interest, for example, a CAR system (or a TCR) described herein, or a vector comprising the polynucleotide is introduced into a T cell. The T cell expressing the CAR or TCR binds to a specific antigen via the extracellular targeting moiety of the CAR or TCR, thereby a signal via the intracellular signaling domain (s) is transmitted into the T cell, and as a result, the T cell is activated. The activated CAR T cell changes its behavior including release of a cytotoxic cytokine (e.g., a tumor necrosis factor, and lymphotoxin, etc.), improvement of a cell proliferation rate, change in a cell surface molecule, or the like. Such changes cause destruction of a target cell expressing the antigen recognized by the CAR or TCR. In addition, release of a cytokine or change in a cell surface molecule stimulates other immune cells, for example, a B cell, a dendritic cell, a NK cell, and a macrophage.

The CAR introduced into a T cell may be a first-generation CAR including only the intracellular signaling domain from TCR CD3zeta, or a second-generation CAR including the intracellular signaling domain from TCR CD3zeta and a costimulatory signaling domain, or a third-generation CAR including the intracellular signaling domain from TCR CD3zeta and two or more costimulatory signaling domains, or a split CAR system, or an on/off switch CAR system. In one example, the expression of the CAR or TCR is controlled by a transcription factor, wherein the transcription factor or component thereof is operably linked to a DRD, which in the absence of a stabilizing ligand will result in the little to no accumulation of transcription factor. The payload has a polynucleotide binding sequence specific to the transcription factor or component thereof, therefore, without the stabilizing ligand, little to no protein of interest is produced. When stabilizing ligand is administered to the cell comprising the transcription factor system, the transcription factor is rescued from degradation when coupled to the DRD, and the transcription factor then binds to its cognate polynucleotide binding sequence immediately adjacent the protein of interest, which then is transcribed. The transcribed mRNA is then translated to produce the polypeptide/protein of interest. In some exemplary embodiments, the presence or absence of the DRD stabilizing ligand is used to tune the CAR or TCR expression in transduced T cells or NK cells.

In some embodiments, CAR T cells of the disclosure may be further modified to express another one, two, three or more immunotherapeutic agents. The immunotherapeutic agents may be another CAR or TCR specific to a different target molecule; a cytokine such as IL2, IL12, IL15 and IL18, or a cytokine receptor such as IL15Ra; a chimeric switch receptor that converts an inhibitory signal to a stimulatory signal; a homing receptor that guides adoptively transferred cells to a target site such as the tumor tissue; an agent that optimizes the metabolism of the immune cell; or a safety switch gene (e.g., a suicide gene) that kills activated T cells when a severe event is observed after adoptive cell transfer or when the transferred immune cells are no-longer needed. These molecules may be included in the same constructs or in separate constructs.

In one embodiment, the CAR T cell (including TCR T cell) of the disclosure may be an “armed” CAR T cell which is transfected or transduced with one or more components of the transcription factor system comprising a CAR payload and either the same or a different transcription factor system encoding a cytokine under control of the same or different transcription factor operably linked to the same or different DRD. The inducible or constitutively secreted active cytokines further arm CAR T cells to improve efficacy and persistence. In this context, such CAR T cell is also referred to as “armored CAR T cell”. The “armor” molecule may be selected based on the tumor microenvironment and other elements of the innate and adaptive immune systems. In some embodiments, the molecule may be a stimulatory factor such as IL2, IL12, IL15, IL18, type I IFN, CD40L and 4-1BBL which have been shown to further enhance CAR T cell efficacy and persistence in the face of a hostile tumor microenvironment via different mechanisms.

Chimeric Antigen Receptor engineered T cells (CAR-T) therapies have yet to be successfully applied to solid tumors. Enhancing CAR-T cell functionality and selectively delivering cargo to the site of solid tumors represent key tactics to achieve effective CAR-T therapy for solid tumors. In one embodiment, a payload or protein of interest may include Interleukin 12 (IL12) may be utilized to enhance the effectiveness of CAR-T cells, especially since it has the potential to remodel the tumor microenvironment. IL12 has been previously shown to be effective in enhancing efficacy of CAR or TCR modified T-cells as well as tumor infiltrating lymphocytes (TILs) in preclinical and clinical models. However, constitutive production of IL12 can compromise safety and/or efficacy; therefore, on demand, local delivery of the cytokine may be a preferred approach. In some embodiments, transcription factor system of the present disclosure, or components thereof, may be utilized to exogenously control IL12 expression to enable the use of IL12 in adoptive cell therapy.

In some embodiments, transcription factor regulated systems of the present disclosure may be used to regulate a payload such as Flexi IL12 (or other IL12 constructs such as membrane bound IL12) expression in transformed immune cells to improve the efficacy of the CARs, especially in solid tumor settings, by providing a controlled local signal for tumor microenvironment remodeling and epitope spreading. Transcription factor regulation as described herein also provides rapid, dose dependent, and local production of IL12, upon addition of DRD specific stabilizing ligands.

In some aspects, the armed CAR T cell of the disclosure is modified to express a CD19 CAR and a payload such as IL12, which is regulated using a transcription factor system or composition of the present disclosure. Such T cells, after CAR mediated activation in the tumor, release inducible IL12 which augments T-cell activation and attracts and activates innate immune cells to eliminate CD19-positive cancer cells.

In one embodiment, T cells of the disclosure may be modified to incorporate a transcription factor system comprising a CAR payload encoded by the transcription factor system or component thereof and a nucleic acid sequence encoding a suicide gene.

In one embodiment, the CAR T cell (including TCR T cell) of the disclosure may be transfected or transduced with one or more components of a transcription factor system comprising a cytokine and a safety switch gene (e.g., suicide gene). The suicide gene may be an inducible caspase such as caspase 9 which induces apoptosis, when activated by an extracellular stabilizing ligand of a DRD encoded by the transcription factor system. Such induced apoptosis eliminates transferred cell as required to decrease the risk of direct toxicity and uncontrolled cell proliferation.

In one embodiment, the transcription factor system, and components thereof that tune expression levels and activities of any described payloads or proteins of interest (used interchangeably) may be used for immunotherapy. As non-limiting examples, an immunotherapeutic agent may be an antibody and fragments and variants thereof, a cancer specific T cell receptor (TCR) and variants thereof, an anti-tumor specific chimeric antigen receptor (CAR), a chimeric switch receptor, an inhibitor of a co-inhibitory receptor or ligand, an agonist of a co-stimulatory receptor and ligand, a cytokine, chemokine, a cytokine receptor, a chemokine receptor, a soluble growth factor, a metabolic factor, a suicide gene, a homing receptor, or any agent that induces an immune response in a cell and a subject.

In some embodiments, the composition for inducing or suppressing an immune response may comprise one or more components of a transcription factor system, or one or more polypeptides encoded by a transcription factor system. In some embodiments, the transcription factor system may comprise a first polynucleotide comprising a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD); wherein at least one of the transcription factor activation domain, the transcription factor DNA binding domain, and the combination of the transcription factor activation domain and the transcription factor DNA binding domain is operably linked to the DRD; and a second polynucleotide comprising a fourth nucleic acid sequence that encodes a protein of interest, the fourth nucleic acid sequence being operably linked to an inducible promoter comprising the specific polynucleotide binding site; wherein the transcription factor activation domain and the transcription factor DNA binding domain interact to form a transcription factor; and wherein binding of the transcription factor to the specific polynucleotide binding site is required for regulating transcription of the fourth nucleic acid sequence by the transcription factor.

In one aspect, the payload may be an immunotherapeutic agent.

In some embodiments, a transcription factor system, and compositions of the present disclosure relate to transcriptional regulation of protein (protein of interest or payload) function, including for example, anti-tumor immune responses of immunotherapeutic agents. In some embodiments, the immunotherapeutic agents may include cytokines, chemokines, antibodies, integrins, integral proteins, membrane proteins, extracellular proteins that may be used to upregulate, or improve the function of one or more immune cell types, or down regulate the activity of one or more immune cell types. In various embodiments, the immunotherapeutic agents useful in the treatment of a disease, condition or disorder can include cytokines, for example interleukins. In various embodiments, the transcription factor system provides a protein of interest or payload that includes an interleukin, for example, IL-2, IL-6, IL12, IL15, IL18 and other immunotherapeutic agents that promotes or upregulates the longevity and activity of one or more immune cell types useful to treat a disease, condition or disorder or a symptom associate with any of these.

In some embodiments, cells which are genetically modified to encode and express at least one transcription factor operable to permit transcription of a protein of interest linked to a transcription factor polynucleotide binding site (immunotherapeutic agent) may be used for adoptive cell therapy (ACT, also referred to as “adoptive cell transfer”). As used herein, adoptive cell transfer refers to the administration of immune cells (from autologous, allogenic or genetically modified hosts) with direct anticancer activity. ACT has shown promise in clinical application against malignant and infectious disease. For example, T cells genetically engineered to recognize CD19 have been used to treat follicular B cell lymphoma (Kochenderfer et al., Blood, 2010, 116:4099-4102; and Kochenderfer and Rosenberg, Nat Rev Clin Oncol., 2013, 10(5): 267-276) and ACT using autologous lymphocytes genetically-modified to express anti-tumor T cell receptors has been used to treat metastatic melanoma (Rosenberg and Dudley, Curr. Opin. Immunol. 2009, 21: 233-240).

According to the present disclosure, the one or more components of a transcription factor system may be used in the development and implementation of cell therapies such as adoptive cell therapy. In some embodiments, one or more components of a transcription factor system, may be used in cell therapies to effect CAR therapies, in the manipulation or regulation of TILs, in allogeneic cell therapy, in combination T cell therapy with other treatment lines (e.g. radiation, cytokines), to encode engineered TCRs, or modified TCRs, or to enhance T cells other than TCRs (e.g. by introducing cytokine genes, genes for the checkpoint inhibitors PD1, CTLA4).

Provided herein are methods for use in adoptive cell therapy. The methods involve preconditioning a subject in need thereof, modulating immune cells with one or more components of a transcription factor system, and/or compositions of the present disclosure; administering to a subject engineered immune cells expressing compositions of the disclosure and the successful engraftment of engineered cells within the subject.

In some embodiments, regulatable transcription factor expression constructs and compositions of the present disclosure, may be used to minimize preconditioning regimens associated with adoptive cell therapy. As used herein “preconditioning” refers to any therapeutic regimen administered to a subject to improve the outcome of adoptive cell therapy. Preconditioning strategies include but are not limited to total body irradiation and/or lymphodepleting chemotherapy. Adoptive therapy clinical trials without preconditioning have failed to demonstrate any clinical benefit, indicating its importance in ACT. Yet, preconditioning is associated with significant toxicity and limits the subject cohort that is suitable for ACT. In some instances, immune cells for ACT may be engineered to express cytokines such as IL-2, IL-6, IL12 and IL15 as payload using transcription factors described herein to permit selective expression of the protein of interest which may be tuned using a stabilizing ligand of the present disclosure to reduce the need for preconditioning (Pengram et al. (2012) Blood 119 (18): 4133-41; the contents of which are incorporated by reference in their entirety).

In some embodiments, immune cells for ACT may be dendritic cells, T cells such as CD8+ T cells and CD4+ T cells, natural killer (NK) cells, NK T cells, Cytotoxic T lymphocytes (CTLs), tumor infiltrating lymphocytes (TILs), lymphokine activated killer (LAK) cells, memory T cells, regulatory T cells (Tregs), helper T cells, cytokine-induced killer (CIK) cells, and any combination thereof. In other embodiments, immune stimulatory cells for ACT may be generated from embryonic stem cell (ESC) and induced pluripotent stem cell (iPSC). In some embodiments, autologous or allogeneic immune cells are used for ACT.

In some embodiments, cells used for ACT may be T cells engineered to express CARs comprising an antigen-binding domain specific to an antigen on tumor cells of interest. In other embodiments, cells used for ACT may be NK cells engineered to express CARs comprising an antigen-binding domain specific to an antigen on tumor cells of interest. In addition to adoptive transfer of genetically modified T cells (e.g., CAR T cells) for immunotherapy, alternate types of CAR-expressing leukocytes, either alone, or in combination with CAR T cells may be used for adoptive immunotherapy. In one example, a mixture of T cells and NK cells may be used for ACT. The expression level of CARs in T cells and NK cells, according to the present disclosure, is tuned and controlled by a small molecule that binds to the DRD(s) operably linked to a transcription factor or components thereof, which enables selective transcription of the CAR in the transfected or transduced T cells and NK cells. In this scenario, the CAR is encoded by a nucleic acid sequence operably inked to an inducible promoter comprising the specific polynucleotide binding site of the transcription factor.

In some embodiments, NK cells engineered to express one or more components of a transcription factor system may be used for ACT. NK cell activation induces perforin/granzyme-dependent apoptosis in target cells. NK cell activation also induces cytokine secretion such as IFN 7, TNF-α and GM-CSF. These cytokines enhance the phagocytic function of macrophages and their antimicrobial activity and augment the adaptive immune response via up-regulation of antigen presentation by antigen presenting cells such as dendritic cells (DCs) (Reviewed by Vivier et al., Nat. Immunol., 2008, 9(5): 503-510).

Other examples of genetic modification may include the introduction of chimeric antigen receptors (CARs) and the down-regulation of inhibitory NK cell receptors such as NKG2A.

NK cells may also be genetically reprogrammed to circumvent NK cell inhibitory signals upon interaction with tumor cells. For example, using CRISPR, ZFN, or TALEN to genetically modify NK cells to silence their inhibitory receptors may enhance the anti-tumor capacity of NK cells.

Immune cells can be isolated and expanded ex vivo using a variety of methods known in the art. For example, methods of isolating and expanding cytotoxic T cells are described in U.S. Pat. Nos. 6,805,861 and 6,531,451; US Patent Publication NO. US20160348072A1 and International Patent Publication NO. WO2016168595A1; the contents of each of which are incorporated herein by reference in their entirety. Isolation and expansion of NK cells is described in US Patent Publication NO. US20150152387A1, U.S. Pat. No. 7,435,596; and Oyer, J. L. (2016). Cytotherapy. 18(5):653-63; the contents of each of which are incorporated by reference herein in its entirety. Specifically, human primary NK cells may be expanded in the presence of feeder cells e.g. a myeloid cell line that has been genetically modified to express membrane bound IL15, IL21, IL12 and 4-1BBL.

In some instances, sub populations of immune cells may be enriched for ACT. Methods for immune cell enrichment are taught in International Patent Publication No. WO2015039100A1. In another example, T cells positive for B and T lymphocyte attenuator marker BTLA) may be used to enrich for T cells that are anti-cancer reactive as described in U.S. Pat. No. 9,512,401 (the content of each of which are incorporated herein by reference in their entirety).

In some embodiments, immune cells for ACT may be depleted of select sub populations to enhance T cell expansion. For example, immune cells may be depleted of Foxp3+T lymphocytes to minimize the anti-tumor immune response using methods taught in US Patent Publication NO. US 20160298081A1; the contents of which are incorporated by reference herein in their entirety.

In some embodiments, activation and expansion of T cells for ACT is achieved antigenic stimulation of a transiently expressed Chimeric Antigen Receptor (CAR) on the cell surface. Such activation methods are taught in International Patent NO. WO2017015427, the content of which are incorporated herein by reference in their entirety.

In some embodiments, immune cells may be activated by antigens associated with antigen presenting cells (APCs). In some embodiments, the APCs may be dendritic cells, macrophages or B cells that are antigen specific or nonspecific. The APCs may autologous or homologous in their organ. In some embodiments, the APCs may be artificial antigen presenting cells (aAPCs) such as cell based aAPCs or acellular aAPCs. Cell based aAPCs may be selected from either genetically modified allogeneic cells such as human erythroleukemia cells or xenogeneic cells such as murine fibroblasts and Drosophila cells. Alternatively, the APCs maybe be acellular wherein the antigens or costimulatory domains are presented on synthetic surfaces such as latex beads, polystyrene beads, lipid vesicles or exosomes.

In some embodiments, cells of the disclosure, specifically T cells may be expanded using artificial cell platforms. In one embodiment, the mature T cells may be generated using artificial thymic organoids (ATOs) described by Seet C S et al. 2017. Nat Methods. 14, 521-530 (the contents of which are incorporated herein by reference in their entirety). ATOs are based on a stromal cell line expressing delta like canonical notch ligand (DLL1). In this method, stromal cells are aggregated with hematopoietic stem and progenitor cells by centrifugation and deployed on a cell culture insert at the air-fluid interface to generate organoid cultures. ATO-derived T cells exhibit naive phenotypes, a diverse T cell receptor (TCR) repertoire and TCR-dependent function.

In some embodiments, adoptive cell therapy is carried out by autologous transfer, wherein the cells are derived from a subject in need of a treatment and the cells, following isolation and processing are administered to the same subject. In other instances, ACT may involve allogenic transfer wherein the cells are isolated and/or prepared from a donor subject other than the recipient subject who ultimately receives cell therapy. The donor and recipient subject may be genetically identical, or similar or may express the same HLA class or subtype.

In some embodiments, the multiple immunotherapeutic agents introduced into the immune cells for ACT (e.g., T cells and NK cells) may be controlled by the same or different transcription factor systems. In one example, each of the two payloads, for example, a cytokine such as IL12 and a CAR construct such as CD19 CAR that are transcribed by one or more transcription factor(s) on the same or different transcription factor systems, wherein the transcription factors(s) are linked to the same or different DRDs. The payloads are transcribed and translated when the DRD(s) is/are stabilized with a stabilizing ligand specific for the DRD(s). The expression of IL12 and CD19 CAR is tuned using one or more stabilizing ligands. In other embodiments, the multiple immunotherapeutic agents introduced into the immune cells for ACT (e.g., T cells and NK cells) may be controlled by different transcription factor systems. In one example, a cytokine such as IL12 and a CAR construct such as CD19 CAR are each transcribed by one of two different transcription factors, each transcription factor is operably linked to a different DRDs, and thereby can be tuned separately using different stimuli. In another example, a suicide gene and a CAR construct may be transcriptionally activated by two different transcription factors.

Following genetic modulation using one or more components of a transcription factor system and compositions of the disclosure, cells are administered to the subject in need thereof. Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338; the contents of each of which are incorporated herein by reference in their entirety.

In some embodiments, immune cells for ACT may be modified to express one or more immunotherapeutic agents (proteins of interest) which facilitate immune cells activation, infiltration, expansion, survival and anti-tumor functions. The immunotherapeutic agents may be a second CAR or TCR specific to a different target molecule; a cytokine or a cytokine receptor; a chimeric switch receptor that converts an inhibitory signal to a stimulatory signal; a homing receptor that guides adoptively transferred cells to a target site such as the tumor tissue; an agent that optimizes the metabolism of the immune cell; or a safety switch gene (e.g., a suicide gene) that kills activated T cells when a severe event is observed after adoptive cell transfer or when the transferred immune cells are no-longer needed.

In some embodiments, immune cells used for adoptive cell transfer can be genetically manipulated to improve their persistence, cytotoxicity, tumor targeting capacity, and ability to home to disease sites in vivo, with the overall aim of further improving upon their capacity to kill tumors in cancer patients. One example is to introduce one or more components of a transcription factor system of the disclosure encoding a cytokine, such as a gamma-cytokine (e.g. IL2 and IL15) into immune cells to promote immune cell proliferation and survival. Transduction of cytokine genes (e.g., gamma-cytokines IL2 and IL15) encoded by a transcription factor system into immune cells will enable the immune cells, e.g. NK cells to propagate without addition of exogenous cytokines such that the cytokine expressing NK cells have enhanced tumor cytotoxicity.

In some embodiments, one or more components of a transcription factor system may be utilized to prevent T cell exhaustion. As used herein, “T cell exhaustion” refers to the stepwise and progressive loss of T cell function caused by chronic T cell activation. T cell exhaustion is a major factor limiting the efficacy of antiviral and antitumor immunotherapies. Exhausted T cells have low proliferative and cytokine producing capabilities concurrent with high rates of apoptosis and high surface expression of multiple inhibitory receptors. T cell activation leading to exhaustion may occur either in the presence or absence of the antigen.

In some embodiments, one or more components of a transcription factor system may be utilized to prevent T cell exhaustion in the context of Chimeric Antigen Receptor-T cell therapy (CAR-T). In this context, exhaustion in some instances, may be caused by the oligomerization of the scFvs of the CAR on the cell surface which leads to continuous activation of the intracellular domains of the CAR. As a non-limiting example, CARs of the present disclosure may include scFvs that are unable to oligomerize. As another non-limiting example, CARs that are rapidly internalized and re-expressed following antigen exposure may also be selected to prevent chronic scFv oligomerization on cell surface. In one embodiment, the framework region of the scFvs may be modified to prevent constitutive CAR signaling (Long et al. 2014. Cancer Research. 74(19) Si; the contents of which are incorporated by reference in their entirety). One or more components of a transcription factor system of the present disclosure may also be used to regulate the surface expression of the CAR on the T cell surface to prevent chronic T cell activation. The CARs of the disclosure may also be engineered to minimize exhaustion. As a non-limiting example, the 41-BB signaling domain may be incorporated into CAR design to ameliorate T cell exhaustion. In some embodiments, any of the strategies disclosed by Long H A et al. may be utilized to prevent exhaustion (Long A H et al. (2015) Nature Medicine 21, 581-590; the contents of which are incorporated herein by reference in their entirety).

In some embodiments, the tunable nature of the transcription factor system of the present disclosure may be utilized to reverse human T cell exhaustion observed with tonic CAR signaling. Reversibly silencing the biological activity of adoptively transferred cells using compositions of the present disclosure may be used to reverse tonic signaling which, in turn, may reinvigorate the T cells. Reversal of exhaustion may be measured by the downregulation of multiple inhibitory receptors associated with exhaustion.

In some embodiments, T cell metabolic pathways may be modified to diminish the susceptibility of T cells to exhaustion. Metabolic pathways may include, but are not limited to glycolysis, urea cycle, citric acid cycle, beta oxidation, fatty acid biosynthesis, pentose phosphate pathway, nucleotide biosynthesis, and glycogen metabolic pathways. As a non-limiting example, payloads that reduce the rate of glycolysis may be utilized to restrict or prevent T cell exhaustion (Long et al. Journal for Immunotherapy of Cancer 2013, 1(Suppl 1): P21; the contents of which are incorporated by reference in their entirety). In one embodiment, T cells of the present disclosure may be used in combination with inhibitors of glycolysis such as 2-deoxyglucose, and rapamycin.

In some embodiments, payloads or proteins of interest of the disclosure may be used in conjunction with antibodies or fragments that target T cell surface markers associated with T cell exhaustion. T-cell surface markers associated with T cell exhaustion that may be used include, but are not limited to, CTLA-1, PD-1, TGIT, LAG-3, 2B4, BTLA, TIM3, VISTA, and CD96. In some embodiments, one or more components of a transcription factor system may be utilized to prevent T cell exhaustion.

In some embodiments, the compositions of the present disclosure may be utilized to alter TIL (tumor infiltrating lymphocyte) populations in a subject. In one embodiment, any of the payloads described herein may be utilized to change the ratio of CD4 positive cells to CD8 positive populations. In some embodiments, TILs may be sorted ex vivo and engineered to express any of the cytokines described herein. Payloads of the disclosure may be used to expand CD4 and/or CD8 populations of TILs to enhance TIL mediated immune response.

Parameters for improving CAR-T therapy outcome are described in Finney et al. JCI. 2019; 129(5):2123-2132 (the contents of which are herein incorporated by reference in their entirety). The levels of biomarker LAG3 (high)/TNF-α (low) in peripheral blood CD8+ T cells at the time of apheresis may also predict a subsequent dysfunctional response in subjects with high antigen load who do not achieve complete response that is durable for more than a few weeks. T cell-intrinsic features that are a consequence of the starting T cell repertoire and the effects of the manufacturing process converge with CD19 antigen-induced activation following adoptive transfer may also play a role in the outcome of CAR-T therapy. The starting T cell repertoire may in part be affected by the timing of the apheresis. In one embodiment, the apheresis may be performed prior to chemotherapy. Cumulative burden of CD19 expressing leukemic and normal B cells, as evaluated in the bone marrow prior to lymphodepleting chemotherapy may be important for determining CAR-T therapy outcome. According to Finney et al., increase antigen burden improves CAR-T therapy outcome. To increase CD19 antigen burden in vivo, subjects may also be infused with expanded subject derived T cells genetically modified to express CD19 (also referred to as T-APCs).

In some embodiments, regulatable transcription factor expression constructs, payloads of interest (e.g., immunotherapeutic agents), vectors, cells and compositions of the present disclosure may be used in conjunction with cancer vaccines.

In some embodiments, cancer vaccine may comprise peptides and/or proteins derived from tumor associated antigen (TAA). Such strategies may be utilized to evoke an immune response in a subject, which in some instances may be a cytotoxic T lymphocyte (CTL) response. Peptides used for cancer vaccines may also modified to match the mutation profile of a subject. For example, EGFR derived peptides with mutations matched to the mutations found in the subject in need of therapy have been successfully used in patients with lung cancer (Li F et al. (2016) Oncoimmunology. October 7; 5(12): e1238539; the contents of which are incorporated herein by reference in their entirety).

In one embodiment, cancer vaccines of the present disclosure may include superagonist altered peptide ligands (APL) derived from tumor associated antigens (TAAs). These are mutant peptide ligands deviate from the native peptide sequence by one or more amino acids, which activate specific CTL clones more effectively than native epitopes. These alterations may allow the peptide to bind better to the restricting Class I MHC molecule or interact more favorably with the TCR of a given tumor-specific CTL subset. APLs may be selected using methods taught in US Patent Publication NO. US20160317633A1, the contents of which are incorporated herein by reference in their entirety.

In some embodiments, effector immune cells genetically modified to encode the components of the transcription factor system, and payloads of the disclosure may be combined with the biological adjuvants described herein. Dual regulation of CAR and cytokines and ligands to segregate the kinetic control of target-mediated activation from intrinsic cell T cell expansion. Such dual regulation also minimizes the need for pre-conditioning regimens in patients. As a non-limiting example, DRD regulated transcription factors which transcribe a payload, for example, a CAR e.g. CD19 CAR may be combined with cytokines e.g. IL12 to enhance the anti-tumor efficacy of the CAR (Pegram H. J., et al. Tumor-targeted T cells modified to secrete IL12 eradicate systemic tumors without need for prior conditioning. Blood. 2012; 119:4133-41; the contents of each of which are incorporated herein by reference in their entirety). As another non-limiting example, Merchant et al. combined dendritic cell-based vaccinations with recombinant human IL7 to improve outcome in high-risk pediatric sarcomas patients (Merchant, M. S. et. al. Adjuvant immunotherapy to Improve Outcome in High-Risk Pediatric Sarcomas. Clin Cancer Res. 2016. 22(13):3182-91; the contents of each of which are incorporated herein by reference in their entirety).

In some embodiments, effector immune cells modified to express one or more antigen-specific TCRs or CARs may be combined with compositions of the disclosure comprising immunotherapeutic agents that convert the immunosuppressive tumor microenvironment.

In one aspect, effector immune cells modified to express CARs specific to different target molecules on the same cell may be combined. In another aspect, different immune cells modified to express the same CAR construct such as NK cells and T cells may be used in combination for a tumor treatment, for instance, a T cell modified to express a CD19 CAR may be combined with a NK cell modified to express the same CD19 CAR to treat B cell malignancy.

In other embodiments, immune cells modified to express CARs may be combined with checkpoint blockade agents.

In some embodiments, effector immune cells genetically modified to express one or more components of the transcription factor system, for example a payload of the disclosure, may be combined with cancer vaccines and other immunotherapeutics and adjuvant treatments of the disclosure.

In some embodiments, methods of the disclosure may include combination of the compositions of the disclosure with other agents effective in the treatment of cancers, infection diseases and other immunodeficient disorders, such as anti-cancer agents. As used herein, the term “anti-cancer agent” refers to any agent which is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

In some embodiments, anti-cancer agent or therapy may be a chemotherapeutic agent, or radiotherapy, immunotherapeutic agent, surgery, or any other therapeutic agent which, in combination with the present disclosure, improves the therapeutic efficacy of treatment.

In one embodiment, one or more components of a transcription factor system comprising a CD19 CAR may be used in combination with amino pyrimidine derivatives such as the Burkit's tyrosine receptor kinase (BTK) inhibitor using methods taught in International Patent Application NO. WO2016164580, the contents of which are incorporated herein by reference in their entirety.

In some embodiments, compositions of the present disclosure may be used in combination with immunotherapeutics other than the inventive therapy described herein, such as antibodies specific to some target molecules on the surface of a tumor cell.

Exemplary chemotherapies include, without limitation, Acivicin; Aclarubicin; Acodazole hydrochloride; Acronine; Adozelesin; Aldesleukin; Altretamine; Ambomycin; Ametantrone acetate; Amsacrine; Anastrozole; Anthramycin; Asparaginase; Asperrin, Sulindac, Curcumin, alkylating agents including: Nitrogen mustards such as mechlor-ethamine, cyclophosphamide, ifosfamide, melphalan and chlorambucil; nitrosoureas such as carmustine (BC U), lomustine (CCNU), and semustine (methyl-CC U); thylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrrolidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics, such as actimomycin D, daunomycin (rubidomycin), doxorubicin, mitoxantrone, idarubicin, bleomycins, plicamycin (mithramycin), mitomycinC, and actinomycin; enzymes such as L-asparaginase, cytokines such as interferon (IFN)-gamma, tumor necrosis factor (TNF)-alpha, TNF-beta and GM-CSF, anti-angiogenic factors, such as angiostatin and endostatin, inhibitors of FGF or VEGF such as soluble forms of receptors for angiogenic factors, including soluble VGF/VEGF receptors, platinum coordination complexes such as cisplatin and carboplatin, anthracenediones such as mitoxantrone, substituted urea such as hydroxyurea, methylhydrazine derivatives including N-methylhydrazine (MIFf) and procarbazine, adrenocortical suppressants such as mitotane (o,ρ′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; kinase inhibitors, histone deacetylase inhibitors, methylation inhibitors, proteasome inhibitors, monoclonal antibodies, oxidants, anti-oxidants, telomerase inhibitors, BH3 mimetics, ubiquitin ligase inhibitors, stat inhibitors and receptor tyrosin kinase inhibitors such as imatinib mesylate (marketed as Gleevac or Glivac) and erlotinib (an EGF receptor inhibitor) now marketed as Tarveca; anti-virals such as oseltamivir phosphate, Amphotericin B, and palivizumab; Sdi 1 mimetics; Semustine; Senescence derived inhibitor 1; Sparfosic acid; Spicamycin D; Spiromustine; Splenopentin; Spongistatin 1; Squalamine; Stipiamide; Stromelysin inhibitors; Sulfinosine; Superactive vasoactive intestinal peptide antagonist; Velaresol; Veramine; Verdins; Verteporfin; Vinorelbine; Vinxaltine; Vitaxin; Vorozole; Zanoterone; Zeniplatin; Zilascorb; and Zinostatin stimalamer; PI3Kβ small-molecule inhibitor, GSK2636771; pan-PI3K inhibitor (BKM120); BRAF inhibitors. Vemurafenib (Zelboraf) and dabrafenib (Tafinlar); or any analog or derivative and variant of the foregoing.

Radiotherapeutic agents and factors include radiation and waves that induce DNA damage for example, T-irradiation, X-rays, UV-irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

In some embodiments, the chemotherapeutic agent may be an immunomodulatory agent such as lenalidomide (LEN). Recent studies have demonstrated that lenalidomide can enhance antitumor functions of CAR modified T cells (Otahal et al., Oncoimmunology, 2015, 5(4): e1115940). Some examples of anti-tumor antibodies include tocilizumab, siltuximab.

Other agents may be used in combination with compositions of the disclosure may also include, but not limited to, agents that affect the upregulation of cell surface receptors and their ligands such as Fas/Fas ligand, DR4 or DR5/TRAIL and GAP junctions, cytostatic and differentiation agents, inhibitors of cell adhesion such as focal adhesion kinase (FAKs) inhibitors and Lovastatin, or agents that increase the sensitivity of the hyper proliferative cells to apoptotic inducers such as the antibody C225.

The combinations may include administering the compositions of the disclosure and other agents at the same time or separately. Alternatively, the present immunotherapy may precede or follow the other agent/therapy by intervals ranging from minutes, days, weeks to months.

Provided in the present disclosure is a method of reducing a tumor volume or burden in a subject in need, the method comprising introducing into the subject a composition of the disclosure.

The present disclosure also provides methods for treating a cancer in a subject, comprising administering to the subject an effective amount of effector immune cells genetically modified to comprise a transcription factor system of the present disclosure.

### Cancer

Various cancers may be treated with pharmaceutical compositions, transcription factor system components, regulatable transcription factor expression constructs including their DRDs or payloads of the present disclosure. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

Types of carcinomas which may be treated with the compositions of the present disclosure include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma.

Types of sarcomas which may be treated with the compositions of the present disclosure include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

### Infectious Diseases

In some embodiment, transcription factor system of the disclosure may be used for the treatment of infectious diseases. Transcription factor systems of the disclosure may be introduced in cells suitable for adoptive cell transfer such as macrophages, dendritic cells, natural killer cells, and or T cells. Infectious diseases treated by the transcription factor system of the disclosure may include diseases caused by viruses, bacteria, fungi, and/or parasites. IL15-IL15Ra payloads of the disclosure may be used to increase immune cell proliferation and/or persistence of the immune cells useful in treating infectious diseases.

“Infectious diseases” herein refer to diseases caused by any pathogen or agent that infects mammalian cells, preferably human cells and causes a disease condition. Examples thereof include bacteria, yeast, fungi, protozoans, mycoplasma, viruses, prions, and parasites. Examples include those involved in (a) viral diseases such as, for example, diseases resulting from infection by an adenovirus, a herpesvirus (e.g., HSV-I, HSV-II, CMV, or VZV), a poxvirus (e-g-, an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenza virus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV); (b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella; (c) other infectious diseases, such Chlamydia, fungal diseases including but not limited to candidiasis, aspergillosis, histoplasmosis, cryptococcal meningitis, parasitic diseases including but not limited to malaria, Pneumocystis carnii pneumonia, leishmaniasis, cryptosporidiosis, toxoplasmosis, and trypanosome infection and prions that cause human disease such as Creutzfeldt-Jakob Disease (CJD), variant Creutzfeldt-Jakob Disease (vCJD), Gerstmann-Straüssler-Scheinker syndrome, Fatal Familial Insomnia and kuru.

### Immuno-Oncology and Cell Therapies

Recent progress in the field of cancer immunology has allowed the development of several approaches to help the immune system keep the cancer at bay. Such immunotherapy approaches include the targeting of cancer antigens through monoclonal antibodies or through adoptive transfer of ex vivo engineered T cells (e.g., which contain chimeric antigen receptors or engineered T cell receptors).

In some embodiments, pharmaceutical compositions, transcription factor systems, regulatable transcription factor expression constructs, regulatable transcription factor expression construct components, regulatable transcription factor expression constructs including their payloads of the present disclosure may be used in the modulation or alteration or exploitation of the immune system to target one or more cancers. This approach may also be considered with other such biological approaches, e.g., immune response modifying therapies such as the administration of interferons, interleukins, colony-stimulating factors, other monoclonal antibodies, vaccines, gene therapy, and nonspecific immunomodulating agents are also envisioned as anti-cancer therapies to be combined with the pharmaceutical compositions, transcription factor systems, regulatable transcription factor expression constructs, regulatable transcription factor expression constructs components, regulatable transcription factor expression constructs including their payloads of the present disclosure.

Cancer immunotherapy refers to a diverse set of therapeutic strategies designed to induce the patient's own immune system to fight the cancer. In some embodiments, pharmaceutical compositions, transcription factor systems, regulatable transcription factor expression constructs, regulatable transcription factor expression construct components, regulatable transcription factor expression constructs including their transcription factors and/or payloads of the present disclosure are designed as immune-oncology therapeutics. Cell Therapies

There are several types of cellular immunotherapies, including tumor infiltrating lymphocyte (TIL) therapy, genetically engineered T cells bearing chimeric antigen receptors (CARs), and recombinant TCR technology.

According to the present disclosure, the transcription factor system may be used in the development and implementation of cell therapies such as adoptive cell therapy. The transcription factor system, and their transcription factors and payloads may be used in cell therapies to effect TCR removal-TCR gene disruption, TCR engineering, to regulate epitope tagged receptors, in APC platforms for stimulating T cells, as a tool to enhance ex vivo APC stimulation, to improve methods of T cell expansion, in ex vivo stimulation with antigen, in TCR/CAR combinations, in the manipulation or regulation of TILs, in allogeneic cell therapy, in combination T cell therapy with other treatment lines (e.g. radiation, cytokines), to encode engineered TCRs, or modified TCRs, or to enhance T cells other than TCRs (e.g. by introducing cytokine genes, genes for the checkpoint inhibitors PD1, CTLA4).

In some embodiments, improved response rates are obtained in support of cell therapies.

Expansion and persistence of cell populations may be achieved through regulation or fine tuning of the payloads, e.g., the receptors or pathway components in T cells, NK cells or other immune-related cells. In some embodiments, transcription factor systems of the present disclosure are designed to spatially and/or temporally control the expression of proteins which enhance T-cell or NK cell responses. In some embodiments, transcription factor systems are designed to spatially and/or temporally control the expression of proteins which inhibit T-cell or NK cell response.

In some embodiments, cells that have been genetically modified to comprise a transcription factor system as described herein may be designed to reduce, mitigate or eliminate the CAR cytokine storm. In some embodiments, such reduction, mitigation and/or elimination occurs in solid tumors or tumor microenvironments.

In some embodiments, the transcription factor system may encode one or more cytokines, for example, interleukins, such as, IL2, IL6, IL12, IL15 and IL21.

In one embodiment, the payload of the disclosure may comprise IL2. In one aspect, transcription factor systems of the disclosure may encode a transcription factor that selectively transcribes IL2, IL12, IL15 and other interleukin immunotherapeutic agents, which may be carefully tuned using the stabilizing ligands selective for the DRD used in the transcription factor systems.

In one aspect, the transcription factor systems of the disclosure may encode a transcription factor that selectively transcribes a payload, for example, an IL12 fusion polypeptide. Regulatable IL12 fusion polypeptides may be directly used as an immunotherapeutic agent or be transduced into an effector immune cell (T cells and TIL cells) to generate modified T cells with greater in vivo expansion and survival capabilities for adoptive cell transfer. The need for harsh preconditioning regimens in current adoptive cell therapies may be minimized using regulated IL12. IL12 may be utilized to modify tumor microenvironment and increase persistence in solid tumors that are currently refractory to tumor antigen targeted therapy. In some embodiments, CAR expressing T cells may be armored with transcription factor regulated IL12 to relieve immunosuppression without systemic toxicity.

In some embodiments, the IL12 may be a Flexi IL12, wherein both p35 and p40 subunits, are encoded by a single cDNA that produces a single chain polypeptide.

In some embodiments, an illustrative transcription factor systems may encode, or be tuned or induced to produce, one or more cytokines for expansion of cells of the disclosure. In such cases the cells may be tested for actual expansion. Expansion may be at least 5%, 10%, 20%, 30%, 40%, 50%, 60%, 70%, 80%, 90% or more. In some embodiments, the cytokine is IL15. Transcription factor systems encoding IL15 may be designed to induce proliferation in cytotoxic populations and avoid stimulation of T regs. In other cases, the transcription factor systems which induce proliferation in cytotoxic populations may also stimulate NK and NKT cells. Interleukin 15 is a potent immune stimulatory cytokine and an essential survival factor for T cells, and Natural Killer cells. Preclinical studies comparing IL2 and I15, have shown than IL15 is associated with less toxicity than IL2. In some embodiments, the transcription factor system of the disclosure may encode a IL15 fusion polypeptide. IL15 polypeptides may also be modified to increase its binding affinity for the I5 receptor. For example, the asparagine may be replaced by aspartic acid at position 72 of IL15 (SEQ ID NO. 2 of US patent publication US20140134128; the contents of which are incorporated by reference in their entirety).

The immune system can be harnessed for the treatment of diseases beyond cancer. Transcription factor systems, their components or regulatable transcription factor expression constructs may be utilized in immunotherapy for the treatment of diseases including, but not limited to, autoimmune diseases, allergies, graft versus host disease, and diseases and disorders that may result in immunodeficiency such as acquired immune deficiency syndrome (AIDS).

In some embodiments, payloads of the present disclosure may be a chimeric antigen receptor (CAR), which when transduced into immune cells (e.g., T cells and NK cells), can re-direct the immune cells against the target (e.g., a tumor cell) which expresses a molecule recognized by the extracellular target moiety of the CAR.

In some embodiments, pharmaceutical compositions comprising a transcription factor system, including their payloads or protein of interest may be used in the modulation or alteration or exploitation of the immune system to target one or more self-reactive immune components such as auto antibodies and self-reactive immune cells to attenuate autoimmune diseases.

In some embodiments, transcription factor systems may be utilized in immunotherapy-based treatments to attenuate or mitigate Graft vs. Host disease (GVHD). GVHD refers to a condition following stem cell or bone marrow transplant where in the allogeneic donor immune cells react against host tissue. In some embodiments, a transcription factor system may be designed to encode a cytokine or immunological agent designed to modulate Tregs for the treatment of GVHD.

In some embodiments, transcription factor systems may be significantly less immunogenic than other biocircuits or switches in the art due to the expression of human native or wild-type proteins of interest.

Various autoimmune diseases and autoimmune-related diseases may be treated with pharmaceutical compositions comprising a transcription factor systems of the present disclosure. As used herein, the term “autoimmune disease” refers to a disease in which the body produces antibodies that attack its own tissues.

Autoimmune diseases include, without limitation, Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosis, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatoid arthritis, Sarcoidosis, Schmidt syndrome, Scleritis, Scleroderma, Sjogren's syndrome, Sperm & testicular autoimmunity, Stiff person syndrome, Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis and Vitiligo.

Various kidney diseases may be treated with pharmaceutical compositions comprising one or more components of a transcription factor system of the present disclosure.

Various cardiovascular diseases may be treated with pharmaceutical compositions comprising one or more components of a transcription factor system of the present disclosure.

Various antibody deficiencies may be treated with pharmaceutical compositions comprising one or more components of a transcription factor system of the present disclosure.

Various neurological diseases may be treated with pharmaceutical compositions comprising one or more components of a transcription factor system of the present disclosure.

Various lung diseases may be treated with pharmaceutical compositions comprising one or more components of a transcription factor system of the present disclosure.

Various bone diseases may be treated with pharmaceutical compositions comprising one or more components of a transcription factor system of the present disclosure.

Various blood diseases may be treated with pharmaceutical compositions comprising one or more components of a transcription factor system of the present disclosure.

In some embodiments, pharmaceutical compositions comprising one or more components of a transcription factor system of the present disclosure may be used in the modulation or alteration or exploitation of proteins in the central nervous system including cerebrospinal (CSF) proteins.

In some examples, pharmaceutical compositions comprising one or more components of a transcription factor system of the present disclosure may be used to provide tunable ERT (enzyme replacement therapy) products to the central nervous system. Many lysosomal storage diseases (LSD) involve the CNS symptoms, such as mental retardation, seizures, profound neurodegeneration, behavioral abnormalities, and psycho-motor defects. ERT for LSDs is one of the true success stories in modern molecular medicine. The successful application of ERT relies on controlled lysosomal proteins (e.g., enzymes) and delivery to CNS cells.

### Gene Editing

In some embodiments, the transcription factor system includes a payload which comprises a nuclease that contains a DNA binding domain to selectively target specific DNA sequences for gene editing and gene therapy. In some embodiments, the transcription factor system comprises polynucleotides which encode zinc finger nucleases, TALES or CRISPR nucleases as payloads modulated by the transcription factor in the systems.

The CRISPR-Cas9 system is a novel genome editing system which has been rapidly developed and implemented in a multitude of model organisms and cell types, and supplants other genome editing technologies, such as TALENs and ZFNs. CRISPRs are sequence motifs are present in bacterial and archaeal genomes and are composed of short (about 24-48 nucleotide) direct repeats separated by similarly sized, unique spacers (Grissa et al. BMC Bioinformatics 8, 172 (2007)). They are generally flanked by a set of CRISPR-associated (Cas) protein-coding genes that are required for CRISPR maintenance and function (Barrangou et al., Science 315, 1709 (2007), Brouns et al., Science 321, 960 (2008), Haft et al. PLoS Comput Biol 1, e60 (2005)). CRISPR-Cas systems provide adaptive immunity against invasive genetic elements (e.g., viruses, phages and plasmids) (Horvath and Barrangou, Science, 2010, 327: 167-170; Bhaya et al., Annu. Rev. Genet., 2011, 45: 273-297; and Brrangou R, RNA, 2013, 4: 267-278). Three different types of CRISPR-Cas systems have been classified in bacteria and the type II CRISPR-Cas system is most studied. In the bacterial Type II CRISPR-Cas system, small CRISPR RNAs (crRNAs) processed from the pre-repeat-spacer transcript (pre-crRNA) in the presence of a trans-activating RNA (tracrRNA)/Cas9 can form a duplex with the tracrRNA/Cas9 complex. The mature complex is recruited to a target double strand DNA sequence that is complementary to the spacer sequence in the tracrRNA: crRNA duplex to cleave the target DNA by Cas9 endonuclease (Garneau et al., Nature, 2010, 468: 67-71; Jinek et al., Science, 2012, 337: 816-821; Gasiunas et al., Proc. Natl Acad. Sci. USA., 109: E2579-2586; and Haurwitz et al., Science, 2010, 329: 1355-1358). Target recognition and cleavage by the crRNA: tracrRNA/Cas9 complex in the type II CRISPR-CAS system not only requires a sequence in the tracrRNA: crRNA duplex that is complementary to the target sequence (also called “protospacer” sequence) but also requires a protospacer adjacent motif (PAM) sequence located 3′end of the protospacer sequence of a target polynucleotide. The PAM motif can vary between different CRISPR-Cas systems.

CRISPR-Cas9 systems have been developed and modified for use in genetic editing and prove to be a high effective and specific technology for editing a nucleic acid sequence even in eukaryotic cells.

However, controlling the effects and activity of the CRISPR-Cas system (e.g., guide RNA and nuclease) has been challenging and often can be problematic.

The transcription factor system of the present disclosure and/or any of their components may be utilized in regulating or tuning the CRISPR/Cas9 system in order to optimize its utility.

In some embodiments, the payloads of the regulatable transcription factor expression constructs of the disclosure may include alternative isoforms or orthologs of the Cas9 enzyme.

The most commonly used Cas9 is derived from Streptococcus pyogenes and the RuvC domain can be inactivated by a D10A mutation and the HNH domain can be inactivated by an H840A mutation. In addition to Cas9 derived from S. pyogenes, other RNA guided endonucleases (RGEN) may also be used for programmable genome editing. Cas9 sequences have been identified in more than 600 bacterial strains. Though Cas9 family shows high diversity of amino acid sequences and protein sizes, All Cas9 proteins share a common architecture with a central HNH nuclease domain and a split RuvC/RHase H domain.

In some embodiments, the payload of the present disclosure may be a split Cas-9 (Zetsche B et al. A split-Cas9 architecture for inducible genome editing and transcription modulation. Nat Biotechnol. 2015 February; 33(2):139-42; the contents of which are incorporated by reference in their entirety).

In addition to Cas9 orthologs, other Cas9 variants such as fusion proteins of inactive dCas9 and effector domains with different functions may serve as a platform for genetic modulation. Any of the foregoing enzymes may be useful in the present disclosure.

CRISPR/Cas9 based Regulatable transcription factor expression constructs may be generated by any of the methods taught in International Publication No.: WO2016106244 and Gao Y et al. Complex transcriptional modulation with orthogonal and inducible dCas9 regulators. Nat Methods. 2016 December; 13(12):1043-1049; the contents of each of which are incorporated herein by reference in their entirety).

The CRISPR/Cas9 system may also be utilized to modulate gene expression, which may be combined with its gene editing utility. In some embodiments, the payloads of the regulatable transcription factor system of the disclosure may include CRISPR associated transcriptional activators e.g. VP64-p65-Rta (VPR); associated with the CRISPR/Cas9 system.

### Additional Applications and Uses

Stem Cell Applications

The transcription factor system of the present disclosure and/or their components may be utilized in the regulated reprogramming of cells, stem cell engraftment or other application where controlled or tunable expression of such reprogramming factors are useful.

The regulatable transcription factor expression constructs of the present disclosure may be used in reprogramming cells including stem cells or induced stem cells. Induction of induced pluripotent stem cells (iPSC) was first achieved by Takahashi and Yamanaka (Cell, 2006. 126(4):663-76; herein incorporated by reference in its entirety) using viral vectors to express KLF4, c-MYC, OCT4 and SOX2 otherwise collectively known as KMOS.

Excisable lentiviral and transposon vectors, repeated application of transient plasmid, episomal and adenovirus vectors have also been used to try to derive iPSC (Chang, C.-W., et al., Stem Cells, 2009. 27(5):1042-1049; Kaji, K., et al., Nature, 2009. 458(7239):771-5; Okita, K., et al., Science, 2008. 322(5903):949-53; Stadtfeld, M., et al., Science, 2008. 322(5903):945-9; Woltjen, K., et al., Nature, 2009; Yu, J., et al., Science, 2009:1172482; Fusaki, N., et al., Proc Jpn Acad Ser B Phys Biol Sci, 2009. 85(8):348-62; each of which is herein incorporated by reference in its entirety).

DNA-free methods to generate human iPSC has also been derived using serial protein transduction with recombinant proteins incorporating cell-penetrating peptide moieties (Kim, D., et al., Cell Stem Cell, 2009. 4(6): 472-476; Zhou, H., et al., Cell Stem Cell, 2009. 4(5):381-4; each of which is herein incorporated by reference in its entirety), and infectious transgene delivery using the Sendai virus (Fusaki, N., et al., Proc Jpn Acad Ser B Phys Biol Sci, 2009. 85(8): p. 348-62; herein incorporated by reference in its entirety).

The regulatable transcription factor expression constructs of the present disclosure may include a payload comprising any of the genes including, but not limited to, OCT such as OCT4, SOX such as SOX1, SOX2, SOX3, SOX15 and SOX18, NANOG, KLF such as KLF1, KLF2, KLF4 and KLF5, MYC such as c-MYC and n-MYC, REM2, TERT and LIN28 and variants thereof in support of reprogramming cells. Sequences of such reprogramming factors are taught in for example International Application PCT/US2013/074560, the contents of which are incorporated herein by reference in their entirety.

The regulatable transcription factor expression constructs of the present disclosure may include a payload comprising any of factors that contribute stem cell mobilization. In autologous stem cell therapy, sources of stem cells for transplantation may include the bone marrow, peripheral blood mononuclear cells and cord blood. Stem cells are stimulated out of these sources (e.g., the bone marrow) into the blood stream. So sufficient stem cells are available for collection for future reinfusion. One or a combination of cytokines strategies may be used to mobilize the stem cells including but not limited to G-CSF (filgrastim), GM-CSF, and chemotherapy preceding with cytokines (chemomobilization).

Metabolic Peptides and Hormones

In some embodiments, the transcription factor systems of the present disclosure and/or any of their components may be used to regulate peptides, natural or synthetic. Naturally occurring peptides may include but are not limited to, peptide hormones, natriuretic peptides, food peptides, and derivatives and precursors.

The transcription factor systems of the present disclosure and/or any of their components may also be utilized for pulsatile release of hormones or other peptide drugs.

Enzyme Replacement Therapy (ERT)

Enzyme replacement therapy (ERT) is a medical treatment replacing an enzyme in a patient. ERT provides therapeutic interventions that address the underlying metabolic defect in many disorders caused by defective enzymes. Such disorders include, but are not limited to, lysosomal storage diseases (LSDs), congenital disorders of glycosylation, and metabolic disorders characterized by missing or reduced enzyme activity in the cytoplasm.

Coagulation

Coagulation defects often cause hemorrhage and/or thrombosis. The best-known coagulation factor disorders are the hemophilias. The three main forms are hemophilia A (factor VIII deficiency), hemophilia B (factor IX deficiency or “Christmas disease”) and hemophilia C (factor XI deficiency, mild bleeding tendency). Other disorders caused by defective coagulation factors also include, but are not limited to, Von Willebrand disease (caused by a defect in von Willebrand factor (vWF), Bernard-Soulier syndrome (caused by a defect or deficiency in GPIb, a receptor of vWF), thrombophlebitis (caused by mutations in Factor XII), Congenital afibrinogenemia, Familial renal amyloidosis (caused by mutations in Factor I), congenital proconvertin/factor VII deficiency, Thrombophilia (caused by Factor II deficiency), Congenital Factor X deficiency, Congenital Factor XIIIa/b deficiency, Prekallikrein/Fletcher Factor deficiency, Kininogen deficiency, Glomerulopathy with fibronectin deposits, Heparin cofactor II deficiency, Protein C deficiency, Protein S deficiency, Protein Z deficiency, Antithrombin III deficiency, Plasminogen deficiency, type I (ligneous conjunctivitis), Antiplasmin deficiency, Plasminogen activator inhibitor-1 deficiency, and Quebec platelet disorder.

Gene therapy for coagulation factor replacement is a medical treatment of disorders caused be coagulation deficiency. In accordance with the present disclosure, the regulatable transcription factor expression constructs of the present disclosure and/or any of their components may also be utilized to regulate a coagulation factor used for gene therapy. In some examples, the coagulation factor may be selected from Factor I (fibrinogen), Factor II (prothrombin), Factor III (tissue factor), Factor IV, Factor V (proaccelerin), Factor VI, Factor VII (stable factor), Factor VIII (antihemophilic factor A), Factor IX (antihemophilic factor B), Factor X (Stuart-Prower factor), Factor XI (plasma thromboplastin antecedent), Factor XII (Hageman factor), Factor XIII (fibrin-stabilizing factor), von Willebrand factor, Prekallikrein (Fletcher factor), high-molecular-weight kininogen (HMWK) (Fitzgerald factor), fibronectin, antithrombin III, heparin cofactor II, protein C, protein S, protein Z, protein Z related protease inhibitor (ZPI), plasminogen, tissue plasminogen activator (tPA), urokiase, plasminogen, plasminogen activator inhibitor 1 (PAI1), and plasminogen activator inhibitor 2 (PAI2)

In one embodiment, the coagulate factor is Factor VIII for gene therapy of hemophilia, including wild type factor VIII, engineered Factor VIII, activated fVIII (fVIIIa), or the equivalent. Exemplary engineered Factor VIII may include those discussed by Roberts et al (J. Genet. Syndr. Gene Ther., 2011, 1: S1-006; the contents of which are incorporated herein by reference in their entirety).

In another embodiment, the coagulate factor may be Factor IX for gene therapy of hemophilia B. The factor IX may be a recombinant factor IX as disclosed in U.S. Pat. Nos. 7,575,897; 7,700,734; 7,888,067; and 8, 168,425; PCT patent application publication NO.. WO2016/075473; the contents of each of which are incorporated herein by reference in their entirety.

In some embodiments, the regulatable transcription factor expression constructs of the present disclosure and/or any of their components may comprise any of factors that play a role in protein processing and modification. Protein post-translational modification may include, but are not limited to, addition of hydrophobic groups by an enzyme (e.g., myristoylation, palmitoylation, isoprenylation, prenylation, farnesylation, geranylgeranylation, glypiation, and glycosylphosphatidylinositol (GPI) anchor); attachment of cofactors for enhanced function (e.g., lipoylation, flavin, phosphopantetheinylation, and heme C); addition of small chemical groups (e.g., acylation, formylation, alkylation, phosphorylation, methylation, arginylation, polyglutamylation, polyglycylation, butyrylation, glycosylation, propionylation, S-glutathionylation, S-nitrosylation, S-sulfenylation, succinylation, sulfation, and acetylation); linkage of other proteins and/or peptides such as ISGylation, SUMOylation, ubiquitination, neddylation, and pupylation; chemical modification of amino acids; and structural changes.

Liver Targeting

The liver is an important organ that produces proteins and involves blood clotting and a number of metabolic functions. A variety of diseases can affect liver and targeting the liver for disease treatment has been a promising approach, especially liver-targeted gene therapy. In some embodiments, the regulatable transcription factor expression constructs of the present disclosure and/or any of their components may be utilized to regulate liver targeted gene therapy and gene transfer.

Proteins that can be targeted to the liver and constructed to the present regulatable transcription factor expression constructs for regulation may include those in liver cancers such as hepatocellular carcinoma (HCC), Fibrolamellar HCC, Cholangiocarcinoma, Angiosarcoma and secondary liver cancer; inherited disorders caused by defective genes such as hemochromatosis, Wilson disease, tyrosinemia, alpha 1 antitrypsin deficiency, glycogen storage disease; metabolic disorders due to enzyme deficiency such as Gilbert's syndrome, lysosomal acid lipase deficiency (LALD) and Gaucher disease; autoimmune hepatitis; fatty liver diseases; and viral hepatitis (A, B and C). In some examples, the present Regulatable transcription factor expression constructs may be used to direct IL12 for hepatocellular carcinoma (HCC), and IL10 for diabetic neuropathy.

In some embodiments, the present regulatable transcription factor expression constructs may be used to control liver specific gene products for gene therapy.

In some embodiments, the present regulatable transcription factor expression constructs may be used to control liver proteins that are secreted (e.g., to blood).

Tools and Agents for Making Therapeutics

Provided in the present disclosure are tools and agents that may be used in generating therapeutics such as, but not limited to, immunotherapeutics for reducing a tumor volume or burden in a subject in need. A considerable number of variables are involved in producing a therapeutic agent, such as structure of the payload, type of cells, method of gene transfers, method and time of ex vivo expansion, pre-conditioning and the amount and type of tumor burden in the subject. Such parameters may be optimized using tools and agents described herein.

Cell Lines

The present disclosure provides a mammalian cell that has been genetically modified with the compositions of the disclosure. Suitable mammalian cells include primary cells and immortalized cell lines. Suitable mammalian cell lines include but are not limited to Human embryonic kidney cell line 293, fibroblast cell line NIH 3T3, human colorectal carcinoma cell line HCT116, ovarian carcinoma cell line SKOV-3, immortalized T cell lines (e.g. Jurkat cells and SupT1 cells), lymphoma cell line Raji cells, NALM-6 cells, K562 cells, HeLa cells, PC12 cells, HL-60 cells, NK cell lines (e.g. NKL, NK92, NK962, and YTS), and the like. In some instances, the cell is not an immortalized cell line, but instead a cell obtained from an individual and is herein referred to as a primary cell. For example, the cell is a T lymphocyte obtained from an individual. Other examples include, but are not limited to cytotoxic cells, stem cells, peripheral blood mononuclear cells or progenitor cells obtained from an individual.

Cellular Assays

In some embodiments, the effectiveness of the compositions of the disclosures as immunotherapeutic agents may be evaluated using cellular assays. Levels of expression and/or identity of the compositions of the disclosure may be determined according to any methods known in the art for identifying proteins and/or quantitating proteins levels. In some embodiments, such methods may include Western Blotting, flow cytometry, and immunoassays.

Provided herein are methods for functionally characterizing cells transfected or transduced with a regulatable transcription factor expression construct of the present disclosure and compositions of the disclosure. In some embodiments, functional characterization is carried out in primary immune cells or immortalized immune cell lines and may be determined by expression of cell surface markers. Examples of cell surface markers for T cells include, but are not limited to, CD3, CD4, CD8, CD 14, CD20, CD11b, CD16, CD45 and HLA-DR, CD 69, CD28, CD44, IFNgamma. Markers for T cell exhaustion include PD1, TIM3, BTLA, CD160, 2B4, CD39, and LAG3. Examples of cell surface markers for antigen presenting cells include, but are not limited to, MHC class I, MHC Class II, CD40, CD45, B7-1, B7-2, IFN γ receptor and IL2 receptor, ICAM-1 and/or Fcγ receptor. Examples of cell surface markers for dendritic cells include, but are not limited to, MHC class I, MHC Class II, B7-2, CD18, CD29, CD31, CD43, CD44, CD45, CD54, CD58, CD83, CD86, CMRF-44, CMRF-56, DCIR and/or Dectin-1 and the like; while in some cases also having the absence of CD2, CD3, CD4, CD8, CD14, CD15, CD16, CD 19, CD20, CD56, and/or CD57. Examples of cell surface markers for NK cells include, but are not limited to, CCL3, CCL4, CCL5, CCR4, CXCR4, CXCR3, NKG2D, CD71, CD69, CCR5, Phospho JAK/STAT, phospho ERK, phospho p38/MAPK, phospho AKT, phospho STAT3, Granulysin, Granzyme B, Granzyme K, IL10, IL22, IFNg, LAP, Perforin, and TNFα.

In some embodiments, any of the strategies disclosed by Long H A et al. may be utilized to prevent exhaustion (Long A H et al. (2015) Nature Medicine 21, 581-590; the contents of which are incorporated herein by reference in their entirety). In some embodiments, T cell metabolic pathways may be modified to diminish the susceptibility of T cells to exhaustion. Metabolic pathways may include, but are not limited to glycolysis, urea cycle, citric acid cycle, beta oxidation, fatty acid biosynthesis, pentose phosphate pathway, nucleotide biosynthesis, and glycogen metabolic pathways. As a non-limiting example, payloads that reduce the rate of glycolysis may be utilized to restrict or prevent T cell exhaustion (Long et al. Journal for Immunotherapy of Cancer 2013, 1(Suppl 1): P21; the contents of which are incorporated by reference in their entirety). In one embodiment, T cells of the present disclosure may be used in combination with inhibitors of glycolysis such as 2-deoxyglucose, and rapamycin.

In some embodiments, regulatable transcription factor expression constructs of the present disclosure, useful for immunotherapy may be placed under the transcriptional control of the T cell receptor alpha locus constant (TRAC) locus in the T cells. Eyquem et al. have shown that expression of the CAR from the TRAC locus prevents T cell exhaustion and the accelerated differentiation of T cells caused by excessive T cell activation (Eyquem J. et al (2017) Nature. 543(7643):113-117; the contents of which are incorporated herein by reference in their entirety).

In some embodiments, payloads of the disclosure may include, antibodies or fragments that target T cell surface markers associated with T cell exhaustion. T-cell surface markers associated with T cell exhaustion that may be used as payloads include, but are not limited to, CTLA-1, PD-1, TGIT, LAG-3, 2B4, BTLA, TIM3, VISTA, and CD96.

In one embodiment, the payload of the disclosure may be a CD276 CAR (with CD28, 4-IBB, and CD3 zeta intracellular domains), that does not show an upregulation of the markers associated with early T cell exhaustion (see International patent publication No. WO2017044699; the contents of which are incorporated by reference in their entirety).

Cells

In accordance with the present disclosure, cells genetically modified to express at least one protein of interest or payload under the regulation of the encoded transcription factor and DRD ligand of the disclosure, are provided. Cells of the disclosure may include, without limitation, immune cells, stem cells and tumor cells. In some embodiments, immune cells are effector immune cells, including, but not limiting to, T cells such as CD8+ T cells and CD4+ T cells (e.g., Th1, Th2, Th17, Foxp3+ cells), memory T cells such as T memory stem cells, central T memory cells, and effector memory T cells, terminally differentiated effector T cells, natural killer (NK) cells, NK T cells, tumor infiltrating lymphocytes (TILs), cytotoxic T lymphocytes (CTLs), regulatory T cells (Tregs), and dendritic cells (DCs), other immune cells that can elicit an effector function, or the mixture thereof. T cells may be Tap cells and T76 cells. In some embodiments, stem cells may be from human embryonic stem cells, mesenchymal stem cells, and neural stem cells. In some embodiments, T cells may be depleted endogenous T cell receptors (See U.S. Pat. Nos. 9,273,283; 9,181,527; and 9,028,812; the contents of each of which are incorporated herein by reference in their entirety).

In some embodiments, cells of the disclosure may be autologous, allogeneic, syngeneic, or xenogeneic in relation to a particular individual subject.

In some embodiments, cells of the disclosure may be mammalian cells, particularly human cells. Cells of the disclosure may be primary cells or immortalized cell lines.

Engineered immune cells can be accomplished by method comprising introducing into a cell a nucleic acid molecule comprising:

a. a first nucleic acid sequence that encodes at least one of a transcription factor DNA binding domain and a transcription factor activation domain; and

b. a second nucleic acid sequence that encodes a drug responsive domain (DRD). In some embodiments, the cell may also be genetically modified to insert a third nucleic acid sequence encoding a protein of interest operably linked to an inducible promoter comprising the transcription factor polynucleotide binding site. In some embodiments, the third nucleic acid sequence is on the same nucleic acid molecule as the first and second nucleic acid sequences.

Alternatively, the third nucleic acid sequence is on a different nucleic acid molecule as the first and second nucleic acid sequences. As used herein the first nucleic acid sequence and the second nucleic acid sequence can represent a first and second polynucleotides on one vector or each polynucleotide in separate vectors.

The vector may be a viral vector such as a lentiviral vector, a gamma-retroviral vector, a recombinant AAV, an adenoviral vector and an oncolytic viral vector. In other aspects, non-viral vectors for example, nanoparticles and liposomes may also be used. In some embodiments, immune cells of the disclosure are genetically modified to express at least one immunotherapeutic agent of the disclosure which is tunable using a stabilizing ligand. In some examples, two, three or more immunotherapeutic agents constructed in the same regulatable transcription factor expression constructs are introduced into a cell. In other examples, two, three, or more regulatable transcription factor expression constructs may be introduced into a cell.

In some embodiments, immune cells of the disclosure may be NK cells modified to express an antigen-specific T cell receptor (TCR), or an antigen specific chimeric antigen receptor (CAR) taught herein.

Natural killer (NK) cells are members of the innate lymphoid cell family and characterized in humans by expression of the phenotypic marker CD56 (neural cell adhesion molecule) in the absence of CD3 (T-cell co-receptor). NK cells are potent effector cells of the innate immune system which mediate cytotoxic attack without the requirement of prior antigen priming, forming the first line of defense against diseases including cancer malignancies and viral infection.

Several pre-clinical and clinical trials have demonstrated that adoptive transfer of NK cells is a promising treatment approach against cancers such as acute myeloid leukemia (Ruggeri et al., Science; 2002, 295: 2097-2100; and Geller et al., Immunotherapy, 2011, 3: 1445-1459). Adoptive transfer of NK cells expressing CAR such as DAP12-Based Activating CAR revealed improved eradication of tumor cells (Topfer et al., J Immunol. 2015; 194:3201-3212). NK cell engineered to express a CS-1 specific CAR also displayed enhanced cytolysis and interferon-y (IFN-y) production in multiple myeloma (Chu et al., Leukemia, 2014, 28(4): 917-927).

NK cell activation is characterized by an array of receptors with activating and inhibitory functions. The important activation receptors on NK cells include CD94/NKG2C and NKG2D (the C-type lectin-like receptors), and the natural cytotoxicity receptors (NCR) NKp30, NKp44 and NKp46, which recognize ligands on tumor cells or virally infected cells. NK cell inhibition is essentially mediated by interactions of the polymorphic inhibitory killer cell immunoglobulin-like receptors (KIRs) with their cognate human-leukocyte-antigen (HLA) ligands via the alpha-1 helix of the HLA molecule. The balance between signals that are generated from activating receptors and inhibitory receptors mainly determines the immediate cytotoxic activation.

NK cells may be isolated from peripheral blood mononuclear cells (PBMCs) or derived from human embryonic stem (ES) cells and induced pluripotent stem cells (iPSCs). The primary NK cells isolated from PBMCs may be further expanded for adoptive immunotherapy. Strategies and protocols useful for the expansion of NK cells may include interleukin 2 (IL2) stimulation and the use of autologous feeder cells, or the use of genetically modified allogeneic feeder cells. In some aspects, NK cells can be selectively expanded with a combination of stimulating ligands including IL15, IL21, IL2, 41BBL, IL12, IL18, MICA, 2B4, LFA-1, and BCM1/SLAMF2 (e.g., US patent publication NO. US20150190471).

### Definitions

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference and understanding, and the inclusion of such definitions herein should not necessarily be construed to mean a substantial difference over what is generally understood in the art. Commonly understood definitions of molecular biology terms and/or methods and/or protocols can be found in Rieger et al., Glossary of Genetics: Classical and Molecular, 5th edition, Springer-Verlag: New York, I991; Lewin, Genes V, Oxford University Press: New York, I994; Sambrook et al., Molecular Cloning, A Laboratory Manual (3d ed. 2001) and Ausubel et al., Current Protocols in Molecular Biology (1994), Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929.

As appropriate, procedures involving the use of commercially available kits and/or reagents are generally carried out in accordance with manufacturer's guidance and/or protocols and/or parameters unless otherwise noted.

“Affinity” refers to the strength of binding: increased binding affinity being correlated with a lower Kd.

The terms “Adoptive cell therapy” or “Adoptive cell transfer”, as used herein, refer to a cell therapy involving the transfer of cells into a patient, wherein cells may have originated from the patient, or from another individual, and are engineered (altered) before being transferred back into the patient. The therapeutic cells may be derived from the immune system, such as effector immune cells: CD4+ T cell; CD8+ T cell, Natural Killer cell (NK cell); and B cells and tumor infiltrating lymphocytes (TILs) derived from the resected tumors. Most commonly transferred cells are autologous anti-tumor T cells after ex vivo expansion or manipulation. For example, autologous peripheral blood lymphocytes can be genetically engineered to recognize specific tumor antigens by expressing T-cell receptors (TCR) or chimeric antigen receptor (CAR).

As used herein, the term “agent” refers to a biological, pharmaceutical, or chemical compound. Non-limiting examples include simple or complex organic or inorganic molecule, a peptide, a protein, an oligonucleotide, an antibody, an antibody derivative, antibody fragment, a receptor, and soluble factor.

The term “agonist” as used herein, refers to a compound that, in combination with a receptor, can produce a cellular response. An agonist may be a ligand that directly binds to the receptor. Alternatively, an agonist may combine with a receptor indirectly by, for example, (a) forming a complex with another molecule that directly binds to the receptor, or (b) otherwise resulting in the modification of another compound so that the other compound directly binds to the receptor. An agonist may be referred to as an agonist of a particular receptor or family of receptors, e.g., agonist of a co-stimulatory receptor.

The term “antagonist” as used herein refers to any agent that inhibits or reduces the biological activity of the target(s) it binds.

As used herein, the term “approximately” or “about,” as applied to one or more values of interest, refers to a value that is similar to a stated reference value. In certain embodiments, the term “approximately” or “about” refers to a range of values that fall within 25, 20, 19, 18, 17, 16, 15, 14, 13, 12, 11, 10, 9, 8, 7, 6, 5, 4, 3, 2, 1, or less in either direction (greater than or less than) of the stated reference value unless otherwise stated or otherwise evident from the context.

As used herein, the terms “associated with,” “conjugated,” “linked,” “attached,” and “tethered,” when used with respect to two or more moieties, mean that the moieties are physically associated or connected with one another, either directly or via one or more additional moieties that serve as linking agents, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

The term “autologous” as used herein is meant to refer to any material derived from the same individual to which it is later to be re-introduced into the individual.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (Kd) of 10-6 M or lower.

A “binding protein” is a protein that is able to bind to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

The terms “cassette,” “expression cassette” and “gene expression cassette” refer to a segment of DNA that can be inserted into a nucleic acid or polynucleotide at specific sites (e.g., restriction sites or by homologous recombination). The segment of DNA comprises a polynucleotide that encodes a polypeptide of interest, and the cassette and restriction sites are designed to ensure insertion of the cassette in the proper reading frame for transcription and translation. Cassettes, expression cassettes, and gene expression cassettes may also comprise elements that allow for enhanced expression of a polynucleotide encoding a polypeptide of interest in a host cell. These elements may include, but are not limited to: a promoter, a minimal promoter, an enhancer, a response element, a terminator sequence, a polyadenylation sequence, and the like.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A coding sequence is “under the control” of transcriptional and translational control sequences in a cell when RNA polymerase transcribes the coding sequence into mRNA, which is then RNA spliced (if the coding sequence contains introns) and translated into the protein encoded by the coding sequence.

The term “construct” and “nucleic acid construct” are used interchangeably and refer to a polynucleotide comprising a nucleic acid sequence encoding one or more of a peptide, polypeptide or protein. A “construct” may be any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication. Constructs may include but are not limited to additional regulatory nucleic acid elements from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid elements may be derived from a source that is native or heterologous with respect to the other elements present on the construct.

The term “cytokines”, as used herein, refers to a family of small soluble factors with pleiotropic functions that are produced by many cell types that can influence and regulate the function of the immune system.

The term “delivery” as used herein refers to the act or manner of delivering a compound, substance, entity, moiety, cargo or payload. A “delivery agent” refers to any agent which facilitates, at least in part, the delivery of one or more substances (including, but not limited to a compound and/or composition of the present disclosure) to a cell, subject or other biological system.

As used herein, the phrase “derived from” refers to a polypeptide or polynucleotide that originates from the stated parent molecule or region or domain thereof or the stated parent sequence (e.g., nucleic acid sequence or amino acid sequence) and retains similarity to one or more structural and/or functional characteristics of the parent molecule or region or domain thereof or parent sequence. A parent molecule may be a polypeptide or a nucleic acid molecule. For example, a parent molecule may be a native protein (comprising a native amino acid sequence) or a wild-type protein, and may be referred to as a “parent protein”. As another example, a parent molecule may be a nucleic acid molecule comprising a native nucleic acid sequence or a sequence that encodes a wild-type protein. In some embodiments, a polypeptide or polynucleotide is derived from either (i) a full-length wild-type parent molecule or sequence; or (ii) a region or domain of a full-length wild-type parent molecule or sequence and retains the structural and/or functional characteristics of either (i) the full-length wild-type parent molecule or sequence; or (ii) the region or domain thereof, respectively. Structural characteristics include an amino acid sequence, a nucleic acid sequence, or a protein structure (e.g., such as a secondary protein structure, a tertiary protein structure, and/or quaternary protein structure). Functional characteristics include biological activity such as catalytic activity, binding ability, and/or subcellular localization. As a non-limiting example, a polypeptide or polynucleotide retains similarity to a parent molecule or sequence if it has at least about 70% identity, preferably at least about 75% or 80% identity, more preferably at least about 85%, 86%, 87%, 88%, 89% or 90% identity, and further preferably at least about 91%, 92%, 93%, 94%, 95%, 96%, 97%, 98% or 99% identity to a parent nucleic acid sequence or amino acid sequence, over the entire length of the parent molecule or sequence. As another non-limiting example, a polypeptide retains similarity to a parent molecule or sequence if it comprises a region of amino acids that shares 100% identity to a parent amino acid sequence and the region ranges from 10-1,000 amino acids in length (e.g., greater than 20, 30, 40, 45, 50, 55, 60, 70, 80, 90, 100, 120, 140, 160, 180, 200, 250, 300, 350, 400, 450, 500, 600, 700, 800, and 900 amino acids or at least 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, and 1,000 amino acids). As another non-limiting example, a polypeptide retains similarity to a parent molecule or amino acid sequence if it comprises one, two, three, four, or five amino acid mutations as compared to the parent amino acid sequence. In some embodiments, a polypeptide or polynucleotide is considered to retain similarity to a parent molecule or region or domain thereof or a parent sequence if it has substantially the same biological activity as compared to the parent molecule or region or domain thereof or the parent sequence. In some embodiments, a polypeptide or polynucleotide is considered to retain similarity to a parent molecule or region or domain thereof or a parent sequence if there is overlap of at least one biological activity as compared to the parent molecule or region or domain thereof or parent sequence. In some embodiments, a polypeptide or polynucleotide is considered to retain similarity to a parent molecule or region or domain thereof or a parent sequence if it has improvement or optimization of one or more biological activities as compared to the parent molecule or region or domain thereof or parent sequence. For example, a DRD may be derived from a domain or region of a naturally occurring protein and is modified in any of the ways taught herein to optimize DRD function. In some embodiments, biological activity may be optimized for a specified purpose, such as by retaining or enhancing certain activity while reducing or eliminating another activity as compared to a parent molecule. In some embodiments, a DRD that is derived from the stated parent molecule or region or domain thereof or the stated parent sequence is a variant of the stated parent molecule or region or domain thereof or the stated parent sequence. For example, in some embodiments, a DRD derived from human carbonic anhydrase 2 (hCA2) is a variant of hCA2.

As used herein, the term “destable,” “destabilize,” “destabilizing region” or “destabilizing domain” means a region or molecule that is less stable than a starting, reference, wild-type or native form of the same region or molecule.

A DNA “coding sequence” or “coding region” refers to a double-stranded DNA sequence that encodes a polypeptide and can be transcribed and translated into a polypeptide in a cell, ex vivo, in vitro or in vivo when placed under the control of suitable regulatory sequences. “Suitable regulatory sequences” refers to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites and stem-loop structures. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from mRNA, genomic DNA sequences, and even synthetic DNA sequences. If the coding sequence is intended for expression in an eukaryotic cell, a polyadenylation signal and transcription termination sequence will usually be located 3′ to the coding sequence.

The term “downstream” refers to a nucleotide sequence that is located 3′ to a reference nucleotide sequence. In particular, downstream nucleotide sequences generally relate to sequences that follow the starting point of transcription. For example, the translation initiation codon of a gene is located downstream of the start site of transcription.

The term “upstream” refers to a nucleotide sequence that is located 5′ to a reference nucleotide sequence. In particular, upstream nucleotide sequences generally relate to sequences that are located on the 5′ side of a coding sequence or starting point of transcription. For example, most promoters are located upstream of the start site of transcription.

As used herein, embodiments of the disclosure are “engineered” when they are designed to have a feature or property, whether structural or chemical, that varies from a starting point, wild type or native molecule.

An “exogenous” molecule is a molecule that is not normally present in a cell but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylates, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

The exogenous nucleic acid sequence can comprise, for example, one or more genes or cDNA molecules, or any type of coding or noncoding sequence, as well as one or more control elements (e.g., promoters). In addition, the exogenous nucleic acid sequence may produce one or more RNA molecules (e.g., small hairpin RNAs (shRNAs), inhibitory RNAs (RNAis), microRNAs (miRNAs), etc.).

By contrast, an “endogenous” molecule is one that is normally present in a particular cell at a particular developmental stage under particular environmental conditions. For example, an endogenous nucleic acid can comprise a chromosome, the genome of a mitochondrion, or other organelle, or a naturally occurring episomal nucleic acid. Additional endogenous molecules can include proteins, for example, transcription factors and enzymes.

An “episome” is a replicating nucleic acid, nucleoprotein complex or other structure comprising a nucleic acid that is not part of the chromosomal karyotype of a cell. Examples of episomes include plasmids and certain viral genomes.

“Eukaryotic” cells include, but are not limited to, fungal cells (such as yeast), plant cells, animal cells, mammalian cells and human cells (e.g., T-cells).

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

The term “fragment,” as applied to polynucleotide sequences, refers to a nucleotide sequence of reduced length relative to the reference nucleic acid and comprising, over the common portion, a nucleotide sequence identical to the reference nucleic acid. Such a nucleic acid fragment according to the invention may be, where appropriate, included in a larger polynucleotide of which it is a constituent. Such fragments comprise, or alternatively consist of, oligonucleotides ranging in length from at least 6, 8, 9, 10, 12, 15, 18, 20, 21, 22, 23, 24, 25, 30, 39, 40, 42, 45, 48, 50, 51, 54, 57, 60, 63, 66, 70, 75, 78, 80, 90, 100, 105, 120, 135, 150, 200, 300, 500, 720, 900, 1000, 1500, 2000, 3000, 4000, 5000, or more consecutive nucleotides of a nucleic acid according to the invention.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

As used herein, a “functional” biological molecule is a biological entity with a structure and in a form in which it exhibits a property and/or activity by which it is characterized.

A “fusion” molecule is a molecule in which two or more subunit molecules are linked, preferably covalently. The subunit molecules can be the same chemical type of molecule or can be different chemical types of molecules. Examples of the first type of fusion molecule include, but are not limited to, fusion proteins, for example, a fusion between a DNA-binding domain (e.g., ZFP, TALE and/or meganuclease DNA-binding domains) and a nuclease (cleavage) domain (e.g., endonuclease, meganuclease, etc.) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. RNA splicing, polypeptide cleavage and polypeptide ligation can also be involved in expression of a protein in a cell. Methods for polynucleotide and polypeptide delivery to cells are presented elsewhere in this disclosure.

A “gene” refers to a polynucleotide comprising nucleotides that encode a functional molecule including functional molecules produced by transcription only (e.g., a bioactive RNA species) or by transcription and translation (e.g., a polypeptide). The term “gene” encompasses cDNA and genomic DNA nucleic acids. “Gene” also refers to a nucleic acid fragment that expresses a specific RNA, protein or polypeptide, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and/or coding sequences that are not found together in nature. Accordingly, a chimeric gene may comprise regulatory sequences and coding sequences that are derived from different sources, or regulatory sequences and coding sequences derived from the same source but arranged in a manner different than that found in nature. A chimeric gene may comprise coding sequences derived from different sources and/or regulatory sequences derived from different sources. “Endogenous gene” refers to a native gene in its natural location in the genome of an organism. A “foreign” gene or “heterologous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome. For example, the interleukin-12 (IL-12) gene encodes the IL-12 protein. IL-12 is a heterodimer of a 35-kD subunit (p35) and a 40-kD subunit (p40) linked through a disulfide linkage to make fully functional IL-12p70. The IL-12 gene encodes both the p35 and p40 subunits.

The transcribed polynucleotide can have a sequence encoding a polypeptide, such as a functional protein, which can be translated into the encoded polypeptide when placed under the control of an appropriate regulatory region. A gene may comprise several operably linked fragments, such as a promoter, a 5′ leader sequence, a coding sequence and a 3′ nontranslated sequence, such as a polyadenylation site, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

A chimeric or recombinant gene is a gene not normally found in nature, such as a gene in which, for example, the promoter is not associated in nature with part or all of the transcribed DNA region. “Expression of a gene” refers to the process wherein a gene is transcribed into an RNA and/or translated into a functional protein.

“Gene delivery” or “gene transfer” refers to methods for introduction of recombinant or foreign DNA into host cells. The transferred DNA can remain non-integrated or preferably integrates into the genome of the host cell. Gene delivery can take place for example by transduction, using viral vectors, or by transformation or transfection of cells, using known methods, such as electroporation, cell bombardment.

The term “genome” includes chromosomal as well as mitochondrial, chloroplast and viral DNA or RNA.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “heterologous protein” (used interchangeably with “exogenous protein”) or “heterologous protein of interest” is a protein or protein of interest, respectively, that is encoded by a heterologous nucleic acid. A “homologous” DNA sequence is a DNA sequence that is naturally associated with a host cell into which it is introduced.

“Heterologous DNA” refers to DNA not naturally located in the cell, or in a chromosomal site of the cell. The heterologous DNA may include a gene foreign to the cell.

The term “an immune cell”, as used herein, refers to any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells). Exemplary immune system cells include a CD4+ T cell, a CD8+ T cell, a CD4− CD8− double negative T cell, a T γδ cell, a Tαβ cell, a regulatory T cell, a natural killer cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

The term “immunotherapy” as used herein, refers to a type of treatment of a disease by the induction or restoration of the reactivity of the immune system towards the disease.

The term “immunotherapeutic agent” as used herein, refers to a biological, pharmaceutical, or chemical compound that may be used for immunotherapy.

The term “isolated” for the purposes of the invention designates a biological material (cell, nucleic acid or protein) that has been removed from its original environment (the environment in which it is naturally present). For example, a polynucleotide present in the natural state in a plant or an animal is not isolated, however the same polynucleotide separated from the adjacent nucleic acids in which it is naturally present, is considered “isolated.”

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression.

As used herein, the term “modified” refers to a changed state or structure of a molecule or entity as compared with a parent or reference molecule or entity. Molecules may be modified in many ways including chemically, structurally, and functionally. In some embodiments, compounds and/or compositions of the present disclosure are modified by the introduction of non-natural amino acids.

As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids e.g., polynucleotides). In some embodiments, wherein mutations comprise the addition and/or substitution of amino acids and/or nucleotides, such additions and/or substitutions may comprise 1 or more amino acid and/or nucleotide residues and may include modified amino acids and/or nucleotides. The resulting construct, molecule or sequence of a mutation, change or alteration may be referred to herein as a mutant.

“Nucleic acid,” “nucleic acid molecule,” “oligonucleotide,” “nucleotide,” and “polynucleotide” are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA.

As used herein, the phrase “operably linked” refers to a functional connection between two or more molecules, constructs, transcripts, entities, moieties or the like.

“Operably-linked” or “functionally linked” as it refers to nucleic acid sequences and polynucleotides refers to the association of nucleic acid sequences so that the function of one is affected by the other, while the nucleic acid sequences need not necessarily be adjacent or contiguous to each other, but may have intervening sequences between them. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. A transcriptional regulatory sequence is generally operably linked in cis with a coding sequence, but need not be directly adjacent to it. For example, an enhancer is a transcriptional regulatory sequence that is operably linked to a coding sequence, even though they are not contiguous, or, a promoter is operably linked to a gene of interest if the promoter regulates or mediates transcription of the gene of interest in a cell.

For example, an EF-1 promoter or enhancer sequence, is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In an association between two or more polypeptides or domains thereof to create a fusion polypeptide, the term “operably linked” means that the state or function of one polypeptide in the fusion protein is affected by the other polypeptide in the fusion protein. For example, with respect to a fusion protein comprising a DRD and a transcription factor or a domain thereof, the DRD and the transcription factor or domain thereof are operably linked if stabilization of the DRD with a ligand results in stabilization of the transcription factor or domain thereof, while destabilization of the DRD in the absence of a ligand results in destabilization of the transcription factor or domain thereof. With respect to a fusion polypeptide in which a DNA-binding domain is fused to an activation domain, the DNA-binding domain and the activation domain are operably linked if, in the fusion polypeptide, the DNA-binding domain portion is able to bind to its specific binding site, and thus enable the activation domain to upregulate gene expression.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally occurring amino acids. The term, as used herein, refers to proteins, polypeptides, and peptides of any size, structure, or function. In some instances, the polypeptide is smaller than about 50 amino acids and the polypeptide may then be termed a “peptide”. Polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The terms also include post-expression modifications of the polypeptide, for example, glycosylation, sialylation, acetylation, phosphorylation, and the like.

The term “plasmid” refers to a genetic element often carrying a gene that is not part of the chromosome of the host cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source. A plasmid may comprise a number of nucleotide sequences have been joined or recombined into a unique construction which is capable of introducing a promoter fragment and DNA sequence for a selected gene product along with appropriate 3′ untranslated sequence into a cell. Starting plasmids disclosed herein are either commercially available, publicly available on an unrestricted basis, or can be constructed from available plasmids by routine application of well-known published procedures. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

“Promoter” and “promoter sequence” are used interchangeably and refer to a DNA sequence capable of controlling the expression of a coding sequence or functional RNA. In general, a coding sequence is located 3′ to a promoter sequence. Promoters may be derived in their entirety from a native gene or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths may have identical promoter activity. The promoter sequence is typically bounded at its 3′ terminus by the transcription initiation site and extends upstream (5′ direction) to include the minimum number of bases or elements necessary to initiate transcription at levels detectable above background. Within the promoter sequence is found a transcription initiation site (conveniently defined for example, by mapping with nuclease Si), as well as protein binding domains (consensus sequences) responsible for the binding of RNA polymerase.

The promoter region of a gene includes the transcription regulatory elements that typically lie 5′ to a structural gene. If a gene is to be activated, proteins known as transcription factors attach to the promoter region of the gene. This assembly resembles an “on switch” by enabling an enzyme to transcribe a second genetic segment from DNA into RNA. In most cases the resulting RNA molecule serves as a template for synthesis of a specific protein; sometimes RNA itself is the final product. The promoter region may be a normal cellular promoter or an oncopromoter.

As an example of disease-specific promoters, useful promoters for treating cancer include the promoters of oncogenes, including promoters for treating anemia. Examples of classes of oncogenes include, but are not limited to, growth factors, growth factor receptors, protein kinases, programmed cell death regulators and transcription factors.

Examples of promoter sequences and other regulatory elements (e.g., enhancers) that are known in the art and are useful as therapeutic switch promoters in the present invention are disclosed in U.S. Pat. No. 9,402,919, Ser. No. 14/001,943, filed on Mar. 2, 2012.

The term “payload” refers to any protein or polypeptide or compound whose function or amount is to be altered. In the context of the present disclosure, a payload may be a polypeptide that is encoded by a nucleic acid sequence whose transcriptional activity is regulated by a transcription factor of the present disclosure. Binding of the transcription factor to the corresponding specific polynucleotide binding site enables transcription of the encoded polypeptide and the transcribed nucleic acid molecule (transcript) can then be translated and expressed, thereby resulting in expression of the payload.. A payload may be a protein, a fusion protein, or product of a non-coding gene, or variants and fragments thereof. When amino acid based, a payload may be referred to as a “protein of interest”.

The term “pharmaceutically acceptable excipient,” as used herein, refers to any ingredient other than active agents (e.g., as described herein) present in pharmaceutical compositions and having the properties of being substantially nontoxic and non-inflammatory in subjects. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

“Pharmaceutically acceptable salts” of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments, a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, I7th ed., Mack Publishing Company, Easton, Pa., I985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety. Pharmaceutically acceptable solvate: The term “pharmaceutically acceptable solvate,” as used herein, refers to a crystalline form of a compound wherein molecules of a suitable solvent are incorporated in the crystal lattice. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N, N′-dimethylformamide (DMF), N, N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.” In some embodiments, the solvent incorporated into a solvate is of a type or at a level that is physiologically tolerable to an organism to which the solvate is administered (e.g., in a unit dosage form of a pharmaceutical composition).

The term “recombinant” has the usual meaning in the art, and refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. When used with reference to a cell, the term indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette” or simply an “expression cassette” is a nucleic acid construct, generated recombinantly or synthetically, that has control elements that are capable of affecting expression of a structural gene that is operably linked to the control elements in hosts compatible with such sequences. Expression cassettes include at least promoters and optionally, transcription termination signals. Typically, the recombinant expression cassette includes at least a nucleic acid to be transcribed and a promoter. Additional factors necessary or helpful in effecting expression can also be used as described herein. For example, transcription termination signals, enhancers, and other nucleic acid sequences that influence gene expression, can also be included in an expression cassette.

“Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e, the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide. In some embodiments, “homologous recombination” refers to the insertion of a foreign DNA sequence into another DNA molecule, e.g., insertion of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

The term “reporter gene” refers to a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include: luciferase (Luc), green fluorescent protein (GFP), chloramphenicol acetyltransferase (CAT), beta-galactosidase (LacZ), beta-glucuronidase (Gus), and the like. Selectable marker genes may also be considered reporter genes.

The term “response element” refers to one or more cis-acting DNA elements which confer responsiveness on a promoter mediated through interaction with the DNA-binding domains of a transcription factor. In some embodiments, response elements provide binding sites for RNA polymerase and transcription factors. This DNA element may be palindromic (perfect or imperfect) in its sequence or composed of sequence motifs or half sites separated by a variable number of nucleotides. The half sites can be similar or identical and arranged as either direct or inverted repeats or as a single half site or multimers of adjacent half sites in tandem. The response element may comprise a minimal promoter isolated from different organisms depending upon the nature of the cell or organism into which the response element is incorporated. The DNA binding domain of the transcription factor binds to the DNA sequence of a response element to initiate or suppress transcription of downstream gene(s) under the regulation of this response element.

The term “sequence” when used in reference to a nucleic acid molecule refers to a nucleotide sequence of any length, which can be DNA or RNA; can be linear, circular or branched and can be either single-stranded or double stranded.

The term “selectable marker” refers to an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like.

As used herein, the term “stabilize”, “stabilized,” “stabilized region” means to make a polypeptide or region thereof become or remain stable. In some embodiments, stability is measured relative to an absolute value. For example, the stability of a polypeptide comprising a DRD bound to its ligand may be compared to the stability of the wild type polypeptide. In some embodiments, stability is measured relative to a different status or state of the same polypeptide. For example, the stability of a polypeptide comprising a DRD bound to its ligand may be compared to the stability of the polypeptide comprising a DRD in the absence of its ligand.

As used herein, the term “standard CAR” refers to the standard design of a chimeric antigen receptor. The components of a CAR fusion protein including the extracellular scFv fragment, transmembrane domain and one or more intracellular domains are linearly constructed as a single fusion protein.

The terms “subject” and “patient” are used interchangeably and refer to mammals such as human patients and non-human primates, as well as experimental animals such as rabbits, dogs, cats, rats, mice, and other animals. Accordingly, the term “subject” or “patient” as used herein means any patient or subject (e.g. mammalian) to which the nucleic acids, polynucleotides, payloads, compositions, vectors or cells of the disclosure can be administered.

A “T cell” is an immune cell that produces T cell receptors (TCRs). T cells can be naïve (not exposed to antigen; increased expression of CD62L, CCR7, CD28, CD3, CD127, and CD45RA, and decreased expression of CD45RO as compared to TCM), memory T Cells™ (antigen-experienced and long-lived), and effector cells (antigen-experienced, cytotoxic). TM can be further divided into subsets of central memory T cells (TCM, increased expression of CD62L, CCR7, CD28, CD127, CD45RO, and CD95, and decreased expression of CD54RA as compared to naïve T cell and effector memory T cells (TEM, decreased expression of CD62L, CCR7, CD28, CD45RA, and increased expression of CD127 as compared to naïve T cells or TCM). Effector T cells (TE) refers to antigen-experienced CD8+ cytotoxic T lymphocytes that have decreased expression of CD62L, CCR7, CD28, and are positive for granzyme and perforin as compared to TCM. Other exemplary T cells include regulatory T cells, such as CD4+CD25+(Foxp3+) regulatory T cells and Treg17 cells, as well as Tr1, Th3, CD8+CD28−, and Qa-1 restricted T cells.

T cell receptor (TCR) refers to an immunoglobulin superfamily member having a variable antigen binding domain, a constant domain, a transmembrane region, and a short cytoplasmic tail, which is capable of specifically binding to an antigen peptide bound to a MHC receptor. A TCR can be found on the surface of a cell or in soluble form and generally is comprised of a heterodimer having a and p chains (also known as TCRα and TCRβ, respectively), or γ and δ chains (also known as TCRγ and TCRδ, respectively). The extracellular portion of TCR chains (e.g., α-chain, β-chain) contains two immunoglobulin domains, a variable domain (e.g., α-chain variable domain or Vα, β-chain variable domain or Vβ) at the N terminus, and one constant domain (e.g., α-chain constant domain or Cα and β-chain constant domain or Cβ,) adjacent to the cell membrane. Similar to immunoglobulin, the variable domains contain complementary determining regions (CDRs) separated by framework regions (FRs). A TCR is usually associated with the CD3 complex to form a TCR complex. As used herein, the term “TCR complex” refers to a complex formed by the association of CD3 with TCR. For example, a TCR complex can be composed of a CD3γ chain, a CD3δ chain, two CD3ε chains, a homodimer of CD3ζ chains, a TCRα chain, and a TCRβ chain. Alternatively, a TCR complex can be composed of a CD3γ chain, a CD3δ chain, two CD3ε chains, a homodimer of CD3ζ chains, a TCRγ chain, and a TCRS chain. A “component of a TCR complex,” as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3F and CD3δ, a complex of CD37 and CD3R, or a sub-TCR complex of TCRα, TCRβ, CD3γ, CD3δ, and two CD3ε chains.

As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

As used herein, the terms “treatment” or “treating” denote an approach for obtaining a beneficial or desired result including and preferably a beneficial or desired clinical result. Such beneficial or desired clinical results include, but are not limited to, one or more of the following: reducing the proliferation of (or destroying) cancerous cells or other diseased cells, reducing metastasis of cancerous cells found in cancers, shrinking the size of the tumor, decreasing symptoms resulting from the disease, increasing the quality of life of those suffering from the disease, decreasing the dose of other medications required to treat the disease, delaying the progression of the disease, and/or prolonging survival of individuals.

As used herein, the term “tune” means to adjust, balance or adapt one thing in response to a stimulus or toward a particular outcome. In one non-limiting example, the DRDs of the present disclosure adjust, balance or adapt the function or structure of compositions to which they are appended, attached or associated with in response to particular stimuli and/or environments.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein.

A “target site” or “target sequence” is a nucleic acid sequence that defines a portion of a nucleic acid to which a binding molecule will bind, provided sufficient conditions for binding exist. An “intended” target site is one that the DNA-binding molecule is designed and/or selected to bind to.

“Transcription” refers to the process involving the interaction of an RNA polymerase with a gene, which directs the expression as RNA of the structural information present in the coding sequences of the gene. The process includes, but is not limited to the following steps: (1) transcription initiation, (2) transcript elongation, (3) transcript splicing, (4) transcript capping, (5) transcript termination, (6) transcript polyadenylation, (7) nuclear export of the transcript, (8) transcript editing, and (9) stabilizing the transcript.

A transcription regulatory element or sequence include, but is not limited to, a promoter sequence (e.g., the TATA box), an enhancer element, a signal sequence, or an array of transcription factor binding sites. It controls or regulates transcription of a gene operably linked to it.

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Transgene” refers to a gene that has been introduced into a host cell. The transgene may comprise sequences that are native to the cell, sequences that do not occur naturally in the cell, or combinations thereof. A transgene may contain sequences coding for one or more proteins that may be operably linked to appropriate regulatory sequences for expression of the coding sequences in the cell.

“Transduction” refers to the delivery of a nucleic acid molecule into a recipient host cell, such as by a gene delivery vector, such as a lentiviral vector, or a rAAV. For example, transduction of a target cell by a rAAV virion leads to transfer of the rAAV vector contained in that virion into the transduced cell. “Host cell” or “target cell” refers to the cell into which the nucleic acid delivery takes place.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999).

The term “transfection” refers to the uptake of exogenous or heterologous nucleic acids by a cell. A cell has been “transfected” by nucleic acids when such nucleic acids have been introduced inside the cell. The transforming nucleic acids can be integrated (covalently linked) into chromosomal DNA making up the genome of the cell.

“Transcriptional and translational control sequences” refer to nucleic acid regulatory sequences, such as promoters, enhancers, terminators, and the like, that provide for the expression of a coding sequence in a host cell. In eukaryotic cells, polyadenylation signals are control sequences.

As used herein, the term “variant” when used in reference to a polypeptide refers to molecules which differ in their amino acid sequence from a native or reference sequence. The amino acid sequence variants may possess substitutions, insertions, additions, deletions and/or covalent modifications at certain positions within the amino acid sequence, as compared to a native or reference sequence. As used herein, a “deletion” also includes a truncation at the N- or C-terminus of a polypeptide. Ordinarily, variants will possess at least about 50% identity (homology) to a native or reference sequence, and preferably, they will be at least about 80%, more preferably at least about 90% identical (homologous) to a native or reference sequence. As used herein, the terms “native” or “starting” or “reference” when referring to sequences are relative terms referring to an original molecule against which a comparison may be made. Native or starting or reference sequences should not be confused with wild-type sequences. Native sequences or molecules may represent the wild-type (that sequence found in nature) but do not have to be identical to the wild-type sequence.

A “vector” refers to any vehicle for the cloning of and/or transfer of a nucleic acid into a host cell. Such a nucleic acid may be referred to as being “carried” in or by the vector. A vector may be a replicon to which another DNA segment may be attached so as to bring about the replication of the attached segment. A “replicon” refers to any genetic element (e.g., plasmid, phage, cosmid, chromosome, virus) that functions as an autonomous unit of DNA replication in vivo, i.e., capable of replication under its own control. The term “vector” includes both viral and nonviral vehicles for introducing the nucleic acid into a cell in vitro, ex vivo or in vivo. A large number of vectors known in the art may be used to manipulate nucleic acids, incorporate response elements and promoters into genes, etc. Possible vectors include, for example, plasmids or modified viruses including, for example bacteriophages such as lambda derivatives, or plasmids such as pBR322 or pUC plasmid derivatives, or the Bluescript vector. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified, or any site may be produced by ligating nucleotide sequences (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker. Common vectors include plasmids, viral genomes, and (primarily in yeast and bacteria) “artificial chromosomes.” “Expression vectors” are vectors that comprise elements that provide for or facilitate transcription of nucleic acids that are cloned into the vectors. Such elements can include, e.g., promoters and/or enhancers operably coupled to a nucleic acid of interest. A vector that comprises a

A “cloning vector” refers to a “replicon,” which is a unit length of a nucleic acid, preferably DNA, that replicates sequentially and which comprises an origin of replication, such as a plasmid, phage or cosmid, to which another nucleic acid segment may be attached so as to bring about the replication of the attached segment. Cloning vectors may be capable of replication in one cell type and expression in another (“shuttle vector”). Cloning vectors may comprise one or more sequences that can be used for selection of cells comprising the vector and/or one or more multiple cloning sites for insertion of sequences of interest.

The term “expression vector” refers to a vector, plasmid or vehicle designed to enable the expression of an inserted nucleic acid sequence. The cloned gene, i.e., the inserted nucleic acid sequence, is usually placed under the control of control elements such as a promoter, a minimal promoter, an enhancer, or the like. Initiation control regions or promoters, which are useful to drive expression of a nucleic acid in the desired host cell are numerous and familiar to those skilled in the art. Virtually any promoter capable of driving expression of these genes can be used in an expression vector, including but not limited to, viral promoters, bacterial promoters, animal promoters, mammalian promoters, synthetic promoters, constitutive promoters, tissue specific promoters, pathogenesis or disease related promoters, developmental specific promoters, inducible promoters, light regulated promoters; CYC1, HIS3, GAL1, GAL4, GAL10, ADH1, PGK, PHO5, GAPDH, ADC1, TRP1, URA3, LEU2, ENO, TP1, alkaline phosphatase promoters (useful for expression in Saccharomyces); AOX1 promoter (useful for expression in Pichia); beta-lactamase, lac, ara, tet, trp, lPL, lPR, T7, tac, and trc promoters (useful for expression in Escherichia coli); light regulated-, seed specific-, pollen specific-, ovary specific-, cauliflower mosaic virus 35S, CMV 35S minimal, cassava vein mosaic virus (CsVMV), chlorophyll a/b binding protein, ribulose 1,5-bisphosphate carboxylase, shoot-specific, root specific, chitinase, stress inducible, rice tungro bacilliform virus, plant super-promoter, potato leucine aminopeptidase, nitrate reductase, mannopine synthase, nopaline synthase, ubiquitin, zein protein, and anthocyanin promoters (useful for expression in plant cells); animal and mammalian promoters known in the art including, but are not limited to, the SV40 early (SV40e) promoter region, the promoter contained in the 3′ long terminal repeat (LTR) of Rous sarcoma virus (RSV), the promoters of the E1A or major late promoter (MLP) genes of adenoviruses (Ad), the cytomegalovirus (CMV) early promoter, the herpes simplex virus (HSV) thymidine kinase (TK) promoter, a baculovirus 1E1 promoter, an elongation factor 1 alpha (EF1) promoter, a phosphoglycerate kinase (PGK) promoter, a ubiquitin (Ubc) promoter, an albumin promoter, the regulatory sequences of the mouse metallothionein-L promoter and transcriptional control regions, the ubiquitous promoters (HPRT, vimentin, .alpha.-actin, tubulin and the like), the promoters of the intermediate filaments (desmin, neurofilaments, keratin, GFAP, and the like), the promoters of therapeutic genes (of the MDR, CFTR or factor VIII type, and the like), pathogenesis or disease related-promoters, and promoters that exhibit tissue specificity and have been utilized in transgenic animals, such as the elastase I gene control region which is active in pancreatic acinar cells; insulin gene control region active in pancreatic beta cells, immunoglobulin gene control region active in lymphoid cells, mouse mammary tumor virus control region active in testicular, breast, lymphoid and mast cells; albumin gene, Apo AI and Apo AII control regions active in liver, alpha-fetoprotein gene control region active in liver, alpha 1-antitrypsin gene control region active in the liver, beta-globin gene control region active in myeloid cells, myelin basic protein gene control region active in oligodendrocyte cells in the brain, myosin light chain-2 gene control region active in skeletal muscle, and gonadotropic releasing hormone gene control region active in the hypothalamus, pyruvate kinase promoter, villin promoter, promoter of the fatty acid binding intestinal protein, promoter of the smooth muscle cell .alpha.-actin, and the like. In addition, these expression sequences may be modified by addition of enhancer or regulatory sequences and the like.

Vectors may be introduced into the desired host cells, by methods known in the art, e.g., transfection, electroporation, microinjection, transduction, cell fusion, DEAF dextran, calcium phosphate precipitation, lipofection (lysosome fusion), use of a gene gun, or a DNA vector transporter (see, e.g., Wu et al., J. Biol. Chem. 267:963 (1992); Wu et al., J. Biol. Chem. 263:14621 (1988); and Hartmut et al., Canadian Patent Application No. 2,012,311).

Viral vectors, and particularly lentiviral and retroviral vectors, have been used in a wide variety of gene delivery applications in cells, as well as living animal subjects. Viral vectors that can be used include, but are not limited to, retrovirus, adeno-associated virus, pox, baculovirus, vaccinia, herpes simplex, Epstein-Barr, adenovirus, geminivirus, and caulimovirus vectors. Non-viral vectors include plasmids, liposomes, electrically charged lipids (cytofectins), DNA-protein complexes, and biopolymers. In addition to a nucleic acid, a vector may also comprise one or more regulatory regions, and/or selectable markers useful in selecting, measuring, and monitoring nucleic acid transfer results (transfer to which tissues, duration of expression, etc.).

Several methods known in the art may be used to propagate a polynucleotide according to the invention. Once a suitable host system and growth conditions are established, recombinant expression vectors can be propagated and prepared in quantity. As described herein, the expression vectors which can be used include, but are not limited to, the following vectors or their derivatives: human or animal viruses such as lentiviruses, vaccinia virus or AAV, or adenovirus; insect viruses such as baculovirus; yeast vectors; bacteriophage vectors (e.g., lambda), and plasmid and cosmid DNA vectors, to name but a few. A vector of the invention may also be administered to a subject by any route of administration, including, but not limited to, intramuscular administration.

A polynucleotide according to the disclosure can also be introduced in vivo by lipofection. There has been increasing use of liposomes for encapsulation and transfection of nucleic acids in vitro. Synthetic cationic lipids designed to limit the difficulties and dangers encountered with liposome-mediated transfection can be used to prepare liposomes for in vivo transfection of a gene. The use of cationic lipids may promote encapsulation of negatively charged nucleic acids, and also promote fusion with negatively charged cell membranes. Particularly useful lipid compounds and compositions for transfer of nucleic acids are described in WO95/18863, WO96/17823 and U.S. Pat. No. 5,459,127. The use of lipofection to introduce exogenous genes into the specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney, and the brain. Lipids may be chemically coupled to other molecules for the purpose of targeting. Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931).

It is also possible to introduce a vector in vivo as a naked DNA plasmid (see U.S. Pat. Nos. 5,693,622, 5,589,466 and 5,580,859). Receptor-mediated DNA delivery approaches can also be used.

In addition, the recombinant vector comprising a polynucleotide according to the invention may include one or more origins for replication in the cellular hosts in which their amplification or their expression is sought, markers or selectable markers.

“Wild-type” refers to a nucleic acid sequence, nucleic acid molecule, amino acid sequence, polypeptide, virus or organism found in nature without any known mutation. The term may also be used to describe the properties of a wild-type nucleic acid sequence, nucleic acid molecule, amino acid sequence, polypeptide, virus or organism.

### EXAMPLES

### Example 1: Design and Testing of Transcription Factor Systems

The present example provides methods for designing, preparing and evaluating transcription factor systems taught by the present disclosure.

Design of transcription factor system: As described above, a transcription factor system is a modular system, in that the polynucleotides or nucleic acid constructs of a transcription factor system may comprise different arrangements of nucleic acid sequences, and/or may be uniquely combined as part of the transcription factor system, so long as the resulting combination of polynucleotides or nucleic acid constructs comprises (1) one or more nucleic acid sequences that encode a transcription factor that is able to bind to a specific polynucleotide binding site and activate transcription; (2) a nucleic acid sequence that encodes a drug responsive domain (DRD), wherein the transcription factor is operably linked to the DRD; and (3) a nucleic acid sequence that encodes a payload and is operably linked to an inducible promoter comprising the specific polynucleotide binding site.

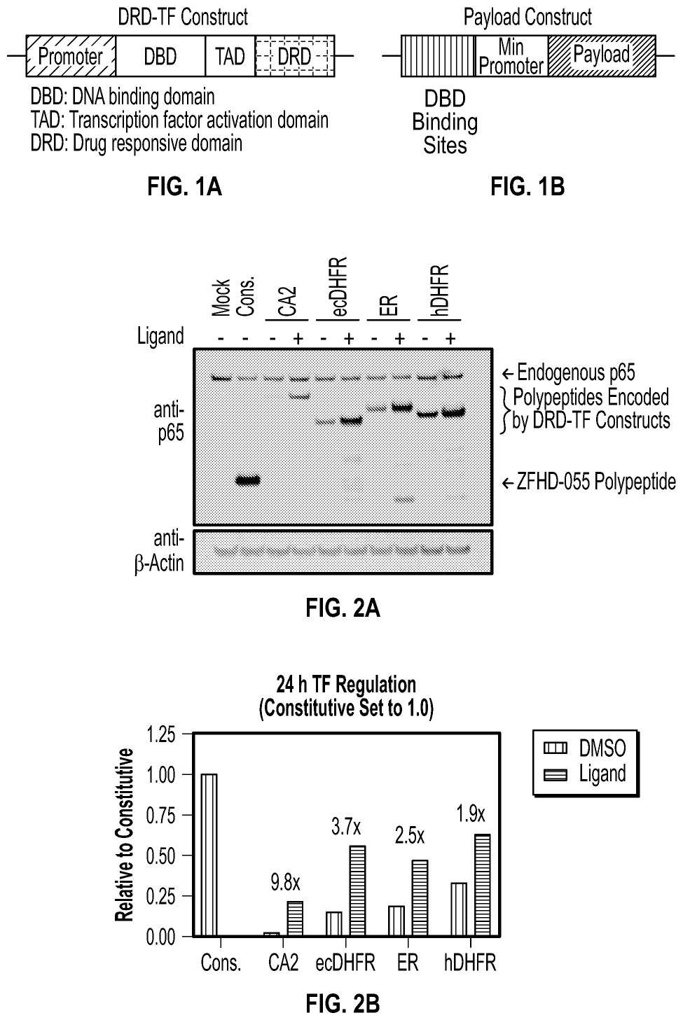

The present example demonstrates an approach for designing an illustrative transcription factor system. The transcription factor system of this example is encoded by nucleic acid constructs that comprise: (i) a first nucleic acid construct that encodes a transcription factor DNA binding domain; a transcription factor activation domain; and a drug responsive domain (DRD) and (2) a second nucleic acid construct that encodes a payload, wherein expression of the payload is driven by an inducible promoter that comprises binding sites for the transcription factor DNA binding domain (FIG. 1A-FIG. 1). As shown in FIG. 1A, a promoter drives expression of the transcription factor DNA binding domain, transcription factor activation domain and DRD. The transcription factor DNA binding domain and the transcription factor activation domain of the present example are expressed as a transcription factor fusion protein, wherein the fusion protein is operably linked to a DRD (DRD-TF). The levels of the transcription factor fusion protein can be regulated with the DRD ligand. Additionally, the regulated transcription factor fusion protein can induce expression of the payload in a ligand-dependent manner. As discussed above and further exemplified in the subsequent examples, workable variations on this design scheme include: varying the positioning of construct components; inclusion of additional nucleic acid sequences as part of the constructs (e.g., such as linker sequences, regulatory elements, polyadenylation sequences, and ribosome skipping elements); and design of a single nucleic acid construct encoding the transcription factor system.

Each component of the transcription factor system of this example can be selected separately. For example, the transcription factor DNA binding domain may be selected from an engineered zinc finger binding protein, engineered TAL effector, or other natural or engineered DNA binding domain. The transcription factor activation domain may be selected from the activation domains of p65, VP64, p300, SAM, VPR, or other activation domains. The promoter driving expression of the transcription factor DNA binding domain, transcription factor activation domain and DRD may be selected from a constitutive promoter, a tissue-specific promoter, a cell-specific promoter, a cell differentiation-specific promoter, and/or a disease-specific promoter. Optionally, the promoter driving expression of the transcription factor DNA binding domain, transcription factor activation domain and DRD may be selected from EF1a, CMV, EFS, RSV, SFFV, PGK, CAG, and SV40. The payload may be selected from any protein of interest, such as an intracellular protein, a membrane-bound protein, or a secreted protein. Optionally, the payload may be a therapeutic protein. Non-limiting examples of payload include: a cytokine (e.g., IL2, IL12, or IL15), an antibody, a coagulation factor, an enzyme (e.g., Cas9, ZFN, or Cre), a gene editing protein, a T cell receptor (TCR) and a chimeric antigen receptor (CAR). The inducible promoter that is operably linked to the nucleic acid sequence encoding the payload may be designed by selecting from known regulatory sequences that enable formation of the initiation complex for nucleic acid transcription. Such regulatory sequences coupled with the specific polynucleotide binding site of the transcription factor system can be used to design the inducible promoter of the transcription factor systems described herein. For example, Ede, C. et al. describe promoters known in the art that may be used in the design of a transcription factor system of the present disclosure (Ede, C. et al. ACS Synth Biol. 2016 May 20; 5(5): 395-404).

For the illustrative transcription factor system of the present example, the transcription factor DNA binding domain is selected to correspond to an inducible promoter that comprises binding sites for the binding domain and drives expression of the payload. The DNA binding domain of the present example may comprise or be derived from the DNA binding domain of a DNA binding protein for which a corresponding specific polynucleotide binding site is known or can be determined. Pairs of DNA binding domains of DNA binding proteins and their corresponding polynucleotide binding sites that are known in the art may be used in the nucleic acid constructs of the present example. For example, a nucleic acid construct of the illustrative transcription factor system may comprise a zinc finger array provided by Khalil A. S., et al. Alternatively, the nucleic acid construct may comprise a TAL effector repeat region provided by Zhang, F., et al. Additionally, methods for designing interacting pairs of DNA binding domains of transcription factors and their corresponding polynucleotide binding sites are available to one of skill in the art. For example, methods of identifying binding protein-DNA recognition site pairs include oligomerized pool engineering methods and phage display methods, as well as other methods discussed in Khalil A. S., et al., Pabo, C. O., et al., and Zhang, F. et al. (Khalil A. S., et al. Cell 2012, 150, 647-658; Pabo, C. O., et al. Annu. Rev. Biochem. 2001, 70:313-40).

Preparations of transcription factor systems: As illustrated herein, a transcription factor system can be prepared by introducing nucleic acid constructs encoding the system into a cell. These constructs may be introduced on one or more nucleic acid molecules and may be transiently expressed or stably integrated. For transient expression, cells are transfected with transfer vectors prepared as described in Example 2. For stable integration, transduced cell lines with stably integrated constructs are prepared and selected according to methods described in Example 2. Cell lines to be transduced may be selected from cells including, but not limited to, U2OS, Jurkat, HEK293T, or other cell line cells.

Evaluating transcription factor systems: As described above, for example, in the “Characterization of ligand-dependent activity of a transcription factor system” section, various methods can be used to evaluate a transcription factor system. The present example provides methods of evaluating a transcription factor system prepared by stable integration of constructs discussed above. These methods can also be used to evaluate a transcription factor system prepared by transient expression of a transcription factor system.

Cells to be evaluated may include untransduced (parental) cells and cells transduced with lentivirus made from the following constructs: (1) DRD-TF construct, such as shown in FIG. 1A; (2) payload construct, such as shown in FIG. 1B; and (3) both lentivirus made from a DRD-TF construct and lentivirus made from a payload construct. Each cell line is treated with media containing ligand or DMSO. Cells are incubated for about 24-48 hours, collected and analyzed. Analysis techniques may include flow cytometry and immunoassays such as immunoblot analysis, ELISA, or an electrochemiluminescence method such as that of the Meso Scale Discovery platform. For example, ligand-dependent regulation of the transcription factor polypeptide encoded by the DRD-TF construct can be evaluated by immunoblot analysis with antibodies directed against the DNA binding domain and/or the transcription activation domain of the transcription factor polypeptide. Payloads may also be evaluated by immunoblot analysis with an antibody directed against the payload. Flow cytometry may also be used to evaluate payload levels, for example with the use of a labeled antibody that recognizes the payload. Analysis techniques such as ELISA and Meso Scale Discovery can be used to evaluate levels of secreted payload.

It is expected that ligand-treated cells compared to cells treated with the DMSO condition will have increased levels of the transcription factor polypeptide encoded by the DRD-TF construct as well as increased levels of the payload. These results would confirm that levels of the transcription factor fusion protein can be regulated with the DRD ligand and that the regulated transcription factor fusion protein can induce expression of the payload in a ligand-dependent manner.

The present examples demonstrates the modularity in designing transcription factor systems of the present disclosure and methods of preparing and evaluating these systems. As shown herein, various construct engineering schemes can be employed to design a transcription factor system. Additional engineering schemes are described elsewhere by this disclosure.

### Example 2: Construct Assembly, Virus Production and Cell Line Generation

The following materials and methods were used to prepare constructs, viruses and cell lines used in the examples of the present disclosure.

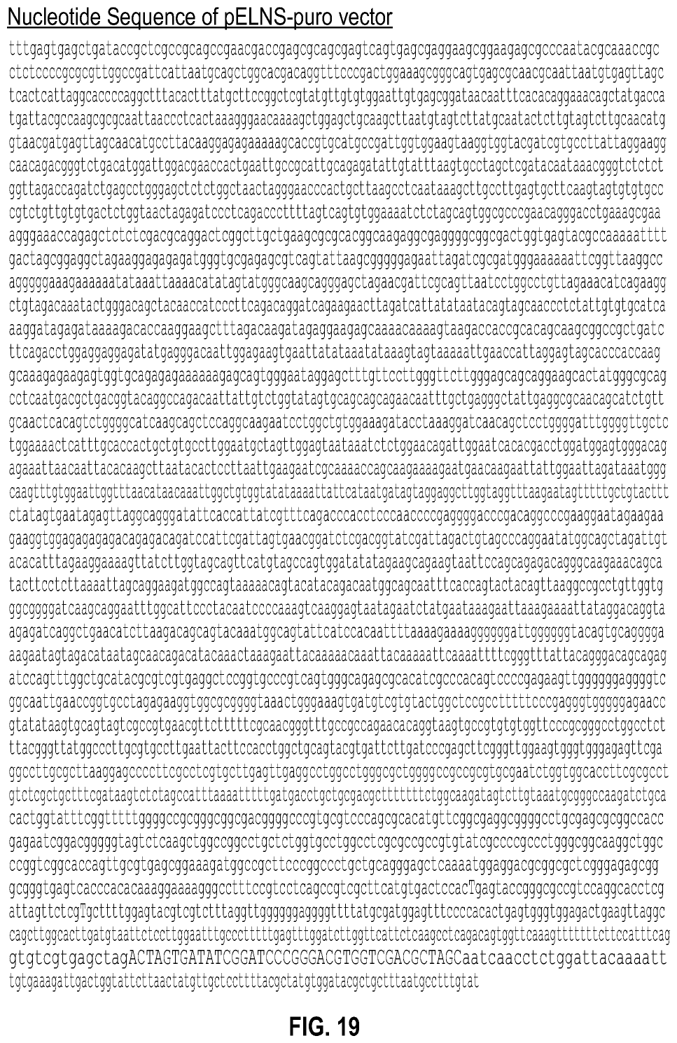

Cloning Methods: Constructs listed in Table 1 were prepared by Gibson assembly with NEBuilder HiFi DNA Assembly Master Mix (New England BioLabs, Inc., Ipswich, Mass.). DNA pieces for assembly were either purchased and synthesized de novo or PCR copied out of previously made constructs. All constructs in Table 1 were introduced into transfer vector backbones. The transfer vectors used included pELDS-puro (for which a sequence is provided herein, FIG. 18 and SEQ ID NO: 68). pELDS-blast, pELDS-Thy1.2, and pELDS-Thy1.1 transfer vectors were created by swapping the puromycin resistance gene in the pELDS-puro vector for blasticidin resistance (pLenti6.3, Thermo Fisher Scientific, Waltham, Mass.); Thy1.2 cDNA (Origene, Rockville, Md.); or Thy1.1 cDNA (Origene, Rockville, Md.), respectively. Table 5 shows transfer vectors that were used for the indicated constructs.

TABLE 5
Transfer Vectors

| Construct | Transfer vector |
|---|---|
| ZFHD-004 | pELDS-blast |
| ZFHD-005 | pELDS-blast |
| ZFHD-007 | pELDS-puro |
| ZFHD-008 | pELDS-blast |
| ZFHD-009 | pELDS-blast |
| ZFHD-012 | pELDS-puro |
| ZFHD-013 | pELDS-puro |
| ZFHD-017 | pELDS-puro |
| ZFHD-018 | pELDS-puro |
| ZFHD-019 | pELDS-blast |
| ZFHD-022 | pELDS-Thy1.2 |
| ZFHD-036 | pELDS-Thy1.2 |
| ZFHD-048 | pELDS-Thy1.1 |
| ZFHD-010 | pELDS-puro |
| ZFHD-059 | pELDS-Thy1.1 |
| ZFHD-060 | pELDS-Thy1.1 |
| ZFHD-054 | pELDS-Thy1.1 |
| ZFHD-055 | pELDS-Thy1.1 |

The assembled plasmids were transformed into E. coli (NEB Stable Competent E. coli, New England BioLabs, Inc., Ipswich, Mass.) for amplification and sequences were confirmed before proceeding with virus production.

Lentivirus production: HEK293T cells were seeded on collagen coated tissue culture plates and maintained in growth media (DMEM supplemented with 5% FBS and 1% penicillin-streptomycin) until 70% confluent. Prior to transfection, media was replaced with SFM4Transfx-293 media. Cells were transfected with transfer vectors prepared as described above, as well as packaging plasmids (pRSV.REV, pMDLg/p.RRE and pMD2.G) using Lipofectamine 3000 transfection reagent (Thermo Fisher Scientific, Waltham, Mass.) in Opti-MEM medium (Thermo Fisher Scientific, Waltham, Mass.). Media was replaced 6-8 hours post-transfection with fresh SFM4Transfx-293 media. Supernatants containing virus were harvested 24 hours post-transfection, fresh media was added, and supernatants were harvested again at 48 hours post-transfection. Viral supernatants were filtered to remove debris and concentrated by ultracentrifugation in 20% sucrose gradient. Virus was resuspended in Opti-MEM, aliquoted and stored at −80° C.

Stable Cell Line Generation: Cells were transduced with lentivirus made from the corresponding construct. Transduced U2OS cells were selected by culturing for 2 weeks with 2 pg/mL of puromycin if expressing a payload construct or single vector construct or with 10 pg/mL of Blasticidin if expressing a transcription factor construct. Transduced ARPE-19 cells were selected by culturing for 2 weeks with 2 pg/mL of puromycin if expressing a payload construct or single vector construct or with 20 pg/mL of Blasticidin if expressing a transcription factor construct. Cells that were transduced with both a lentivirus made from a transfer vector comprising a payload construct and a lentivirus made from a transfer vector comprising a transcription factor construct were placed under a combination selection. Transduced Jurkat cells were isolated by fluorescence activated cell sorting (FACS) after staining for Thy1.1 and/or Thy1.2 depending on which lentivirus(es) they were transduced with.

### Example 3: Ligand-Dependent Regulation of Transcription Factors

The present example demonstrates regulation of transcription factors using different drug responsive domains (DRDs) and ligands. In the present example, the transcription factors are encoded by nucleic acid constructs that encode a transcription factor DNA binding domain, a transcription factor activation domain, and a drug responsive domain (DRD) (FIG. 1A). The transcription factor DNA binding domain and the transcription factor activation domain of the present example are expressed as a transcription factor fusion protein, wherein the fusion protein is operably linked to a DRD that is derived from an ecDHFR, CA2, hDHFR, or ER parent protein. As shown here, levels of the transcription factor fusion proteins can be regulated with ligand.

Cell lines: The present example analyzed untransfected (parental) HEK293T cells and HEK293T cells transfected with plasmid DNA of the following constructs: (1) ZFHD-059, (2) ZFHD-060, (3) ZFHD-054, (4) ZFHD-048, or (5) ZFHD-055. Cells were cultured in DMEM medium supplemented with 10% FBS and transfected as follows: HEK293T cells were seeded on tissue culture plates and maintained in growth media (DMEM supplemented with 10% FBS and 1% penicillin-streptomycin) until 70-80% confluent. Cells were transfected with transfer vectors prepared as described above using Lipofectamine 3000 transfection reagent (Thermo Fisher Scientific, Waltham, Mass.) in Opti-MEM medium (Thermo Fisher Scientific, Waltham, Mass.). Media was replaced 24 hours post-transfection with growth media with either 10 μM TMP, 1 μM bazedoxifene, 100 μM acetazolamide, 50 μM trimethoprim or 0.1% DMSO. After 24 hours, cells were collected for immunoblot assay. A description of the plasmids and corresponding ligand treatment conditions is provided in Table 6. The “OT-” label in Table 6 indicates reference to a plasmid comprising the indicated construct (e.g., ZFHD-059, ZFHD-060, etc.). Components shown in the description of Table 6 include a selection of components on the plasmids used to transfect cells in this example, as well as the name of the transfer vector backbone (pELDS). In the “Description” column of Table 6, the term “ZFHD1” refers to a ZFDH1 DNA binding domain and the term “p65” refers to a p65 activation domain.

TABLE 6
Plasmid descriptions and ligand treatment conditions

| Plasmid | Description | DRD | ligand | Concentration | |
|---|---|---|---|---|---|
| OT-ZFHD-059 | pELDS-EF1a-ZFHD1-p65-GGSGGGSGG | hDHFR | TMP | 50 | μM |
| | (SEQ ID NO: 59)-hDHFR(Y122I)-WPRE- | | | | |
| | SV40-thy1.1-ampR | | | | |
| OT-ZFHD-060 | pELDS-EF1a-ZFHD1-p65-GGSGGGSGG | ER | bazedoxifene | 1 | μM |
| | (SEQ ID NO: 59)-ER(Q502R)-WPRE- | | | | |
| | SV40-thy1.1-ampR | | | | |
| OT-ZFHD-054 | pELDS-EF1a-ZFHD1-p65- | ecDHFR | TMP | 10 | μM |
| | ecDHFR(12Y, 100I)-WPRE-SV40-Thy1.1 | | | | |
| OT-ZFHD-048 | pELDS-EF1a-ZFHD1-p65-CA2(L156H)- | CA2 | ACZ | 100 | μM |
| | WPRE-SV40-Thy1.1 | | | | |

| | | | | |
|---|---|---|---|---|
| OT-ZFHD-055 | pELDS-EF1a-ZFHD1-p65-WPRE-SV40- | none | none | vehicle |
| | Thy1.1 | | | only |

Ligand treatment: Each cell line was plated and cells were allowed to adhere overnight. Media was removed and 1 mL of media with appropriate ligand or 0.1% DMSO (Table 6) was added. Cells were incubated for 24 hours and removed from the plates with 0.25% trypsin for collection. Collected cells were pelleted, media was removed, and cell pellets were stored at −20° C. for later immunoblot assay.

Immunoblot assay: Cell pellets were resuspended in lysis buffer (T-PER™ Tissue Protein Extraction Reagent, Thermo Fisher Scientific, Waltham, Mass.) containing protease inhibitors. Cell lysates were added to NuPAGE LDS Sample buffer (Thermo Fisher Scientific, Waltham, Mass.) containing beta-mercaptoethanol or Reducing reagent (Thermo Fisher Scientific, Waltham, Mass.) and incubated at 96° C. for 6 minutes with 300 rpm agitation. Samples were run on a NuPAGE 4-12% Bis-Tris gel in BOLT MES SDS running buffer with BOLT antioxidant (Thermo Fisher Scientific, Waltham, Mass.). Proteins were transferred to nitrocellulose membranes and probed with the following antibodies: rabbit anti-NF kappaB-p65 antibody (1:1000; Cell Signaling Technology, Danvers, Mass.) and mouse anti-P actin antibody (1:2000; Cell Signaling Technology, Danvers, Mass.). Secondary antibodies were IRDye® 680RD donkey anti-mouse (1:4000; LI-COR, Lincoln, Nebr.) or IRDye® 800CW donkey anti-rabbit (1:3000; LI-COR).

Results: Immunoblot analysis of HEK293T cells transfected with DRD-TF constructs revealed that the transcription factor and DRD polypeptide encoded by the transcription factor construct ZFHD-059, ZFHD-060, ZFHD-054, and ZFHD-048 was present at higher levels in ligand-treated cells compared to DMSO-treated cells (FIG. 2A-FIG. 2B). This data indicates that protein levels of a transcription factor operably linked to a DRD can be regulated with a ligand for the DRD.

### Example 4: Ligand-Dependent Activity of a Transcription Factor System Comprising an ecDHFR DRD-Regulated Transcription Factor

The present example demonstrates ligand-dependent activity of a transcription factor system. In the present example, the transcription factor system is encoded by nucleic acid constructs that comprise: (1) a first nucleic acid construct that encodes a transcription factor DNA binding domain, a transcription factor activation domain, and a drug responsive domain (DRD) and (2) a second nucleic acid construct that encodes a payload, wherein expression of the payload is driven by an inducible promoter that comprises binding sites for the transcription factor DNA binding domain (FIG. 3A-FIG. 3B). The transcription factor DNA binding domain and the transcription factor activation domain of the present example are expressed as a transcription factor fusion protein, wherein the fusion protein is linked to a DRD that is derived from an ecDHFR parent protein. As shown here, levels of the transcription factor fusion protein can be regulated with an ecDHFR ligand. Additionally, the regulated transcription factor fusion protein can induce expression of the payload in a ligand-dependent manner.

Cell lines: The present example analyzed untransduced (parental) U2OS cells and U2OS cells transduced with lentivirus made from the following constructs: (1) ZFHD-005, (2) ZFHD-007, (3) both lentivirus made from construct ZFHD-005 and lentivirus made from construct ZFHD-007, or (4) both lentivirus made from construct ZFHD-004 and lentivirus made from construct ZFHD-007. Transduced cell lines with stably integrated constructs were prepared and selected according to methods described in Example 2. Cells were cultured in McCoy's 5A medium supplemented with 10% FBS.

Ligand treatment: Each cell line was plated in 6-8 wells in 24-well plates at a density of 25,000 cells/well and allowed to adhere overnight. Media was removed and 1 mL of media with 10 μM Trimethoprim (TMP) was added to half of the wells of each cell line. The remaining wells received 1 mL of media with 0.1% DMSO. Cells were incubated for 48 hours and removed from the plates with 0.25% trypsin for collection. One well of each treatment condition was analyzed by flow cytometry. With the remaining samples, collected cells were pelleted, media was removed, and cell pellets were stored at −20° C. for later immunoblot analysis.

Flow cytometry: Collected cells were washed one time with PBS, resuspended in PBS and analyzed by flow cytometry.

Immunoblot assay: Cell pellets were resuspended in lysis buffer (T-PER™ Tissue Protein Extraction Reagent, Thermo Fisher Scientific, Waltham, Mass.) containing protease inhibitors. Cell lysates were added to NuPAGE LDS Sample buffer (Thermo Fisher Scientific, Waltham, Mass.) containing beta-mercaptoethanol and incubated at 96° C. for 6 minutes with 300 rpm agitation. Samples were run on a NuPAGE 4-12% Bis-Tris gel in BOLT MES SDS running buffer with BOLT antioxidant (Thermo Fisher Scientific, Waltham, Mass.). Proteins were transferred to nitrocellulose membranes and probed with the following antibodies: rabbit anti-NF kappaB-p65 antibody (1:1000; Cell Signaling Technology, Danvers, Mass.) and mouse anti-P actin antibody (1:2000; Cell Signaling Technology, Danvers, Mass.). Secondary antibodies were IRDye® 680RD donkey anti-mouse (1:4000; LI-COR, Lincoln, Nebr.) or IRDye® 800CW donkey anti-rabbit (1:3000; LI-COR).

Results: Immunoblot analysis of U2OS cells with stably integrated constructs ZFHD-005 and ZFHD-007 revealed that the transcription factor and DRD polypeptide encoded by the transcription factor construct ZFHD-005 was present at higher levels in TMP-treated cells compared to DMSO-treated cells (FIG. 3D). This data indicates that protein levels of a transcription factor operably linked to a DRD can be regulated with a ligand for the DRD. By comparison, U2OS cells with a stably integrated constitutive transcription factor construct (ZFHD-004, depicted in FIG. 3C) showed that the transcription factor polypeptide encoded by construct ZFHD-004 is detected when the cells are treated with either DMSO or TMP.

Flow cytometry analysis of U2OS cells with stably integrated constructs ZFHD-005 and ZFHD-007 indicated that payload expression, as measured by GFP median fluorescence intensity (MFI), was higher in TMP-treated cells compared to DMSO-treated cells (FIG. 3E). This data indicates that a DRD-regulated transcription factor can induce expression of a payload in a ligand-dependent manner.

### Example 5: Dose-Dependent Activity of a Transcription Factor System Comprising an ecDHFR DRD-Regulated Transcription Factor

The present example demonstrates dose-responsive behavior of a transcription factor system in response to ligand. The transcription factor system of this example is described above in Example 4. As shown in the present example, protein levels of a transcription factor operably linked to a DRD can be regulated in a dose-dependent manner in response to a ligand for the DRD. Additionally, expression of a payload, which is encoded by a nucleic acid sequence operably linked to an inducible promoter comprising the transcription factor binding site, is also dependent on the dose of ligand.

Cell lines: The present example analyzed untransduced (parental) U2OS cells and U2OS cells transduced with both lentivirus made from construct ZFHD-005 and lentivirus made from construct ZFHD-007. The transduced cell line with stably integrated constructs was prepared and selected as described above in Example 2. Cells were cultured in McCoy's 5A medium supplemented with 10% FBS.

Ligand treatment: Cells were plated in 24-well plates at a density of 25,000 cells/well. Plated cells were allowed to adhere overnight.

To prepare media for the 10-point dose response curve, media with 10 μM Trimethoprim (TMP) was diluted with 8 serial dilutions of 1:3 in plain media. For the 10^{th} point in the dose response curve, media was prepared with 0.1% DMSO.

Media was removed from the plates and replaced with either DMSO media or media with TMP. Half of the parental U2OS wells received replacement media with 10 μM TMP. The other half of the parental U2OS wells received replacement media with 0.1% DMSO. U2OS cells with stably integrated constructs ZFHD-005 and ZFHD-007 received replacement media with 0.1% DMSO or each dose of TMP (2-3 wells for each condition). Treated cells were incubated for 48 hours. Cells were removed from plates with 0.25% trypsin and collected. For immunoblot sample analysis, collected cells were pelleted, media was removed, and cells were stored at −20° C. until procedure for western blot, as described above in Example 4. For flow cytometry sample analysis, collected cells were washed one time with PBS, resuspended in PBS and analyzed by flow cytometry.

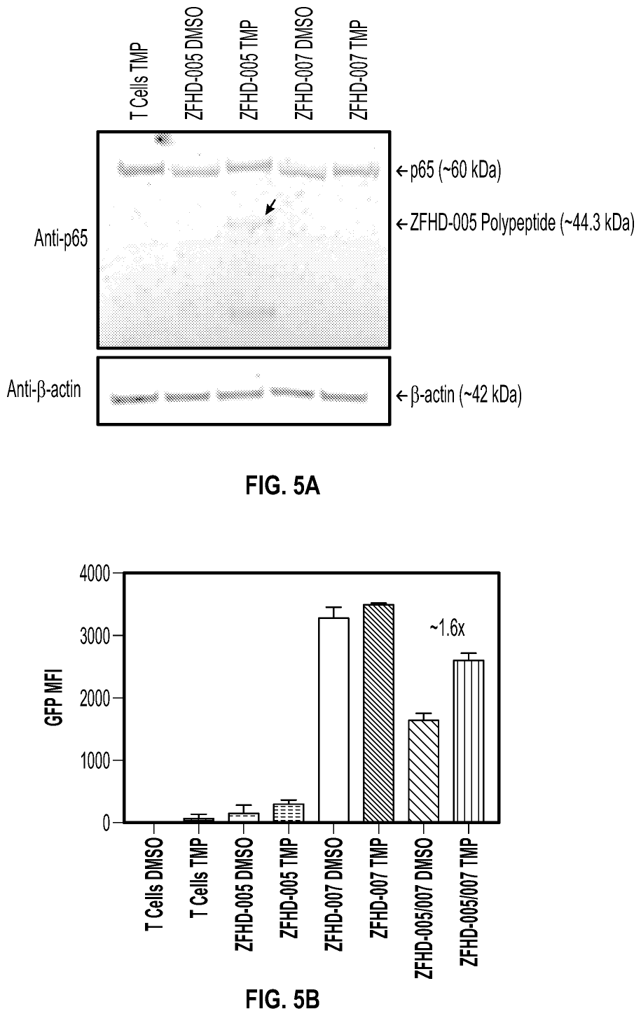

Results: Higher concentrations of TMP resulted in higher levels of the transcription factor fusion protein (FIG. 4A-FIG. 4B). This data indicates that TMP dose-dependently regulates protein levels of a transcription factor that is operably linked to an ecDHFR DRD.

Higher concentrations of TMP resulted in higher levels of payload expression as assessed by GFP MFI (FIG. 4C). The EC₅₀ of the payload response was similar to the EC₅₀ of the transcription factor response. This data indicates that an ecDHFR DRD-regulated transcription factor can dose-dependently drive expression of a payload in response to varying doses of TMP.

### Example 6: Ligand-Dependent Activity of a Transcription Factor System Comprising an ecDHFR DRD-Regulated Transcription Factor in T Cells

The present example demonstrates ligand-dependent activity of a transcription factor system in T cells. Components of the transcription factor system of this example are described above in Example 4.

T cell transduction and ligand treatment: On day 0, frozen human T cells were thawed and resuspended in complete T cell media (RPMI supplemented with Glutamax, 10% FBS, 1% penicillin-streptomycin, NEAA (1% from 100× stock), HEPES (1% from 100× stock), sodium pyruvate (1% from 100× stock), and mercaptoethanol (1× from 1000× stock)). Cells were washed, counted and plated in 24-well plates (500 μL per well at a density of 1×10\hat{ }6 cells/mL). Dynabeads (T-expander CD3/CD28) were washed with sterile PBS or media and added at 1.5×10\hat{ }6 beads/per well. Cells were incubated overnight.

On day 1, virus (OTLV-ZFHD-007, OTLV-ZFHD-005, OTLV-EGFP-001, or both OTLV-ZFHD-005 and OTLV-ZFHD-007) was added to the activated T cells. As used herein, the “OTLV-” label refers to a lentivirus produced from a plasmid comprising the indicated construct (e.g., ZFHD-007 and ZFHD-005). On day 2, 1 mL of fresh complete T cell media was added per well. On day 3, T cells were treated with ligand as follows: 8 wells each of each transduced T cell were plated (75 μL/well) in a 96-well flat bottom plate and 75 μL of media with 20 μM TMP was added to half of the wells and media with equivalent DMSO to the other half On day 5, one well of each transduction/treatment was collected, spun, removed of supernatant and stored at −20° C. until procedure for western blot, as described above in Example 4. The remaining samples were spun and removed of supernatant. Samples were washed once with cell staining buffer (Biolegend, San Diego, Calif.) and stained in 50 μL of 1:1000 fixable viability dye (Thermo Fisher Scientific, Waltham, Mass.) in cell staining buffer for 20 minutes at 4° C. Samples were washed twice with cell staining buffer and cells were resuspended in 200 μL of fixation buffer (Biolegend, San Diego, Calif.) and stored at 4° C. overnight. On day 6, cells were spun and resuspended in cell staining buffer with 1% QSol™ buffer (Intellicyt Corporation, Albuquerque, N. Mex.), and analyzed by flow cytometry.

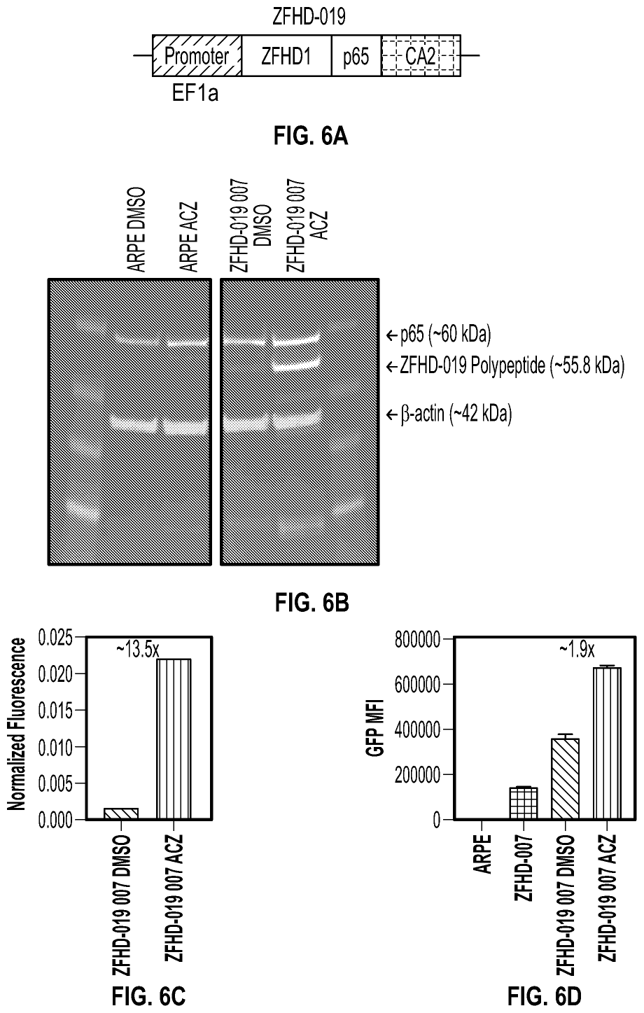

Results: Immunoblot analysis indicated that the transcription factor and DRD polypeptide encoded by the transcription factor construct ZFHD-005 was present at higher levels in T cells transduced with the OTLV-ZFHD-005 virus and treated with TMP compared to the same cells treated with DMSO (FIG. 5A). This data indicates that protein levels of a transcription factor operably linked to a DRD can be regulated with a ligand for the DRD in T cells.

Flow cytometry analysis indicated that payload expression, as measured by GFP MFI, increased by approximately 1.6-fold in T cells transduced with both the OTLV-ZFHD-005 and OTLV-ZFHD-007 viruses and treated with TMP compared to the same cells treated with DMSO (FIG. 5B). This data indicates that a DRD-regulated transcription factor can induce expression of a payload in a ligand-dependent manner in T cells.

These results indicate that a transcription factor system described by the present disclosure can regulate payload expression in T cells. The observed low fold change in payload expression may be because the cells were not 100% transduced and were not sorted for any transduction marker.

### Example 7: Ligand-Dependent Activity of a Transcription Factor System Comprising a CA2 DRD-Regulated Transcription Factor in ARPE-19 Cells

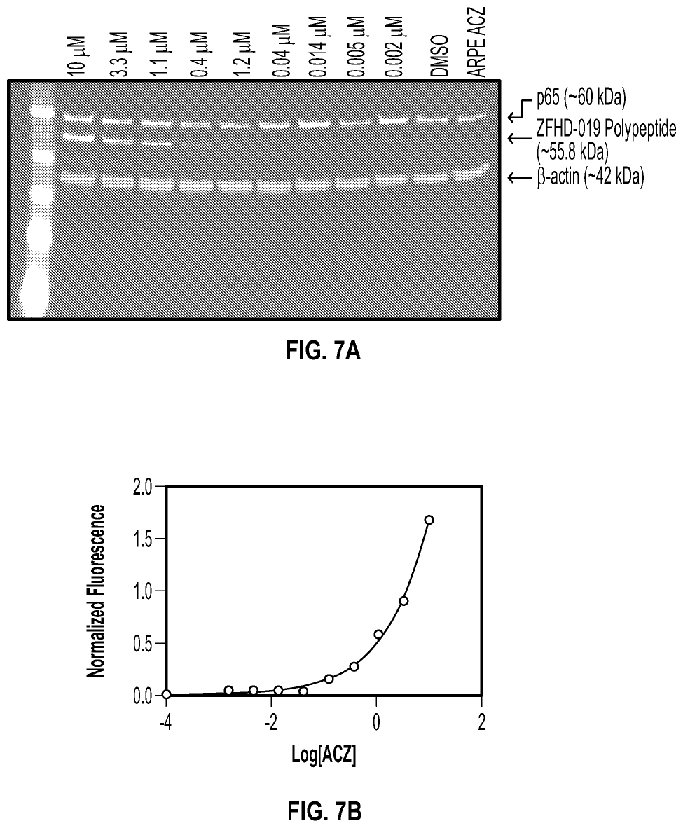

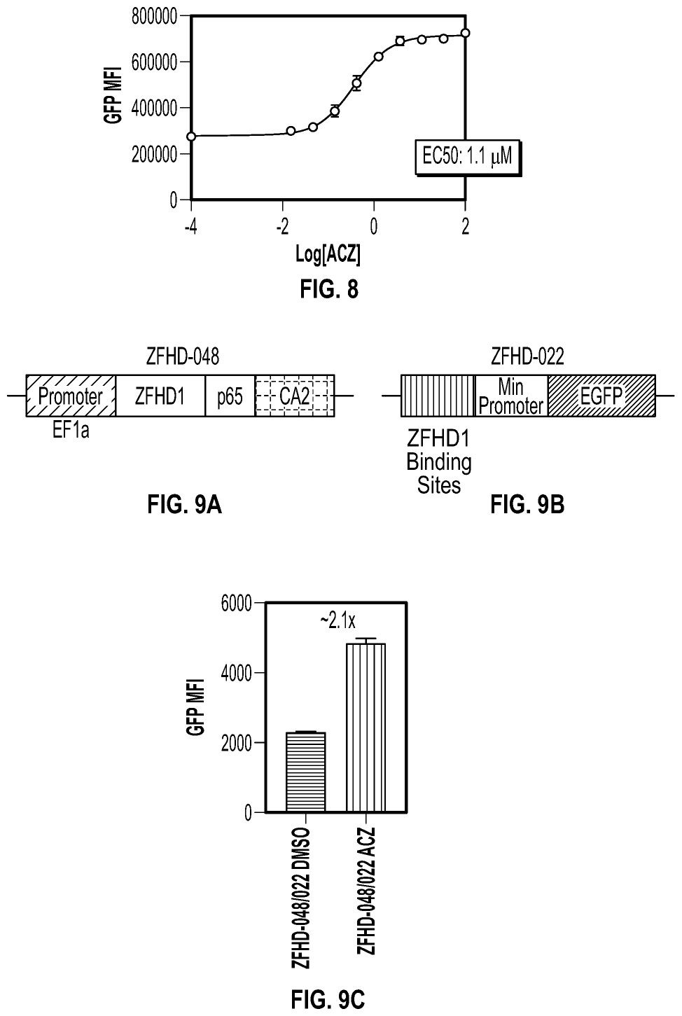

The present example demonstrates ligand-dependent activity of a transcription factor system in ARPE-19 cells. In the present example, the transcription factor system is encoded by nucleic acid constructs that comprise: (1) a first nucleic acid construct that encodes a transcription factor DNA binding domain, a transcription factor activation domain, and a drug responsive domain (DRD) and (2) a second nucleic acid construct that encodes a payload, wherein expression of the payload is driven by an inducible promoter that comprises binding sites for the transcription factor DNA binding domain (FIG. 3B and FIG. 6A). The transcription factor DNA binding domain and the transcription factor activation domain of the present example are expressed as a transcription factor fusion protein, wherein the fusion protein is linked to a DRD that is derived from a CA2 parent protein. As shown here, levels of the transcription factor fusion protein can be regulated with a CA2 ligand. Additionally, the regulated transcription factor fusion protein can induce expression of the payload in a ligand-dependent manner.

Cell lines: The present example analyzed untransduced (parental) ARPE-19 cells and ARPE-19 cells transduced with lentivirus made from the following constructs: (1) ZFHD-007, (2) ZFHD-019, and (3) both lentivirus made from construct ZFHD-007 and lentivirus made from construct ZFHD-019. Transduced cell lines with stably integrated constructs were prepared and selected according to methods described in Example 2. Cells were cultured in DMEM-F12 media supplemented with 10% FBS.

Ligand treatment: Each cell line was plated at a density of 15,000 cells/well in 6-8 wells of a 24-well plate. Cells were allowed to adhere overnight. The following day, media in half the wells of each cell type was replaced with 1 mL of media with 10 μM Acetazolamide (ACZ) and media in the remaining wells was replaced with 1 mL of media with 0.1% DMSO. After 48 hours incubation, cells were removed from the plate with 0.25% trypsin and collected. For immunoblot assay, collected cells were pelleted, media was removed, and cell pellets were stored at −20° C. until procedure for western blot, as described above in Example 4. For flow cytometry, samples were washed once with cell staining buffer (Biolegend, San Diego, Calif.), resuspended in cell staining buffer with 1% QSol™ buffer (Intellicyt Corporation, Albuquerque, N. Mex.), and analyzed by flow cytometry.

Results: Immunoblot analysis of ARPE-19 cells with stably integrated constructs ZFHD-019 and ZFHD-007 revealed an approximately 13.5-fold increase in levels of the transcription factor and DRD polypeptide encoded by the transcription factor construct ZFHD-019 with ACZ treatment as compared to DMSO treatment (FIG. 6B-FIG. 6C). This data indicates that protein levels of a transcription factor operably linked to a CA2 DRD can be regulated with CA2 ligand in ARPE-19 cells.

Flow cytometry analysis indicated that payload expression, as measured by GFP MFI, increased by approximately 1.9-fold with ACZ treatment compared to DMSO treatment in ARPE-19 cells with stably integrated constructs ZFHD-019 and ZFHD-007 (FIG. 6D). This data indicates that a CA2 DRD-regulated transcription factor can induce expression of a payload in a ligand-dependent manner in ARPE-19 cells.

These results demonstrate that a transcription factor system with a transcription factor construct comprising a CA2 DRD can regulate payload expression in ARPE-19 cells.

### Example 8: Dose-Dependent Activity of a Transcription Factor System Comprising a CA2 DRD-Regulated Transcription Factor in ARPE-19 Cells

The present example demonstrates ligand dose-responsive behavior of a transcription factor system comprising a CA2 DRD-regulated transcription factor and analyzed for protein levels of the transcription factor. The transcription factor system of this example is described above in Example 7. As shown in the present example, transcription factor protein levels can be regulated in a ligand dose-dependent manner.

Cell lines: The present example analyzed untransduced (parental) ARPE-19 cells and ARPE-19 cells transduced with both lentivirus made from construct ZFHD-007 and lentivirus made from construct ZFHD-019. The transduced cell line with stably integrated constructs was prepared and selected according to methods described in Example 2. Cells were cultured in DMEM-F12 medium supplemented with 10% FBS.

Ligand treatment: Cells were plated in 24-well plates at a density of 15,000 cells/well. Cells were allowed to adhere overnight.

To prepare media for the 10-point dose response curve, media with 10 μM Acetazolamide (ACZ) was diluted with 8 serial dilutions of 1:3 in plain media. For the 10^{th} point in the dose response curve, media was prepared with 0.1% DMSO.

Media was removed from the plates and replaced with either DMSO media or media with ACZ. Half of the parental ARPE-19 wells received replacement media with 10 μM ACZ. The other half of the parental ARPE-19 wells received replacement media with 0.1% DMSO. Transduced ARPE-19 cells received replacement media with 0.1% DMSO or each dose of ACZ (2-3 wells for each condition). Treated cells were incubated for 48 hours. Cells were removed from plates with 0.25% trypsin and collected. For immunoblot sample analysis, collected cells were pelleted, media was removed, and cells were stored at −20° C. until procedure for western blot, as described above in Example 4.

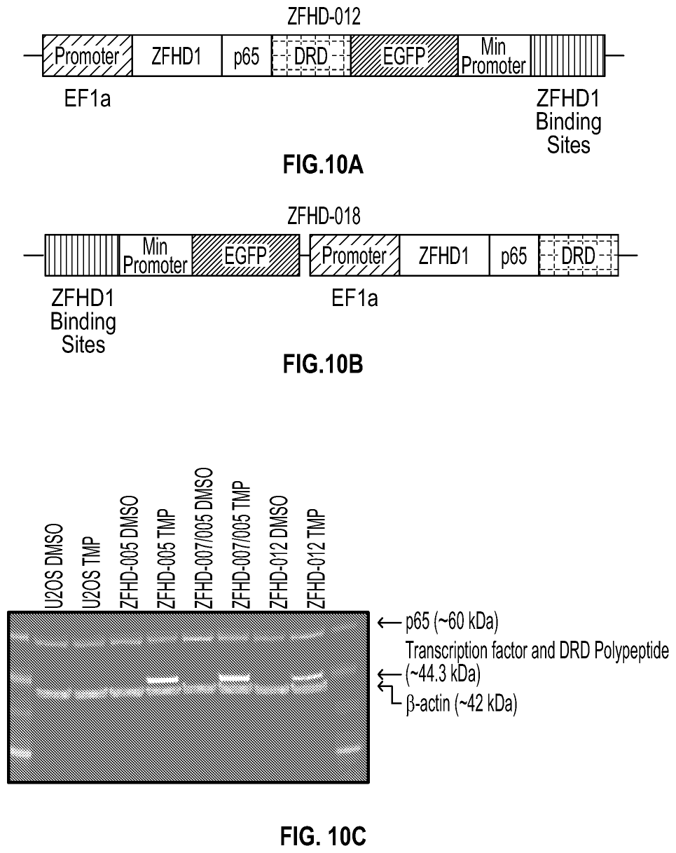

Results: Higher concentrations of ACZ resulted in higher levels of transcription factor fusion protein (FIG. 7A-FIG. 7B). This data indicates that ACZ dose-dependently regulates protein levels of a transcription factor that is operably linked to a CA2 DRD.

### Example 9: Dose-Dependent Activity of a Transcription Factor System Comprising a CA2 DRD-Regulated Transcription Factor in U2OS Cells

The present example demonstrates ligand dose-responsive behavior of a transcription factor system comprising a CA2 DRD-regulated transcription factor and analyzed for payload expression. The transcription factor system of this example is described above in Example 7. As shown in the present example, expression of the payload can be regulated in a ligand dose-dependent manner.

Cell lines: The present example analyzed U2OS cells transduced with both lentivirus made from construct ZFHD-019 and lentivirus made from construct ZFHD-007. The transduced cells with stably integrated constructs were prepared and selected according to methods described in Example 2 with the addition of enough virus to transduce >30% of cells. Cells were cultured in McCoy's 5A medium supplemented with 10% FBS.

Ligand treatment: Cells were plated at a density of 25,000 cells/well in 24-well plates. Plated cells were allowed to adhere overnight.

To prepare media for the 10-point dose response curve, media with 10 μM Acetazolamide (ACZ) was diluted with 8 serial dilutions of 1:3 in plain media. For the 10^{th} point in the dose response curve, media was prepared with 0.1% DMSO.

Media was removed from the plates and replaced with replacement media with 0.1% DMSO or each dose of ACZ (2-3 wells for each condition). Treated cells were incubated for 48 hours. Cells were removed from plates with 0.25% trypsin and collected. Cells were washed one time with PBS, resuspended in PBS and analyzed by flow cytometry.

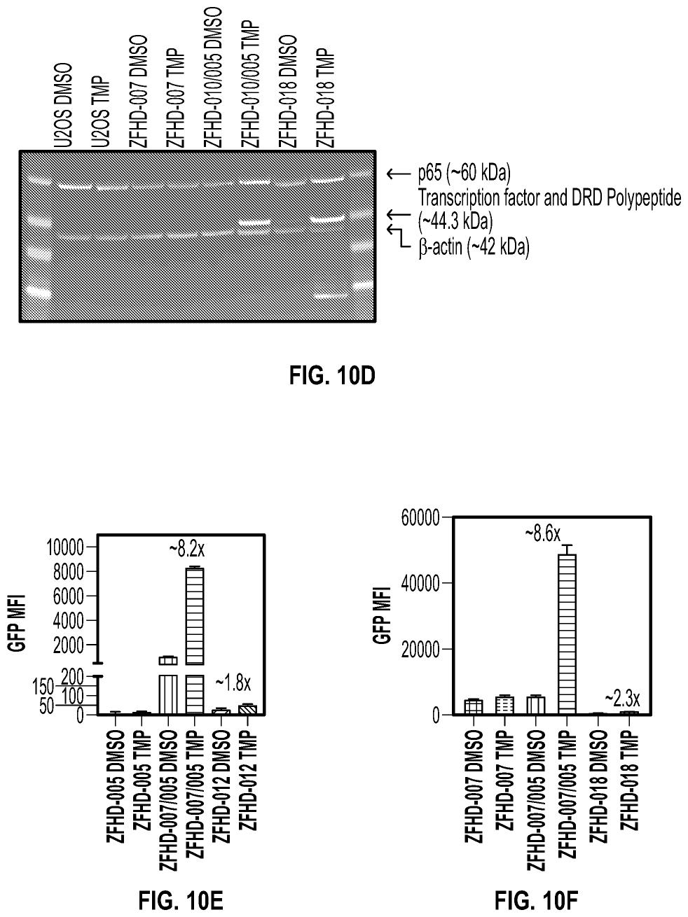

Results: Higher concentrations of ACZ resulted in higher levels of payload as assessed by MFI (FIG. 8). The EC50 of the payload response was 1.1 μM. This data indicates that a CA2 DRD-regulated transcription factor can dose-dependently drive expression of a payload in response to varying doses of ACZ.

### Example 10: Ligand-Dependent Activity of a Transcription Factor System Comprising a CA2 DRD-Regulated Transcription Factor in Jurkat Cells

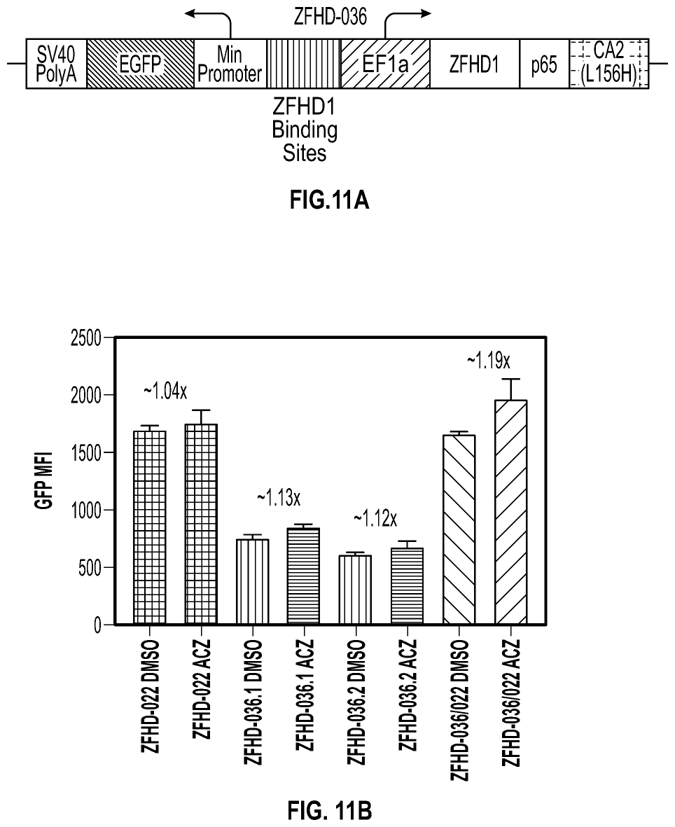

The present example demonstrates ligand-dependent activity of a transcription factor system in Jurkat cells. In the present example, the transcription factor system is encoded by nucleic acid constructs that comprise: (1) a first nucleic acid construct that encodes a transcription factor DNA binding domain, a transcription factor activation domain, and a drug responsive domain (DRD) and (2) a second nucleic acid construct that encodes a payload, wherein expression of the payload is driven by an inducible promoter that comprises binding sites for the transcription factor DNA binding domain (FIG. 9A-FIG. 9B). The transcription factor DNA binding domain and the transcription factor activation domain of the present example are expressed as a transcription factor fusion protein, wherein the fusion protein is operably linked to DRD that is derived from a CA2 parent protein. As shown here, the regulated transcription factor fusion protein can induce expression of the payload in a ligand-dependent manner.

Cell lines: The present example analyzed Jurkat cells transduced with both lentivirus made from construct ZFHD-022 and lentivirus made from construct ZFHD-048. The transduced cells with stably integrated constructs were prepared and selected according to methods described in Example 2. Cells were cultured in RPMI media supplemented with 10% FBS.

Ligand treatment: Cells were plated at a density of 1e6 cells/well in 100 μL in a 96-well u-bottom plate. Media with either 20 μM Acetazolamide (ACZ) or 0.2% DMSO in 100 μL was added. Cells were incubated for 48 hours and collected. Collected cells were pelleted; washed with cell staining buffer (Biolegend, San Diego, Calif.); resuspended and incubated in cell staining buffer with transduction marker antibodies; washed; resuspended in cell staining buffer with 1% QSol™ buffer (Intellicyt Corporation, Albuquerque, N. Mex.); and analyzed by flow cytometry. Antibodies used for staining were 1:1000 APC anti-rat CD90/mouse CD90.1 (Thy-1.1) antibody and 1:100 Brilliant Violet 421 anti-mouse CD90.2 (Thy-1.2) Antibody.

Results: Flow cytometry analysis indicated that payload expression, as measured by GFP median fluorescence intensity (MFI), increased by approximately 2.1-fold in Jurkat cells with stably integrated constructs ZFHD-048 and ZFHD-022, treated with ACZ compared to the same cell line treated with DMSO (FIG. 9C). This data indicates that a CA2 DRD-regulated transcription factor can induce expression of a payload in a ligand-dependent manner in Jurkat cells.

### Example 11: Ligand-Dependent Activity of Single Vector Transcription Factor Systems Comprising ecDHFR DRD-Regulated Transcription Factors

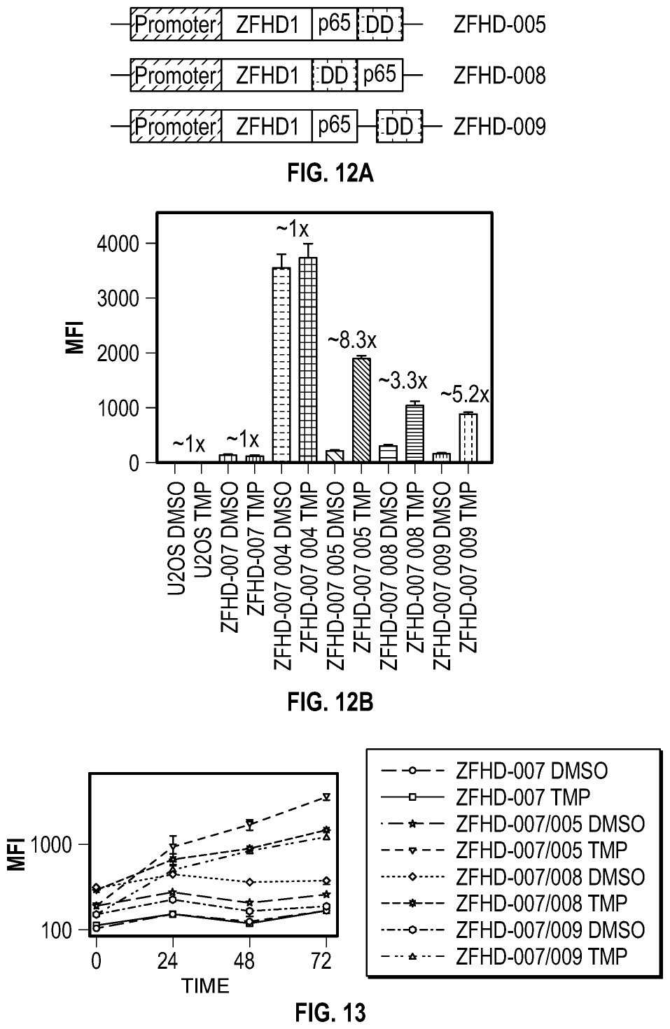

The present example demonstrates ligand responses of transcription factor systems that are encoded by single nucleic acid constructs. Two exemplary single vector transcription factor systems are analyzed in the present example (FIG. 10A-FIG. 10B). Both nucleic acid constructs of the present example each comprise: a nucleic acid sequence that encodes a transcription factor DNA binding domain, a nucleic acid sequence that encodes a transcription factor activation domain, a nucleic acid sequence that encodes a drug responsive domain (DRD), and a nucleic acid sequence that encodes a payload, wherein expression of the payload is driven by an inducible promoter that comprises binding sites for the transcription factor DNA binding domain. The transcription factor DNA binding domain and the transcription factor activation domain of the present example are expressed as a transcription factor fusion protein, wherein the fusion protein is operably linked to a DRD that is derived from an ecDHFR parent protein. As shown here, levels of the transcription factor fusion protein in a cell can be regulated with an ecDHFR ligand. Additionally, the regulated transcription factor fusion protein can induce expression of the payload in a ligand-dependent manner.

Cell lines: The present example analyzed untransduced (parental) U2OS cells and U2OS cells transduced with lentivirus made from the following constructs: (1) ZFHD-005; (2) ZFHD-007; (3) both lentivirus made from construct ZFHD-005 and lentivirus made from construct ZFHD-007; (4) both lentivirus made from construct ZFHD-005 and lentivirus made from construct ZFHD-010; (5) ZFHD-012; and (6) ZFHD-018. Transduced cell lines with stably integrated constructs were prepared and selected according to methods described in Example 2. Cells were cultured in McCoy's 5A medium supplemented with 10% FBS.

Ligand treatment: Each cell line was plated in 6-8 wells in 24-well plates at a density of 25,000 cells/well and allowed to adhere overnight. Media was removed and 1 mL of media with 10 μM Trimethoprim (TMP) was added to half of the wells of each cell line. The remaining wells received 1 mL of media with 0.1% DMSO. Cells were incubated for 48 hours and removed from the plates with 0.25% trypsin for collection. Three wells of each treatment condition was analyzed by flow cytometry. With the remaining samples, collected cells were pelleted, media was removed, and cell pellets were stored at −20° C. for later immunoblot analysis.

Flow cytometry: Collected cells were washed one time with PBS, resuspended in PBS and analyzed by flow cytometry.

Immunoblot assay: procedures for immunoblot assay were followed as described above in Example 4.

Results: Immunoblot analysis revealed that cells transduced with lentivirus corresponding to single nucleic acid constructs (i.e., ZFHD-012 or ZFHD-018, as demonstrated herein) showed higher levels of the transcription factor polypeptide with TMP treatment compared to DMSO treatment (FIG. 10C-10D). This data indicates that protein levels of a transcription factor operably linked to an ecDHFR DRD can be regulated with an ecDHFR DRD ligand in a single vector transcription factor system.

The single vector systems showed increased GFP expression as measured by median fluorescent intensity (MFI) with TMP treatment compared to DMSO treatment (FIG. 10E-10F). However, basal expression of payload, induced expression of payload, and fold-change of MFI were lower with the single vector system compared to the two-vector system. This data indicates that an ecDHFR DRD-regulated transcription factor can induce some expression of a payload in a ligand-dependent manner in a single vector transcription factor system.

This example shows that expression of a transcription factor and a payload from a single vector can exhibit ligand-dependent activity.

### Example 12: Ligand-Dependent Activity of Single Vector Transcription Factor Systems Comprising a CA2 DRD-Regulated Transcription Factor in Jurkat Cells

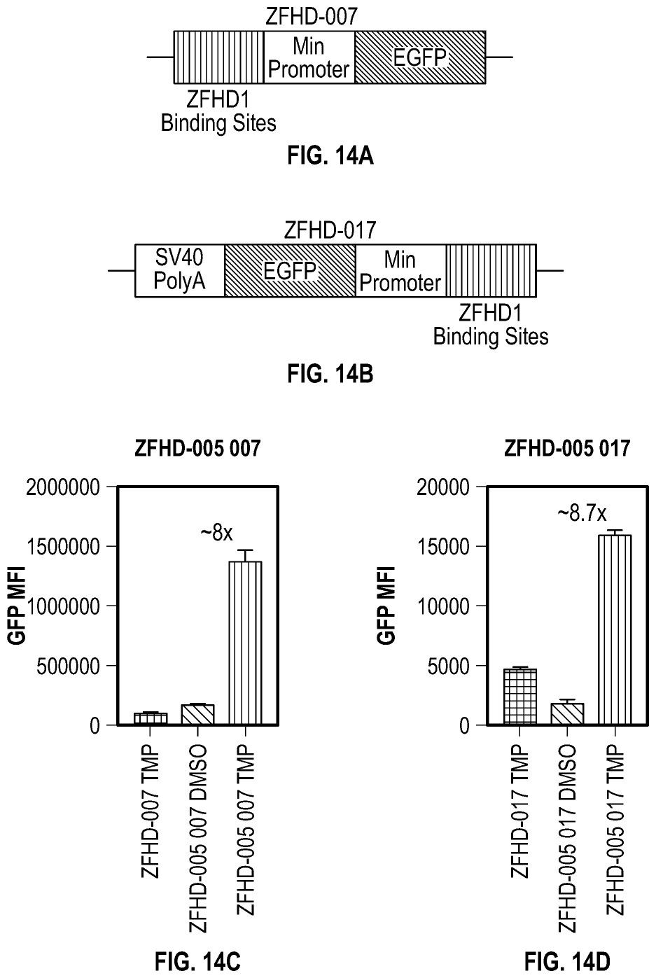

The present example characterizes ligand response of a single vector transcription factor system comprising a CA2-DRD in Jurkat cells. In the present example, the transcription factor system is encoded by a nucleic acid construct that comprises: a nucleic acid sequence that encodes a transcription factor DNA binding domain, a nucleic acid sequence that encodes a transcription factor activation domain, a nucleic acid sequence that encodes a drug responsive domain (DRD), and a nucleic acid sequence that encodes a payload, wherein expression of the payload is driven by an inducible promoter that comprises binding sites for the transcription factor DNA binding domain (FIG. 11A). The transcription factor DNA binding domain and the transcription factor activation domain of the present example are expressed as a transcription factor fusion protein, wherein the fusion protein is operably linked to a DRD that is derived from a CA2 parent protein. The construct also comprises a polyadenylation (Poly-A) signal linked to the payload sequence.

Cell lines: The present example analyzed Jurkat cell lines transduced with lentivirus made from the following constructs: (1) ZFHD-022, (2) Jurkat ZHFD-036, and (3) both lentivirus made from construct ZHFD-036 and lentivirus made from construct ZFHD-022. Transduced cell lines with stably integrated constructs were prepared and selected according to methods described in Example 2. Cells were cultured in RPMI media supplemented with 10% FBS.

Ligand treatment: Each cell line was plated at a density of 1e6 cells/well in 100 μL in a 96-well u-bottom plate. Media with either 20 μM Acetazolamide (ACZ) or 0.2% DMSO in 100 μL was added. Cells were incubated for 48 hours and collected; pelleted; washed with cell staining buffer (Biolegend, San Diego, Calif.); resuspended and incubated in cell staining buffer with transduction marker antibodies; washed; resuspended in cell staining buffer with 1% QSol™ buffer (Intellicyt Corporation, Albuquerque, N. Mex.); and analyzed by flow cytometry. Antibodies used for staining were 1:100 Brilliant Violet 421 anti-mouse CD90.2 (Thy-1.2) Antibody.

Results: Flow cytometry analysis indicated some ligand-dependent activity with the single vector transcription factor system comprising the Poly-A signal in unsorted Jurkat cells (FIG. 111B); however, the observed ligand-dependent activity was lower compared to two vector systems such as shown in Example 10.

### Example 13: Characterization of Transcription Factor Construct Variants

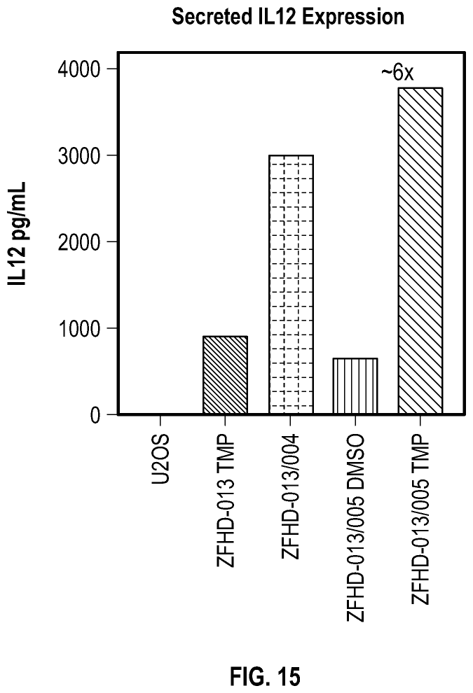

The present example characterizes ligand response of transcription factor systems comprising different variants of transcription factor constructs. In the present example, three transcription factor constructs are studied. All three transcription factor constructs encode a transcription factor DNA binding domain, a transcription factor activation domain, and a drug responsive domain (DRD) derived from an ecDHFR parent protein (FIG. 12A). Construct ZFHD-005, which is also exemplified above, comprises a nucleic acid sequence that encodes a transcription factor DNA binding domain that is 5′ to a nucleic acid sequence that encodes a transcription factor activation domain, which itself is 5′ to a nucleic acid sequence that encodes a DRD. Construct ZFHD-008 comprises a nucleic acid sequence that encodes a transcription factor DNA binding domain that is 5′ to a nucleic acid sequence that encodes a DRD, which itself is 5′ to a nucleic acid sequence that encodes a transcription factor activation domain. Construct ZFHD-009 comprises nucleic acid sequences having the same arrangement as construct ZFHD-005, and further comprises a nucleic acid sequence encoding a linker between the transcription factor activation domain and DRD. The transcription factor constructs in the present example were analyzed as part of transcription factor systems comprising the same payload construct, ZFHD-007. As shown here, all three transcription factor systems comprising an ecDHFR-DRD show induced expression of the payload in a ligand-dependent manner.

Cell lines: The present example analyzed untransduced (parental) U2OS cells and U2OS cells transduced with lentivirus made from the following constructs: (1) ZFHD-007, (2) both lentivirus made from construct ZFHD-004 and lentivirus made from construct ZFHD-007, (3) both lentivirus made from construct ZFHD-005 and lentivirus made from construct ZFHD-007, (4) both lentivirus made from construct ZFHD-008 and lentivirus made from construct ZFHD-007, and (5) both lentivirus made from construct ZFHD-009 and lentivirus made from construct ZFHD-007. Transduced cell lines with stably integrated constructs were prepared and selected according to methods described in Example 2. Cells were cultured in McCoy's 5A medium supplemented with 10% FBS.

Ligand treatment: Each cell line was plated in 6-8 wells in 24-well plates at a density of 25,000 cells/well and allowed to adhere overnight. Media was removed and 1 mL of media with 10 μM Trimethoprim (TMP) was added to half of the wells of each cell line. The remaining wells received 1 mL of media with 0.1% DMSO. Cells were incubated for 48 hours and removed from the plates with 0.25% trypsin for collection. Collected cells were washed one time with PBS, resuspended in PBS and analyzed by flow cytometry.

Results: Each of the three transcription factor systems comprising a DRD in the present example achieved ligand-dependent induction of payload expression as measured by MFI (FIG. 12B). Rank order analysis of the observed regulation across the different cell lines tested was not done; however, such an analysis could be performed after accounting for differing copy numbers of constructs.

This example demonstrates that transcription factor construct variants (e.g., including a variant that comprises a nucleic acid sequence encoding a linker positioned between a nucleic acid sequence encoding a transcription factor activation domain and a nucleic acid sequence encoding a DRD as well as a variant comprising a nucleic acid sequence encoding a DRD positioned between a nucleic acid sequence encoding a transcription factor DNA binding domain and a nucleic acid sequence encoding a transcription factor activation domain) can induce expression of a payload in a transcription factor system in a ligand-dependent manner.

### Example 14: Time Course of Ligand-Dependent Activity of Transcription Factor Systems with Variant ecDHFR-Regulated Transcription Factor Constructs

The present example shows ligand response time courses of transcription factor systems comprising different variants of transcription factor constructs. In the present example, transcription factor constructs ZFHD-005, ZFHD-008, and ZFHD-009, described above in Example 13, were studied. The transcription factor constructs in the present example were combined with the same payload construct, ZFHD-007, and introduced into U2OS cells as separate transcription factor systems. As shown here, all three transcription factor systems comprising an ecDHFR-DRD showed induced expression of the payload in a ligand-dependent manner and also showed an increase in payload expression over time.

Cell lines: The present example analyzed the same cell lines as described above in Example 13. Cells were cultured in McCoy's 5A medium supplemented with 10% FBS.

Ligand treatment: Each cell line was plated in 12-well plates at a density of 80,000 cells/well and allowed to adhere overnight. Media was removed and replacement media either with 10 μM Trimethoprim (TMP) or 0.1% DMSO was added. Cells were incubated for 24 hour, 48 hour, and 72 hour timepoints. At each timepoint, cells were removed from the plates with 0.25% trypsin for collection. A 0 hour timepoint was also collected before addition of TMP or DMSO. Collected cells were washed one time with PBS, resuspended in PBS and analyzed by flow cytometry.

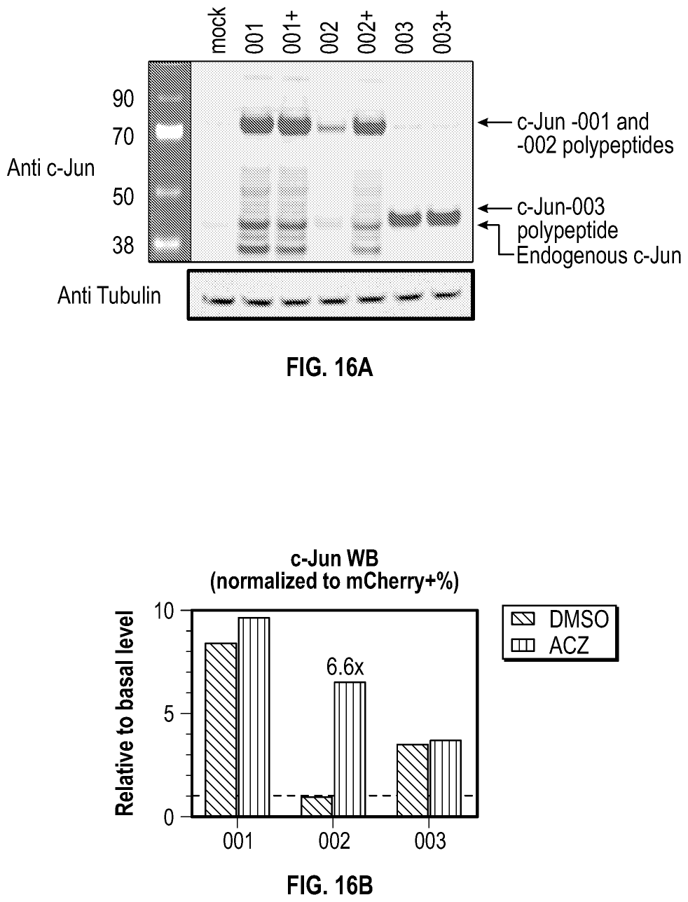

Results: Each of the three transcription factor systems comprising a DRD in the present example showed ligand-dependent induction of payload expression at the indicated timepoints (FIG. 13). Fold change in MFI for TMP versus DMSO treated conditions is shown in Table 7.

TABLE 7
Time course of transcription factor systems
with transcription factor variants

| Sample ID | MFI fold change |
|---|---|
| U2OS | 0 |
| 007/004 | 0.894396 |
| 0 h 007 | 1.067418 |
| 0 h 007/005 | 0.987225 |
| 0 h 007/008 | 0.932676 |
| 0 h 07/009 | 1 |
| 24 h 007 | 0.984313 |
| 24 h 007/005 | 3.113796 |
| 24 h 007/008 | 1.450984 |
| 24 h 07/009 | 2.135991 |
| 48 h 007 | 0.959293 |
| 48 h 007/005 | 7.113078 |
| 48 h 007/008 | 2.315987 |
| 48 h 07/009 | 4.548889 |
| 72 h 007 | 1 |
| 72 h 007/005 | 11.4449 |
| 72 h 007/008 | 3.568371 |
| 72 h 07/009 | 5.712837 |

As shown in Table 7 and in FIG. 13, the fold change in MFI increased for each transcription factor system over the 72-hour timecourse. This data shows that the tested transcription factor construct variants continue to induce expression of a payload in a transcription factor system after addition of ligand.

### Example 15: Characterization of Payload Construct Orientation Variants

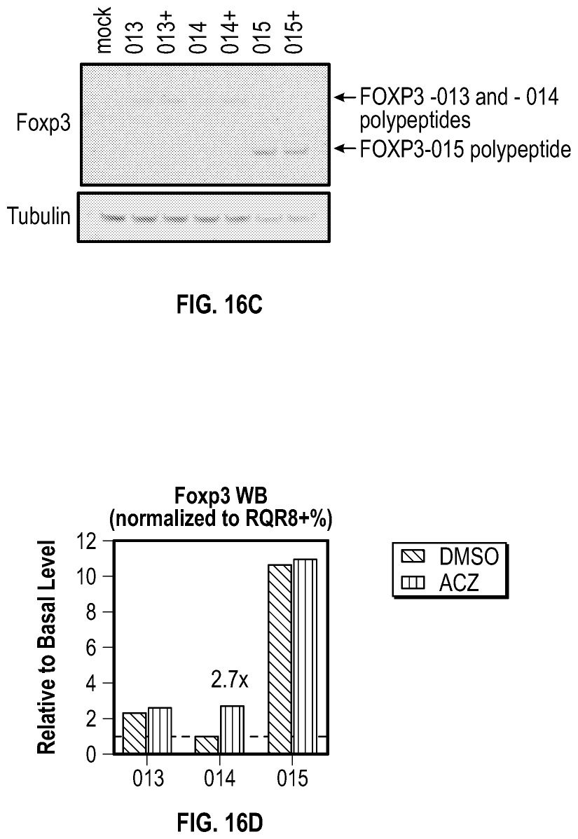

The present example characterizes ligand response of transcription factor systems comprising different variants of payload constructs. In the present example, two payload constructs are studied (FIG. 14A-FIG. 1444B). Payload construct ZFHD-007 comprises a nucleic acid sequence encoding a payload domain that is 3′ to a promoter and transcription factor binding sites. Payload construct ZFHD-017 comprises a nucleic acid sequence encoding a payload domain that is 3′ to a PolyA signal and 5′ to a promoter and transcription factor binding sites. The two payload constructs in the present example were analyzed as part of transcription factor systems comprising the same transcription factor construct, ZFHD-005.

Cell lines: The present example analyzed untransduced (parental) U2OS cells and U2OS cells transduced with lentivirus made from the following constructs: (1) ZFHD-005, (2) ZFHD-007, (3) both lentivirus made from construct ZFHD-005 and lentivirus made from construct ZFHD-007, (4) ZFHD-017, and (5) both lentivirus made from construct ZFHD-005 and lentivirus made from construct ZFHD-017. Transduced cell lines with stably integrated constructs were prepared and selected according to methods described in Example 2. Cells were cultured in McCoy's 5A medium supplemented with 10% FBS.

Ligand treatment: Each cell line was plated in 6-8 wells in 24-well plates at a density of 25,000 cells/well and allowed to adhere overnight. Media was removed and 1 mL of media with 10 μM Trimethoprim (TMP) was added to half of the wells of each cell line. The remaining wells received 1 mL of media with 0.1% DMSO. Cells were incubated for 48 hours and removed from the plates with 0.25% trypsin for collection. Collected cells were washed one time with PBS, resuspended in PBS and analyzed by flow cytometry.

Results: Both transcription factor systems of the present example achieved ligand-dependent induction of payload expression (FIG. 14C-FIG. 14D). Expression of the payload from construct ZFHD-017 was reduced by approximately 100-fold compared to expression from construct ZFHD-007. The difference in payload expression from these two exemplified transcription factor systems is illustrated by the 100-fold scale difference in FIG. 14C compared to FIG. 14D.

The present example demonstrates that positioning a nucleic acid sequence encoding a payload domain 5′ to a promoter and transcription factor binding sites (e.g., as with the payload construct ZFHD-017) maintains induction of payload expression in response to ligand in a transcription factor system; however, expression of payload is reduced compared to a payload construct comprising a nucleic acid sequence encoding a payload domain 3′ to a promoter and transcription factor binding sites (e.g., as with payload construct ZFHD-007).

### Example 16: Ligand-Dependent Activity of a Transcription Factor System Comprising a Secreted IL12 Payload

The present example demonstrates ligand-dependent response by a transcription factor system comprising a secreted IL12 payload. In the present example, the transcription factor system is encoded by transcription factor construct ZFHD-005, which is described above in Example 4, and payload construct ZFHD-013, which comprises a nucleic acid sequence encoding secreted IL12.

Cell lines: The present example analyzed untransduced (parental) U2OS cells and U2OS cells transduced with lentivirus made from the following constructs: (1) ZFHD-013, (2) ZFHD-005, (3) both lentivirus made from construct ZFHD-013 and lentivirus made from construct ZFHD-004, and (4) both lentivirus made from construct ZFHD-013 and lentivirus made from construct ZFHD-005.

Ligand treatment: Each cell line was plated in 6-8 wells in 24-well plates at a density of 25,000 cells/well and allowed to adhere overnight. Media was removed and 1 mL of media with 10 μM Trimethoprim (TMP) was added to half of the wells of each cell line. The remaining wells received 1 mL of media with 0.1% DMSO. Cells were incubated for 72 hours. After incubation, 200 μL of conditioned media was collected from each well and stored at −20° C. for later Meso Scale Discovery (MSD) analysis. Collected supernatants were analyzed by IL12 MSD biomarker assay following the manufacturer's protocol (V-PLEX Plus Human IL-12p70 Kit, Meso Scale Diagnostics, Rockville, Md.).

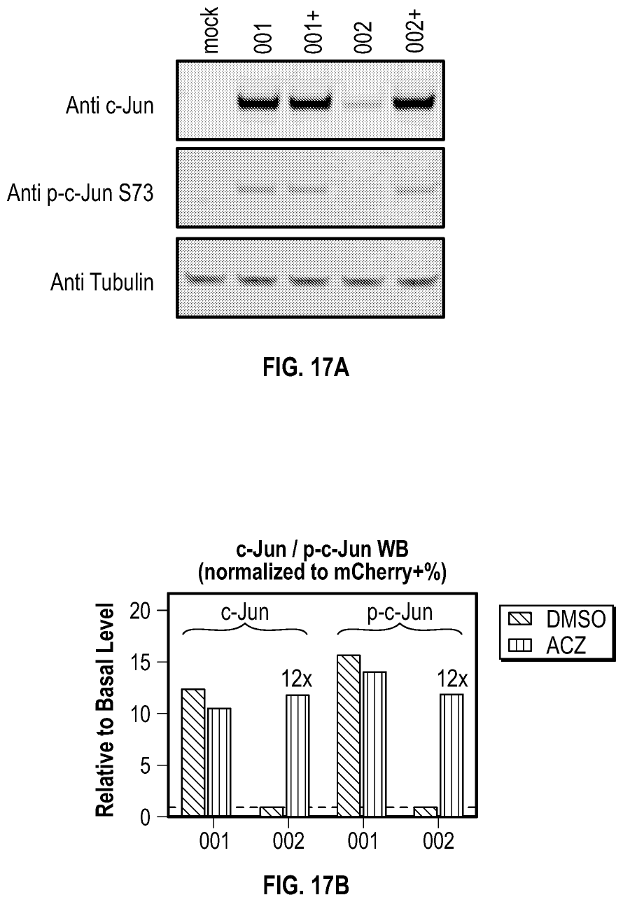

Results: Cells with stably integrated constructs ZFHD-005 and ZFHD-013 showed increased levels of secreted IL12 with TMP treatment compared to cells treated with DMSO (FIG. 15). This data demonstrates that a DRD-regulated transcription factor can induce expression of a secreted IL12 payload in a ligand-dependent manner.

### Example 17: Ligand-Dependent Regulation of c-Jun and FOXP3 Transcription Factors

The present example provides results showing regulation of two transcription factors that are structurally different compared to the engineered transcription factors of the above examples. This example demonstrates that the design and preparation of DRD-TF constructs according to the instant disclosure enables regulation of structurally diverse transcription factors with DRD technology.

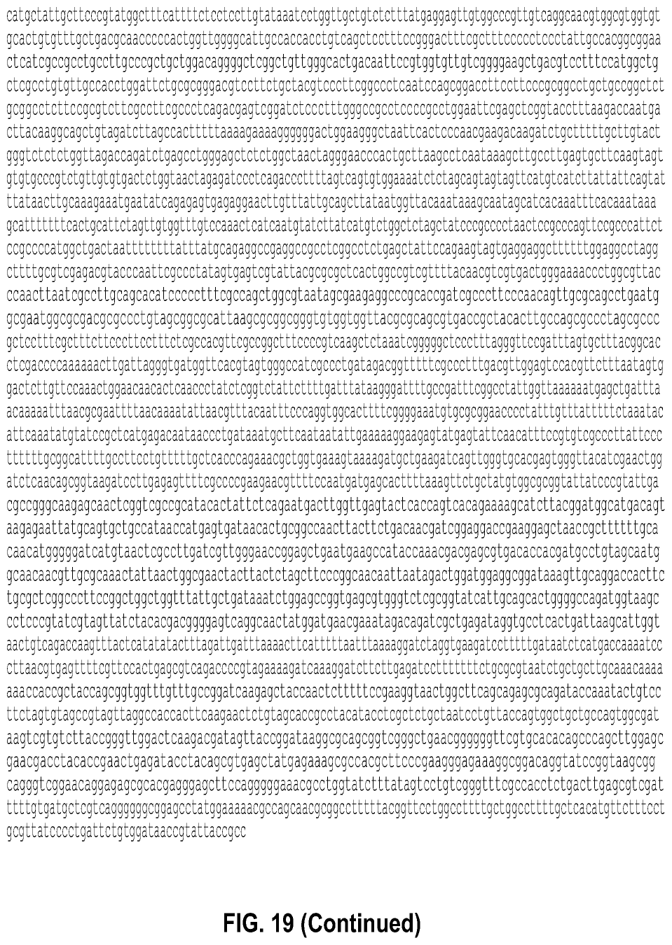

The transcription factors tested in the present example were c-Jun and FOXP3, each of which were operably linked to a DRD derived from a CA2 parent protein. A description of the DRD-TF constructs, corresponding control constructs, and construct components tested in the present example are provided in Table 2. The promoter for each of the constructs in Table 2 was the EF1a promoter. Constructs listed in Table 2 were prepared according to the cloning methods described in Example 2, except that the transfer vector used for the constructs in Table 2 was pELNS (FIG. 19 and SEQ ID NO: 69).

Cell lines: The present example analyzed parental HEK293T cells and HEK293T cells transfected with plasmid DNA of the following constructs: (1) cjun-001, (2) cjun-002, (3) cjun-003, (4) FOXP3-013, (5) FOXP3-014, or (6) FOXP3-015. Cells were cultured in DMEM medium supplemented with 10% FBS and transfected as follows: HEK293T cells were seeded on tissue culture plates and maintained in growth media (DMEM supplemented with 10% FBS and 1% penicillin-streptomycin) until 70-80% confluent. Cells were transfected with transfer vectors prepared as described above using Lipofectamine 3000 transfection reagent (Thermo Fisher Scientific, Waltham, Mass.) in Opti-MEM medium (Thermo Fisher Scientific, Waltham, Mass.). Media was replaced 24 hours post-transfection with growth media with either 100 μM acetazolamide or 0.1% DMSO. After 24 hours, cells were collected for immunoblot assay.

Ligand treatment: Each cell line was plated in 12 or 24-well plates and allowed to adhere overnight. Media was removed and 1 mL of media with ligand or 0.1% DMSO was added. Cells were incubated for 24 hours and removed from the plates with 0.25% trypsin for collection. Collected cells were pelleted, media was removed, and cell pellets were stored at −20° C. for later immunoblot assay.

Immunoblot assay: Cell pellets were resuspended in lysis buffer (T-PER™ Tissue Protein Extraction Reagent, Thermo Fisher Scientific, Waltham, Mass.) containing protease inhibitors. Cell lysates were added to NuPAGE LDS Sample buffer (Thermo Fisher Scientific, Waltham, Mass.) containing beta-mercaptoethanol or Reducing reagent (Thermo Fisher Scientific, Waltham, Mass.) and incubated at 96° C. for 6 minutes with 300 rpm agitation. Samples were run on a NuPAGE 4-12% Bis-Tris gel in BOLT MES SDS running buffer with BOLT antioxidant (Thermo Fisher Scientific, Waltham, Mass.). Proteins were transferred to nitrocellulose membranes and probed with the following antibodies: Rabbit anti-c-Jun antibody (1:1000; Cell Signaling Technology, Danvers, Mass.), rabbit anti-FoxP3 antibody (1:1000; Cell Signaling Technology, Danvers, Mass.), and mouse anti-tubulin antibody (1:4000; Sigma Aldrich). Secondary antibodies were IRDye® 680RD donkey anti-mouse (1:4000; LI-COR, Lincoln, Nebr.) or IRDye® 800CW donkey anti-rabbit (1:3000; LI-COR).

Analysis: Constructs were designed to co-express a marker (mCherry for cJun constructs, RQR8 for FoxP3 constructs) that could be quantified by flow cytometry to allow normalization of difference in transfection or transduction efficiency between samples. In parallel to the Western analysis, cells were analyzed by flow cytometry to determine % mCherry or % RQR8 positive cells. Western blot was quantified and signal was normalized to tubulin signal and % mCherry positive (c-Jun constructs) or % RQR8 positive (FoxP3 constructs) as measured by flow cytometry. The normalized signal for cJun-002 with DMSO or FOXP3-014 with DMSO was set to 1.

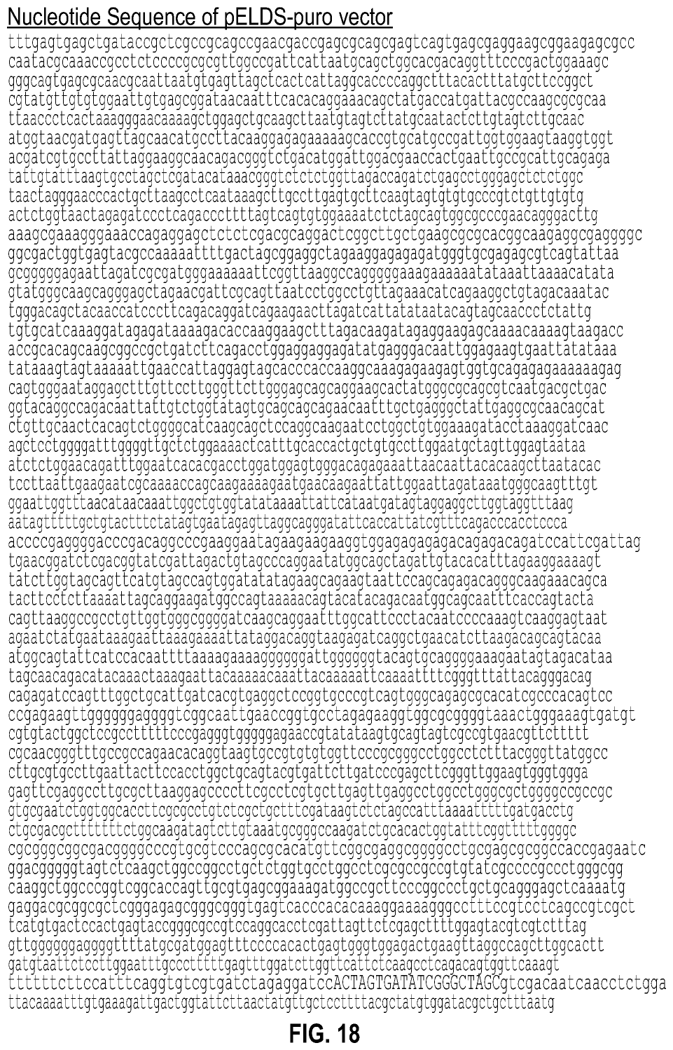

Results: HEK293T cells transfected with plasmid comprising the cjun-002 construct showed that the encoded transcription factor and DRD polypeptide was present at higher levels in ligand-treated cells compared to DMSO-treated cells (FIG. 16A-FIG. 16B). By comparison, HEK293T cells transfected with plasmid comprising the construct cjun-001 (which comprises a wild-type CA2 component) or cjun-003 (which does not comprise any CA2 component), showed that c-Jun protein was detected in both the DMSO and ligand-treated conditions, with minimal to no change between the two conditions.

HEK293T cells transfected with plasmid comprising the FOXP3-014 construct showed that the encoded transcription factor and DRD polypeptide was present at higher levels in ligand-treated cells compared to DMSO-treated cells (FIG. 16C-FIG. 16D). By comparison, HEK293T cells transfected with plasmid comprising the construct FOXP3-013 (which comprises a wild-type CA2 component) or FOXP3-015 (which does not comprise any CA2 component), showed that FOXP3 protein was detected in both the DMSO and ligand-treated conditions, with minimal to no change between the two conditions.

The present example demonstrated ligand-dependent expression of transcription factor constructs comprising structurally diverse transcription factors operably linked to a DRD. These data provide additional support for the modularity of the transcription factor systems of the instant disclosure and confirm that protein levels of different transcription factors operably linked to a DRD can be regulated with a ligand for the DRD. Accordingly, transcription factor systems of the present disclosure may be designed to comprise DRD-TF constructs with other structurally diverse transcription factors, including engineered transcription factors such as ones discussed in Example 1 and elsewhere in the present disclosure.

### Example 18: Ligand-Dependent Regulation of c-Jun Transcription Factor Construct Stably Integrated in Jurkat Cells

The present example demonstrates ligand-dependent regulation of a c-Jun transcription factor construct after stable integration in Jurkat cells. Constructs cjun-001 and cjun-002, prepared in transfer vector as described above in Example 17, were tested and compared.

Cell lines: The present example analyzed untransduced (parental) Jurkat cells and Jurkat cells transduced with lentivirus made from constructs cjun-001 and cjun-002. Cells were cultured in RPMI medium supplemented with 10% FBS. Cells were cultured for 8 days post-transduction, then plated in 6-well plates and treated with either 100 μM acetazolamide or 0.1% DMSO. After 24 hours, cells were collected for immunoblot assay.

Immunoblot assay: Procedures for immunoblot assay were followed as described above in Example 17. The following antibodies were used: Rabbit anti-c-Jun antibody (1:1000; Cell Signaling Technology, Danvers, Mass.), Rabbit anti-Phospho-c-Jun antibody (1:1000; Cell Signaling Technology, Danvers, Mass.), and mouse anti-tubulin antibody (1:4000; Sigma Aldrich). Secondary antibodies were IRDye® 680RD donkey anti-mouse (1:4000; LI-COR, Lincoln, Nebr.) or IRDye® 800CW donkey anti-rabbit (1:3000; LI-COR).

Analysis: Quantification of immunoblot signal was normalized to % mCherry positive cells as described in Example 17.

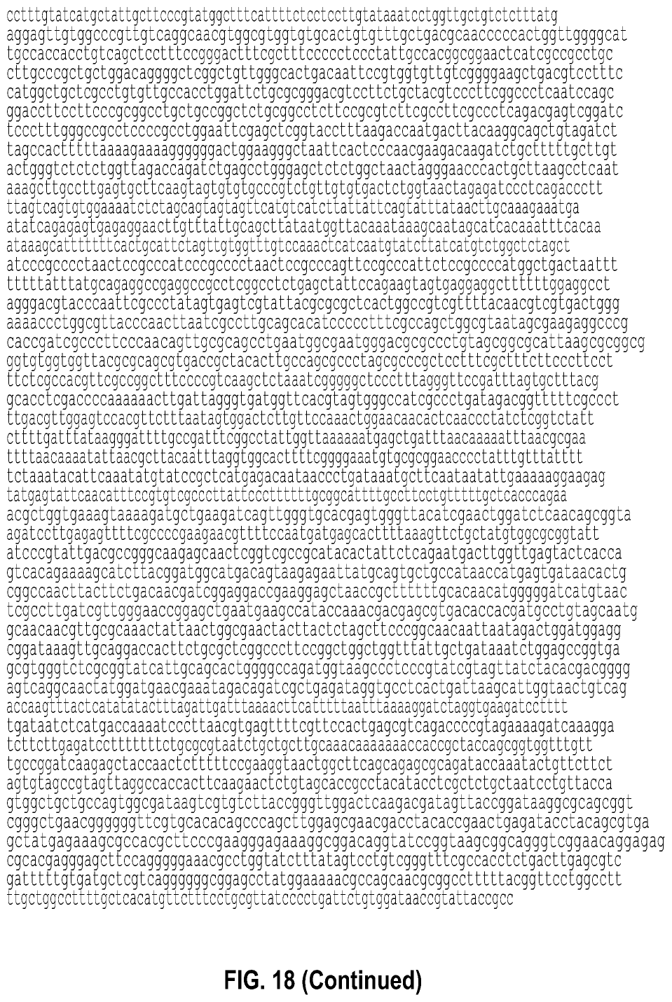

Results: Jurkat cells transduced with lentivirus made from construct cjun-002 showed that the encoded c-Jun and DRD polypeptide was present at higher levels in ligand-treated cells compared to DMSO-treated cells (FIG. 17A-FIG. 17B). A corresponding increase in c-Jun phosphorylation was also observed with ligand treatment. By comparison, parental Jurkat cells and Jurkat cells transduced with lentivirus made from construct cjun-001 showed that c-Jun and phosphorylated c-Jun was detected in both the DMSO and ligand-treated conditions, with minimal to no change between the two conditions. This data confirms that protein levels of a transcription factor operably linked to a DRD can be regulated with a ligand for the DRD.

### Example 19: Treatment of Cancer or Autoimmune Diseases

The present example sets forth an illustrative method that applies the principles and components of transcription factor systems, described in the above detailed description, for treating or preventing a disease in a subject in need thereof. While the applications and uses of transcription factor systems may be applied in a variety of methods described above, such as in the “Applications and uses” section of the present disclosure, the instant example demonstrates a method for the treatment of subjects having cancer or autoimmune diseases.

A variety of methods for treating or preventing cancer or autoimmune diseases that employ transcription factor systems or components thereof are provided above. The illustrative method of the instant example comprises: (a) providing a population of cells; (b) introducing at least one nucleic acid molecule into at least one cell in the population of cells, wherein the resulting cell(s) will comprise a combination of one or more polynucleotides of a transcription factor system; (c) delivering the resulting cell(s) into the subject; and (d) administering a ligand to the subject that stabilizes a DRD encoded by the polynucleotides of the transcription factor system. Below are additional details on each of these (a)-(d) parts of the exemplified method. While the additional details below provide more specificity for each part of the method for the instant example, these particular details are not to be construed as limiting upon other variations of each part (a)-(d) that may be employed in a method for treating or preventing a disease according to the present disclosure, which variations are further elaborated in the above detailed description.

A. Providing a Population of Cells:

A population of autologous or allogeneic immune cells is provided. The cells may be selected from a T cell, a natural killer cell (NK cell), or a tumor infiltrating lymphocyte (TIL). If the population is of autologous cells, the cells are derived from the subject to be treated and, following isolation and processing, are administered to the same subject. If the population is of allogeneic cells, the cells are isolated and/or prepared from a donor subject other than the subject to be treated. Methods of deriving cells from a subject are known in the art, and may include isolation of peripheral blood T cells via leukapheresis or isolation of TILs from a resected tumor.

B. Introducing at Least One Nucleic Acid Molecule into at Least One Cell in the Population of Cells:

A variety of methods for introducing nucleic acid molecules into cells are available, including viral and non-viral methods of delivery and other methods described above, such as in the “Dosing, delivery and administration” section of the detailed description above. For the instant example, the delivery method may be a delivery by one or more viral vectors selected from a lentivirus vector, a gamma retrovirus vector, adeno-associated virus (AAV) vector, adenovirus vector, and a herpes virus vector.

Upon successful delivery, the resulting cell(s) will comprise a combination of one or more polynucleotides of a transcription factor system. For the instant example, the combination of one or more polynucleotides comprises: a first nucleic acid sequence that encodes a transcription factor activation domain; a second nucleic acid sequence that encodes a transcription factor DNA binding domain that binds to a specific polynucleotide binding site; a third nucleic acid sequence that encodes a drug responsive domain (DRD), wherein the transcription factor activation domain and/or the transcription factor DNA binding domain is operably linked to the DRD; and a fourth nucleic acid sequence that encodes a protein of interest (also referred to as “payload”) that treats the cancer or autoimmune disease, the fourth nucleic acid sequence being operably linked to an exogenous inducible promoter comprising the specific polynucleotide binding site. Polynucleotides comprising the first, second, third, and fourth nucleic acid sequences may be delivered to cells on a single vector or on multiple vectors, such as a first vector comprising the first, second, and third nucleic acid sequences and a second vector comprising the fourth nucleic acid sequence.

The encoded transcription factor activation domain; transcription factor DNA binding domain; drug responsive domain (DRD), and protein of interest may be selected separately. Additional details on the modularity of selecting these components of a transcription factor system are provided elsewhere throughout the present disclosure. For the instant example, the payload is a therapeutic protein that may be selected from a cytokine, an antibody, a T cell receptor (TCR) or a chimeric antigen receptor (CAR). Cytokine payloads include IL2, IL12, IL5, or variants thereof, including membrane-bound forms of these cytokines.

C. Delivering the Resulting Cell(s) into the Subject:

Methods of delivering cells to a subject are known in the art and include methods provided above in the detailed description, such as in the “Administration” section. For the instant example, the cells may be delivered into the subject by infusion.

D. Administering a Ligand to the Subject

To achieve a therapeutic effect for the subject, a ligand that stabilizes the DRD is administered to the subject. As described above in part (B) of the present illustrative method, the transcription factor activation domain and/or the transcription factor DNA binding domain is operably linked to the DRD. The ligand is administered to the subject such that it stabilizes the DRD sufficiently to enable expression of the transcription factor activation domain and the transcription factor DNA binding domain in an amount sufficient to form a transcription factor that binds to the specific polynucleotide binding site and enables expression of the protein of interest. Thus, expression of the protein of interest is regulated by the presence of ligand in the subject. The ligand should be administered to the subject in an amount and/or duration is sufficient to produce a therapeutically effective amount of the protein of interest. ACZ may be used as a ligand with a hCA2 DRD, methotrexate may be used with an hDHFR DRD, and trimethoprim may be used with an ecDHFR DRD. The ligand may be administered to a subject or to cells, using any amount and any route of administration effective for producing a therapeutically effective amount of the protein of interest. The total daily dosage of ligand may be decided by the attending physician within the scope of sound medical judgment.

As illustrated by the instant example, transcription factor systems described herein can be used to deliver and regulate a therapeutic payload for treatment of subjects having cancer or autoimmune diseases. As already stated above, variations on the provided method and other methods are also contemplated for application of transcription factor systems of the present disclosure.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present disclosure.

Section headings, the materials, methods, and examples are illustrative only and not intended to be limiting.

What is claimed is:

1. (canceled)

2. A modified cell comprising a first polynucleotide, said first polynucleotide comprising a first nucleic acid sequence that encodes a transcription factor activation domain; a second nucleic acid sequence that encodes a transcription factor DNA binding domain that binds to a specific polynucleotide binding site; and a third nucleic acid sequence that encodes a drug responsive domain (DRD);
wherein the transcription factor activation domain, the transcription factor DNA binding domain, or the combination of the transcription factor activation domain and the transcription factor DNA binding domain is operably linked to the DRD;
wherein the transcription factor activation domain and the transcription factor DNA binding domain interact to form a transcription factor that is able to activate transcription of a fourth nucleic acid sequence upon binding to the specific polynucleotide binding site; and
wherein the fourth nucleic acid sequence encodes a protein of interest and is operably linked to the specific polynucleotide binding site.

3. The modified cell of claim 2, wherein the fourth nucleic acid sequence is operably linked to an exogenous inducible promoter comprising the specific polynucleotide binding site.

4-6. (canceled)

7. The modified cell of claim 2, wherein the transcription factor DNA binding domain is selected from the group consisting of c-Jun, FOXP3, ZFHD1, Cas9, Cas12, and TAL.

8. The modified cell of claim 2, wherein the transcription factor activation domain is p65.

9. (canceled)

10. A modified cell comprising a polynucleotide comprising a first nucleic acid sequence encoding a drug responsive domain (DRD) and a second nucleic acid sequence encoding a transcription factor, wherein the transcription factor is operably linked to the DRD; and
wherein the transcription factor is able to bind to a specific polynucleotide binding site and activate transcription of a third nucleic acid sequence encoding a protein of interest, wherein the third nucleic acid sequence is operably linked to the specific polynucleotide binding site.

11. The modified cell of claim 10, wherein the third nucleic acid sequence is operably linked to an exogenous inducible promoter comprising the specific polynucleotide binding site.

12-14. (canceled)

15. The modified cell of claim 10, wherein the DRD is derived from a parent protein selected from the group comprising: human carbonic anhydrase 2 (CA2), human DHFR, E. coli DHFR (ecDHFR), human estrogen receptor (ER), FKBP, human protein FKBP, and human PDE5.

16. The modified cell of claim 10, wherein the DRD is stabilized in the presence of a ligand selected from the group consisting of Acetazolamide (ACZ), Methotrexate (MTX), and Trimethoprim (TMP).

17-21. (canceled)

22. The modified cell of claim 10, wherein the cell is a T cell, a natural killer cell (NK cell), or a tumor infiltrating lymphocyte (TIL).

23-28. (canceled)

29. A nucleic acid molecule comprising:
a. a first nucleic acid sequence encoding a transcription factor able to bind to a specific polynucleotide binding site and activate transcription; and
b. a second nucleic acid sequence encoding a drug responsive domain (DRD); wherein the transcription factor is operably linked to the DRD.

30. The nucleic acid molecule of claim 29, further comprising:
c. a third nucleic acid sequence encoding a protein of interest, said third nucleic acid sequence being operably linked to an inducible promoter comprising the specific polynucleotide binding site.

31. The nucleic acid molecule of claim 29, wherein the DRD is derived from a parent protein selected from the group comprising: human carbonic anhydrase 2 (CA2), human DHFR, ecDHFR, human estrogen receptor (ER), FKBP, human protein FKBP, and human PDE5.

32. The nucleic acid molecule of claim 29, wherein the DRD is stabilized in the presence of a ligand selected from the group comprising: Acetazolamide (ACZ), Methotrexate (MTX), and Trimethoprim (TMP).

33-37. (canceled)

38. A vector comprising the nucleic acid molecule according to claim 29.

39-50. (canceled)

51. A method of producing a modified cell, said method comprising introducing into a cell a nucleic acid molecule comprising:
a. a first nucleic acid sequence that encodes a transcription factor DNA binding domain that binds to a specific polynucleotide binding site; and
b. a second nucleic acid sequence that encodes a drug responsive domain (DRD).

52. The method of claim 51, wherein the nucleic acid molecule further comprises a third nucleic acid sequence that encodes a transcription factor activation domain.

53. The method of claim 52, wherein either (i) the transcription factor DNA binding domain is operably linked to the DRD; (ii) the transcription factor activation domain is operably linked to the DRD; or (iii) the combination of the transcription factor DNA binding domain and the transcription factor activation domain is operably linked to the DRD.

54. The method according to claim 53, further comprising introducing into the cell:
a fourth nucleic acid sequence encoding a protein of interest, said fourth nucleic acid sequence being operably linked to an inducible promoter comprising the specific polynucleotide binding site.

55. (canceled)

56. The method according to claim 54, wherein the fourth nucleic acid sequence is on the same nucleic acid molecule as the first, second and third nucleic acid sequences.

57. The method according to claim 54, wherein the fourth nucleic acid sequence is on a different nucleic acid molecule than the first, second and third nucleic acid sequences.

58-60. (canceled)

61. The method according to claim 51, wherein the nucleic acid molecule is introduced into the cell by a plasmid or a viral vector.

62. (canceled)

63. The method according to claim 61, wherein the viral vector is selected from the group consisting of a lentivirus vector, a gamma retrovirus vector, adeno-associated virus (AAV) vector, adenovirus vector, and a herpes virus vector.

64. The method according to claim 51, wherein the nucleic acid molecule is introduced into the cell by a non-viral delivery method.

65. The method of claim 51, wherein the cell is a T cell, a natural killer cell (NK cell), or a tumor infiltrating lymphocyte (TIL).

66-68. (canceled)

69. A method for introducing a modified cell into a subject in need of disease treatment or prevention, the method comprising:
a. providing a population of cells;
b. introducing the nucleic acid molecule of claim 29 into at least one cell in the population of cells; and
c. delivering the cell into the subject.

70-73. (canceled)

74. The method according to claim 69, wherein the cell is a T cell, a natural killer cell (NK cell), or a tumor infiltrating lymphocyte (TIL).

75-78. (canceled)

79. The modified cell of claim 10, wherein the transcription factor comprises a DNA binding domain and wherein the DNA binding domain is selected from the group consisting of c-Jun, FOXP3, ZFHD1, Cas9, Cas12, and TAL.

80. The modified cell of claim 10, wherein the transcription factor comprises a transcription factor activation domain comprising p65.

81. The nucleic acid molecule of claim 29, wherein the transcription factor comprises a DNA binding domain and wherein the DNA binding domain is selected from the group consisting of c-Jun, FOXP3, ZFHD1, Cas9, Cas12, and TAL.

82. The nucleic acid molecule of claim 29, wherein the transcription factor comprises a transcription factor activation domain comprising p65.
